SEQUENCE LISTING (122 sequences)

SEQ ID NO: 1 - protein, 5 aa, synthetic construct
  source 1..5: note = Variable Heavy Chain CDR1
SYGMH

SEQ ID NO: 2 - protein, 17 aa, synthetic construct
  source 1..17: note = Variable Heavy Chain CDR2
IIWFDGSSTYYADSVRG

SEQ ID NO: 3 - protein, 9 aa, synthetic construct
  source 1..9: note = Variable Heavy Chain CDR3
ELGRRYFDL

SEQ ID NO: 4 - protein, 11 aa, synthetic construct
  source 1..11: note = Variable Light Chain CDR1
RASQSVSSYLA

SEQ ID NO: 5 - protein, 7 aa, synthetic construct
  source 1..7: note = Variable Light Chain CDR2
DASKRAT

SEQ ID NO: 6 - protein, 10 aa, synthetic construct
  source 1..10: note = Variable Light Chain CDR3
QQRSKWPPWT

SEQ ID NO: 7 - DNA, 15 bp, synthetic construct
  source 1..15: mol_type = other DNA; note = teprotumumab (anti-IGF-1R #1) VH CDR 1
tcctacgggatgcat

SEQ ID NO: 8 - DNA, 51 bp, synthetic construct
  source 1..51: mol_type = other DNA; note = teprotumumab (anti-IGF-1R #1) VH CDR 2
atcatttggttcgacggatcttcaacttattacgccgatagtgttcgaggg

SEQ ID NO: 9 - DNA, 27 bp, synthetic construct
  source 1..27: mol_type = other DNA; note = teprotumumab (anti-IGF-1R #1) VH CDR 3
gagctgggccgccggtattttgacctt

SEQ ID NO: 10 - DNA, 15 bp, synthetic construct
  source 1..15: mol_type = other DNA; note = Modified teprotumumab (anti-IGF-1R #2) VH CDR 1
tcctatgggatgcat

SEQ ID NO: 11 - DNA, 51 bp, synthetic construct
  source 1..51: mol_type = other DNA; note = Modified teprotumumab (anti-IGF-1R #2) VH CDR 2
atcatttggtttgatggatcttcaacttattatgctgatagtgttagaggg

SEQ ID NO: 12 - DNA, 27 bp, synthetic construct
  source 1..27: mol_type = other DNA; note = Modified teprotumumab (anti-IGF-1R #2) VH CDR 3
gagctgggccgcagatattttgacctt

SEQ ID NO: 13 - DNA, 15 bp, synthetic construct
  source 1..15: mol_type = other DNA; note = Modified teprotumumab (anti-IGF-1R #3) VH CDR 1
tcctatgggatgcat

SEQ ID NO: 14 - DNA, 51 bp, synthetic construct
  source 1..51: mol_type = other DNA; note = Modified teprotumumab (anti-IGF-1R #3) VH CDR 2
atcatttggtttgatggatcttcaacttattatgctgatagtgttaggggg

SEQ ID NO: 15 - DNA, 27 bp, synthetic construct
  source 1..27: mol_type = other DNA; note = Modified teprotumumab (anti-IGF-1R #3) VH CDR 3
gagctgggcagaaggtattttgacctt

SEQ ID NO: 16 - DNA, 33 bp, synthetic construct
  source 1..33: mol_type = other DNA; note = teprotumumab (anti-IGF-1R #1) VL CDR 1
agggcctcccagtctgtttcaagttatctggct

SEQ ID NO: 17 - DNA, 21 bp, synthetic construct
  source 1..21: mol_type = other DNA; note = teprotumumab (anti-IGF-1R #1) VL CDR 2
gacgcaagcaaacgagccacc

SEQ ID NO: 18 - DNA, 30 bp, synthetic construct
  source 1..30: mol_type = other DNA; note = teprotumumab (anti-IGF-1R #1) VL CDR 3
cagcagcggagtaagtggccaccctggaca

SEQ ID NO: 19 - DNA, 33 bp, synthetic construct; identical to SEQ ID NO: 16
  source 1..33: mol_type = other DNA; note = Modified teprotumumab (anti-IGF-1R #2) VL CDR 1

SEQ ID NO: 20 - DNA, 21 bp, synthetic construct
  source 1..21: mol_type = other DNA; note = Modified teprotumumab (anti-IGF-1R #2) VL CDR 2
gatgcaagcaaaagggccaca

SEQ ID NO: 21 - DNA, 30 bp, synthetic construct; identical to SEQ ID NO: 18
  source 1..30: mol_type = other DNA; note = Modified teprotumumab (anti-IGF-1R #2) VL CDR 3

SEQ ID NO: 22 - DNA, 33 bp, synthetic construct; identical to SEQ ID NO: 16
  source 1..33: mol_type = other DNA; note = Modified teprotumumab (anti-IGF-1R #3) VL CDR 1

SEQ ID NO: 23 - DNA, 21 bp, synthetic construct
  source 1..21: mol_type = other DNA; note = Modified teprotumumab (anti-IGF-1R #3) VL CDR 2
gatgcaagcaaaagagccaca

SEQ ID NO: 24 - DNA, 30 bp, synthetic construct
  source 1..30: mol_type = other DNA; note = Modified teprotumumab (anti-IGF-1R #3) VL CDR 3
cagcagagaagtaagtggccaccctggaca

SEQ ID NO: 25 - DNA, 354 bp, synthetic construct
  source 1..354: mol_type = other DNA; note = teprotumumab (anti-IGF-1R #1) VH
caggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctca

SEQ ID NO: 26 - DNA, 354 bp, synthetic construct
  source 1..354: mol_type = other DNA; note = Modified teprotumumab (anti-IGF-1R #2) VH
caggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctca

SEQ ID NO: 27 - DNA, 354 bp, synthetic construct
  source 1..354: mol_type = other DNA; note = Modified teprotumumab (anti-IGF-1R #3) VH
caggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctca

SEQ ID NO: 28 - protein, 118 aa, synthetic construct
  source 1..118: note = Variable Heavy Chain
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 29 - DNA, 324 bp, synthetic construct
  source 1..324: mol_type = other DNA; note = teprotumumab (anti-IGF-1R #1) VL
gagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaag

SEQ ID NO: 30 - DNA, 324 bp, synthetic construct
  source 1..324: mol_type = other DNA; note = Modified teprotumumab (anti-IGF-1R #2) VL
gagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaag

SEQ ID NO: 31 - DNA, 324 bp, synthetic construct
  source 1..324: mol_type = other DNA; note = Modified teprotumumab (anti-IGF-1R #3) VL
gagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaag

SEQ ID NO: 32 - protein, 108 aa, synthetic construct
  source 1..108: note = Variable Light Chain
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 33 - DNA, 321 bp, synthetic construct
  source 1..321: mol_type = other DNA; note = LC Constant #1
agaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgt

SEQ ID NO: 34 - DNA, 990 bp, synthetic construct
  source 1..990: mol_type = other DNA; note = HC Constant #1
gcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaa

SEQ ID NO: 35 - DNA, 1398 bp, synthetic construct
  source 1..1398: mol_type = other DNA; note = HC #1
atgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaa

SEQ ID NO: 36 - DNA, 1397 bp, synthetic construct
  source 1..1397: mol_type = other DNA; note = HC #2
atgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgagcgctacacaaccattatacccagaagtctctcagtctgtcacctggcaaa

SEQ ID NO: 37 - DNA, 1398 bp, synthetic construct
  source 1..1398: mol_type = other DNA; note = HC #3
atgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaa

SEQ ID NO: 38 - protein, 448 aa, synthetic construct
  source 1..448: note = Heavy Chain Amino Acid Sequence
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 705 bp, synthetic construct
  source 1..705: mol_type = other DNA; note = LC #1
atgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgt

SEQ ID NO: 40 - DNA, 705 bp, synthetic construct
  source 1..705: mol_type = other DNA; note = LC #2
atgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgt

SEQ ID NO: 41 - DNA, 705 bp, synthetic construct
  source 1..705: mol_type = other DNA; note = LC #3
atgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgt

SEQ ID NO: 42 - protein, 215 aa, synthetic construct
  source 1..215: note = Light Chain Amino Acid Sequence
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 585 bp, synthetic construct
  source 1..585: mol_type = other DNA; note = IRES Sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccaca

SEQ ID NO: 44 - DNA, 24 bp, synthetic construct
  source 1..24: mol_type = other DNA; note = Furin Cleavage Site
agaaagagaaggagtggctcagga

SEQ ID NO: 45 - DNA, 72 bp, synthetic construct
  source 1..72: mol_type = other DNA; note = F2A site
gcccctgtgaaacagaccctgaactttgacctcttgaagcttgctggggatgtggagtctaatcctggtcca

SEQ ID NO: 46 - DNA, 97 bp, synthetic construct
  source 1..97: mol_type = other DNA; note = SV40 intron
gtaagtttagtctttttgtcttttatttcaggtcccggatccggtggtggtgcaaatcaaagaactgctcctcagtggatgttgcctttacttctag

SEQ ID NO: 47 - DNA, 230 bp, synthetic construct
  source 1..230: mol_type = other DNA; note = EF-1(alpha)
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 48 - DNA, 380 bp, synthetic construct
  source 1..380: mol_type = other DNA; note = CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 49 - DNA, 218 bp, synthetic construct
  source 1..218: mol_type = other DNA; note = CMV promoter (CMVp)
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 50 - DNA, 598 bp, synthetic construct
  source 1..598: mol_type = other DNA; note = CMV enhancer and promoter (CMVe/p)
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 51 - DNA, 1725 bp, synthetic construct
  source 1..1725: mol_type = other DNA; note = CMV enhancer - CBA promoter - CAG intron
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaa

SEQ ID NO: 52 - DNA, 49 bp, synthetic construct
  source 1..49: mol_type = other DNA; note = Synthetic poly(A)
aataaaagatctttattttcattagatctgtgtgttggttttttgtgtg

SEQ ID NO: 53 - DNA, 224 bp, synthetic construct
  source 1..224: mol_type = other DNA; note = BGH poly(A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 54 - DNA, 92 bp, synthetic construct
  source 1..92: mol_type = other DNA; note = Pause element
aacatacgctctccatcaaaacaaaacgaaacaaaacaaactagcaaaataggctgtccccagtgcaagtgcaggtgccagaacatttctct

SEQ ID NO: 55 - DNA, 269 bp, synthetic construct
  source 1..269: mol_type = other DNA; note = 5' LTR (truncated)
ggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 56 - DNA, 133 bp, synthetic construct
  source 1..133: mol_type = other DNA; note = Intron (chimera of human betaglobin intron and immunoglobulin heavy chain intron)
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 57 - DNA, 2202 bp, synthetic construct
  source 1..2202: mol_type = other DNA; note = ORF #1
atgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaaagaaagagaaggagtggctcaggagcccctgtgaaacagaccctgaactttgacctcttgaagcttgctggggatgtggagtctaatcctggtccaatgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttag

SEQ ID NO: 58 - DNA, 2202 bp, synthetic construct
  source 1..2202: mol_type = other DNA; note = ORF #2
atgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcacctggcaaaagaaagagaaggagtggctcaggagcccctgtgaaacagaccctgaactttgacctcttgaagcttgctggggatgtggagtctaatcctggtccaatgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttag

SEQ ID NO: 59 - DNA, 1401 bp, synthetic construct
  source 1..1401: mol_type = other DNA; note = ORF #3
atgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatag

SEQ ID NO: 60 - DNA, 708 bp, synthetic construct
  source 1..708: mol_type = other DNA; note = ORF #4
atgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttag

SEQ ID NO: 61 - DNA, 1401 bp, synthetic construct
  source 1..1401: mol_type = other DNA; note = ORF #5
atgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaatag

SEQ ID NO: 62 - DNA, 708 bp, synthetic construct
  source 1..708: mol_type = other DNA; note = ORF #6
atgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttag

SEQ ID NO: 63 - DNA, 1401 bp, synthetic construct
  source 1..1401: mol_type = other DNA; note = ORF #7
atgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcacctggcaaatag

SEQ ID NO: 64 - DNA, 1401 bp, synthetic construct; identical to SEQ ID NO: 63
  source 1..1401: mol_type = other DNA; note = ORF #8

SEQ ID NO: 65 - DNA, 1401 bp, synthetic construct; identical to SEQ ID NO: 59
  source 1..1401: mol_type = other DNA; note = ORF #9

SEQ ID NO: 66 - DNA, 708 bp, synthetic construct
  source 1..708: mol_type = other DNA; note = ORF #10
atgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttag

SEQ ID NO: 67 - DNA, 708 bp, synthetic construct; identical to SEQ ID NO: 66
  source 1..708: mol_type = other DNA; note = ORF #11

SEQ ID NO: 68 - DNA, 4479 bp, synthetic construct
  source 1..4479: mol_type = other DNA; note = EC #1
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaaagaaagagaaggagtggctcaggagcccctgtgaaacagaccctgaactttgacctcttgaagcttgctggggatgtggagtctaatcctggtccaatgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 69 - DNA, 4799 bp, synthetic construct
  source 1..4799: mol_type = other DNA; note = EC #2
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaatagtgacaattgaataaaagatctttattttcattagatctgtgtgttggttttttgtgtggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 70 - DNA, 4288 bp, synthetic construct
  source 1..4288: mol_type = other DNA; note = EC #3
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagaaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtagcccggttaattaagccaccatgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttaggcatgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattctcagaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcgaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagtttaaaccaccatgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaatagcctaggaataaaagatctttattttcattagatctgtgtgttggttttttgtgtgctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 71 - DNA, 4479 bp, synthetic construct
  source 1..4479: mol_type = other DNA; note = EC #4
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcacctggcaaaagaaagagaaggagtggctcaggagcccctgtgaaacagaccctgaactttgacctcttgaagcttgctggggatgtggagtctaatcctggtccaatgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 72 - DNA, 4795 bp, synthetic construct
  source 1..4795: mol_type = other DNA; note = EC #5
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcaccggcaaatagcaattgaataaaagatctttattttcattagatctgtgtgttggttttttgtgtggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 73 - DNA, 4288 bp, synthetic construct
  source 1..4288: mol_type = other DNA; note = EC #6
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagaaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtagcccggttaattaagccaccatgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcatgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattctcagaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcgaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagtttaaaccaccatgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcacctggcaaatagcctaggaataaaagatctttattttcattagatctgtgtgttggttttttgtgtgctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 74 - DNA, 4488 bp, synthetic construct
  source 1..4488: mol_type = other DNA; note = EC #7
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatagggcaaaagaaagagaaggagtggctcaggagcccctgtgaaacagaccctgaactttgacctcttgaagcttgctggggatgtggagtctaatcctggtccaatgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttagcaattgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 75 - DNA, 4796 bp, synthetic construct
  source 1..4796: mol_type = other DNA; note = EC #8
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaagaattcaagcttccagctagcgccaccatgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatagcaattgaataaaagatctttattttcattagatctgtgtgttggttttttgtgtggagctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 76 - DNA, 4288 bp, synthetic construct
  source 1..4288: mol_type = other DNA; note = EC #9
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttagtattaaacgcgtgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagaaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtagcccggttaattaagccaccatgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcatgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattctcagaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcgaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagtttaaaccaccatgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatagcctaggaataaaagatctttattttcattagatctgtgtgttggttttttgtgtgctcgagaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcagatctg

SEQ ID NO: 77 - skipped

SEQ ID NO: 78 - DNA, 12 bp, synthetic construct
  source 1..12: mol_type = other DNA; note = Kozak Sequence
gcggccgccatg

SEQ ID NO: 79-81 - skipped (3 entries)

SEQ ID NO: 82 - DNA, 1064 bp, synthetic construct
  source 1..1064: mol_type = other DNA; note = CAG intron
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggtctagactctgctaaccatgttcatgccttcttctctttcctacagctcctgggcaacgtgctggttgttgtgctgtctcatcattttggcaaa

SEQ ID NO: 83 - DNA, 276 bp, synthetic construct
  source 1..276: mol_type = other DNA; note = CBA promoter #1
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 84 - DNA, 1557 bp, synthetic construct
  source 1..1557: mol_type = other DNA; note = ColE1 origin of replication and kanamycin resistance
gccagcaaaaggccaggaaccgtaaaaaggccgcgttgctggcgtttttccataggctccgcccccctgacggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcat

SEQ ID NO: 85 - DNA, 321 bp, synthetic construct
  source 1..321: mol_type = other DNA; note = LC Constant #2
agaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgt

SEQ ID NO: 86 - DNA, 321 bp, synthetic construct
  source 1..321: mol_type = other DNA; note = LC Constant #3
agaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgt

SEQ ID NO: 87 - DNA, 990 bp, synthetic construct
  source 1..990: mol_type = other DNA; note = HC Constant #2
gcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcacctggcaaa

SEQ ID NO: 88 - DNA, 990 bp, synthetic construct
  source 1..990: mol_type = other DNA; note = HC Constant #3
gcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaa

SEQ ID NO: 89 - protein, 17 aa, synthetic construct
  source 1..17: note = Variable Heavy Chain CDR2
IIWFDGSSKYYGDSVKG

SEQ ID NO: 90 - protein, 7 aa, synthetic construct
  source 1..7: note = Variable Light Chain CDR2
DASNRAT

SEQ ID NO: 91 - protein, 118 aa, synthetic construct
  source 1..118: note = Variable Heavy Chain
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 92 - protein, 108 aa, synthetic construct
  source 1..108: note = Variable Light Chain
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 93 - DNA, 278 bp, synthetic construct
  source 1..278: mol_type = other DNA; note = CBA promoter #2
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 94 - DNA, 2694 bp, synthetic construct
  source 1..2694: mol_type = other DNA; note = ORF #12
atgtaccggatgcagctgctcagctgcatcgccttgtcccttgctctggtgaccaactctgagattgtcttaacacaatcacccgcaactctgagtctatcgcctggcgaaagagcgacgctcagctgtagggcctcccagtctgtttcaagttatctggcttggtaccagcaaaagccaggacaggccccgcgcttgctgatatatgacgcaagcaaacgagccaccgggatccccgctcgtttctccggttctggcagcggaacagattttactcttaccatttcctcactcgagcctgaagacttcgcagtgtactattgccagcagcggagtaagtggccaccctggacatttgggcaaggcaccaaagtagagagcaagagaactgtggccgcgccttctgtcttcatctttccaccctccgatgaacagctgaaatcgggtacggcttcagtggtttgtctgctcaataacttctacccgagggaggccaaggtgcagtggaaggtcgacaatgcattgcagagcggaaacagtcaagagtctgtaacagaacaggattccaaagacagcacctattcactttccagtactctgaccttatctaaggccgattacgagaaacacaaggtgtacgcttgcgaagttacacatcaggggctgagctcccctgtgaccaaaagctttaatcgcggcgagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctccgccctgctctgctgtttggtgcttctgaccggcgtccgggctcaggttgagttagtggaatctggagggggtgtagtgcaacccggcagatcacagaggctgagttgcgcagcgtcgggattcacatttagctcctacgggatgcattgggtccgccaggcccctggcaagggtctagagtgggtggctatcatttggttcgacggatcttcaacttattacgccgatagtgttcgagggcgttttacgataagccgggacaactccaaaaataccctctatctgcaaatgaactctttgagagcagaagatacagccgtgtacttctgtgctagggagctgggccgccggtattttgacctttggggcagaggaactctcgtcagcgtatcctcagcaagtaccaaggggccaagcgtgttcccgctggccccctcttccaaatcgacatcaggtggcaccgcggctctgggatgcctcgttaaggattactttcctgaaccagtgactgtcagctggaatagtggggccttgacgtctggcgtgcacacattccccgcagtccttcagtccagcggtctgtattcattatccagtgtggttaccgtgccttctagctccctgggaactcagacctacatctgtaacgtaaatcataaaccaagcaacacaaaggtggacaagaaagtcgagcccaagtcttgcgataaaacccacacttgtccgccttgcccagcccccgagctactcgggggcccttcagtttttctgttcccacccaagcctaaagacacattgatgattagtaggacgcccgaagtgacctgtgtcgtggtggatgtttcccatgaggacccagaagtaaagttcaattggtacgtggatggcgtcgaggtgcacaacgctaagactaaaccgcgcgaagagcagtataatagcacataccgagtcgtgtcggttctgaccgtgcttcaccaagactggctcaacggaaaggagtataaatgcaaggtatctaataaggccctgcccgcacctatcgaaaaaactatttcaaaggctaaagggcagccacgtgagccccaggtctacaccctgcctccatcccgggacgaattaacaaagaaccaggtgagtctcacgtgcctggttaaaggtttttatcccagcgatatcgccgtggagtgggagtctaacggccaaccggaaaataactacaagaccacacctccagtcttggactccgatggaagcttctttctttactcaaagctgactgtggacaaaagtagatggcagcaggggaatgtgttcagctgttccgttatgcatgaggcgctacacaaccattatacccagaagtctctctcgctgtcacccggcaaatag

SEQ ID NO: 95 - DNA, 2700 bp, synthetic construct
  source 1..2700: mol_type = other DNA; note = ORF #13
atgtacagaatgcagctgctcagctgcattgccttgtcccttgctctggtgaccaactctgagattgtcttaacacaatcacccgcaactctgagtctaagtcctggagaaagagcaactctcagctgtagggcctcccagtctgtttcaagttatctggcttggtaccagcaaaagccaggacaggccccgcgcttgctgatatatgatgcaagcaaaagggccacagggatccccgctagattcagcggttctggcagcggaacagattttactcttaccatttcctcacttgagcctgaagactttgcagtgtactattgccagcagcggagtaagtggccaccctggacatttgggcaaggcaccaaagtagagagcaagagaactgtggccgcgccttctgtcttcatctttccaccctctgatgaacagctgaaatcaggtactgcttcagtggtttgtctgctcaataacttctacccaagggaggccaaggtgcagtggaaggttgacaatgcattgcagagcggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctccgccctgctctgctgtttggtgcttctgacaggagtcagggctcaggttgagttagtggaatctggagggggtgtagtgcaacctggcagatcacagaggctgagttgcgcagcaagtggattcacatttagctcctatgggatgcattgggtccgccaggcccctggcaagggtctagagtgggtggctatcatttggtttgatggatcttcaacttattatgctgatagtgttagagggcgctttactataagcagggacaactccaaaaataccctctatctgcaaatgaactctttgagagcagaagatacagctgtgtacttctgtgctagggagctgggccgcagatattttgacctttggggcagaggaactcttgtctctgtatcctcagcaagtaccaaggggccatctgtgttcccgctggccccctcttccaaaagtacatcaggtggcaccgcggctctgggatgcttggttaaggattactttcctgaaccagtgactgtcagctggaatagtggggccttgacttctggggtgcacacattccccgcagtccttcagtccagcggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccgccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccgagggaagagcagtataatagcacatacagagttgtgtcagttctgacagtgcttcaccaagactggctcaatggaaaggagtataaatgcaaggtatctaataaggccctgcccgcacctattgaaaaaactatttcaaaggctaaagggcagccaagggagccccaggtctacaccctgcctccatccagagatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcgctacacaaccattatacccagaagtctctcagtctgtcaccggcaaatagcaattga

SEQ ID NO: 96 - DNA, 2211 bp, synthetic construct
  source 1..2211: mol_type = other DNA; note = ORF #14
atgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatagggcaaaagaaagagaaggagtggctcaggagcccctgtgaaacagaccctgaactttgacctcttgaagcttgctggggatgtggagtctaatcctggtccaatgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttag

SEQ ID NO: 97 - DNA, 2694 bp, synthetic construct
  source 1..2694: mol_type = other DNA; note = ORF #15
atgtacagaatgcagctgctcagctgcattgccttgtcccttgctctggtgaccaactctgagattgtcttaacacaatcacctgcaactctgagtctaagtcctggagaaagagcaactctcagctgtagggcctcccagtctgtttcaagttatctggcttggtaccagcaaaagccaggacaggccccaaggttgctgatatatgatgcaagcaaaagagccacagggatccctgctaggttctctggttctggctctggaacagattttactcttaccatttcctcacttgagcctgaagactttgcagtgtactattgccagcagagaagtaagtggccaccctggacatttgggcaaggcaccaaagtagagagcaagagaactgtggctgcaccttctgtcttcatctttccaccctctgatgaacagctgaaatcaggtactgcttcagtggtttgtctgctcaataacttctacccaagggaggccaaggtgcagtggaaggttgacaatgcattgcagtctggaaacagtcaagagtctgtaacagaacaggattccaaagacagcacctattcactttccagtactctgaccttatctaaggctgattatgagaaacacaaggtgtatgcttgtgaagttacacatcaggggctgagctcccctgtgaccaaaagctttaataggggggagtgttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaatgcacagctctgccctgctctgctgtttggtgcttctgacaggagtcagggctcaggttgagttagtggaatctggagggggtgtagtgcaacctggcagatcacagaggctgagttgtgcagcaagtggattcacatttagctcctatgggatgcattgggtcagacaggcccctggcaagggtctagagtgggtggctatcatttggtttgatggatcttcaacttattatgctgatagtgttagggggagatttactataagcagggacaactccaaaaataccctctatctgcaaatgaactctttgagagcagaagatacagctgtgtacttctgtgctagggagctgggcagaaggtattttgacctttggggcagaggaactcttgtctctgtatcctcagcaagtaccaaggggccatctgtgttcccactggccccctcttccaaaagtacatcaggtggcacagcagctctgggatgcttggttaaggattactttcctgaaccagtgactgtcagctggaatagtggggccttgacttctggggtgcacacattccctgcagtccttcagtcctctggtctgtattcattatccagtgtggttacagtgccttctagctccctgggaactcagacctacatctgtaatgtaaatcataaaccaagcaacacaaaggtggacaagaaagttgagcccaagtcttgtgataaaacccacacttgtccaccttgcccagcccctgagctacttgggggcccttcagtttttctgttcccacccaagcctaaagacacattgatgattagtaggactcctgaagtgacctgtgttgtggtggatgtttcccatgaggacccagaagtaaagttcaattggtatgtggatggagttgaggtgcacaatgctaagactaaaccaagagaagagcagtataatagcacatacagggttgtgtcagttctgacagtgcttcaccaagactggctcaatggaaaggagtataaatgcaaggtatctaataaggccctgcctgcacctattgaaaaaactatttcaaaggctaaagggcagccaagagagccccaggtctacaccctgcctccatccagggatgaattaacaaagaaccaggtgagtctcacttgcctggttaaaggtttttatccctctgatattgctgtggagtgggagtctaatggccaaccagaaaataactacaagaccacacctccagtcttggactctgatggaagcttctttctttactcaaagctgactgtggacaaaagtagatggcagcaggggaatgtgttcagctgttctgttatgcatgaggcactacacaaccattatacccagaagtctctcagtctgtcacctggcaaatag

SEQ ID NO: 98 - DNA, 20 bp, synthetic construct
  source 1..20: mol_type = other DNA; note = Heavy Chain Forward Primer
tctcacgtgcctggttaaag

SEQ ID NO: 99 - DNA, 23 bp, synthetic construct
  source 1..23: mol_type = other DNA; note = Heavy Chain Reverse Primer
ggaggtgtggtcttgtagttatt

SEQ ID NO: 100 - DNA, 24 bp, synthetic construct
  source 1..24: mol_type = other DNA; note = Heavy Chain Probe
tatcgccgtggagtgggagtctaa

SEQ ID NO: 101 - DNA, 22 bp, synthetic construct
  source 1..22: mol_type = other DNA; note = Light Chain Forward Primer
cgccttctgtcttcatctttcc

SEQ ID NO: 102 - DNA, 22 bp, synthetic construct
  source 1..22: mol_type = other DNA; note = Light Chain Reverse Primer
cctccctcgggtagaagttatt

SEQ ID NO: 103 - DNA, 24 bp, synthetic construct
  source 1..24: mol_type = other DNA; note = Light Chain Probe
tacggcttcagtggtttgtctgct

SEQ ID NO: 104 - DNA, 20 bp, synthetic construct
  source 1..20: mol_type = other DNA; note = Forward Primer - F2A link -1
cctgtgaaacagaccctgaa

SEQ ID NO: 105 - DNA, 18 bp, synthetic construct
  source 1..18: mol_type = other DNA; note = Probe - F2A link -1
tgacctcttgaagcttgc

SEQ ID NO: 106 - DNA, 20 bp, synthetic construct
  source 1..20: mol_type = other DNA; note = Reverse Primer - F2 link -1
tggaccaggattagactcca

SEQ ID NO: 107 - DNA, 20 bp, synthetic construct
  source 1..20: mol_type = other DNA; note = Forward Primer - F2A link -2
cctgtgaaacagaccctgaa

SEQ ID NO: 108 - DNA, 18 bp, synthetic construct
  source 1..18: mol_type = other DNA; note = Probe - F2A link -2
tgacctcttgaagcttgc

SEQ ID NO: 109 - DNA, 20 bp, synthetic construct
  source 1..20: mol_type = other DNA; note = Reverse Primer - F2A link -2
tggaccaggattagactcca

SEQ ID NO: 110 - DNA, 24 bp, synthetic construct
  source 1..24: mol_type = other DNA; note = Forward Primer - mHPRT1
gttaagcagtacagccccaaaatg

SEQ ID NO: 111 - DNA, 30 bp, synthetic construct
  source 1..30: mol_type = other DNA; note = Probe - mHPRT1
agcttgctggtgaaaaggacctctcgaagt

SEQ ID NO: 112 - DNA, 24 bp, synthetic construct
  source 1..24: mol_type = other DNA; note = Reverse Primer - mHPRT1
aaatccaacaaagtctggcctgta

SEQ ID NO: 113 - protein, 673 aa, synthetic construct
  source 1..673: note = VRDN-01100
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 113 aa, synthetic construct
  source 1..113: note = VRDN-01100 VL
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 115 - protein, 124 aa, synthetic construct
  source 1..124: note = VRDN-01100 VH
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 116 - protein, 665 aa, synthetic construct
  source 1..665: note = VRDN-02700
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 113 aa, synthetic construct
  source 1..113: note = VRDN-02700 VL
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKR

SEQ ID NO: 118 - protein, 117 aa, synthetic construct [sequence omitted]

SEQ ID NO: 119 - protein, 18 aa, synthetic construct
  source 1..18: note = signal peptide IL-10
MHSSALLCCLVLLTGVRA

SEQ ID NO: 120 - protein, 20 aa, synthetic construct
  source 1..20: note = signal peptide IL-2
MYRMQLLSCIALSLALVTNS

SEQ ID NO: 121 - DNA, 54 bp, synthetic construct
  source 1..54: mol_type = other DNA; note = signal peptide IL-10
atgcacagctccgccctgctctgctgtttggtgcttctgaccggcgtccgggct

SEQ ID NO: 122 - DNA, 60 bp, synthetic construct
  source 1..60: mol_type = other DNA; note = signal peptide IL-2
atgtaccggatgcagctgctcagctgcatcgccttgtcccttgctctggtgaccaactct

(19) United States
(12) Patent Application Publication
(10) Pub. No.: US 2025/0084434 A1
(43) Pub. Date: Mar. 13, 2025

(54) VECTOR CONSTRUCTS FOR DELIVERY OF NUCLEIC ACIDS ENCODING THERAPEUTIC ANTI-IGF-1R ANTIBODIES AND METHODS OF USING THE SAME

(71) Applicant: Kriya Therapeutics, Inc., Morrisville, NC (US)

(72) Inventors: Michele STONE, Apex, NC (US); Theresa HEAH, Hoboken, NJ (US); Nachiketa GUPTA, San Francisco, CA (US); Weiran SHEN, Mountain View, CA (US); Diana CEPEDA, San Diego, CA (US); Ruth CASTELLANOS, Raleigh, NC (US)

(21) Appl. No.: 18/727,201

(22) PCT Filed: Jan. 9, 2023
(86) PCT No.: PCT/US2023/060329

§ 371 Date: Jul. 8, 2024

Related U.S. Application Data
(60) Provisional application No. 63/297,787, filed on Jan. 9, 2022.
(60) Provisional application No. 63/374,878, filed on Sep. 7, 2022.

(51) Int. Cl. C12N 15/86; A61K 39/00; A61K 48/00; A61P 27/02; A61P 35/00; C07K 14/54; C07K 14/55; C07K 16/28

(52) U.S. Cl. CPC C12N 15/86; A61K 48/0041; A61K 48/0058; A61P 27/02; A61P 35/00; C07K 14/5428; C07K 14/55; C07K 16/2863; A61K 2039/505; C07K 2317/565; C07K 2319/02; C07K 2319/50; C12N 2750/14122; C12N 2750/14143; C12N 2830/50; C12N 2840/007; C12N 2840/203

(57) ABSTRACT

The present disclosure provides gene therapy vectors, antibody expression cassettes, and vectors comprising a nucleic acid encoding a therapeutic anti-IGF-1R antibody (e.g., a monoclonal antibody) or an antigen binding fragment thereof, delivery vectors (e.g., viral vectors) comprising gene therapy constructs a nucleic acid encoding a therapeutic anti-IGF-1R antibody (e.g., a monoclonal antibody) or an antigen binding fragment thereof, compositions comprising the same, and methods of using the same. Some aspects of the disclosure are directed to a recombinant adeno-viral vector (rAAV) particle for delivery of a nucleic acid encoding a therapeutic anti-IGF-1R antibody (e.g., a monoclonal antibody) or an antigen binding fragment thereof.

### CROSS-REFERENCE TO RELATED APPLICATIONS

The present application claims the priority benefit of U.S. Provisional Application No. 63/297,787, filed Jan. 9, 2022 and U.S. Provisional Application No. 63/374,878, filed Sep. 7, 2022, each of which is incorporated by reference herein in its entirety.

### REFERENCE TO SEQUENCE LISTING SUBMITTED ELECTRONICALLY

The content of the electronically submitted sequence listing (Name: 4525_074PC02_Seqlisting_ST26.xml; Size: 214,621 bytes; and Date of Creation: Jan. 9, 2023), filed with the application, is incorporated herein by reference in its entirety.

### FIELD OF DISCLOSURE

The present disclosure pertains to the medical field, including AAV gene therapy.

### BACKGROUND

Autoimmune diseases are the third most common human pathology after cancer and cardiovascular disease. The etiology of autoimmune disease is multifactorial, with genetic and environmental triggers arising from a background of critical defects in immune regulation. The autoimmune diseases Graves' disease and Hashimoto's thyroiditis are the most prevalent of the autoimmune conditions in the Western world. In Graves' disease, hyperthyroidism is due to activation of the thyroid gland by agonistic antibodies to the thyroid-stimulating hormone receptor (TSHR). The TSHR is also expressed by orbital fibroblasts, and binding of TSHR antibodies to orbital fibroblasts leads to hyaluronan production and differentiation to adipocytes and myofibroblast (Eckstein et al. Endocrine 68:265-70, 2020). The consequence is an increase in orbital fat and fibrosis of the orbital connective tissue, especially in the extraocular muscles, which leads to thyroid eye disease (TED), also called Graves' Orbitopathy (GO) (Eckstein et al. Endocrine 68:265-70, 2020). Overexpression of the insulin-like growth factor 1 receptor (IGF-1R) and its interaction with the thyrotropin receptor (TSH-R) is a key pathogenic feature of this disease.

Inhibition of IGF-1R can be achieved with anti-IGF-1R monoclonal antibodies, e.g., teprotumumab (aka teprotumumab-trbw; RG-1507) (sold under the brand name Tepezza). Binding of teprotumumab inhibits signaling through the IGF-1R/TSH-R complex and downstream pathways. Teprotumumab was first approved in the U.S. in 2020 for the treatment of acute and chronic TED. Teprotumumab is a fully human IgG1 type monoclonal antibody, which is administered by intravenous infusion. For most indications, the maintenance treatment requires repeated infusion, e.g., 8 total infusions, with the first recommended at 10 mg/kg followed by doubling the dose to 20 mg/kg for 7 more infusions, each spaced three weeks apart. Infusions are generally given over 60 to 90 minutes in a clinical setting, imposing a significant treatment burden on patients.

Gene therapy involves delivery of nucleic acids into a patient's cells to treat disease. Advances in the field of gene therapy have been achieved using viruses to deliver therapeutic genetic material. Although a variety of physical and chemical methods have been developed for introducing exogenous DNA into eukaryotic cells, viruses have generally been shown to be more efficient for this purpose. Several DNA-containing viruses such as parvoviruses, adenoviruses, herpesviruses and poxviruses, and RNA-containing viruses, such as retroviruses, have been used to develop eukaryotic cloning and expression vectors. Some challenges with the viral vectors include low efficiency, DNA packaging capacity, and a lack of target cell specificity.

### BRIEF SUMMARY

Certain aspects of the disclosure relate to the development of polynucleotides (e.g., antibody expression cassettes) encoding an anti-insulin-like growth factor 1 receptor (anti-IGF-1R) antibody or an antigen-binding fragment thereof for use in gene therapy; vectors (e.g., viral vectors) comprising the same; recombinant adeno-associated virus (rAAV) particles comprising the same; compositions comprising the same, which are suitable for delivery of the polynucleotide encoding the anti-IGF-1R antibody or antigen-binding fragment thereof to a target site of interest; and methods of using the same. In some aspects, the disclosure is directed to rAAV delivery of antibody expression cassettes encoding an anti-IGF-1R antibody or antigen-binding fragment thereof to a subject in need thereof (e.g., a subject suffering from Graves's orbitopathy).

Certain aspects of the disclosure are directed to a recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a nucleic acid sequence encoding a heavy chain variable region (VH) of an anti-insulin-like growth factor 1 receptor (anti-IGF-1R) antibody or an antigen-binding fragment thereof, and (c) a nucleic acid sequence encoding a light chain variable region (VL) of an anti-IGF-1R antibody or an antigen-binding fragment thereof.

In some aspects, the nucleic acid sequence encoding the VH of the anti-IGF-1R antibody or an antigen-binding fragment thereof comprises (i) a nucleic acid encoding a VH complementary determining region (CDR) 1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10 (or a VH CDR1 coding sequence disclosed in Table 3 or Table 5), (ii) a nucleic acid encoding a VH CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14 (or a VH CDR2 coding sequence disclosed in Table 3 or Table 5), and (iii) a nucleic acid encoding a VH CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15 (or a VH CDR3 coding sequence disclosed in Table 3 or Table 5); and the nucleic acid sequence encoding the VL of the anti-IGF-1R antibody or an antigen-binding fragment thereof comprises (i) a nucleic acid sequence comprising VL CDR1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16 (or a VL CDR1 coding sequence disclosed in Table 4 or Table 7), (ii) a nucleic acid sequence comprising a VL CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23 (or a VL CDR2 coding sequence disclosed in Table 4 or Table 7), and (iii) a nucleic acid sequence comprising a VL CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24 (or a VL CDR3 coding sequence disclosed in Table 4 or Table 7).

Certain aspects of the disclosure are directed to a vector (e.g., a vector genome) comprising an antibody expression cassette comprising:
(a) a promoter;
(b) a nucleic acid sequence encoding a heavy chain variable region (VH) of an anti-insulin-like growth factor 1 receptor (anti-IGF-1R) antibody or an antigen-binding fragment thereof comprising (i) a nucleic acid encoding a VH complementary determining region (CDR) 1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10 (or a VH CDR1 coding sequence disclosed in Table 3 or Table 5), (ii) a nucleic acid encoding a VH CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14 (or a VH CDR2 coding sequence disclosed in Table 3 or Table 5), and (iii) a nucleic acid encoding a VH CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15 (or a VH CDR3 coding sequence disclosed in Table 3 or Table 5);
(c) a nucleic acid sequence encoding a light chain variable region (VL) of an anti-IGF-1R antibody or an antigen-binding fragment thereof comprising (i) a nucleic acid sequence comprising VL CDR1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16 (or a VL CDR1 coding sequence disclosed in Table 4 or Table 7), (ii) a nucleic acid sequence comprising a VL CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23 (or a VL CDR2 coding sequence disclosed in Table 4 or Table 7), and (iii) a nucleic acid sequence comprising a VL CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24 (or a VL CDR3 coding sequence disclosed in Table 4 or Table 7).

In some aspects, the antibody expression cassette comprises a nucleic acid sequence encoding a signal peptide. In some aspects, the antibody expression cassette comprises a nucleic acid sequence encoding a first signal peptide, and a nucleic acid sequence encoding a second signal peptide. In some aspects, the first and the second signal peptide are the same. In some aspects, the first and the second signal peptide are different. In some aspects, the first signal peptide (e.g., HC signal peptide) comprises a human IL-10 signal sequence. In some aspects, the second signal peptide (e.g., LC signal peptide) comprises a human IL-2 signal sequence.

In some aspects, the signal peptide (e.g. the first signal peptide or the second signal peptide) comprises an amino acid sequence at least 85%, 86%, 87%, 88%, 89%, 90%, 91%, 92%, 93%, 94%, 95%, 96%, 97%, 98%, 99%, or 100% identical to any of SEQ ID NOs: 119 or 120.

In some aspects, the antibody expression cassette comprises a linker sequence selected from an internal ribosome entry site (IRES) sequence, a proteolytic cleavage site, or a combination thereof.

In some aspects, the proteolytic cleavage site comprises a furin cleavage site, a 2A cleavage site, or a combination thereof.

In some aspects, the antibody expression cassette comprises the promoter, the nucleic acid sequence encoding the VH, the IRES, and the nucleic acid sequence encoding the VL in 5′-3′ orientation.

In some aspects, the antibody expression cassette comprises the promoter, the nucleic acid sequence encoding the first signal peptide, the nucleic acid sequence encoding the VH, the IRES, the nucleic acid sequence encoding the second signal peptide, and the nucleic acid sequence encoding the VL in 5′-3′ orientation.

In some aspects, the antibody expression cassette comprises the promoter, the nucleic acid sequence encoding the VL, the IRES, and the nucleic acid sequence encoding the VH in 5′-3′ orientation.

In some aspects, the antibody expression cassette comprises the promoter, the nucleic acid sequence encoding the first signal peptide, the nucleic acid sequence encoding the VL, the IRES, the nucleic acid sequence encoding the second signal peptide, and the nucleic acid sequence encoding the VH in 5′-3′ orientation.

In some aspects, the antibody expression cassette comprises the promoter, the nucleic acid sequence encoding the VH, the proteolytic cleavage site, and the nucleic acid sequence encoding the VL in 5′-3′ orientation.

In some aspects, the antibody expression cassette comprises the promoter, the nucleic acid sequence encoding the first signal peptide, the nucleic acid sequence encoding the VH, the proteolytic cleavage site, the nucleic acid sequence encoding the second signal peptide, and the nucleic acid sequence encoding the VL in 5′-3′ orientation.

In some aspects, the antibody expression cassette comprises the promoter, the nucleic acid sequence encoding the VL, the proteolytic cleavage site, and the nucleic acid sequence encoding the VH in 5′-3′ orientation.

In some aspects, the antibody expression cassette comprises the promoter, the nucleic acid sequence encoding the first signal peptide, the nucleic acid sequence encoding the VL, the proteolytic cleavage site, the nucleic acid sequence encoding the second signal peptide, and the nucleic acid sequence encoding the VH in 5′-3′ orientation.

In some aspects, the IRES comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 43 or an IRES sequence disclosed in Table 15.

In some aspects, the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 44 or a furin cleavage site sequence disclosed in Table 15.

In some aspects, the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 45 or an 2A cleavage site (e.g., F2A) sequence disclosed in Table 15.

In some aspects, the signal peptide is an IL-2 signal peptide or an IL-10 signal peptide. In some aspects, the encoded signal peptide comprises an amino acid sequence at least 85%, 86%, 87%, 88%, 89%, 90%, 91%, 92%, 93%, 94%, 95%, 96%, 97%, 98%, 99%, or 100% identical to any of SEQ ID NOs: 119 or 120. In some aspects, the nucleic acid sequence encoding the signal peptide comprises a nucleic acid sequence at least 85%, 86%, 87%, 88%, 89%, 90%, 91%, 92%, 93%, 94%, 95%, 96%, 97%, 98%, 99%, or 100% identical to any of SEQ ID NOs: 121 or 122.

In some aspects, the antibody expression cassette comprises a second promoter.

In some aspects, the antibody expression cassette comprises the promoter (a first promoter), the nucleic acid sequence encoding the VL, the second promoter, and the nucleic acid sequence encoding the VH in 5′-3′ orientation.

In some aspects, the antibody expression cassette comprises the promoter (a first promoter), the nucleic acid sequence encoding the first signal peptide, the nucleic acid sequence encoding the VL, the second promoter, the nucleic acid sequence encoding the second signal peptide, and the nucleic acid sequence encoding the VH in 5′-3′ orientation.

In some aspects, the antibody expression cassette comprises the promoter (a first promoter), the nucleic acid sequence encoding the VH, the second promoter, and the nucleic acid sequence encoding the VL in 5′-3′ orientation.

In some aspects, the antibody expression cassette comprises the promoter (a first promoter), the nucleic acid sequence encoding the first signal peptide, the nucleic acid sequence encoding the VH, the second promoter, the nucleic acid sequence encoding the second signal peptide, and the nucleic acid sequence encoding the VL in 5′-3′ orientation.

In some aspects, the antibody expression cassette comprises the nucleic acid sequence encoding VH, the promoter (a first promoter), the second promoter, and the nucleic acid sequence encoding the VL in 5′-3′ orientation.

In some aspects, the antibody expression cassette comprises the nucleic acid sequence encoding the nucleic acid sequence encoding the first signal peptide, the VH, the promoter (a first promoter), the second promoter, the nucleic acid sequence encoding the second signal peptide, and the nucleic acid sequence encoding the VL in 5′-3′ orientation.

In some aspects, the antibody expression cassette comprises the nucleic acid sequence encoding a VL, the promoter (a first promoter), the second promoter, and the nucleic acid sequence encoding the VH in 5′-3′ orientation.

In some aspects, the antibody expression cassette comprises the nucleic acid sequence encoding the first signal peptide, the nucleic acid sequence encoding a VL, the promoter (a first promoter), the second promoter, the nucleic acid sequence encoding the second signal peptide, and the nucleic acid sequence encoding the VH in 5′-3′ orientation.

In some aspects, the promoter and/or the second promoter is a constitutively active promoter, a cell-type specific promoter, a synthetic promoter, or an inducible promoter.

In some aspects, the promoter and/or the second promoter is a CBA promoter, a CMV promoter (optionally, a human CMV promoter or a mouse CMV promoter), an EF1α promoter, a CAG promoter, or a tissue specific promoter.

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51, 83, or 93, or a promoter and/or enhancer sequence disclosed in Table 15.

In some aspects, the promoter is a muscle specific promoter. In some aspects, the muscle specific promoter is selected from a desmin (DES) promoter, a human skeletal muscle α-actin (HSA) promoter, a myosin creatine kinase (MCK) promoter, a α myosin heavy chain myosine creatine kinase 7 (HMCK7) promoter, a dual MCK enhancer MCK (dMCK) promoter, a triple MCK enhancer MCK (tMCK) promoter, a dual MCK enhancer muscle-type creatine kinase 8 e (CK8e) promoter, a SPc5-12 promoter, a SP-301 promoter, a α myosin heavy chain (MHC) promoter, a Sk-CRM promoter, or a Sk-CRM4 promoter.

In some aspects, the antibody expression cassette comprises an intron. In some aspects, the intron is a CAG intron, an SV40 intron, MVM intron, or a human beta-globin intron, or any combination thereof.

In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NOs: 46 or 82 or an intron sequence disclosed in Table 15.

In some aspects, the promoter comprises a first and a second promoter which are different.

In some aspects, first and second promoter initiate transcription in the same direction.

In some aspects, first and second promoter initiate transcription in different directions.

In some aspects, the antibody expression cassette comprises a pause element between the first and second promoter.

In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 54 or a pause element sequence disclosed in Table 15.

In some aspects, the anti-IGF-1R antibody is a monoclonal antibody. In some aspects, the VH CDRs 1-3 correspond to the CDRs of teprotumumab and/or the VL CDRs 1-3 correspond to the CDRs of teprotumumab. In some aspects, the anti-IGF-1R antibody is teprotumumab.

In some aspects, the nucleic acid sequence encoding the VH comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 25-27 (or a VH coding sequence disclosed in Table 5). In some aspects, the encoded VH comprises SEQ ID NO: 28 or SEQ ID NO: 91 (or any of the VH amino acid sequences in Table 6).

In some aspects, the nucleic acid sequence encoding the VL comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 29-31 (or a VL coding sequence disclosed in Table 7). In some aspects, the encoded VL comprises SEQ ID NO: 32 or SEQ ID NO: 92 (or any of the VL amino acid sequences in Table 8).

In some aspects, the nucleic acid sequence encoding the having chain (HC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 35-37 (or a HC coding sequence disclosed in Table 11). In some aspects, the encoded HC comprises SEQ ID NO: 38 (or the HC amino acid sequence in Table 12).

In some aspects, the nucleic acid sequence encoding the light chain (LC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39-41 (or a LC coding sequence disclosed in Table 13). In some aspects, the encoded LC comprises SEQ ID NO: 42 (or the LC amino acid sequence in Table 14).

In some aspects, the encoded anti-IGF-1R antibody is teprotumumab.

In some aspects, the antibody expression cassette comprises a poly (A) sequence. In some aspects, the poly(A) sequence is selected from a bGHpA, a hGHpA, a SV40 pA, or a synthetic pA.

In some aspects, the poly (A) comprises a nucleotide sequence at least 85%, 86%, 87%, 88%, 89%, 90%, 91%, 92%, 93%, 94%, 95%, 96%, 97%, 98%, 99%, or 100% identical to SEQ ID NOs: 52 or 53 or a poly (A) sequence disclosed in Table 15.

In some aspects, antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 57-67 and 94-97 or an ORF sequence disclosed in Table 16.

In some aspects, the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 68-76 or an antibody expression cassette sequence disclosed in Table 17.

In some aspects, an inverted terminal repeats (ITR). In some aspects, the AAV ITR comprises a pair of ITRs flanking the antibody expression cassette. In some aspects, the ITRs are of the same serotype as one another. In some aspects, the ITRs are of the AAV2 serotype.

In some aspects, the vector is packaged in an AAV capsid.

In some aspects, AAV capsid serotype is selected from the group consisting of AAV1, AAV2, AAV3, AAV4, AAV5, AAV6, AAV7, AAV8, AAVRh8, AAVrh9, AAV9, AAVrh10, AAV10, AAV11, AAV12, and a modified version thereof.

In some aspects, the AAV capsid serotype is selected from the group consisting of AAV1, AAV2, AAV6, AAV8, AAV9, and a modified version thereof.

Certain aspects of the disclosure are directed to a host cell comprising the rAAV particle or the vector disclosed herein.

Certain aspects of the disclosure are directed to a composition comprising the rAAV particle or the vector disclosed herein and a carrier. In some aspects, the carrier is water or saline.

Certain aspects of the disclosure are directed to a method of expressing an anti-IGF-1R antibody or antigen-binding fragment thereof in a cell, comprising administering to the cell the rAAV particle, the vector, or the composition disclosed herein, thereby expressing the anti-IGF-1R antibody or antigen-binding fragment thereof in the cell.

In some aspects, the cell is a fibroblast cell, an adipocyte cell, a myofibroblast cell, a myocyte cell, a muscle cell, or any combination thereof.

In some aspects, the administration is in vitro.

In some aspects, the administration is in vivo.

Certain aspects of the disclosure are directed to a method of expressing an anti-IGF-1R antibody or antigen-binding fragment thereof in a subject in need thereof, comprising administering to the subject the rAAV particle, the vector, or the composition disclosed herein, thereby expressing the anti-IGF-1R antibody or antigen-binding fragment thereof in the subject.

In some aspects, the subject suffers from a thyroid eye disease selected from active Graves' Orbitopathy and chronic Graves' Orbitopathy.

Certain aspects of the disclosure are directed to a method of treating a thyroid eye disease in a subject in need thereof comprising administering to the subject the rAAV particle, the vector, or the composition disclosed herein, thereby expressing the anti-IGF-1R antibody or antigen-binding fragment thereof in the subject and treating the thyroid eye disease.

In some aspects, the thyroid eye disease selected from active Graves' Orbitopathy and chronic Graves' Orbitopathy.

In some aspects, the administration is suitable for delivery of the rAAV particle or the vector to an ocular delivery site, a retro or a periorbital delivery site, a retrobulbar delivery site, an extra-ocular muscle delivery site, a connective tissue delivery site, or any combination of delivery sites thereof.

In some aspects, the administration is by injection or infusion.

In some aspects, the administration is intramuscular (IM), intravenous (IV), intralymphatic, intraocular, retroorbital, periorbital, retrobulbar, or any combination thereof.

In some aspects, the administration is suitable for delivery to retro or a periorbital fibroblast cells, adipocytes cells, myofibroblast cells, myocyte cells, or any combination thereof.

In some aspects, the administration is to an extra-ocular muscle.

In some aspects, the extra-ocular muscle is a levantor muscle or a glabellar muscle.

In some aspects, the administration is to a connective tissue.

In some aspects, the administration is transconjunctival into the periorbital space.

In some aspects, the administration is intralymphatic to the pre-auricular or submandibular node.

In some aspects, the administration is a single dose. In some aspects, the single dose multiple injections and/or infusions.

In some aspects, the administration is a single dose. In some aspects, the single dose multiple injections and/or infusions.

### BRIEF DESCRIPTION OF THE FIGURES

FIG. 1 shows a plasmid map of a vector including the following ITR to ITR elements: AAV2 ITRs flanking in 5′ to 3′ order a CMV enhancer, a CBA promoter, a CAG intron, a nucleic acid encoding a signal peptide, a nucleic acid encoding teprotumumab heavy chain (Tepro HC), a F2A sequence, a nucleic acid encoding a signal peptide, a nucleic acid encoding teprotumumab light chain (Tepro LC), and a bovine poly adenylation (bGH pA).

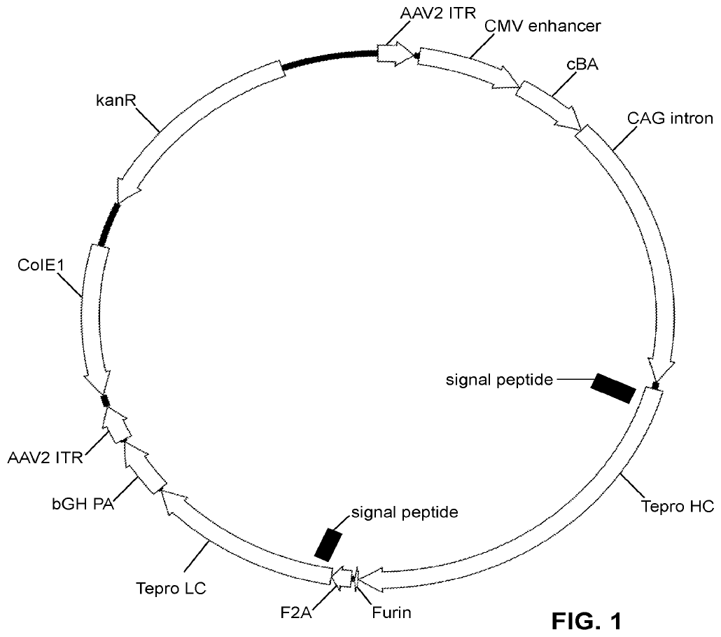

FIG. 2 shows a plasmid map of a vector including the following ITR to ITR elements: AAV2 ITRs flanking in 5′ to 3′ order a CMV enhancer, a CBA promoter, a CAG intron, a nucleic acid encoding a signal peptide, a nucleic acid encoding teprotumumab light chain (Tepro LC), an IRES sequence (IRES2), a nucleic acid encoding a signal peptide, a nucleic acid encoding teprotumumab heavy chain (Tepro HC), and a synthetic poly adenylation site (syn pA).

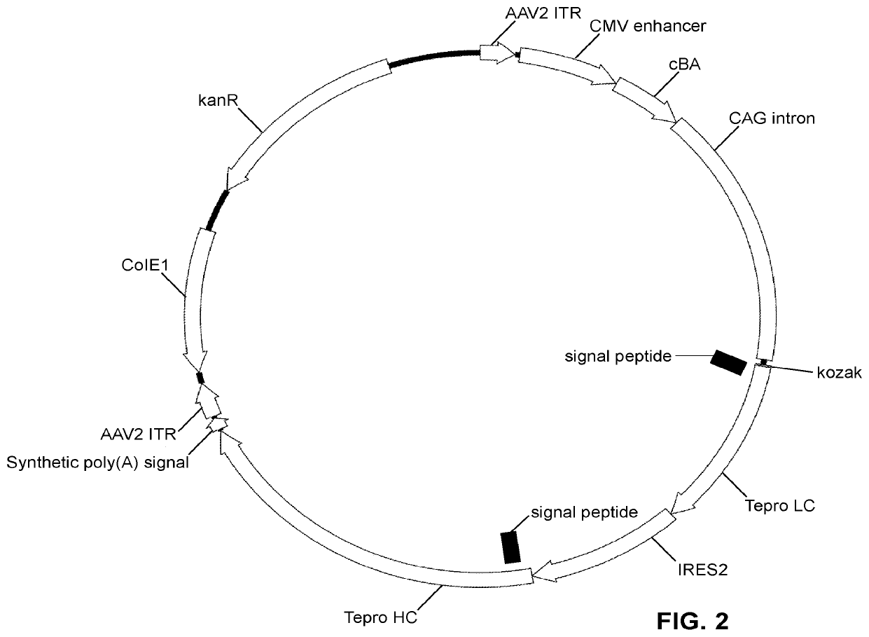

FIG. 3 shows a plasmid map of a vector including the following ITR to ITR elements: AAV2 ITRs flanking in 5′ to 3′ order a CMV enhancer, a CMV promoter, a SV40 intron, a nucleic acid encoding a signal peptide, a nucleic acid encoding teprotumumab light chain (Tepro LC), bovine poly adenylation site (bGH PA), pause site, an EF-1α core promoter, a truncated 5′LTR, chimeric human betaglobin/immunoglobulin heavy chain intron, a nucleic acid encoding a signal peptide, a nucleic acid encoding teprotumumab heavy chain (Tepro HC), and a synthetic poly adenylation site (syn pA).

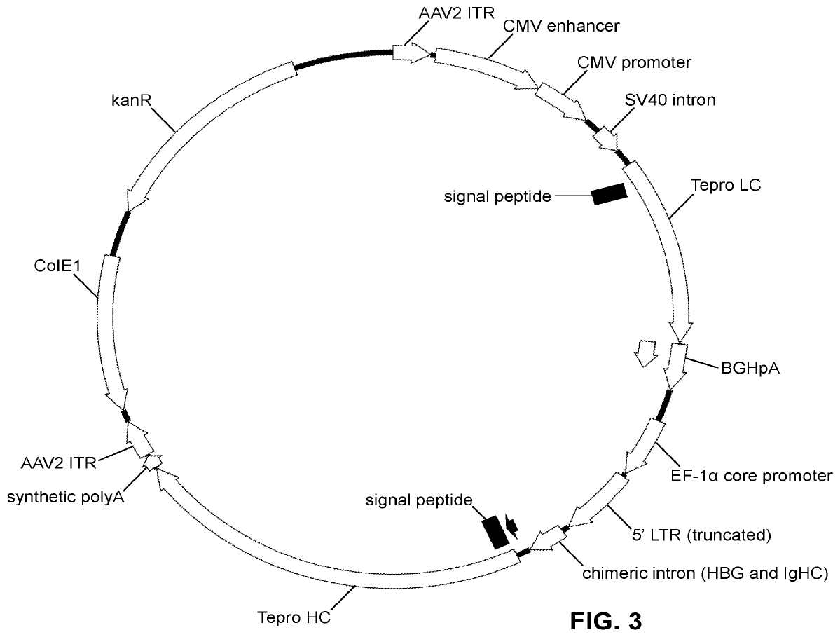

FIGS. 4A-4D show protein and mRNA quantification of teprotumumab secreted from transfected HEK-293 cells. FIG. 4A is a graph showing the amount of teprotumumab secreted from HEK-293 cells transfected with H-F2A-L (ORF #1), H-F2A-L (ORF #2), L-IRES-H (ORF #12), L-IRES-H (ORF #13), Dual promoter (ORFs #4 and 5), or Dual promoter (ORFs #6 and 7) teprotumumab AAV plasmid constructs. FIG. 4B is a graph showing the amount of teprotumumab secreted from HEK-293 cells transfected with H-F2A-L (ORF #1), H-F2A-L (ORF #2), L-IRES-H (ORF #12) or L-IRES-H (ORF #13) teprotumumab AAV plasmid constructs or H-F2A-L (ORF #1) (plasmid) or L-IRES-H (ORFs #4 and 5) (plasmid) teprotumumab non-AAV plasmid constructs. FIG. 4C shows a Western Blot analysis of light and heavy chains of secreted Teprotumumab performed in reducing and non-reducing conditions as indicated. FIG. 4D is a graph showing teprotumumab mRNA levels in HEK-293 cells transfected with H-F2A-L (ORF #1), L-IRES-H (ORF #12), or Dual promoter (ORFs #4 and 5) teprotumumab AAV plasmid constructs.

FIGS. 5A-B show binding of vectorized teprotumumab secreted from transfected HEK-293 cells to IGF-1R. FIG. 5A is a graph showing the IGF-1R binding capacity of teprotumumab secreted from HEK-293 cells transfected with H-F2A-L (ORF #1), H-F2A-L (ORF #2), L-IRES-H (ORF #12) or L-IRES-H (ORF #13) teprotumumab AAV plasmid constructs. FIG. 5B is a graph showing the linearity of the binding of vectorized teprotumumab secreted from transfected HEK-293 cells to IGF-1R in two replicates of the same binding experiment.

FIG. 6 is a graph showing reduction in IGF-1R phosphorylation levels upon treatment with recombinant teprotumumab or teprotumumab secreted from HEK-293 cells transfected with H-F2A-L (ORF #1) or H-F2A-L (ORF #2).

FIGS. 7A-B show total and phospho-IGF-1R expression decreases in HT-1080 (FIG. 7A) and Colo205 (FIG. 7B) cells after 48 hours of recombinant or vectorized Teprotumumab treatment as indicated. All wells in MSD plate were loaded with the same total μg of whole cell lysate of either HT-1080 or Colo205. All values presented are a percentage of Treated value/Mock Treated average value. N=3 technical repeats per sample, bars are mean+/−SD. All statistics are One-Way ANOVAs with post-hoc comparing each Teprotumumab treatment to the Mock Treated control, performed separately for Total or Phospho values. All “Vectorized Teprotumumab” is HEK293T supernatant containing the respective ng of Teprotumumab after transduction with AAV expressing H-F2A-L (ORF #2).

FIGS. 8A-C shows the percentage reduction in expression of insulin growth factor 1 receptor (IGF-1R) protein (FIG. 8A), Insulin Receptor (IR) protein (FIG. 8B) and Insulin receptor substrate 1 (IRS-1) protein (FIG. 8C) in cultured primary fibrocyte cells isolated from either a normal donor (indicated as Normal) or a Graves' Disease donor (indicated as Graves') peripheral blood after treatment with either vectorized Teprotumumab (vTepro) or recombinant Teprotumumab (rTepro) at a concentration of 500 ng/mL. All values presented are a percentage of (Mock Treated average value/Mock Treated average value*100)−(Treated value/Mock Treated average value*100) where each mean value is n=2 technical replicates. Normal sample values are plotted as an average of n=2 biological replicates per sample, bars are mean+/−SEM. Graves' sample values are plotted as N=1 biological replicates. All “Vectorized Teprotumumab” is HEK293T supernatant containing the respective ng of Teprotumumab after transduction with AAV expressing H-F2A-L (ORF #2). Treatment with vectorized Teprotumumab resulted in a substantial decrease in IGF-1R, 1R and IRS-1 protein expression levels in Graves' Disease fibrocyte.

FIG. 9 shows immunocytochemistry of Colo205 cells for IGF-1R with or without 500 ng/mL recombinant Teprotumumab treatment for 24-48 hours showing reduction of IGF-1R expression. Panels A and E show 24 hour untreated cell controls; and panels B and F show cells treated with Teprotumumab for 24 hours. Panels C and G show 48 hour untreated cell controls; and panels D and H show cells treated with Teprotumumab for 48 hours. Panels A, B, C, and D show cells that were not permeabilized; and panels E, F, G and H show cells that were permeabilized with 0.1% Triton X-100.

FIGS. 10A-C show tumor growth in Colo205 xenografted mice treated with AAV1-delivered antibody expression cassette encoding Teproumumab (FIG. 10A), AAV2-delivered antibody expression cassette encoding Teproumumab (FIG. 10B), or AAV9-delivered antibody expression cassette encoding Teproumumab (FIG. 10C), compared to untreated control mice, and mice treated with recombinant teprotumumab (6 mg/kg every 7 days) as indicated in the figure legend.

FIG. 11 shows levels of Teprotumumab in tumors obtained from animals treated with AAV1-delivered antibody expression cassette encoding Teprotumumab, AAV2-delivered antibody expression cassette encoding Teprotumumab, AAV9-delivered antibody expression cassette encoding Teprotumumab from day 7 to day 31, and in tumors obtained from animals treated with recombinant Teprotumumab (6 mg/kg every 7 days) at day 31 . . . . Bars represent mean±SEM. Days 7, 14 and 28 are the sampling arms, day 31 is the efficacy arm.

FIG. 12 shows serum levels of Teprotumumab in animals treated with AAV1-delivered antibody expression cassette encoding Teprotumumab, AAV2-delivered antibody expression cassette encoding Teprotumumab, AAV9-delivered antibody expression cassette encoding Teprotumumab from day 7 to day 31, and in serum of animals treated with recombinant Teprotumumab (6 mg/kg every 7 days) at day 31. Bars represent mean±SEM. Days 7, 14 and 28 are the sampling arms, day 31 is the efficacy arm.

FIG. 13 show levels of IGF-1R in tumors obtained from animals treated with AAV1-delivered antibody expression cassette encoding Teprotumumab, AAV2-delivered antibody expression cassette encoding Teprotumumab, AAV9-delivered antibody expression cassette encoding Teprotumumab from day 7 to day 31, and in tumors obtained from animals treated with recombinant Teprotumumab (6 mg/kg every 7 days) at day 31. Error bars represent mean #SEM. Days 7, 14 and 28 are the sampling arms, day 31 is the efficacy arm. The decrease in IFG-1R levels relative to untreated controls was statistically significant at all timepoints. At day 31, the decrease in IGF-1R levels caused by AAV1-delivered Teprotumumab and AAV9 delivered Teprotumumab was not statistically different from that caused by recombinant Teprotumumab (6 mg/kg every 7 days).

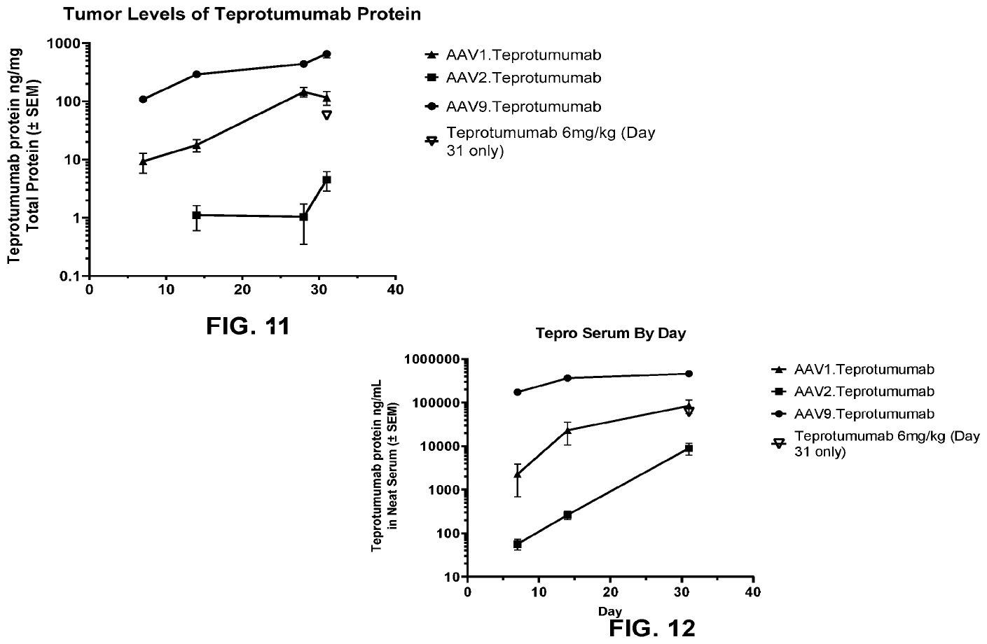

FIG. 14 shows the vector genome copies as copies/μg host genomic DNA in tumors obtained from animals treated with AAV1-delivered antibody expression cassette encoding Teprotumumab, AAV2-delivered antibody expression cassette encoding Teprotumumab, AAV9-delivered antibody expression cassette encoding Teprotumumab and in untreated control animals at days 7, 14, and 28, as indicated in the figure legend.

FIG. 15 shows mRNA expression as single stranded copies/μg host RNA in tumors obtained from animals treated with AAV1-delivered antibody expression cassette encoding Teprotumumab, AAV2-delivered antibody expression cassette encoding Teprotumumab, AAV9-delivered antibody expression cassette encoding Teprotumumab and in untreated control animals at days 7, 14, and 28, as indicated in the figure legend.

FIG. 16 shows the vector genome copies as copies/μg host genomic DNA, and mRNA expression as single stranded copies/μg host RNA, in tumors obtained from animals treated with AAV1-delivered antibody expression cassette encoding Teprotumumab, AAV2-delivered antibody expression cassette encoding Teprotumumab, AAV9-delivered antibody expression cassette encoding Teprotumumab, and in untreated control animals at days 7, 14, and 28, as indicated in the figure legend.

### DETAILED DESCRIPTION OF THE DISCLOSURE

Certain aspects of the disclosure relate to gene therapy vectors and expression constructs encoding anti-insulin-like growth factor 1 receptor (anti-IGF-1R) antibodies or antigen-binding fragments thereof; viral vectors (e.g., rAAV vectors) comprising the same; compositions comprising that same suitable for delivery (e.g., retrobulbar, periorbital, and/or intramuscular administration), and methods of using the same. In some aspects, the disclosure is directed to adeno-associated virus vector (AAV) delivery of antibody expression cassettes encoding anti-IGF-1R antibodies (e.g., monoclonal antibodies) or antigen-binding fragments thereof to a subject in need thereof.

Certain aspects of the disclosure are directed to a polynucleotide (e.g., an antibody expression cassette) comprising a nucleic acid encoding an antibody or antigen-binding fragment thereof which binds insulin-like growth factor 1 receptor (also referred to as IGF-1R or IGF-1R herein). In some aspects, the polynucleotide (e.g., an antibody expression cassette) comprises an open reading frame (ORF) comprising a nucleic acid sequence encoding an anti-IGF-1R heavy chain and a nucleic acid sequence encoding an anti-IGF-1R light chain. In some aspects, the ORF is operably linked to a promoter (e.g., a CBA promoter or a CMV promoter). In some aspects, the ORF is operably linked to an enhancer (e.g., a CMV enhancer) and/or an intron sequence (e.g., a CAG intron or a SV40 intron sequence). In some aspects, the ORF is operably linked to a polyadenylation (polyA) element (e.g., a bGHpA, a hGHpA, a SV40 pA, or a synthetic pA).

In some aspects, the ORF comprises a nucleic acid sequence encoding a signal peptide. In some aspects the signal peptide is an IL-2 signal peptide or and IL-10 signal peptide. In some aspects, the ORF comprises a nucleic acid sequence encoding a first signal peptide, and a nucleic acid sequence encoding a second signal peptide. In some aspects, the first and the second signal peptide are the same. In some aspects, the first and the second signal peptide are different.

In some aspects, the ORF comprises a linker between the nucleic acid sequence encoding the anti-IGF-1R heavy chain and the nucleic acid sequence encoding the anti-IGF-1R light chain. In some aspects, the linker is an internal ribosomal entry sequence (IRES), a proteolytic cleavage site (e.g., a furin and/or 2A cleavage site (e.g., F2A)), or a combination thereof. In some aspects, the OFR further comprises a nucleic acid encoding a signal sequence. In some aspects, the OFR is positioned between two inverted terminal repeats (ITRs).

In some aspects, the polynucleotide (e.g., an antibody expression cassette) comprises a promoter, a nucleic acid sequence encoding an anti-IGF-1R heavy chain region, an IRES, and a nucleic acid sequence encoding an anti-IGF-1R light chain region in 5′-3′ orientation.

In some aspects, the polynucleotide (e.g., an antibody expression cassette) comprises a promoter, a nucleic acid sequence encoding a first signal peptide, a nucleic acid sequence encoding an anti-IGF-1R heavy chain region, an IRES, a nucleic acid sequence encoding a second signal peptide, and a nucleic acid sequence encoding an anti-IGF-1R light chain region in 5′-3′ orientation.

In some aspects, the polynucleotide (e.g., an antibody expression cassette) comprises a promoter, a nucleic acid sequence encoding an anti-IGF-1R light chain region, an IRES, and a nucleic acid sequence encoding an anti-IGF-1R heavy chain region in 5′-3′ orientation.

In some aspects, the polynucleotide (e.g., an antibody expression cassette) comprises a promoter, a nucleic acid sequence encoding a first signal peptide, a nucleic acid sequence encoding an anti-IGF-1R light chain region, an IRES, a nucleic acid sequence encoding a second signal peptide, and a nucleic acid sequence encoding an anti-IGF-1R heavy chain region in 5′-3′ orientation.

In some aspects, the polynucleotide (e.g., an antibody expression cassette) comprises a promoter, a nucleic acid sequence encoding an anti-IGF-1R heavy chain region, an F2A cleavage site, and a nucleic acid sequence encoding an anti-IGF-1R light chain region in 5′-3′ orientation.

In some aspects, the polynucleotide (e.g., an antibody expression cassette) comprises a promoter, a nucleic acid sequence encoding a first signal peptide, a nucleic acid sequence encoding an anti-IGF-1R heavy chain region, an F2A cleavage site, a nucleic acid sequence encoding a second signal peptide, and a nucleic acid sequence encoding an anti-IGF-1R light chain region in 5′-3′ orientation.

In some aspects, the polynucleotide (e.g., an antibody expression cassette) comprises a promoter, a nucleic acid sequence encoding an anti-IGF-1R light chain region, a F2A cleavage site, and a nucleic acid sequence encoding an anti-IGF-1R heavy chain region sequences in 5′-3′ orientation.

In some aspects, the polynucleotide (e.g., an antibody expression cassette) comprises a promoter, a nucleic acid sequence encoding a first signal peptide, a nucleic acid sequence encoding an anti-IGF-1R light chain region, a F2A cleavage site, a nucleic acid sequence encoding a second signal peptide, and a nucleic acid sequence encoding an anti-IGF-1R heavy chain region sequences in 5′-3′ orientation.

In some aspects, the polynucleotide (e.g., an antibody expression cassette) further comprises a second promoter.

In some aspects, the polynucleotide (e.g., an antibody expression cassette) comprises a first promoter, a nucleic acid sequence encoding an anti-IGF-1R light chain, a second promoter, and a nucleic acid sequence encoding an anti-IGF-1R heavy chain in 5′-3′ orientation.

In some aspects, the polynucleotide (e.g., an antibody expression cassette) comprises a first promoter, a nucleic acid sequence encoding a first signal peptide, a nucleic acid sequence encoding an anti-IGF-1R light chain, a second promoter, a nucleic acid sequence encoding a second signal peptide, and a nucleic acid sequence encoding an anti-IGF-1R heavy chain in 5′-3′ orientation.

In some aspects, the polynucleotide (e.g., an antibody expression cassette) comprises a first promoter, a nucleic acid sequence encoding an anti-IGF-1R heavy chain, a second promoter, and a nucleic acid sequence encoding an anti-IGF-1R light chain in 5′-3′ orientation.

In some aspects, the polynucleotide (e.g., an antibody expression cassette) comprises a first promoter, a nucleic acid sequence encoding a first signal peptide, a nucleic acid sequence encoding an anti-IGF-1R heavy chain, a second promoter, a nucleic acid sequence encoding a second signal peptide, and a nucleic acid sequence encoding an anti-IGF-1R light chain in 5′-3′ orientation.

In some aspects, the polynucleotide (e.g., an antibody expression cassette) comprises a nucleic acid sequence encoding an anti-IGF-1R heavy chain, a first promoter, a second promoter, and a nucleic acid sequence encoding an anti-IGF-1R light chain in 5′-3′ orientation.

In some aspects, the polynucleotide (e.g., an antibody expression cassette) comprises a nucleic acid sequence encoding a first signal peptide, a nucleic acid sequence encoding an anti-IGF-1R heavy chain, a first promoter, a second promoter, a nucleic acid sequence encoding a second signal peptide, and a nucleic acid sequence encoding an anti-IGF-1R light chain in 5′-3′ orientation.

In some aspects, the polynucleotide (e.g., an antibody expression cassette) comprises a nucleic acid sequence encoding an anti-IGF-1R light chain, a first promoter, a second promoter, and a nucleic acid sequence encoding an anti-IGF-1R heavy chain in 5′-3′ orientation.

In some aspects, the polynucleotide (e.g., an antibody expression cassette) comprises a nucleic acid sequence encoding a first signal peptide, a nucleic acid sequence encoding an anti-IGF-1R light chain, a first promoter, a second promoter, a nucleic acid sequence encoding a second signal peptide, and a nucleic acid sequence encoding an anti-IGF-1R heavy chain in 5′-3′ orientation.

In some aspects, the promoter is a constitutively active promoter, a cell-type specific promoter, a synthetic promoter, or an inducible promoter. In some aspects, the promoter is selected from a CAG promoter, a CBA promoter, a human CMV promoter, a mouse CMV promoter, an EF1α promoter, an EF1α promoter with a CMV enhancer, a CMV promoter with a CMV enhancer (CMVe/p), a CMV promoter with a SV40 intron, or a tissue specific promoter. In some aspects, the cell type specific promoter is a muscle specific promoter including a DES promoter, a HSA promoter, a MCK promoter, a HMCK7 promoter, a dMCK promoter, a tMCK promoter, a CK8e promoter, a SPc5-12 promoter, a SP-301 promoter, a MH promoter, a Sk-CRM promoter, or a Sk-CRM4 promoter (see, e.g., Skopenkova et al., Acta Naturae 13:47-58, 2012).

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51, 83, or 93, or a promoter and/or enhancer sequence disclosed in Table 15. In some aspects, the nucleic acid sequence comprising the promoter can comprises an intron. In some aspects, the intron is selected from the group consisting of a CAG intron, an SV40 intron, MVM intron, a human betaglobin intron or a chimeric human betaglobin-human immunoglobulin chain intron. In some aspects, the CAG intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 82. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 46. In some aspects, the promoter comprise an intron sequence disclosed in Table 15.

In some aspects, the promoter comprises a first and a second promoter. In some aspects, the first and second promoter are different. In some aspects, the first and second promoter are the same. In some aspects, the first and second promoter initiate transcription in the same direction. In some aspects, the first and second promoter initiate transcription in different directions. In some aspects, the first and/or second promoter in a CMV promoter. In some aspects, the first and/or second promoter is an EF-1α promoter. In some aspects, the first and/or second promoter is a CBA promoter.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 54 or a pause element sequence disclosed in Table 15.

In some aspects the signal peptide is an endogenous signal peptide for HGH and variants thereof; an endogenous signal peptide for interferons and variants thereof, including the signal peptide of type I, II and III interferons and variants thereof; an endogenous signal peptides for known cytokines and variants thereof, such as the signal peptide of erythropoietin (EPO), insulin, TGF-β1, TNF, IL1-α, and IL1-β, and variants thereof. In some embodiments, the signal peptide is a modified signal peptide. In some aspects, the signal peptide is an IL-2 signal peptide. In some aspects, the signal peptide is an IL-10 signal peptide. In some aspects the signal peptide comprises and amino acid sequence with at least 85%, 86%, 87%, 88%, 89%, 90%, 91%, 92%, 93%, 94%, 95%, 96%, 97%, 98%, 99%, or 100% identity to SEQ ID NO: 119 or 120.

In some aspects, the anti-IGF-1R antibody is a monoclonal antibody. In some aspects, the anti-IGF-1R antibody is teprotumumab.

In some aspects, the anti-IGF-1R antibody heavy chain comprises a heavy chain variable region (VH) comprising a complementarity determining region (CDR) 1, a VH CDR2, and a VH CDR3. In some aspects, the VH CDRs 1-3 correspond to the CDRs of teprotumumab. In some aspects, the nucleic acid sequence encoding the VH CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10 (or a VH CDR1 coding sequence disclosed in Table 3 or Table 5); the nucleic acid sequence encoding the VH CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14 (or a VH CDR2 coding sequence disclosed in Table 3 or Table 5); and the nucleic acid sequence encoding the VH CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15 (or a VH CDR3 coding sequence disclosed in Table 3 or Table 5).

In some aspects, the anti-IGF-1R antibody light chain comprises a light chain variable region (VL) comprising a complementarity determining region (CDR) 1, a VL CDR2, and a VL CDR3. In some aspects, the VL CDRs 1-3 correspond to the CDRs of teprotumumab. In some aspects, the nucleic acid sequence encoding the VL CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16 (or a VL CDR1 coding sequence disclosed in Table 4 or Table 7); the nucleic acid sequence encoding the VL CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23 (or a VL CDR2 coding sequence disclosed in Table 4 or Table 7); and the nucleic acid sequence encoding the VL CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24 (or a VL CDR3 coding sequence disclosed in Table 4 or Table 7).

In some aspects, the nucleic acid sequence encoding the heavy chain variable region (VH) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 25-27 (or a VH coding sequence disclosed in Table 5). In some aspects, the encoded VH comprises SEQ ID NO: 28 or SEQ ID NO: 91 (or any of the VH amino acid sequences in Table 6). In some aspects, the nucleic acid sequence encoding the light chain variable region (VL) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 29-31 (or a VL coding sequence disclosed in Table 7). In some aspects, the encoded VL comprises SEQ ID NO: 32 or SEQ ID NO: 92 (or any of the VL amino acid sequences in Table 8).

In some aspects, the nucleic acid sequence encoding the heavy chain (HC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 35-37 (or a HC coding sequence disclosed in Table 11). In some aspects, the encoded HC comprises SEQ ID NO: 38 (or the HC amino acid sequence in Table 12). In some aspects, the nucleic acid sequence encoding the light chain (LC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39-41 (or a LC coding sequence disclosed in Table 13). In some aspects, the encoded LC comprises SEQ ID NO: 42 (or the LC amino acid sequence in Table 14).

In some aspects, the nucleic acid sequence encoding the heavy chain and the nucleic acid sequence encoding the light chain are operably linked. In some aspects, the nucleic acid sequence encoding the heavy chain and the nucleic acid sequence encoding the light chain are operably linked by a linker sequence. In some aspects, the linker sequence is selected from an IRES sequence, a proteolytic cleavage site (e.g., a furin and/or 2A cleavage site, e.g., F2A), or a combination thereof. In some aspects, the IRES comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 43 or an IRES sequence disclosed in Table 15. In some aspects, the furin cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 44 or a furin cleavage site sequence disclosed in Table 15. In some aspects, the 2A cleavage site comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 45 or an 2A cleavage site (e.g., F2A) sequence disclosed in Table 15.

In some aspects, the polynucleotide comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 57-67 and 94-97 or an ORF sequence disclosed in Table 16.

In some aspects, the polynucleotide (e.g., an antibody expression cassette) comprises a poly (A). In some aspects, the polyA sequence comprises a human growth hormone polyA signal sequence. In some aspects, the polyA sequence comprises a bovine growth hormone poly A signal sequence. In some aspects, the polyA sequence comprises a synthetic poly A sequence. In some aspects, the poly A sequence comprises a SV40 polyA signal sequence (SV40 pA). In some aspects, the polynucleotide (e.g., an antibody expression cassette) comprises a poly (A) sequence comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 52 or 53 or a poly (A) sequence disclosed in Table 15.

In some aspects, the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 68-76 or an antibody expression cassette sequence disclosed in Table 17.

Certain aspects of the disclosure are directed to a method of expressing and/or producing an anti-IGF-1R antibody or antigen-binding fragment thereof, comprising administering to a cell a polynucleotide (e.g., an antibody expression cassette), a vector, or a rAAV particle of the disclosure, thereby expressing and/or producing the anti-IGF-1R antibody or antigen-binding fragment thereof in the cell. In some aspects, the cell is a fibroblast cell, an adipocyte cell, a myofibroblast cell, a myocyte cell, a muscle cell, or any combination thereof.

Certain aspects of the disclosure are directed to a composition (e.g., a gene therapy compositions) comprising a polynucleotide (e.g., an antibody expression cassette), a vector, or a rAAV particle of the disclosure.

In some aspects, the polynucleotide (e.g., an antibody expression cassette), vector, rAAV particle, or composition of the disclosure is suitable for delivery to a subject in need thereof (e.g., a subject suffering from Graves' Ophthalmopathy). In some aspects, the polynucleotide (e.g., an antibody expression cassette), vector, rAAV particle, or composition of the disclosure is suitable for delivery to an ocular delivery site, a retro or a periorbital delivery site, a retrobulbar delivery site, an extra-ocular muscle delivery site, a connective tissue delivery site, or any combination of delivery sites thereof. In some aspects, the delivery is by injection or infusion. In some aspects, the delivery is by a route of administration selected from intramuscular (IM), intravenous (IV), intralymphatic, intraocular, retroorbital, periorbital, retrobulbar, or any combination thereof. In some aspects, the administration is suitable for delivery to retro or a periorbital fibroblast cells, adipocytes cells, myofibroblast cells, myocyte cells, or any combination thereof. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the polynucleotide (e.g., an antibody expression cassette), vector, rAAV particle, or composition of the disclosure is suitable for a single dose administration. In some aspects, the single dose multiple injections and/or infusions.

Certain aspects of the disclosure are directed to a method of expressing a therapeutic antibody or antigen-binding fragment thereof that binds IGF-1R in a subject in need thereof comprising administering an effective amount of a polynucleotide (e.g., an antibody expression cassette), vector, rAAV particle, or composition of the disclosure to the subject. In some aspect, the administration is a single dose. In some aspects, the single dose comprises multiple injections to an ocular, retro- or a peri-orbital, retrobulbar, ocular muscle, or any other delivery site disclosed herein.

In some aspects, the delivery or administration can be intramuscular (IM), intravenous (IV), intraocular (IC), intralymphatic, periorbital, retrobulbar, or any combination thereof. In some aspects, the delivery or administration is to or near an eye (e.g., one or both eyes), e.g., intraocular, retro- or a peri-orbital, retrobulbar, intramuscular near the eye (e.g., to a levator muscle and/or a glabellar muscle), to connective tissue near the eye, or any combination thereof. In some aspects, the delivery or administration is to retro- or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

Also provided herein is a method of expressing an anti-IGF-1R antibody or an antigen-binding fragment thereof in a subject in need thereof comprising administering an effective amount of polynucleotide (e.g., an antibody expression cassette), vector, rAAV particle, or composition of the disclosure to the subject, wherein the administration is intramuscular (IM), intravenous (IV), intraocular (IC), intralymphatic, periorbital, retrobulbar, or any combination thereof. In some aspects, the gene therapy composition or AAV capsid is administered by periorbital, retrobulbar, and/or intramuscular injection (e.g., injection to a levator muscle and/or a glabellar muscle). In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the administration results in expression of the anti-IGF-1R antibody or antigen-binding fragment thereof in a cell type selected from the group consisting of fibroblasts, adipocytes, myofibroblasts, myocytes, and any combination thereof.

In some aspects, the subject suffers from thyroid eye disease (TED), e.g., active or chronic Graves' Ophthalmopathy.

Non-limiting examples of the various aspects are shown in the present disclosure.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Numerical values presented herein may be written out, or expressed in scientific notation, i.e. x×10^{y}, or scientific E notation xEy.

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein the terms “intraocular” refers to a location that is within or occurring through the eye.

As used herein the terms “intraorbital refers to a location that is within the orbit. The orbit refers to the cavity in the skull that contains the eye, muscles, glands, blood vessels, nerves, etc. related to the eye.

As used herein the term “periorbital” as used herein refers to a location that is situated around or surrounds the orbit (e.g., tissues surrounding or lining the orbit of the eye).

As used herein the term “retroorbital” or “retrobulbar” as used herein refers to a location situated or occurring behind the eyeball.

As used herein the term “retrobulbar” refers to a location that is situated or occurring behind the eyeball.

As used herein “intralymphatic” refers to a location that is associated with a lymph node or a lymph vessel.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, and a lysosome.

Some aspects of the disclosure are directed to biological vectors, which can include viruses, particularly attenuated and/or replication-deficient viruses.

As used herein, the term “promoter” refers to a DNA sequence recognized by the machinery of the cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible by external signals or agents; such elements can be located in 5′ or 3′ regions of the native gene. In some aspects, the promoter is a constitutively active promoter, a cell-type specific promoter, or an inducible promoter.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

As used herein, the term “regulatable promoter” is any promoter whose activity is affected by a cis or trans acting factor (e.g., an inducible promoter, such as an external signal or agent).

As used herein, the term “constitutive promoter” is any promoter that directs RNA production in many or all tissue/cell types at most times, e.g., the human CMV immediate early enhancer/promoter region that promotes constitutive expression of cloned DNA inserts in mammalian cells.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

As used herein, the term “signal peptide,” refers to a polypeptide sequence or combination of sequences that are sufficient to mediate the translocation of a polypeptide to the cell surface. Without being bound by any particular theory, translocation of a polypeptide to the cell surface can be mediated by the secretory pathway, including the translocation of a polypeptide from the cytosol to the endoplasmic reticulum, and the subsequent transport of the polypeptide through the Golgi, and to the cell membrane, where the protein can remain embedded in the cell membrane, or be secreted from the cell. As used herein, “signal peptides,” include naturally-occurring and synthetic signal sequences, signal “patches” and the like. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. In some aspects, the signal peptide is an IL-2 signal peptide. In some aspects, the signal peptide is an IL-10 signal peptide. In some embodiments, the signal peptide is a modified signal peptide.

As used herein, the term “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15 (12): 477-83 (199); Jackson R J and Kaminski, A. RNA 1 (10): 985-1000 (1995). “Under translational control of an IRES” as used herein means that translation is associated with the IRES and proceeds in a cap-independent manner.

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. The term “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site,” refers to a sequence that is incorporated into an expression construct of the disclosure adjacent a self-processing cleavage site, such as a 2A or 2A like sequence, and provides a means to remove additional amino acids that remain following cleavage by the self-processing cleavage sequence. Exemplary 2A peptides include, but are not limited to, P2A, E2A, F2A, and T2A. Exemplary “additional proteolytic cleavage sites” are described herein and include, but are not limited to, furin cleavage sites with the consensus sequence RXK(R)R. Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “expression vector or construct” means any type of genetic construct containing a nucleic acid in which part or all of the nucleic acid encoding sequence is capable of being transcribed.

As used herein, the term “multicistronic” or “multicistronic vector” refers to a nucleic acid sequence having two or more open reading frames (e.g., genes). An open reading frame in this context is a sequence of codons that is translatable into a polypeptide or protein (e.g. a heavy chain or a light chain). “Bicistronic” or “bicistronic vector” refers to a nucleic acid sequence having two open reading frames (e.g., genes). An open reading frame in this context is a sequence of codons that is translatable into a polypeptide or protein (e.g. a heavy chain or a light chain). In some aspects, the construct of the disclosure is a multicistronic (e.g., bicistronic) construct (e.g., comprising a heavy and a light chain).

A “viral vector” refers to a vector created from at least part of a viral genome which can be used to carry or deliver one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector (e.g., myotropic AAV such as AAVMYO (see Weinmann et al. Nat. Comm. 11:5432, 2020)). The terms “AAV genome” and “AAV vector” can be used interchangeably. In some aspects, the AAV vector is modified relative to the wild-type AAV serotype sequence.

As used herein, an “AAV particle” is an AAV virus that comprises an AAV vector genome having at least one payload region (e.g., a polynucleotide (e.g., an antibody expression cassette) encoding a therapeutic protein or peptide) and at least one inverted terminal repeat (ITR) region. In some aspects, the terms “AAV vectors of the present disclosure” or “AAV vectors” refer to AAV vectors comprising a polynucleotide (e.g., an antibody expression cassette) encoding an antibody, e.g., encapsulated in an AAV particle.

A “coding sequence” or a sequence “encoding” a particular molecule (e.g., a therapeutic protein or peptide) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at 5′ (amino) terminus and a translation stop codon at 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AVV vector) that is derived from a second nucleic acid sequence (e.g., another AVV vector) can include a nucleotide sequence that is identical or substantially similar to the nucleotide sequence of the second nucleic acid sequence.

In the case of a polynucleotide disclosed herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

As used herein, the term “administration” refers to the administration of a composition of the present disclosure (e.g., a polynucleotide (e.g., an antibody expression cassette), an AAV vector, rAAV particle, or a composition disclosed herein) to a subject or system. Administration to an animal subject (e.g., to a human) can be by any appropriate route, such as but not limited to periorbital, retrobulbar, intralymphatic, and/or intramuscular injection.

As used herein, the term “modified” refers to a changed state or structure of a molecule of the disclosure. Molecules can be modified in many ways including chemically, structurally, and functionally. In some aspects, the modification is relative to a reference wild-type molecule.

As used herein, the term “synthetic” means produced, prepared, and/or manufactured by the hand of man. Synthesis of polynucleotides or polypeptides or other molecules of the present disclosure can be chemical or enzymatic.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA,” as used herein, refers to a single stranded RNA that encodes the amino acid sequence of one or more polypeptide chains.

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., a shRNA that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., a shRNA that includes a region that is substantially complementary to a region of the antisense strand as that term is defined herein. The antisense and sense strands of a dsRNA, e.g., a shRNA, are hybridized to form a duplex structure.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

The terms “antibody” and “antibodies” refer to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies (e.g., nanobodies). An antigen-binding fragment of an antibody can be derived from any animal species, such as rodents (e.g., mouse, rat, or hamster) and humans or can be artificially produced.

The term “nanobody” or “nanobodies” or “single-domain antibody” or “sdAb” refers to a class of antigen-binding fragments that is a single chain immunoglobulin molecule consisting of a monomeric variable antibody domain, which recognizes and specifically binds to an antigen.

The term “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

The term “bispecific” or “bifunctional antibody” or antigen-binding fragment thereof refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “multispecific antibody” refers to an antibody having specificities for more than two different epitopes, typically non-overlapping epitopes or an antibody that contains more than two distinct antigen-binding sites.

The term “immunoglobulin” is used herein to include antibodies, functional fragments thereof, Fabs, scFvs, single domain antibodies (e.g., nanobodies), DARTs, F(ab′)2, BITEs, and immunoadhesins. These antibody fragments or artificial constructs can include a single chain antibody, a Fab fragment, a univalent antibody, a bivalent of multivalent antibody, or an immunoadhesin. The binding or neutralizing antibody construct can be a monoclonal antibody, a “humanized” antibody, a multivalent antibody, or another suitable construct. An “immunoglobulin molecule” is a protein containing the immunologically-active portions of an immunoglobulin heavy chain and immunoglobulin light chain covalently coupled together and capable of specifically combining with an antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” can be used interchangeably herein. An “immunoglobulin heavy chain” is a polypeptide that contains at least a portion of the antigen binding domain of an immunoglobulin and at least a portion of a variable region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain. An “immunoglobulin light chain” is a polypeptide that contains at least a portion of the antigen binding domain of an immunoglobulin and at least a portion of the variable region. Thus, the immunoglobulin-derived light chain has significant regions of amino acid homology with a member of the immunoglobulin gene superfamily. An “immunoadhesin” is a chimeric, antibody-like molecule that combines the functional domain of a binding protein, usually a receptor, ligand, cell-adhesion molecule, or 1-2 immunoglobulin variable domains with immunoglobulin constant domains, usually including the hinge or GS linker and Fc regions. A “fragment antigen-binding” (Fab) fragment” is a region on an antibody that binds to antigens. It is composed of one constant and one variable domain of each of the heavy and the light chain. With respect to immunoglobulins or antibodies as described herein, each fragment of an immunoglobulin coding sequence can be derived from one or more sources, or synthesized. Suitable fragments can include the coding region for one or more of, e.g., a heavy chain, a light chain, and/or fragments thereof such as the constant or variable region of a heavy chain (CH1, CH2 and/or CH3) and/or or the constant or variable region of a light chain. Alternatively, variable regions of a heavy chain or light chain can be utilized. Where appropriate, these sequences can be modified from the “native” sequences from which they are derived, as described herein. As used herein, the term “immunoglobulin construct” refers to any of the above immunoglobulins or fragments thereof which are encoded by and included in the expression cassettes and viral vectors described herein.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

As used herein, the term “constant region” or “constant domain” are interchangeable and have the meaning common in the art. The constant region is an antibody portion, e.g., a carboxyl terminal portion of a light and/or heavy chain which is not directly involved in binding of an antibody to antigen but can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

As used herein, the term “heavy chain” or “HC” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” or “LC” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system.

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc includes the various allotypes of Fc (see, e.g., Jefferis et al., (2009) mAbs 1:1; Vidarsson G. et al. Front Immunol. 5:520 (published online Oct. 20, 2014)).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Human IgG1 binds to most human Fc receptors and elicits the strongest Fc effector functions. It is considered equivalent to murine IgG2a with respect to their types of activating Fc receptors that it binds to. Conversely, human IgG4 elicits the least Fc effector functions. Vidarsson G. et al. Front Immunol. 5:520 (published online Oct. 20, 2014).

The constant region can be manipulated, e.g., by recombinant technology, to eliminate one or more effector functions. An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with a binding domain (e.g., an antibody variable domain). Accordingly, the term “a constant region without the Fc function” include constant regions with reduced or without one or more effector functions mediated by Fc region.

Effector functions of an antibody can be reduced or avoided by different approaches. Effector functions of an antibody can be reduced or avoided by using antibody fragments lacking the Fc region (e.g., such as a Fab, F(ab′)2, single chain Fv (scFv), or a sdAb consisting of a monomeric VH or VL domain). Alternatively, the so-called aglycosylated antibodies can be generated by removing sugars that are linked to particular residues in the Fc region to reduce the effector functions of an antibody while retaining other valuable attributes of the Fc region (e.g., prolonged half-life and heterodimerization). Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). See, e.g., U.S. Pub. No. 20120100140. Another approach is to employ Fc regions from an IgG subclass that have reduced effector function. For example, IgG2 and IgG4 antibodies are characterized by having lower levels of Fc effector functions than IgG1 and IgG3. The residues most proximal to the hinge region in the CH2 domain of the Fc part are responsible for effector functions of antibodies as it contains a largely overlapping binding site for C1q (complement) and IgG-Fc receptors (FcγR) on effector cells of the innate immune system. Vidarsson G. et al. Front Immunol. 5:520 (published online Oct. 20, 2014). Accordingly, antibodies with reduced or without Fc effector functions can be prepared by generating, e.g., a chimeric Fc region which comprises a CH2 domain from an IgG antibody of the IgG4 isotype and a CH3 domain from an IgG antibody of the IgG1 isotype, or a chimeric Fc region which comprises hinge region from IgG2 and CH2 region from IgG4 (see, e.g., Lau C. et al. J. Immunol. 191:4769-4777 (2013)), or an Fc region with mutations that result in altered Fc effector functions, e.g., reduced or no Fc functions. Such Fc regions with mutations are known in the art. See, e.g., U.S. Pub. No. 20120100140 and U.S. and PCT applications cited therein and An et al., mAbs 1:6, 572-579 (2009); the disclosures of which are incorporated by reference to their entirety.

In some aspects, the antibody (e.g., a monoclonal antibody) or antigen-binding fragment thereof can be modified so that it does not bind to the Fc region. See e.g., Saunders K., Front. Immunol., 10:1296 (2019).

A “hinge,” “hinge domain,” “hinge region,” or “antibody hinge region” are used interchangeably and refer to the domain of a heavy chain constant region that joins the CH1 domain to the CH2 domain and includes the upper, middle, and lower fragments of the hinge (Roux et al., J. Immunol. 1998 161:4083). The hinge provides varying levels of flexibility between the binding and effector regions of an antibody and also provides sites for intermolecular disulfide bonding between the two heavy chain constant regions.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other antibodies having different antigenic. An isolated antibody that specifically binds to an epitope of a protein can, however, have cross-reactivity to other corresponding proteins from different species.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, a humanized antibody or antigen-binding fragment thereof can comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody.

The term “human” antibody (HuMAb) or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof. In some aspects, a human antibody is a fully human antibody, that is, the antibody has an amino acid sequence that is derived from a human immunoglobulin gene locus and does not contain amino acid sequence from a non-human immunoglobulin gene locus. In some aspects, a human antibody is a partially human antibody, that is, the antibody has amino acid sequences that are derived from a human immunoglobulin gene locus and amino acid sequences that are derived from a non-human immunoglobulin gene locus. In some aspects, a human antibody is a fully human, partially human or a humanized antibody. In some aspects, a human antibody is a chimeric human antibody that is the antibody has about a similar amount of amino acid sequences that are derived from a human immunoglobulin gene locus and amino acid sequences that are derived from a non-human immunoglobulin gene locus.

An antibody that is “blocking” or that “blocks” or that is “inhibitory” of that “inhibits” is an antibody that reduces or inhibits (partially or completely) binding of its target protein to one or more ligands when the antibody is bound to the target protein, and/or that reduces or inhibits (partially or completely) one or more activities or functions of the target protein when the antibody is bound to the target protein.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope. The term “epitope mapping” refers to the process of identification of the molecular determinants for antibody-antigen recognition.

The phrase “contacting a cell” (e.g., contacting a cell with a polynucleotide (e.g., an antibody expression cassette), vector, rAAV particle, or composition of the disclosure) as used herein, includes contacting a cell directly or indirectly. In some aspects, contacting a cell with a polynucleotide (an antibody expression cassette), vector, rAAV particle, or composition includes contacting a cell in vitro with a polynucleotide (an antibody expression cassette), vector, rAAV particle, or composition or contacting a cell in vivo with a polynucleotide (an antibody expression cassette), vector, rAAV particle, or composition. Thus, for example, the polynucleotide (an antibody expression cassette), vector, rAAV particle, or composition can be put into physical contact with the cell by the individual performing the method, or alternatively, the polynucleotide (an antibody expression cassette), vector, rAAV particle, or composition can be put into a situation that will permit or cause it to subsequently come into contact with the cell.

In some aspects, contacting a cell in vitro can be done, for example, by incubating the cell with the polynucleotide (an antibody expression cassette), vector, rAAV particle, or composition. In some aspects, contacting a cell in vivo can be done, for example, by injecting the polynucleotide (an antibody expression cassette), vector, rAAV particle, or composition of the disclosure into or near the tissue where the cell is located (e.g., retrobulbar, periorbital, or ocular muscle), or by injecting the polynucleotide (an antibody expression cassette), vector, rAAV particle, or composition into another area, e.g., the bloodstream or the subcutaneous space, such that the agent will subsequently reach the tissue where the cell to be contacted is located. For example, the AAV vector genome can be encapsulated and/or coupled to a ligand that directs the AAV vector genome to a site of interest. Combinations of in vitro and in vivo methods of contacting are also possible. For example, a cell can be contacted in vitro with a polynucleotide (an antibody expression cassette), vector, rAAV particle, or composition of the disclosure and subsequently transplanted into a subject.

In some aspects, contacting a cell with a polynucleotide (an antibody expression cassette), vector, rAAV particle, or composition of the disclosure includes “introducing” or “delivering” (directly or indirectly) the polynucleotide (an antibody expression cassette), vector, rAAV particle, or composition into the cell by facilitating or effecting uptake or absorption into the cell. Introducing a polynucleotide (an antibody expression cassette), vector, rAAV particle, or composition into a cell can be in vitro and/or in vivo. For example, for in vivo introduction, a polynucleotide (an antibody expression cassette), vector, rAAV particle, or composition can be injected into a specific tissue site (e.g., the locus where a therapeutic effect is desired) or administered systemically (e.g., administering a polynucleotide (an antibody expression cassette), vector, or rAAV particle targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., a polynucleotide, expression cassette, vector, rAAV particle, or composition of the disclosure refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., a polynucleotide (an antibody expression cassette), vector, rAAV particle, or composition disclosed herein) is an amount that results in a beneficial or desired result in a subject as compared to a control.

The amount of a given agent (e.g., a polynucleotide (an antibody expression cassette), vector, rAAV particle, or composition of the disclosure) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., an antibody expression cassette comprising a promoter operably linked to a nucleic acid encoding a therapeutic molecule as disclosed herein) into an individual's cells and/or tissues to treat, reduce the symptoms of, or reduce the likelihood of a disease. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

The term “prophylactically effective amount,” as used herein, includes the amount of an agent, (e.g., a polynucleotide (an antibody expression cassette), vector, rAAV particle, or composition disclosed herein) that, when administered to a subject having or predisposed to have a disease or disorder (e.g., Graves' Orbitopathy). Ameliorating the disease or disorder includes slowing the course of the disease or disorder or reducing the severity of later-developing disease or disorder. The “prophylactically effective amount” can vary depending on the characteristics of the agent, e.g., a polynucleotide (an antibody expression cassette), vector, rAAV particle, or composition of the disclosure, how the agent is administered, the degree of risk of disease, and the history, age, weight, family history, genetic makeup, the types of preceding or concomitant treatments, if any, and other individual characteristics of the patient to be treated.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference. A level of a protein can be expressed in mass/vol (e.g., g/dL, mg/mL, μg/mL, ng/mL) or percentage relative to total protein or mRNA in a sample.

The term “pharmaceutical composition,” as used herein, represents a composition comprising a compound or molecule described herein, e.g., a polynucleotide (an antibody expression cassette), vector, or rAAV particle disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “subject” refers to any organism to which a composition disclosed herein, e.g., a polynucleotide (an antibody expression cassette), vector, rAAV particle, or composition of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. In some aspects, treating reduces or lessens the symptoms associated with a disease or disorder. In some aspects, the treating results in a beneficial or desired clinical result.

As used herein “Graves' Orbitopathy” (GO) refers to active or chronic stages of an autoimmune orbital inflammatory thyroid-associated condition. “Active” GO or “dynamic” GO can last approximately 6 months and up to 24 months and can be followed by “inactive” or “chronic” GO.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years.

### II. Therapeutic Antibodies

The present disclosure provides polynucleotides (e.g., antibody expression cassettes), vectors, and rAAV particles for delivery and expression of therapeutic anti-IGF-1R antibodies to a cell or subject. In some aspects, the antibody expression cassette comprises a promoter operably linked to a nucleic acid encoding an antibody or antigen binding fragment thereof that binds an insulin-like growth factor-1 receptor (also referred to interchangeably herein as an “anti-IGF receptor antibody”, “anti-IGF-1 receptor antibody”, “anti-IGF-1R antibody” and “anti-IGF-1R antibody” herein).

In some aspects, the anti-IGF-1R antibody is an antibody or antigen-binding fragment thereof selected from a monoclonal antibody, a bispecific antibody, or a multispecific antibody or antigen-binding fragment thereof. In some aspects, the therapeutic protein is an antibody fragment selected from a Fab, a Fab′, a F(ab′)2, a Fv fragments, a linear antibody, or a single chain antibody (e.g., a nanobody).

In some aspects, the antibody is selected from the group consisting of a monoclonal antibody, a bispecific antibody, nanobody, and a multispecific antibody.

In some aspects, the antibody is a monoclonal antibody.

In some aspects, the antibody expression cassette disclosed herein comprises a nucleic acid sequence encoding a heavy chain (HC) and/or a light chain (LC). In some aspects, the antibody expression cassette disclosed herein comprises a nucleic acid sequence encoding a variable heavy chain (VH) and/or a variable light chain (VL).

In some aspects, the antibody (e.g., a monoclonal antibody) or antigen-binding fragment thereof is a chimeric antibody.

In some aspects, the antibody (e.g., a monoclonal antibody) or antigen-binding fragment thereof is a humanized antibody.

In some aspects, the antibody (e.g., a monoclonal antibody) or antigen-binding fragment thereof is a human antibody. In some aspects, a human antibody is a fully human antibody that comprises an amino acid sequence that is derived from a human immunoglobulin gene locus and does not contain amino acid sequence from a non-human immunoglobulin gene locus. In some aspects, a human antibody is a partially human antibody that comprises the amino acid sequences that are derived from a human immunoglobulin gene locus and amino acid sequences that are derived from a non-human immunoglobulin gene locus. In some aspects, a human antibody is a fully human, partially human or a humanized antibody. In some aspects, a human antibody is a chimeric human antibody that is the antibody has about a similar amount of amino acid sequences that are derived from a human immunoglobulin gene locus and amino acid sequences that are derived from a non-human immunoglobulin gene locus.

In some aspects, the anti-IGF-1R antibody is teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or fragments, variants, or derivatives thereof.

In some aspects, the anti-IGF-1R antibody is VRDN-01100, or VRDN-02700, or fragments, variants, or derivatives thereof. In some aspects, the anti-IGFR antibody comprises SEQ ID NO: 113 (corresponding to VRDN-01100). In some aspects, the anti-IGFR antibody comprises SEQ ID NO: 116 (corresponding to VRDN-002700).

In some aspects, the anti-IGF-1R antibody is teprotumumab, or fragments, variants, or derivatives thereof.

In some aspects, the antibody expression cassette comprises a nucleic acid encoding a signal peptide operably linked to a nucleic acid encoding an antibody or antigen binding fragment thereof that binds an insulin-like growth factor-1 receptor. In some aspects the signal peptide is an endogenous signal peptide for HGH and variants thereof; an endogenous signal peptide for interferons and variants thereof, including the signal peptide of type I, II and III interferons and variants thereof; an endogenous signal peptides for known cytokines and variants thereof, such as the signal peptide of erythropoietin (EPO), insulin, TGF-β1, TNF, IL1-α, and IL1-β, and variants thereof. In some embodiments, the signal peptide is a modified signal peptide. In some aspects, the signal peptide is an IL-2 signal peptide. In some aspects, the signal peptide is an IL-10 signal peptide. In some aspects the signal peptide comprises and amino acid sequence with at least 85%, 86%, 87%, 88%, 89%, 90%, 91%, 92%, 93%, 94%, 95%, 96%, 97%, 98%, 99%, or 100% identity to SEQ ID NO: 119 or 120. In some aspects, the nucleic acid sequence encoding the signal peptide comprises a nucleic acid sequence at least 85%, 86%, 87%, 88%, 89%, 90%, 91%, 92%, 93%, 94%, 95%, 96%, 97%, 98%, 99%, or 100% identical to any of SEQ ID NOs: 121 or 122.

In certain aspects, a composition comprising a delivery vector, e.g., a viral vector, comprising nucleic acids encoding an immunoglobulin disclosed herein (e.g., an anti-IGF-1R antibody) is suitable for delivery to a subject in need thereof.

In some aspects, an antibody expression cassette comprising a nucleic acid sequence encoding an anti-IGF-1R antibody can be packaged in a viral vector (e.g., an AAV vector) disclosed herein, wherein the nucleic acid sequence encoding an anti-IGF-1R antibody is operably linked with the promoter. In some aspects, the promoter can drive the expression of the anti-IGF-1R antibody in a host cell (e.g., a fibroblast cell, an adipocyte cell, a myofibroblast cell, a myocyte cell, a muscle cell, or any combination thereof). In some aspects, the polynucleotide (e.g., an antibody expression cassette), vector, rAAV particle, or composition comprising the nucleic acid encoding an anti-IGF-1R antibody can be administered to an ocular, a retrobulbar, intralymphatic, a periorbital and/or a muscle tissue (e.g., a levator muscle and/or a glabellar muscle). In some aspects, the polynucleotide (e.g., an antibody expression cassette), vector, rAAV particle, or composition comprising the nucleic acid encoding an anti-IGF-1R antibody can be administered intramuscularly, intralymphatically, intracutaneously, intravenously, intraocularly, retrobulbar, periorbital, or any combination thereof. In some aspects, the polynucleotide (e.g., an antibody expression cassette), vector, rAAV particle, or composition comprising the nucleic acid encoding an anti-IGF-1R antibody can be administered intramuscularly to an extraocular muscle. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the polynucleotide (e.g., an antibody expression cassette), vector, rAAV particle, or composition comprising the nucleic acid encoding an anti-IGF-1R antibody can be administered intralymphatic into a pre-auricular and/or submandibular lymph node.

In some aspects, the polynucleotide (e.g., an antibody expression cassette), vector, rAAV particle, or composition comprising the nucleic acid encoding the anti-IGF-1R antibody is administered to a periorbital tissue. In some aspects, the polynucleotide (e.g., an antibody expression cassette), vector, rAAV particle, or composition comprising the nucleic acid encoding the anti-IGF-1R antibody is administered to periorbital tissue. In some aspects, the polynucleotide (e.g., an antibody expression cassette), vector, rAAV particle, or composition comprising the nucleic acid encoding the anti-IGF-1R antibody is administered intramuscular to a periorbital muscle. In some aspects, the polynucleotide (e.g., an antibody expression cassette), vector, rAAV particle, or composition comprising the nucleic acid encoding the anti-IGF-1R antibody is administered intramuscular to a retrobulbar muscle. In some aspects, the polynucleotide (e.g., an antibody expression cassette), vector, rAAV particle, or composition comprising the nucleic acid encoding the anti-IGF-1R antibody is administered intramuscular to a facial muscle. In some aspects, the polynucleotide (e.g., an antibody expression cassette), vector, rAAV particle, or composition comprising the nucleic acid encoding the anti-IGF-1R antibody is administered to a periorbital or retroorbital connective tissue. In some aspects, the polynucleotide (e.g., an antibody expression cassette), vector, rAAV particle, or composition comprising the nucleic acid encoding the anti-IGF-1R antibody is administered intramuscular to a levator muscle and/or a glabellar muscle. In some aspects, the polynucleotide (e.g., an antibody expression cassette), vector, rAAV particle, or composition comprising the nucleic acid encoding the anti-IGF-1R antibody is administered to a fibroblast in the periorbital or retroorbital connective tissue. In some aspects, the polynucleotide (e.g., an antibody expression cassette), vector, rAAV particle, or composition comprising the nucleic acid encoding the anti-IGF-1R antibody is administered to a myofibroblast in the periorbital or retroorbital connective tissue. In some aspects, the polynucleotide (e.g., an antibody expression cassette), vector, rAAV particle, or composition comprising the nucleic acid encoding the anti-IGF-1R antibody is administered to an adipocyte in the periorbital or retroorbital connective tissue. In some aspects, the polynucleotide (e.g., an antibody expression cassette), vector, rAAV particle, or composition comprising the nucleic acid encoding the anti-IGF-1R antibody is administered to a myocytes in the periorbital or retroorbital connective tissue. In some aspects, the nucleic acid encoding a protein or peptide disclosed herein is suitable for delivery to a periorbital and/or retroorbital tissue. In some aspects, the nucleic acid encoding a protein or peptide disclosed herein is suitable for delivery to periorbital tissue. In some aspects, the nucleic acid encoding a protein or peptide disclosed herein is suitable for intramuscular delivery to a periorbital or retroorbital muscle. In some aspects, the nucleic acid encoding a protein or peptide disclosed herein is suitable for intramuscular delivery to a facial muscle. In some aspects, the nucleic acid encoding a protein or peptide disclosed herein is suitable for delivery to a periorbital or retroorbital connective tissue. In some aspects, the administration is transconjunctival into the periorbital space. In some aspects, the administration is intralymphatic to the pre-auricular or submandibular node.

In some aspects, the nucleic acids encoding a protein or peptide disclosed herein is suitable for delivery to other delivery sites disclosed herein.

### II.A.1 Antibodies

Certain aspects of the disclosure are directed to polynucleotides (e.g., an antibody expression cassette), vectors, rAAV particles, or compositions comprising the nucleic acid encoding antibodies (e.g., monoclonal antibodies) and antigen-binding fragments thereof which specifically bind to an insulin-like growth factor 1 receptor (IGF-1R), such as human IGF-1R. In some aspects, the encoded anti-IGF-1R antibody is an anti-insulin-like growth factor-1 receptor (anti-IGF-1R) antibody. In some aspects, the encoded anti-IGF-1R antibody comprises the amino acid sequence of teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or fragments, variants or derivatives thereof.

In some aspects, the encoded anti-IGF-1R antibody comprises the amino acid sequence of VRDN-01100, or VRDN-02700, or fragments, variants, or derivatives thereof.

In some aspects, the encoded anti-IGF-1R antibody comprises the amino acid sequence of SEQ ID NO: 113 (corresponding to VRDN-01100). In some aspects, the anti-IGFR antibody comprises SEQ ID NO: 116 (corresponding to VRDN-002700).

In some aspects, the encoded anti-IGF-1R antibody comprises the amino acid sequence of teprotumumab, or fragments, variants, or derivatives thereof.

In some aspects, the disclosure is directed to polynucleotides (e.g., an antibody expression cassette), vectors, rAAV particles, or compositions of the disclosure comprising a promoter operably linked to a nucleic acid encoding an immunoglobulin, e.g., an antibody or antigen-binding fragment thereof that binds to IGF-1R.

In some aspects, the polynucleotides (e.g., an antibody expression cassette), vectors, rAAV particles, or compositions of the disclosure used in the methods disclosed herein encode an antibody (e.g., monoclonal antibodies or antigen-binding fragments thereof) having the CDR and/or variable region sequences of teprotumumab or antibodies having at least 80% identity (e.g., at least about 85%, at least about 90%, at least about 95%, at least about 96%, at least about 97%, at least about 98%, at least about 99%, or about 100% identity) to their variable region or CDR sequences of teprotumumab. In some aspects, the gene therapy construct encoding an anti-IGF-1R antibody (e.g., teprotumumab) is a multicistronic (e.g., bicistronic) construct (e.g., comprising a heavy chain and a light chain). In some aspects, the multicistronic (e.g., bicistronic) construct further comprises an F2A or IRES element.

In some aspects, the polynucleotides (e.g., an antibody expression cassette), vectors, rAAV particles, or compositions disclosed herein comprises a nucleic acid encoding an antibody comprising a heavy chain and a light chain of teprotumumab or an antigen-binding fragment thereof. In some aspects, the polynucleotides (e.g., an antibody expression cassette), vectors, rAAV particles, or compositions disclosed herein comprise nucleic acid sequences which are modified relative to wild-type (unmodified) teprotumumab coding sequences.

In some aspects, the anti-IGF-1R antibody comprises a heavy chain and a light chain. In some aspects, the nucleic acid sequence encoding the heavy chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 35-37 (or a HC coding sequence disclosed in Table 11). In some aspects, the encoded HC comprises SEQ ID NO: 38 (or the HC amino acid sequence in Table 12). In some aspects, the nucleic acid sequence encoding the light chain comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39-41 (or a LC coding sequence disclosed in Table 13). In some aspects, the encoded LC comprises SEQ ID NO: 42 (or the LC amino acid sequence in Table 14).

In some aspects, the nucleic acid sequence encoding the heavy chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 25-27 (or a VH coding sequence disclosed in Table 5). In some aspects, the encoded VH comprises SEQ ID NO: 28 or SEQ ID NO: 91 (or any of the VH amino acid sequences in Table 6). In some aspects, the nucleic acid sequence encoding the light chain variable region comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 29-31 (or a VL coding sequence disclosed in Table 7). In some aspects, the encoded VL comprises SEQ ID NO: 32 or SEQ ID NO: 92 (or any of the VL amino acid sequences in Table 8).

In some aspects, the heavy chain comprises a heavy chain variable region (VH) comprising a complementarity determining region (CDR) 1, a VH CDR2, and a VH CDR3. In some aspects, the VH CDRs 1-3 correspond to the CDRs of teprotumumab. In some aspects, the nucleic acid sequence encoding the VH CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10 (or a VH CDR1 coding sequence disclosed in Table 3 or Table 5); the nucleic acid sequence encoding the VH CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14 (or a VH CDR2 coding sequence disclosed in Table 3 or Table 5); and the nucleic acid sequence encoding the VH CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15 (or a VH CDR3 coding sequence disclosed in Table 3 or Table 5).

In some aspects, the light chain comprises a light chain variable region (VL) comprising a complementarity determining region (CDR) 1, a VL CDR2, and a VL CDR3. In some aspects, the VL CDRs 1-3 correspond to the CDRs of teprotumumab. In some aspects, the nucleic acid sequence encoding the VL CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16 (or a VL CDR1 coding sequence disclosed in Table 4 or Table 7); the nucleic acid sequence encoding the VL CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23 (or a VL CDR2 coding sequence disclosed in Table 4 or Table 7); and the nucleic acid sequence encoding the VL CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24 (or a VL CDR3 coding sequence disclosed in Table 4 or Table 7).

In some aspects, the polynucleotide disclosed herein encodes a single-domain antibody (e.g., a nanobody) comprising either (i) a heavy chain variable region (VH) comprising a complementarity determining region (CDR) 1, a VH CDR2, and/or a VH CDR3 or (ii) a light chain variable region (VL) comprising a CDR1, a VL CDR2, and/or a VL CDR3. In some aspects, the encoded VH CDRs and/or VL CDRs are selected from the corresponding CDRs of teprotumumab.

In some aspects, the polynucleotide disclosed herein encodes the amino acid sequence of teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or fragments, variants or derivatives thereof.

In some aspects, the polynucleotide disclosed herein encodes the amino acid sequence of VRDN-01100, or VRDN-02700, or fragments, variants, or derivatives thereof.

In some aspects, the polynucleotide disclosed herein encodes the amino acid sequence of SEQ ID NO: 113 (corresponding to VRDN-01100). In some aspects, the anti-IGFR antibody comprises SEQ ID NO: 116 (corresponding to VRDN-002700).

In some aspects, the polynucleotide disclosed herein encodes the amino acid sequence of teprotumumab, or fragments, variants, or derivatives thereof.

In some aspects, the polynucleotide disclosed herein encodes an antibody or antigen-binding fragment thereof comprising the six CDRs listed in Tables 1 and 2 (i.e., any set of three VH CDRs listed in Table 1 and any set of three VL CDRs listed in Table 2). In some aspects, the polynucleotide disclosed herein comprises the nucleotide sequences of the six CDRs listed in Tables 3 and 4 (i.e., the nucleic acids encoding the three VH CDRs as listed in Table 3 and the nucleic acids encoding the three VL CDRs as listed in Table 4).

TABLE 1
Variable Heavy Chain CDR (VH CDR) Amino Acid
Sequences

| VH-CDR1 | VH-CDR2 | VH-CDR3 |
|---|---|---|
| SYGMH | IIWFDGSSTYYADSVRG | ELGRRYFDL |
| (SEQ ID NO: 1) | (SEQ ID NO: 2) | (SEQ ID NO: 3) |
| SYGMH | IIWFDGSSKYYGDSVKG | ELGRRYFDL |
| (SEQ ID NO: 1) | (SEQ ID NO: 89) | (SEQ ID NO: 3) |

TABLE 2
Variable Light Chain CDR (VL CDR) Amino Acid
Sequences

| VL-CDR1 | VL-CDR2 | VL-CDR3 |
|---|---|---|
| RASQSVSSYLA | DASKRAT | QQRSKWPPWT |
| (SEQ ID NO: 4) | (SEQ ID NO: 5) | (SEQ ID NO: 6) |
| RASQSVSSYLA | DASNRAT | QQRSKWPPWT |
| (SEQ ID NO: 4) | (SEQ ID NO: 90) | (SEQ ID NO: 6) |

TABLE 3
Variable Heavy Chain CDR (VH CDR) Nucleic Acid
Sequences

| Description | VH-CDR1 | VH-CDR2 | VH-CDR3 |
|---|---|---|---|
| anti-IGF- | tcctacgg | atcatttgg | gagctgggccg |
| 1R #1 | gatgcat | ttcgacgga | ccggtattttg |
| | (SEQ ID | catcttact | acctt |
| | NO: 7) | tattacgcc | (SEQ ID |
| | | gatagtgtt | NO: 9) |
| | | cgaggg | |
| | | (SEQ ID | |
| | | NO: 8) | |
| anti-IGF- | tcctatgg | atcatttgg | gagctgggccg |
| 1R #2 | gatgcat | tttgatgga | cagatattttg |
| | (SEQ ID | tcttcaact | acctt |
| | NO: 10) | tattatgct | (SEQ ID |
| | | gatagtgtt | NO: 12) |
| | | agaggg | |
| | | (SEQ ID | |
| | | NO: 11) | |
| anti-IGF- | tcctatgg | atcatttgg | gagctgggcag |
| 1R #3 | gatgcat | tttgatgga | aaggtattttg |
| | (SEQ ID | tcttcaact | acctt |
| | NO: 10) | tattatgct | (SEQ ID |
| | | gatagtgtt | NO: 15) |
| | | aggggg | |
| | | (SEQ ID | |
| | | NO: 14) | |

TABLE 4
Variable Light Chain CDR (VL CDR) Nucleic Acid
Sequences

| Description | VL-CDR1 | VL-CDR2 | VL-CDR3 |
|---|---|---|---|
| anti-IGF- | agggcctc | gacgcaagc | cagcagcgg |
| 1R #1 | ccagtctg | aaacgagcc | agtaagtgg |
| | tttcaagt | acc | ccaccctgg |
| | tatctggc | (SEQ ID | aca |
| | t | NO: 17) | (SEQ ID |
| | (SEQ ID | | NO: 18) |
| | NO: 16) | | |
| anti-IGF- | agggcctc | gatgcaagc | cagcagcgg |
| 1R #2 | ccagtctg | aaaagggcc | agtaagtgg |
| | tttcaagt | aca | ccaccctgg |
| | tatctggc | (SEQ ID | aca |
| | t | NO: 20) | (SEQ ID |
| | (SEQ ID | | NO: 21) |
| | NO: 16) | | |
| anti-IGF- | agggcctc | gatgcaagc | cagcagaga |
| 1R #3 | ccagtctg | aaaagagcc | agtaagtgg |
| | tttcaagt | aca | ccaccctgg |
| | tatctggc | (SEQ ID | aca |
| | t | NO: 23) | (SEQ ID |
| | (SEQ ID | | NO: 24) |
| | NO: 16) | | |

In some aspects, the polynucleotide disclosed herein comprises a nucleic acid sequence listed in Table 5. In some aspects, the polynucleotide disclosed herein encodes an antibody variable heavy chain (VH) sequence listed in Table 6 or antigen-binding fragment thereof.

TABLE 5
Variable Heavy Chain (VH) Nucleic Acid
Sequences

| | VH Nucleic Acid Sequence |
|---|---|
| | (SEQ ID NO) |
| VH #1 | caggttgagttagtggaatctggagggggtgtagt |
| anti-IGF- | gcaacccggcagatcacagaggctgagttgcgcag |
| 1R VH | cgtcgggattcacatttagctcctacgggatgcat |
| | tgggtccgccaggcccctggcaagggtctagagtg |
| | ggtggctatcatttggttcgacggatcttcaactt |
| | attacgccgatagtgttcgagggcgttttacgata |
| | agccgggacaactccaaaaataccctctatctgca |
| | aatgaactctttgagagcagaagatacagccgtgt |
| | acttctgtgctagggagctgggccgccggtatttt |
| | gacctttggggcagaggaactctcgtcagcgtatc |
| | ctca (SEQ ID NO: 25) |
| VH #2 | Caggttgagttagtggaatctggagggggtgtagt |
| anti-IGF- | gcaacctggcagatcacagaggctgagttGcgcag |
| 1R VH | caagtggattcacatttagctcctatgggatgcat |
| | tgggtccgccaggcccctggcaagGgtctagagtg |
| | ggtggctatcatttggtttgatggatcttcaactt |
| | attatgctgatagtgttagAgggcgctttactata |
| | agcagggacaactccaaaaataccctctatctgca |
| | aatgaactctttgagagcagaagatacagctgtgt |
| | acttctgtgctagggagctgggccgcagatatttt |
| | gacctttgg ggcagaggaactcttgtctctgtat |
| | cctca (SEQ ID NO: 26) |
| VH #3 | caggttgagttagtggaatctggagggggtgtagt |
| anti-IGF- | gcaacctggcagatcacagaggctgagttgtgcag |
| 1R VH | caagtggattcacatttagctcctatgggatgcat |
| | tgggtcagacaggcccctggcaagggtctagagtg |
| | tggggctatcatttggtttgatggatcttcaactt |
| | attatgctgatagtgttagggggagatttactata |
| | agcagggacaactccaaaaataccctctatctgca |
| | aatgaactctttgagagcagaagatacagctgtgt |
| | acttctgtgctagggagctgggcagaaggtatttt |
| | gacctttggggcagaggaactcttgtctctgtatc |
| | ctca (SEQ ID NO: 27) |

TABLE 6
Variable Heavy Chain (VH) Amino Acid
Sequences
VH Amino Acid Sequence (SEQ ID NO)
QVELVESGGGVVQPGRSQRLSCAASGFTFSSYGMHWVRQAPGKGLE
WVAIIWFDGSSTYYADSVRGRFTISRDNSKNTLYLQMNSLRAEDTAV
YFCARELGRRYFDLWGRGTLVSVSS
(SEQ ID NO: 28)
QVQLVESGGGVVQPGRSLRLSCAASGFTFSSYGMHWVRQAPGKGLE
WMAIIWFDGSSKYYGDSVKGRFTISRDNSKNTLYLQMNSLRADDTA
VYYCARELGRRYFDLWGRGTLVTVSS(SEQ ID NO: 91)

In some aspects, the polynucleotide disclosed herein comprises a nucleic acid sequence listed in Table 7. In some aspects, the polynucleotide disclosed herein encodes an antibody variable light chain (VL) comprising a sequence listed in Table 8 or antigen-binding fragment thereof.

TABLE 7
Variable Light Chain (VL) Nucleic Acid
Sequences

| | VL Nucleic Acid Sequence (SEQ ID NO) |
|---|---|
| VL #1 | gagattgtcttaacacaatcacccgcaactctgagt |
| anti-IGF- | ctatcgcctggcgaaagagcgacgctcagctgtagg |
| 1R VL | gcctcccagtctgtttcaagttatctggcttggtac |
| | cagcaaaagccaggacaggccccgcgcttgctgata |
| | tatgacgcaagcaaacgagccaccgggatccccgct |
| | cgtttctccggttctggcagcggaacagattttact |
| | cttaccatttcctcactcgagcctgaagacttcgca |
| | gtgtactattgccagcagcggagtaagtggccaccc |
| | tggacatttgggcaaggcaccaaagtagaga |
| | gcaag (SEQ ID NO: 29) |
| VL #2 | gagattgtcttaacacaatcacccgcaactctgagt |
| anti-IGF- | ctaagtcctggagaaagagcaactctcagctgtagg |
| 1R VL | gcctcccagtctgtttcaagttatctggcttggtac |
| | cagcaaaagccaggacaggccccgcgcttgctgata |
| | tatgatgcaagcaaaagggccacagggatccccgct |
| | agattcagcggttctggcagcggaacagattttact |
| | cttaccatttcctcacttgagcctgaagactttgca |
| | gtgtactattgccagcagcggagtaagtggccaccc |
| | tggacatttgggcaaggcaccaaagtagaga |
| | gcaag (SEQ ID NO: 30) |
| VL #3 | Gagattgtcttaacacaatcacctgcaactctgagt |
| anti-IGF- | ctaagtcctggagaaagagcaactctcagctgtagg |
| 1R VL | gcctcccagtctgtttcaagttatctggcttggtac |
| | cagcaaaagccaggacaggccccaaggttgctgata |
| | tatgatgcaagcaaaagagccacagggatccctgct |
| | aggttctctggttctggctctggaacagattttact |
| | cttaccatttcctcacttgagcctgaagactttgca |
| | gtgtactattgccagcagagaagtaagtggccaccc |
| | tggacatttgggcaaggcaccaaagtagagagcaag |
| | (SEQ ID NO: 31) |

TABLE 8
Variable Light Chain (VL) Amino Acid Sequences
VL Amino Acid Sequence (SEQ ID NO)
EIVLTQSPATLSLSPGERATLSCRASQSVSSYLAWYQQKPGQAPRLLI
YDASKRATGIPARFSGSGSGTDFTLTISSLEPEDFAVYYCQQRSKWPP
WTFG QGTKVESK (SEQ ID NO: 32)
EIVLTQSPATLSLSPGERATLSCRASQSVSSYLAWYQQKPGQAPRLLI
YDASNRATGIPARFSGSGSGTDFTLTISSLEPEDFAVYYCQQRSKWPP
WTFGQGTKVEIK (SEQ ID NO: 92)

In some aspects, the polynucleotide disclosed herein comprising a nucleic acid selected from Table 5 (e.g., a nucleic acid encoding a VH of Table 6) and a nucleic acid from Table 7 (e.g., a nucleic acid encoding a VL of Table 8).

In some aspects, the polynucleotide disclosed herein comprises a nucleic acid sequence shown in Table 9.

TABLE 9
Light Chain Constant Region Nucleic Acid
Sequences

| Descrip- | |
|---|---|
| tion | |
| (SEQ ID | Light Chain Constant Region |
| NO) | Nucleic Acid Sequence |
| LC | agaactgtggccgcgccttctgtcttcatctttccacc |
| Constant | ctccgatgaacagctgaaatcgggtacggcttcagtgg |
| #1 | tttgtctgctcaataacttctacccgagggaggccaag |
| (SEQ ID | gtgcagtggaaggtcgacaatgcattgcagagcggaaa |
| NO: 33) | cagtcaagagtctgtaacagaacaggattccaaagaca |
| | gcacctattcactttccagtactctgaccttatctaag |
| | gccgattacgagaaacacaaggtgtacgcttgcgaagt |
| | tacacatcaggggctgagctcccctgtgaccaaaagct |
| | ttaatcgcggcgagtgt |
| LC | agaactgtggccgcgccttctgtcttcatctttccacc |
| Constant | ctctgatgaacagctgaaatcaggtactgcttcagtgg |
| #2 | tttgtctgctcaataacttctacccaagggaggccaag |
| (SEQ ID | gtgcagtggaaggttgacaatgcattgcagagcggaaa |
| NO: 85) | cagtcaagagtctgtaacagaacaggattccaaagaca |
| | gcacctattcactttccagtactctgaccttatctaag |
| | gctgattatgagaaacacaaggtgtatgcttgtgaagt |
| | tacacatcaggggctgagctcccctgtgaccaaaagct |
| | ttaataggggggagtgt |
| LC | agaactgtggctgcaccttctgtcttcatctttccacc |
| Constant | ctctgatgaacagctgaaatcaggtactgcttcagtgg |
| #3 | tttgtctgctcaataacttctacccaagggaggccaag |
| (SEQ ID | gtgcagtggaaggttgacaatgcattgcagtctggaaa |
| NO: 86) | cagtcaagagtctgtaacagaacaggattccaaagaca |
| | gcacctattcactttccagtactctgaccttatctaag |
| | gctgattatgagaaacacaaggtgtatgcttgtgaagt |
| | tacacatcaggggctgagctcccctgtgaccaaaagct |
| | ttaataggggggagtgt |

In some aspects, the polynucleotide disclosed herein comprises a nucleic acid sequence shown in Table 10.

TABLE 10
Heavy Chain Constant Region Nucleic Acid
Sequences

| Descrip- | |
|---|---|
| tion | |
| (SEQ ID | Heavy Chain Constant Region |
| NO) | Nucleic Acid Sequence |
| HC | gcaagtaccaaggggccaagcgtgttcccgctggcccc |
| Constant | ctcttccaaatcgacatcaggtggcaccgcggctctgg |
| #1 | gatgcctcgttaaggattactttcctgaaccagtgact |
| (SEQ ID | gtcagctggaatagtggggccttgacgtctggcgtgca |
| NO: 34) | cacattccccgcagtccttcagtccagcggtctgtatt |
| | cattatccagtgtggttaccgtgccttctagctccctg |
| | ggaactcagacctacatctgtaacgtaaatcataaacc |
| | aagcaacacaaaggtggacaagaaagtcgagcccaagt |
| | cttgcgataaaacccacacttgtccgccttgcccagcc |
| | cccgagctactcgggggcccttcagtttttctgttccc |
| | acccaagcctaaagacacattgatgattagtaggacgc |
| | ccgaagtgacctgtgtcgtggtggatgtttcccatgag |
| | gacccagaagtaaagttcaattggtacgtggatggcgt |
| | cgaggtgcacaacgctaagactaaaccgcgcgaagagc |
| | agtataatagcacataccgagtcgtgtcggttctgacc |
| | gtgcttcaccaagactggctcaacggaaaggagtataa |
| | atgcaaggtatctaataaggccctgcccgcacctatcg |
| | aaaaaactatttcaaaggctaaagggcagccacgtgag |
| | ccccaggtctacaccctgcctccatcccgggacgaatt |
| | aacaaagaaccaggtgagtctcacgtgcctggttaaag |
| | gtttttatcccagcgatatcgccgtggagtgggagtct |
| | aacggccaaccggaaaataactacaagaccacacctcc |
| | agtcttggactccgatggaagcttctttctttactcaa |
| | agctgactgtggacaaaagtagatggcagcaggggaat |
| | gtgttcagctgttccgttatgcatgaggcgctacacaa |
| | ccattatacccagaagtctctctcgctgtcacccggca |
| | aa |
| HC | gcaagtaccaaggggccatctgtgttcccgctggcccc |
| Constant | ctcttccaaaagtacatcaggtggcaccgcggctctgg |
| #2 | gatgcttggttaaggattactttcctgaaccagtgact |
| (SEQ ID | gtcagctggaatagtggggccttgacttctggggtgca |
| NO: 87) | cacattccccgcagtccttcagtccagcggtctgtatt |
| | cattatccagtgtggttacagtgccttctagctccctg |
| | ggaactcagacctacatctgtaatgtaaatcataaacc |
| | aagcaacacaaaggtggacaagaaagttgagcccaagt |
| | cttgtgataaaacccacacttgtccgccttgcccagcc |
| | cctgagctacttgggggcccttcagtttttctgttccc |
| | acccaagcctaaagacacattgatgattagtaggactc |
| | ctgaagtgacctgtgttgtggtggatgtttcccatgag |
| | gacccagaagtaaagttcaattggtatgtggatggagt |
| | tgaggtgcacaatgctaagactaaaccgagggaagagc |
| | agtataatagcacatacagagttgtgtcagttctgaca |
| | gtgcttcaccaagactggctcaatggaaaggagtataa |
| | atgcaaggtatctaataaggccctgcccgcacctattg |
| | aaaaaactatttcaaaggctaaagggcagccaagggag |
| | ccccaggtctacaccctgcctccatccagagatgaatt |
| | aacaaagaaccaggtgagtctcacttgcctggttaaag |
| | gtttttatccctctgatattgctgtggagtgggagtct |
| | aatggccaaccagaaaataactacaagaccacacctcc |
| | agtcttggactctgatggaagcttctttctttactcaa |
| | agctgactgtggacaaaagtagatggcagcaggggaat |
| | gtgttcagctgttctgttatgcatgaggcgctacacaa |
| | ccattatacccagaagtctctcagtctgtcacctggca |
| | aa |
| HC | gcaagtaccaaggggccatctgtgttcccactggcccc |
| Constant | ctcttccaaaagtacatcaggtggcacagcagctctgg |
| #3 | gatgcttggttaaggattactttcctgaaccagtgact |
| (SEQ ID | gtcagctggaatagtggggccttgacttctggggtgca |
| NO: 88) | cacattccctgcagtccttcagtcctctggtctgtatt |
| | cattatccagtgtggttacagtgccttctagctccctg |
| | ggaactcagacctacatctgtaatgtaaatcataaacc |
| | aagcaacacaaaggtggacaagaaagttgagcccaagt |
| | cttgtgataaaacccacacttgtccaccttgcccagcc |
| | cctgagctacttgggggcccttcagtttttctgttccc |
| | acccaagcctaaagacacattgatgattagtaggactc |
| | ctgaagtgacctgtgttgtggtggatgtttcccatgag |
| | gacccagaagtaaagttcaattggtatgtggatggagt |
| | tgaggtgcacaatgctaagactaaaccaagagaagagc |
| | agtataatagcacatacagggttgtgtcagttctgaca |
| | gtgcttcaccaagactggctcaatggaaaggagtataa |
| | atgcaaggtatctaataaggccctgcctgcacctattg |
| | aaaaaactatttcaaaggctaaagggcagccaagagag |
| | ccccaggtctacaccctgcctccatccagggatgaatt |
| | aacaaagaaccaggtgagtctcacttgcctggttaaag |
| | gtttttatccctctgatattgctgtggagtgggagtct |
| | aatggccaaccagaaaataactacaagaccacacctcc |
| | cagtttggactctgatggaagcttctttctttactcaa |
| | agctgactgtggacaaaagtagatggcagcaggggaat |
| | gtgttcagctgttctgttatgcatgaggcactacacaa |
| | ccattatacccagaagtctctcagtctgtcacctggca |
| | aa |

In some aspects, the polynucleotide disclosed herein comprises the nucleic acid sequences listed in Table 11. In some aspects, the polynucleotide disclosed herein encodes an antibody comprising the heavy chain (HC) of an antibody listed in Table 12 or antigen-binding fragment thereof. In some aspects, the polynucleotide disclosed herein encodes a signal peptide listed in Table 12.

TABLE 11
Heavy Chain (HC) Nucleic Acid Sequences

| Descrip- | |
|---|---|
| tion | |
| (SEQ ID | |
| NO) | HC Nucleic Acid Sequence |
| IL-10 | atgcacagctccgccctgctctgctgtttggtgcttct |
| signal | gaccggcgtccgggctcaggttgagttagtggaatctg |
| peptide- | gagggggtgtagtgcaacccggcagatcacagaggctg |
| HC #1 | agttgcgcagcgtcgggattcacatttagctcctacgg |
| (SEQ ID | gatgcattgggtccgccaggcccctggcaagggtctag |
| NO: 35) | agtgggtggctatcatttggttcgacggatcttcaact |
| Full: | tattacgccgatagtgttcgagggcgttttacgataag |
| 1398 | ccgggacaactccaaaaataccctctatctgcaaatga |
| IL-10 | actctttgagagcagaagatacagccgtgtacttctgt |
| signal | gctagggagctgggccgccggtattttgacctttgggg |
| peptide: | cagaggaactctcgtcagcgtatcctcagcaagtacca |
| 1-54 | aggggccaagcgtgttcccgctggccccctcttccaaa |
| (SEQ ID | tcgacatcaggtggcaccgcggctctgggatgcctcgt |
| NO: 121) | taaggattactttcctgaaccagtgactgtcagctgga |
| HC: | atagtggggccttgacgtctggcgtgcacacattcccc |
| 55-1398 | gcagtccttcagtccagcggtctgtattcattatccag |
| | tgtggttaccgtgccttctagctccctgggaactcaga |
| | cctacatctgtaacgtaaatcataaaccaagcaacaca |
| | aaggtggacaagaaagtcgagcccaagtcttgcgataa |
| | aacccacacttgtccgccttgcccagcccccgagctac |
| | tcgggggcccttcagtttttctgttcccacccaagcct |
| | aaagacacattgatgattagtaggacgcccgaagtgac |
| | ctgtgtcgtggtggatgtttcccatgaggacccagaag |
| | taaagttcaattggtacgtggatggcgtcgaggtgcac |
| | aacgctaagactaaaccgcgcgaagagcagtataatag |
| | cacataccgagtcgtgtcggttctgaccgtgcttcacc |
| | aagactggctcaacggaaaggagtataaatgcaaggta |
| | tctaataaggccctgcccgcacctatcgaaaaaactat |
| | ttcaaaggctaaagggcagccacgtgagccccaggtct |
| | acaccctgcctccatcccgggacgaattaacaaagaac |
| | caggtgagtctcacgtgcctggttaaaggtttttatcc |
| | cagcgatatcgccgtggagtgggagtctaacggccaac |
| | cggaaaataactacaagaccacacctccagtcttggac |
| | tccgatggaagcttctttctttactcaaagctgactgt |
| | ggacaaaagtagatggcagcaggggaatgtgttcagct |
| | gttccgttatgcatgaggcgctacacaaccattatacc |
| | cagaagtctctctcgctgtcacccggcaaa |
| IL-10 | atgcacagctccgccctgctctgctgtttggtgcttct |
| signal | gacaggagtcagggctcaggttgagttagtggaatctg |
| peptide- | gagggggtgtagtgcaacctggcagatcacagaggctg |
| HC #2 | agttgcgcagcaagtggattcacatttagctcctatgg |
| (SEQ ID | gatgcattgggtccgccaggcccctggcaagggtctag |
| NO: 36) | agtgggtggctatcatttggtttgatggatcttcaact |
| Full: | tattatgctgatagtgttagagggcgctttactataag |
| 1398 | cagggacaactccaaaaataccctctatctgcaaatga |
| IL-10 | actctttgagagcagaagatacagctgtgtacttctgt |
| signal | gctagggagctgggccgcagatattttgacctttgggg |
| peptide: | cagaggaactcttgtctctgtatcctcagcaagtacca |
| 1-54 | aggggccatctgtgttcccgctggccccctcttccaaa |
| HC: | agtacatcaggtggcaccgcggctctgggatgcttggt |
| 55-1398 | taaggattactttcctgaaccagtgactgtcagctgga |
| | atagtggggccttgacttctggggtgcacacattcccc |
| | gcagtccttcagtccagcggtctgtattcattatccag |
| | tgtggttacagtgccttctagctccctgggaactcaga |
| | cctacatctgtaatgtaaatcataaaccaagcaacaca |
| | aaggtggacaagaaagttgagcccaagtcttgtgataa |
| | aacccacacttgtccgccttgcccagcccctgagctac |
| | ttgggggcccttcagtttttctgttcccacccaagcct |
| | aaagacacattgatgattagtaggactcctgaagtgac |
| | ctgtgttgtggtggatgtttcccatgaggacccagaag |
| | taaagttcaattggtatgtggatggagttgaggtgcac |
| | aatgctaagactaaaccgagggaagagcagtataatag |
| | cacatacagagttgtgtcagttctgacagtgcttcacc |
| | aagactggctcaatggaaaggagtataaatgcaaggta |
| | tctaataaggccctgcccgcacctattgaaaaaactat |
| | ttcaaaggctaaagggcagccaagggagccccaggtct |
| | acaccctgcctccatccagagatgaattaacaaagaac |
| | caggtgagtctcacttgcctggttaaaggtttttatcc |
| | ctctgatattgctgtggagtgggagtctaatggccaac |
| | cagaaaataactacaagaccacacctccagtcttggac |
| | tctgatggaagcttctttctttactcaaagctgactgt |
| | ggacaaaagtagatggcagcaggggaatgtgttcagct |
| | gttctgttatgcatgaggcgctacacaaccattatacc |
| | cagaagtctctcagtctgtcacctggcaaa |
| IL-10 | atgcacagctctgccctgctctgctgtttggtgcttct |
| signal | gacaggagtcagggctcaggttgagttagtggaatctg |
| peptide- | gagggggtgtagtgcaacctggcagatcacagaggctg |
| HC #3 | agttgtgcagcaagtggattcacatttagctcctatgg |
| (SEQ ID | gatgcattgggtcagacaggcccctggcaagggtctag |
| NO: 37) | agtgggtggctatcatttggtttgatggatcttcaact |
| Full: | tattatgctgatagtgttagggggagatttactataag |
| 1398 | cagggacaactccaaaaataccctctatctgcaaatga |
| IL-10 | actctttgagagcagaagatacagctgtgtacttctgt |
| signal | gctagggagctgggcagaaggtattttgacctttgggg |
| peptide: | cagaggaactcttgtctctgtatcctcagcaagtacca |
| 1-54 | aggggccatctgtgttcccactggccccctcttccaaa |
| HC: | agtacatcaggtggcacagcagctctgggatgcttggt |
| 55-1398 | taaggattactttcctgaaccagtgactgtcagctgga |
| | atagtggggccttgacttctggggtgcacacattccct |
| | gcagtccttcagtcctctggtctgtattcattatccag |
| | tgtggttacagtgccttctagctccctgggaactcaga |
| | cctacatctgtaatgtaaatcataaaccaagcaacaca |
| | aaggtggacaagaaagttgagcccaagtcttgtgataa |
| | aacccacacttgtccaccttgcccagcccctgagctac |
| | ttgggggcccttcagtttttctgttcccacccaagcct |
| | aaagacacattgatgattagtaggactcctgaagtgac |
| | ctgtgttgtggtggatgtttcccatgaggacccagaag |
| | taaagttcaattggtatgtggatggagttgaggtgcac |
| | aatgctaagactaaaccaagagaagagcagtataatag |
| | cacatacagggttgtgtcagttctgacagtgcttcacc |
| | aagactggctcaatggaaaggagtataaatgcaaggta |
| | tctaataaggccctgcctgcacctattgaaaaaactat |
| | ttcaaaggctaaagggcagccaagagagccccaggtct |
| | acaccctgcctccatccagggatgaattaacaaagaac |
| | caggtgagtctcacttgcctggttaaaggtttttatcc |
| | ctctgatattgctgtggagtgggagtctaatggccaac |
| | cagaaaataactacaagaccacacctccagtcttggac |
| | tctgatggaagcttctttctttactcaaagctgactgt |
| | ggacaaaagtagatggcagcaggggaatgtgttcagct |
| | gttctgttatgcatgaggcactacacaaccattatacc |
| | cagaagtctctcagtctgtcacctggcaaa |

TABLE 12
Heavy Chain (HC) Amino Acid Sequences

| Descrip- | |
|---|---|
| tion | |
| (SEQ ID | |
| NO) | Sequence |
| HC | QVELVESGGGVVQPGRSQRLSCAASGFTFSSYGMH |
| (SEQ ID | WVRQAPGKGLEWVAIIWFDGSSTYYADSVRGRFTI |
| NO: 38) | SRDNSKNTLYLQMNSLRAEDTAVYFCARELGRRYF |
| | DLWGRGTLVSVSSASTKGPSVFPLAPSSKSTSGGT |
| | AALGCLVKDYFPEPVTVSWNSGALTSGVHTFPAVL |
| | QSSGLYSLSSVVTVPSSSLGTQTYICNVNHKPSNT |
| | KVDKKVEPKSCDKTHTCPPCPAPELLGGPSVFLFP |
| | PKPKDTLMISRTPEVTCVVVDVSHEDPEVKFNWYV |
| | DGVEVHNAKTKPREEQYNSTYRVVSVLTVLHQDWL |
| | NGKEYKCKVSNKALPAPIEKTISKAKGQPREPQVY |
| | TLPPSRDELTKNQVSLTCLVKGFYPSDIAVEWESN |
| | GQPENNYKTTPPVLDSDGSFFLYSKLTVDKSRWQQ |
| | GNVFSCSVMHEALHNHYTQKSLSLSPGK |
| IL-10 | MHSSALLCCLVLLTGVRA |
| signal | |
| peptide | |
| (SEQ ID | |
| NO: 120) | |

In some aspects, the polynucleotide disclosed herein comprises a nucleic acid sequence listed in Table 13. In some aspects, an antibody the polynucleotide disclosed herein encodes comprising the light chain (LC) of an antibody listed in Table 14 or antigen-binding fragment thereof. In some aspects, the polynucleotide disclosed herein encodes a signal peptide listed in Table 14.

TABLE 13
Light Chain (LC) Nucleic Acid Sequences

| Description | |
|---|---|
| (SEQ ID NO) | LC Nucleic Acid Sequence |
| IL-2 signal | atgtaccggatgcagctgctcagctgcatcgccttgtcccttgctctggtgaccaactctgagattgtcttaaca |
| peptide-LC #1 | caatcacccgcaactctgagtctatcgcctggcgaaagagcgacgctcagctgtagggcctcccagtctgttt |
| (SEQ ID NO: 39) | caagttatctggcttggtaccagcaaaagccaggacaggccccgcgcttgctgatatatgacgcaagcaaac |
| Full: 705 | gagccaccgggatccccgctcgtttctccggttctggcagcggaacagattttactcttaccatttcctcactcg |
| IL-2 signal | agcctgaagacttcgcagtgtactattgccagcagcggagtaagtggccaccctggacatttgggcaaggca |
| peptide: 1-60 | ccaaagtagagagcaagagaactgtggccgcgccttctgtcttcatctttccaccctccgatgaacagctgaa |
| (SEQ ID NO: 122) | atcgggtacggcttcagtggtttgtctgctcaataacttctacccgagggaggccaaggtgcagtggaaggtc |
| HC: 61-705 | gacaatgcattgcagagcggaaacagtcaagagtctgtaacagaacaggattccaaagacagcacctattca |
| | ctttccagtactctgaccttatctaaggccgattacgagaaacacaaggtgtacgcttgcgaagttacacatca |
| | ggggctgagctcccctgtgaccaaaagctttaatcgcggcgagtgt |
| IL-2 signal | atgtacagaatgcagctgctcagctgcattgccttgtcccttgctctggtgaccaactctgagattgtcttaaca |
| peptide-LC #2 | caatcacccgcaactctgagtctaagtcctggagaaagagcaactctcagctgtagggcctcccagtctgtttc |
| (SEQ ID NO: 40) | aagttatctggcttggtaccagcaaaagccaggacaggccccgcgcttgctgatatatgatgcaagcaaaag |
| Full: 705 | ggccacagggatccccgctagattcagcggttctggcagcggaacagattttactcttaccatttcctcacttg |
| IL-2 signal | agcctgaagactttgcagtgtactattgccagcagcggagtaagtggccaccctggacatttgggcaaggca |
| peptide: 1-60 | ccaaagtagagagcaagagaactgtggccgcgccttctgtcttcatctttccaccctctgatgaacagctgaa |
| HC: 61-705 | atcaggtactgcttcagtggtttgtctgctcaataacttctacccaagggaggccaaggtgcagtggaaggttg |
| | acaatgcattgcagagcggaaacagtcaagagtctgtaacagaacaggattccaaagacagcacctattcac |
| | tttccagtactctgaccttatctaaggctgattatgagaaacacaaggtgtatgcttgtgaagttacacatcagg |
| | ggctgagctcccctgtgaccaaaagctttaataggggggagtgt |
| IL-2 signal | atgtacagaatgcagctgctcagctgcattgccttgtcccttgctctggtgaccaactctgagattgtcttaaca |
| peptide-LC #3 | caatcacctgcaactctgagtctaagtcctggagaaagagcaactctcagctgtagggcctcccagtctgtttca |
| (SEQ ID NO: 41) | agttatctggcttggtaccagcaaaagccaggacaggccccaaggttgctgatatatgatgcaagcaaaaga |
| Full: 705 | gccacagggatccctgctaggttctctggttctggctctggaacagattttactcttaccatttcctcacttgag |
| IL-2 signal | cctgaagactttgcagtgtactattgccagcagagaagtaagtggccaccctggacatttgggcaaggcacca |
| peptide: 1-60 | aagtagagagcaagagaactgtggctgcaccttctgtcttcatctttccaccctctgatgaacagctgaaatca |
| HC: 61-705 | ggtactgcttcagtggtttgtctgctcaataacttctacccaagggaggccaaggtgcagtggaaggttgaca |
| | atgcattgcagtctggaaacagtcaagagtctgtaacagaacaggattccaaagacagcacctattcactttcc |
| | agtactctgaccttatctaaggctgattatgagaaacacaaggtgtatgcttgtgaagttacacatcaggggctg |
| | agctcccctgtgaccaaaagctttaataggggggagtgt |

TABLE 14
Light Chain (LC) Amino Acid Sequences

| (SEQ ID NO) | Sequence |
|---|---|
| LC | EIVLTQSPATLSLSPGERATLSCRASQSVSSYLAWYQQKPGQAPRLLI |
| (SEQ ID NO: | YDASKRATGIPARFSGSGSGTDFTLTISSLEPEDFAVYYCQQRSKWPP |
| 42) | WTFGQGTKVESKRIVAAPSVFIFPPSDEQLKSGTASVVCLLNNFYPR |
| | EAKVQWK\|\| |
| | [LMF1]VDNALQSGNSQESVTEQDSKDSTYSLSSTLTLSKADYEKH |
| | KVYACEVTHQGLSSPVTKSFNRGEC |
| IL-2 signal | MYRMQLLSCIALSLALVINS |
| peptide | |
| (SEQ ID NO: | |
| 119) | |

In some aspects, the polynucleotide disclosed herein comprises a nucleic acid listed in Tables 11 and 13. In some aspects, the polynucleotide disclosed herein encodes an antibody comprising a HC and a LC of an antibody listed in Tables 12 and 14 (i.e., the HC of the antibody listed in Table 12 and the LC of the same antibody listed in Table 14.

In some aspects, the therapeutic proteins used in the methods disclosed herein are antibodies, (e.g., monoclonal antibodies or antigen-binding fragments thereof) having the VH, VL, HC, and/or LC sequences of teprotumumab as well as antibodies having at least 80% identity, at least about 85%, at least about 90%, at least about 95%, at least about 96%, at least about 97%, at least about 98%, at least about 99%, or about 100% identity to the corresponding VH, VL, HC, and/or LC sequences.

In some aspects, the polynucleotide also comprises a linker sequence operably linked to the nucleic acid sequence encoding the heavy chain and/or the nucleic acid sequence encoding the light chain. In some aspects, the polynucleotide also comprises a pause element sequence operably linked to the nucleic acid sequence encoding the heavy chain and/or the nucleic acid sequence encoding the light chain. In some aspects, the pause element sequence has a nucleotide sequence at least 85%, 86%, 87%, 88%, 89%, 90%, 91%, 92%, 93%, 94%, 95%, 96%, 97%, 98%, 99%, or 100% identical to SEQ ID NO: 54.

In some aspects, the polynucleotide comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a construct comprising any one of SEQ ID NOs: 68-76, wherein the construct further comprises one or more of IRES, furin cleavage site, 2a site, or a dual promoter (e.g., promoter-VH-IRES-VL, etc.).

### II.A.2. Antigen Binding Fragments

In some aspects, an antigen-binding fragment of an antibody described herein, such as e.g., teprotumumab, is encoded by a polynucleotide disclosed herein. Exemplary antigen-binding fragments include but are not limited to Fab, Fab′, F(ab′)2, and scFv, wherein the Fab, Fab′, F(ab′)2, or scFv comprises a heavy chain variable region sequence and a light chain variable region sequence of teprotumumab as described herein. A Fab, Fab′, F(ab′)2, or scFv can be produced by any technique known to those of skill in the art. In some aspects, an antigen-binding fragment, such as a Fab, Fab′, F(ab′)2, or scFv, further comprises a moiety that extends the half-life of the antibody in vivo. The moiety is also termed a “half-life extending moiety.” Any moiety known to those of skill in the art for extending the half-life of an antigen-binding fragment, such as a Fab, Fab′, F(ab′)2, or scFv, in vivo can be used. For example, the half-life extending moiety can include an Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, an antigen-binding fragment, such as a Fab, Fab′, F(ab′)2, or scFv, can be modified by the addition of one or more C-terminal amino acids for attachment of the half-life extending moiety. In some aspects the half-life extending moiety is polyethylene glycol or human serum albumin. In some aspects, an antigen-binding fragment, such as a Fab, Fab′, F(ab′)2, or scFv, is fused to a Fc region.

In some aspects, the antibody or antigen-binding fragments thereof specifically binds to an insulin-like growth factor-1 receptor (IGF-1R), such as human IGF-1R.

In some aspects, the encoded anti-IGF-1R antibody comprises the amino acid sequence of teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or an antigen-binding fragment thereof.

In some aspects, the encoded anti-IGF-1R antibody comprises the amino acid sequence of VRDN-01100 (SEQ ID NO: 113), or VRDN-02700 (SEQ ID NO: 116), or an antigen-binding fragment thereof.

In some aspects, the encoded anti-IGF-1R antibody comprises the amino acid sequence of teprotumumab, or an antigen-binding fragment thereof.

In some aspects, the antibody (e.g., a monoclonal antibody) or antigen binding fragment thereof disclosed herein is modified so that it has enhanced half-life and/or reduced toxicity.

In some aspects, the encoded antibody or antigen-binding fragment thereof is a human antibody, a humanized antibody or a chimeric antibody. In some aspects, the antibody or antigen-binding fragment thereof can be selected from any class of immunoglobulins, including IgM, IgG, IgD, IgA and IgE, and any isotype, including IgG1, IgG2, IgG3 and IgG4. In some aspects, the antibody or antigen-binding fragment thereof is bispecific or multispecific.

### II.A.3. Antibody Expression Cassettes

In certain aspects, provided herein are antibody expression cassettes comprising a nucleotide sequence encoding an antibody or antigen-binding fragment thereof described herein or a domain thereof (e.g., a light chain, a heavy chain, a variable light chain region and/or variable heavy chain region) that specifically binds to an insulin-like growth factor receptor (IGFR) antigen-binding fragments thereof, or any combination thereof, and vectors, e.g., vectors comprising such antibody expression cassettes for expression in a cell, e.g., a fibroblast.

In some aspects, provided herein are antibody expression cassettes comprising nucleotide sequences encoding antibodies or antigen-binding fragments thereof, which specifically bind to an insulin-like growth factor receptor (IGF-1R) antigen-binding fragments thereof, or any combination thereof and comprise an amino acid sequence as described herein, as well as antibodies or antigen-binding fragments that compete with such antibodies or antigen-binding fragments for binding an IGF-1R antigen-binding fragments thereof, or any combination thereof (e.g., in a dose-dependent manner), or which bind to the same epitope as that of such antibodies or antigen-binding fragments.

In some aspects, the antibody expression cassette comprises nucleic acid sequence encoding an antibody that competes for binding to the same epitope as teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or an antigen-binding fragment thereof.

In some aspects, the antibody expression cassette comprises nucleic acid sequence encoding an antibody that competes for binding to the same epitope as VRDN-01100 (SEQ ID NO: 113), or VRDN-02700 (SEQ ID NO: 116), or an antigen-binding fragment thereof.

In some aspects, the antibody expression cassette comprises nucleic acid sequence encoding an antibody that competes for binding to the same epitope as teprotumumab, or an antigen-binding fragment thereof.

Also provided herein is an antibody expression cassette comprising a nucleotide sequence encoding a polypeptide comprising a sequence of any one of SEQ ID NOs: 7-24, 25-27. 29-31, 33-37, 39-41, or 43-76. In some aspects, an antibody or antigen-binding fragment thereof comprising the polypeptide specifically binds to an insulin-like growth factor receptor (IGFR) antigen-binding fragments thereof, or any combination thereof.

Also provided herein are kits, vectors, or host cells comprising (i) a first antibody expression cassette comprising a nucleotide sequence encoding any of SEQ ID NOs: 68-76 and (ii) a delivery vector.

In some aspects, provided herein are antibody expression cassettes comprising a nucleotide sequence comprising three VH domain CDRs, e.g., a nucleotide sequence containing VH CDR1, VH CDR2, and VH CDR3 of any one of the antibodies described herein (e.g., see Table 3), e.g., wherein the three VH domain CDRs are in the context of a VH. In some aspects, provided herein are polynucleotides comprising a nucleotide sequence comprising three VL domain CDRs, e.g., a nucleotide sequence containing VL CDR1, VL CDR2, and VL CDR3 of any one of the antibodies described herein (e.g., see Table 4), e.g., wherein the three VL domain CDRs are in the context of a VL. In some aspects, provided herein are antibody expression cassettes (or combinations of polynucleotides) comprising a nucleotide sequence comprising an antibody or antigen-binding fragment thereof comprising (i) three VH domain CDRs, e.g., a nucleotide sequence containing VH CDR1, VH CDR2, and VH CDR3 of any one of antibodies described herein (e.g., see Table 3) e.g., wherein the three VH domain CDRs are in the context of a VH and (ii) three VL domain CDRs, e.g., a nucleotide sequence containing VL CDR1, VL CDR2, and VL CDR3 of any one of antibodies described herein (e.g., see Table 4), e.g., wherein the three VL domain CDRs are in the context of a VL.

In some aspects, provided herein are antibody expression cassettes comprising a nucleotide sequence encoding three VH domain CDRs, e.g., a polypeptide containing VH CDR1, VH CDR2, and VH CDR3 of any one of the antibodies described herein (e.g., see Table 1), e.g., wherein the three VH domain CDRs are in the context of a VH. In some aspects, provided herein are antibody expression cassettes comprising a nucleotide sequence encoding three VL domain CDRs, e.g., a polypeptide containing VL CDR1, VL CDR2, and VL CDR3 of any one of the antibodies described herein (e.g., see Table 2), e.g., wherein the three VL domain CDRs are in the context of a VL. In some aspects, provided herein are antibody expression cassettes (or combinations of polynucleotides) comprising a nucleotide sequence encoding an antibody or antigen-binding fragment thereof comprising (i) three VH domain CDRs, e.g., a polypeptide containing VH CDR1, VH CDR2, and VH CDR3 of any one of antibodies described herein (e.g., see Table 1) e.g., wherein the three VH domain CDRs are in the context of a VH and (ii) three VL domain CDRs, e.g., a polypeptide containing VL CDR1, VL CDR2, and VL CDR3 of any one of antibodies described herein (e.g., see Table 2), e.g., wherein the three VL domain CDRs are in the context of a VL.

In some aspects, the heavy chain comprises a heavy chain variable region (VH) comprising a complementarity determining region (CDR) 1, a VH CDR2, and a VH CDR3. In some aspects, the modified VH CDRs 1-3 correspond to the CDRs of teprotumumab. In some aspects, the nucleic acid sequence encoding the VH CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10; the nucleic acid sequence encoding the VH CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14; and the nucleic acid sequence encoding the VH CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15.

In some aspects, the light chain comprises a light chain variable region (VL) comprising a complementarity determining region (CDR) 1, a VL CDR2, and a VL CDR3. In some aspects, the VL CDRs 1-3 correspond to the CDRs of teprotumumab. In some aspects, the nucleic acid sequence encoding the VL CDR1 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16; the nucleic acid sequence encoding the VL CDR2 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 21 or 23; and the nucleic acid sequence encoding the VL CDR3 comprises a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or

Also provided herein are antibody expression cassettes encoding an antibody or antigen-binding fragment thereof described herein or a domain thereof that are optimized, e.g., by codon/RNA optimization, replacement with heterologous signal sequences, and elimination of mRNA instability elements. Methods to generate optimized nucleic acids encoding an antibody or antigen-binding fragment thereof described herein or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, each of which is incorporated herein by reference in its entirety.

Antibody expression cassettes comprising a nucleic acid sequence encoding an anti-IGF-1R antibody or antigen-binding fragment thereof described herein or a domain thereof can be generated from nucleic acid from a suitable source using methods well known in the art (e.g., PCR and other molecular cloning methods). For example, PCR amplification using synthetic primers hybridizable to 3′ and 5′ ends of a known sequence can be performed using genomic DNA obtained from hybridoma cells producing the antibody of interest. In some aspects, the hybridoma cell line has the accession number DSM ACC 2587 (deposited Oct. 4, 2003). In some aspects, the hybridoma cell line has the accession number DSM ACC 2594 (deposited Sep. 5, 2003). Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Antibody expression cassettes provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the antibody expression cassette is a cDNA or a DNA lacking one more endogenous introns. In some aspects, an antibody expression cassettes is a non-naturally occurring antibody expression cassette. In some aspects, an antibody expression cassette is recombinantly produced. In some aspects, the antibody expression cassettes are isolated. In some aspects, the antibody expression cassettes are substantially pure. In some aspects, an antibody expression cassette is purified from natural components.

In some aspects, a viral vector disclosed herein comprises an antibody expression cassette comprising coding regions for two or more polypeptides, e.g., a heavy chain and a light chain.

When it is desired the antibody expression cassette includes coding regions for two or more individual polypeptide chains, each additional coding region beyond the first is preferably linked to an element that facilitates co-expression of the proteins in host cells, such as an internal ribosomal entry sequence (IRES) element (See e.g., U.S. Pat. No. 4,937,190), furin cleavage site, a 2A element, or promoter(s). In some aspects, IRES furin cleavage sites, or 2A elements can be used when a single vector comprises sequences encoding each subunit of a multi-subunit protein. In the case when the protein of interest is immunoglobulin with a desired specificity, for example, the first coding region (encoding either the heavy or light chain of immunoglobulin) is located downstream from the promoter. The second coding region (encoding the remaining chain of immunoglobulin) can be located downstream from the first coding region, and an IRES, furin cleavage site, or 2A element can be disposed between the two coding regions, e.g., immediately preceding the second coding region. In some aspects, the incorporation of an IRES, furin cleavage site, or 2A element between the sequences of a first and second gene (encoding the heavy and light chains, respectively) can allow both chains to be expressed from the same promoter at about the same level in the cell.

In some aspects, the protein of interest comprises two or more subunits, for example an immunoglobulin (Ig). In some aspects, a delivery vector of the disclosure can include a coding region for each of the subunits. For example, the viral vector can include both the coding region for the Ig heavy chain (or the variable region of the Ig heavy chain) and the coding region for the Ig light chain (or the variable region of the Ig light chain). In some aspects, the vectors include a first coding region for the heavy chain variable region of an antibody, and a second coding region for the light chain variable region of the antibody. In some aspects, the two coding regions can be separated, for example, by a 2A self-processing sequence to allow multi-cistronic transcription of the two coding regions.

The viral vector can include coding regions for two or more proteins of interest. For example, the viral vector can include the coding region for a first protein of interest and the coding region for a second protein of interest. The first protein of interest and the second protein of interest can be the same or different.

The Kozak consensus sequence, Kozak consensus or Kozak sequence, is known as a sequence which occurs on eukaryotic mRNA and has the consensus (gcc) gccRccAUGG, where R is a purine (adenine or guanine) three bases upstream of the start codon (AUG), which is followed by another “G.” In some aspects, the vector comprises a nucleotide sequence having at least about 85%, at least about 90%, at least about 95% sequence identity, or more to the Kozak consensus sequence. In some aspects, the vector comprises a Kozak consensus sequence. In some aspects, the vector includes a Kozak consensus sequence after the polynucleotide encoding one or more proteins of interest is inserted into the vector, e.g., at the restrict site downstream of the promoter. For example, the vector can include a nucleotide sequence of GCCGCCATG (SEQ ID NO: 77), where the ATG is the start codon of the protein of interest. In some aspects, the vector comprises a nucleotide sequence of GCGGCCGCCATG (SEQ ID NO: 78), where the ATG is the start codon of the protein of interest.

In certain aspects, a composition comprising a delivery vector, e.g., a viral vector, comprising nucleic acids encoding an anti-IGF-1R antibody or an antigen binding fragment are provided for herein.

In some aspects, the delivery vector, e.g., a viral vector, comprises a nucleic acid encoding an anti-IGF-1R antibody (e.g., a monoclonal antibody) or an antigen-binding fragment thereof disclosed herein is secreted from the salivary gland and swallowed. In some aspects, the therapeutic effect of the secreted anti-IGF-1R antibody or antigen-binding fragment thereof is local, systemic, or both.

In some aspects, the delivery vector (e.g., a delivery vector comprising an antibody or antigen-binding fragments thereof specifically binds to insulin-like growth factor receptor (IGFR), such as human IGF-1R is suitable for delivery to or near an eye (e.g., one or both eyes), e.g., intraocular, retro- or a peri-orbital, retrobulbar, intramuscular near the eye, to connective tissue near the eye, or any combination thereof. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro- or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the antibody expression cassette comprises a nucleic acid sequence encoding an anti-IGF-1R antibody or antigen-binding fragment thereof comprising (i) VH CDRs 1-3 (e.g., SEQ ID NOs: 7-9, 10-12, or 13-15) and VL CDRs 1-3 (e.g., SEQ ID NOs: 16-18, 19-21, or 22-24) of modified teprotumumab; (ii) VH (e.g., SEQ ID NOs: 26 or 27) and VL (e.g., SEQ ID NOs: 30 or 31) of modified teprotumumab; (iii) HC (e.g., SEQ ID NOs: 36 or 37) and LC (e.g., SEQ ID NOs: 40 or 41) of modified teprotumumab; or (iv) a sequence comprising any one of SEQ ID NOs: 68-76, wherein the sequence further comprises one or more of IRES, furin cleavage site, 2a site, or a dual promoter (e.g., promoter-VH-IRES-VL, etc.). In some aspects, the antibody expression cassette further comprises a sequence encoding a signal peptide (e.g., an IL-2 or an IL-10 signal peptide). In some aspects, the signal peptide is an IL-2 signal peptide or an IL-10 signal peptide. In some aspects, the encoded signal peptide comprises an amino acid sequence at least 85%, 86%, 87%, 88%, 89%, 90%, 91%, 92%, 93%, 94%, 95%, 96%, 97%, 98%, 99%, or 100% identical to any of SEQ ID NOs: 119 or 120. In some aspects, the nucleic acid sequence encoding the signal peptide comprises a nucleic acid sequence at least 85%, 86%, 87%, 88%, 89%, 90%, 91%, 92%, 93%, 94%, 95%, 96%, 97%, 98%, 99%, or 100% identical to any of SEQ ID NOs: 121 or 122.

### III. Vector Constructs

Some aspects of the disclosure are directed to a vector construct or an expression construct (e.g., an antibody expression cassette) having a eukaryotic promoter operably linked to a DNA of interest that encodes an anti-IGF-1R antibody (e.g., a monoclonal antibody) or an antigen binding fragment thereof disclosed herein. In some aspects, the vector constructs or expression constructs containing the DNA sequence (or the corresponding RNA sequence) which can be used in accordance with the disclosure can be any eukaryotic expression construct containing the DNA or the RNA sequence of interest. For example, a plasmid or viral construct (e.g. an AAV vector) can be cleaved to provide linear DNA having ligatable termini. These termini are bound to exogenous DNA having complementary, like ligatable termini to provide a biologically functional recombinant DNA molecule having an intact replicon and a desired phenotypic property. In some aspects, the vector construct or expression construct is capable of replication in both eukaryotic and prokaryotic hosts, which constructs are known in the art and are commercially available.

In some aspects, the vector construct or expression construct of the disclosure encoding an anti-IGF-1R antibody (e.g., teprotumumab) is a multicistronic (e.g., bicistronic) construct (e.g., comprising a heavy chain and a light chain). In some aspects, the multicistronic (e.g., bicistronic) construct further comprises an F2A or IRES element.

In some aspects, the anti-IGF-1R antibody is teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or an antigen-binding fragment thereof.

In some aspects, the anti-IGF-1R antibody is VRDN-01100 (SEQ ID NO: 113), or VRDN-02700 (SEQ ID NO: 116), or an antigen-binding fragment thereof.

In some aspects, the anti-IGF-1R antibody is teprotumumab, or an antigen-binding fragment thereof.

The exogenous (i.e., donor) DNA used in the disclosure is obtained from suitable cells, and the vector constructs or expression constructs prepared using techniques well known in the art. Likewise, techniques for obtaining expression of exogenous DNA or RNA sequences in a genetically altered host cell are known in the art (see e.g., Kormal et al., Proc. Natl. Acad. Sci. USA, 84:2150-2154 (1987); Sambrook et al. Molecular Cloning: a Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

In some aspects, the vector construct or expression construct contains a promoter to facilitate expression of the DNA of interest within a secretory cell. In some aspects, the promoter is a strong, eukaryotic promoter such as a promoter from human cytomegalovirus (CMV), mouse CMV promoter, mouse mammary tumor virus (MMTV), Rous sarcoma virus (RSV), or adenovirus. Exemplary promoters include, but are not limited to the promoter from the immediate early gene of human CMV (Boshart et al., Cell 41:521-530 (1985) and the promoter from the long terminal repeat (LTR) of RSV (Gorman et al., Proc. Natl. Acad. Sci. USA 79:6777-6781 (1982)). In some aspects, the promoter is a CMV early enhancer/chicken β actin (CBA) promoter, CAG promoter, CMV, EF1a, EF1α with a CMV enhancer, a CMV promoter with a CMV enhancer (CMVe/p), a CBA promoter with a CMV enhancer, a CMV promoter with a SV40 intron, a CBA promoter with a CMV enhancer and a CAG intron, EF1α promoter with a truncated 5′ LTR and a chimeric HBG and IgHC intron, or a tissue specific promoter. In some aspects, the tissue specific promoter is a muscle specific promoter. In some aspects, the muscle specific promoter is a DES promoter, a HSA promoter, a MCK promoter, a HMCK7 promoter, a dMCK promoter, a tMCK promoter, a CK8e promoter, a SPc5-12 promoter, a SP-301 promoter, a MH promoter, a Sk-CRM promoter, or a Sk-CRM4 promoter.

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38. In some aspects, the nucleic acid sequence comprising the promoter can comprises an intron. In some aspects, the intron is selected from the group consisting of an SV40 intron, MVM intron, or a human betaglobin intron. In some aspects a CMVp promoter is fused to a SV40 intron. In some aspects, SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 46.

Alternatively, the promoter used can be a tissue-specific promoter. For example, where the cell is a fibroblast, the tissue-specific promoter can be an insulin-like growth factor binding protein 2 (IGFBP2) promoter, a fibroblast activation protein (FAP) promoter, or fibroblast specific protein 1 (FSP1) promoter. Where the cell is a myocyte, the tissue-specific promoter can be a muscle creatine kinase (MCK) promoter, troponin I (TNNI2) promoter, skeletal alpha-actin (ASKA) promoter, a DES promoter, a HSA promoter, a MCK promoter, a HMCK7 promoter, a dMCK promoter, a tMCK promoter, a CK8e promoter, a SPc5-12 promoter, a SP-301 promoter, a MH promoter, a Sk-CRM promoter, or a Sk-CRM4 promoter. Where the cell is an adipocyte, the tissue-specific promoter can be an adiponectin promoter or adipocyte fatty acid-binding protein (AP2) promoter.

In some aspects, the vector construct or expression construct contains a first promoter and a second promoter. In some aspects, the first and second promoter are different. In some aspects, the first and second promoter are the same. In some aspects, the first and second promoter initiate transcription in the same direction. In some aspects, the first and second promoter initiate transcription in different directions. In some aspects, the first or second promoter in a CMV promoter. In some aspects, the first or second promoter is an EF-1α promoter.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 54.

Given the genome size limitations of AAV, expression of multicistronic vectors (multiple genes or multiple open reading frames) of large proteins under the control of a single promoter can be a challenge.

In some aspects, the multicistronic vectors disclose herein comprise an internal ribosome entry site (IRES) sequences or 2A peptides. In some aspects, the constructs of the disclosure can also include proteolytic cleavage sites. In some aspects, the proteolytic cleavage sites are furin cleavage sites and/or 2A cleavage sites.

In some aspects, the vector constructs or expression constructs of the disclosure can also include other components such as a marker (e.g., an antibiotic resistance gene (such as an ampicillin resistance gene) or β-galactosidase) to aid in selection of cells containing and/or expressing the construct, an origin of replication for stable replication of the construct in a bacterial cell (preferably, a high copy number origin of replication), a nuclear localization signal, or other elements which facilitate production of the DNA construct, the protein encoded thereby, or both. In some aspects, the vector constructs of the disclosure can comprise an antibiotic resistance gene including, but not limited to, neomycin, kanamycin, puromycin, and/or zeocin. In some aspects, the vector constructs of the disclosure can comprise a ColE1, f1, pUC, p15A or pMB1 origin of replication.

In some aspects, the vector construct of the disclosure contains a backbone comprising a ColE1 origin of replication and/or a kanamycin resistance gene of SEQ ID NO: 84.

In some aspects, the vector construct can comprise an ColE1 origin of replication and kanamycin resistance such as

(SEQ ID NO: 84)
5′-gccagcaaaaggccaggaaccgtaaaaaggccgcgttgctggcgtttttccatag
gctccgcccccctgacggcgtttccccctggaagctccctcgtgcgctctcctgttccgaccctgccgcttaccggatacctgtcc
gcctttctcccttcgggaagcgtggcgctttctcatagctcacgctgtaggtatctcagttcggtgtaggtcgttcgctccaagctgg
gctgtgtgcacgaaccccccgttcagcccgaccgctgcgccttatccggtaactatcgtcttgagtccaacccggtaagacacga
cttatcgccactggcagcagccactggtaacaggattagcagagcgaggtatgtaggcggtgctacagagttcttgaagtggtg
gcctaactacggctacactagaagaacagtatttggtatctgcgctctgctgaagccagttaccttcggaaaaagagttggtagct
cttgatccggcaaacaaaccaccgctggtagcggtggtttttttgtttgcaagcagcagattacgcgcagaaaaaaaggatctcaa
gaagatcctttgatcttttctacggggtctgacgctcagtggaacgaaaactcacgttaagggattttggtcatgagattatcaaaaa
ggatcttcacctagatccttttaaattaaaaatgaagttttaaatcaagcccaatctgaataatgttacaaccaattaaccaattctgatt
agaaaaactcatcgagcatcaaatgaaactgcaatttattcatatcaggattatcaataccatatttttgaaaaagccgtttctgtaatg
aaggagaaaactcaccgaggcagttccataggatggcaagatcctggtatcggtctgcgattccgactcgtccaacatcaataca
acctattaatttcccctcgtcaaaaataaggttatcaagtgagaaatcaccatgagtgacgactgaatccggtgagaatggcaaaa
gtttatgcatttctttccagacttgttcaacaggccagccattacgctcgtcatcaaaatcactcgcatcaaccaaaccgttattcattc
gtgattgcgcctgagcgagacgaaatacgcgatcgctgttaaaaggacaattacaaacaggaatcgaatgcaaccggcgcagg
aacactgccagcgcatcaacaatattttcacctgaatcaggatattcttctaatacctggaatgctgtttttccggggatcgcagtggt
gagtaaccatgcatcatcaggagtacggataaaatgcttgatggtcggaagaggcataaattccgtcagccagtttagtctgacca
tctcatctgtaacatcattggcaacgctacctttgccatgtttcagaaacaactctggcgcatcgggcttcccatacaagcgatagat
tgtcgcacctgattgcccgacattatcgcgagcccatttatacccatataaatcagcatccatgttggaatttaatcgcggcctcgac
gtttcccgttgaatatggctcat-3′

For eukaryotic expression, the vector construct or expression construct can comprise at a minimum a eukaryotic promoter operably linked to a DNA of interest, which is in turn operably linked to a polyadenylation sequence. The polyadenylation signal sequence can be selected from any of a variety of polyadenylation signal sequences known in the art. In some aspects, the polyadenylation signal sequence is the SV40 early polyadenylation signal sequence. In some aspects, the polyadenylation signal sequence is the bovine growth hormone polyadenylation signal sequence (bGHpA). In some aspects, the polyadenylation signal sequence is the human growth hormone polyadenylation signal sequence (hGHpA). In some aspects, the polyadenylation signal sequence is the SV40 polyadenylation signal sequence (SV40 pA). The construct can also include one or more introns, which can increase levels of expression of the DNA of interest, particularly where the DNA of interest is a cDNA (e.g., contains no introns of the naturally-occurring sequence). Any of a variety of introns known in the art can be used (e.g., the human β-globin intron, which is inserted in the vector construct or expression construct at a position 5′ to the DNA of interest). In some aspects, the intron is an SV40 intron. In some aspects, the intron is from an immunoglobulin heavy chain. In some aspects, the intron is a chimera between the human β-globin and immunoglobin heavy chain gene. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 46, 56, or 82.

In some aspects, the polynucleotide comprises a poly (A). In some aspects, the poly (A) is a synthetic poly (A) or a bovine growth hormone (BGH) poly (A). In some aspects, the polynucleotide comprises a poly (A) sequence comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 52-53.

When it is desired to include coding regions for two or more individual polypeptide chains, or two or more subunits of a protein of interest in one viral vector, each additional coding region beyond the first is preferably linked to an element that facilitates co-expression of the proteins in host cells, such as an internal ribosomal entry sequence (IRES) element (See e.g., U.S. Pat. No. 4,937,190), or a 2A element. In some aspects, IRES, furin cleavage site, or 2A elements can be used when a single vector comprises sequences encoding each subunit of a multi-subunit protein. In the case when the protein of interest is immunoglobulin with a desired specificity, for example, the first coding region (encoding either the heavy or light chain of immunoglobulin) is located downstream from the promoter. The second coding region (encoding the remaining chain of immunoglobulin) can be located downstream from the first coding region, and an IRES, furin cleavage site, or 2A element can be disposed between the two coding regions, e.g., immediately preceding the second coding region. In some aspects, the incorporation of an IRES, furin cleavage site, or 2A element between the sequences of a first and second gene (encoding the heavy and light chains, respectively) can allow both chains to be expressed from the same promoter at about the same level in the cell.

In some aspects, the nucleic acid sequence of the vector construct or expression construct comprises a promoter, heavy chain, IRES, and light chain sequences in 5′-3′ orientation. In some aspects, the nucleic acid sequence of the construct comprises a promoter, light variable, IRES, and heavy chain sequences in 5′-3′ orientation.

In some aspects, the nucleic acid sequence vector construct or expression construct comprises proteolytic cleavage sites. For example, the nucleic acid sequence may comprise a sequence that is incorporated into a vector construct or expression construct of the disclosure adjacent a self-processing cleavage site, such as a 2A or 2A like sequence, and provides a means to remove additional amino acids that remain following cleavage by the self-processing cleavage sequence. Exemplary proteolytic cleavage sites are described herein and include, but are not limited to, furin cleavage sites with the consensus sequence RXK(R)R (SEQ ID NO: 79). Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

In some aspects, the nucleic acid sequence vector construct or expression construct comprises a nucleic acid encoding a signal peptide operably linked to a nucleic acid encoding an antibody or antigen binding fragment thereof that binds an insulin-like growth factor-1 receptor. In some aspects the signal peptide is an endogenous signal peptide for HGH and variants thereof; an endogenous signal peptide for interferons and variants thereof, including the signal peptide of type I, II and III interferons and variants thereof; an endogenous signal peptides for known cytokines and variants thereof, such as the signal peptide of erythropoietin (EPO), insulin, TGF-β1, TNF, IL1-α, and IL1-β, and variants thereof. In some embodiments, the signal peptide is a modified signal peptide. In some aspects, the signal peptide is an IL-2 signal peptide. In some aspects, the signal peptide is an IL-10 signal peptide. In some aspects the signal peptide comprises and amino acid sequence with at least 85%, 86%, 87%, 88%, 89%, 90%, 91%, 92%, 93%, 94%, 95%, 96%, 97%, 98%, 99%, or 100% identity to SEQ ID NO: 119 or 120. In some aspects, the nucleic acid sequence encoding the signal peptide comprises a nucleic acid sequence at least 85%, 86%, 87%, 88%, 89%, 90%, 91%, 92%, 93%, 94%, 95%, 96%, 97%, 98%, 99%, or 100% identical to any of SEQ ID NOs: 121 or 122.

In some aspects, the nucleic acid sequence vector construct or expression construct comprises a promoter, heavy chain, furin cleavage site, 2A cleavage site, and light chain sequences in 5′-3′ orientation.

In some aspects, the nucleic acid sequence vector construct or expression construct comprises a promoter, a first signal peptide, heavy chain, furin cleavage site, 2A cleavage site, a second peptide, and light chain sequences in 5′-3′ orientation.

In some aspects, the nucleic acid sequence vector construct or expression construct comprises a promoter, light chain, furin cleavage site, 2A cleavage site, and heavy chain sequences in 5′-3′ orientation.

In some aspects, the nucleic acid sequence vector construct or expression construct comprises a promoter, a first signal peptide, light chain, furin cleavage site, 2A cleavage site, a second signal peptide, and heavy chain sequences in 5′-3′ orientation.

In some aspects, the nucleic acid sequence vector construct or expression construct comprises a promoter, heavy chain sequences of an anti-IGF-1R antibody or antigen-binding fragment thereof, IRES, and light chain sequence of an anti-IGF-1R antibody or antigen-binding fragment thereof s in 5′-3′ orientation.

In some aspects, the nucleic acid sequence vector construct or expression construct comprises a promoter, a first signal peptide, heavy chain sequences of an anti-IGF-1R antibody or antigen-binding fragment thereof, IRES, a second signal peptide, and light chain sequence of an anti-IGF-1R antibody or antigen-binding fragment thereof s in 5′-3′ orientation.

In some aspects, the nucleic acid sequence vector construct or expression construct comprises a promoter, light chain sequences of an anti-IGF-1R antibody or antigen-binding fragment thereof, IRES, and heavy chain sequences of an anti-IGF-1R antibody or antigen-binding fragment thereof in 5′-3′ orientation.

In some aspects, the nucleic acid sequence vector construct or expression construct comprises a promoter, a first signal peptide, light chain sequences of an anti-IGF-1R antibody or antigen-binding fragment thereof, IRES, a second signal peptide, and heavy chain sequences of an anti-IGF-1R antibody or antigen-binding fragment thereof in 5′-3′ orientation.

In some aspects, the vector construct or expression construct comprises a first promoter, a nucleic acid sequence encoding a light chain, a second promoter, and a nucleic acid sequence encoding a heavy chain in 5′-3′ orientation.

In some aspects, the vector construct or expression construct comprises a first promoter, a nucleic acid sequence encoding a first signal peptide, a nucleic acid sequence encoding a light chain, a second promoter, a nucleic acid sequence encoding a second signal peptide, and a nucleic acid sequence encoding a heavy chain in 5′-3′ orientation.

In some aspects, the vector construct or expression construct comprises a first promoter, a nucleic acid sequence encoding a heavy chain, a second promoter, and a nucleic acid sequence encoding a light chain in 5′-3′ orientation.

In some aspects, the vector construct or expression construct comprises a first promoter, a nucleic acid sequence encoding a first signal peptide, a nucleic acid sequence encoding a heavy chain, a second promoter, a nucleic acid sequence encoding a second signal peptide, and a nucleic acid sequence encoding a light chain in 5′-3′ orientation.

In some aspects, the vector construct or expression construct comprises a nucleic acid sequence encoding a heavy chain, a first promoter sequence, a second promoter sequence, and a nucleic acid sequence encoding a light chain in 5′-3′ orientation.

In some aspects, the vector construct or expression construct comprises a nucleic acid sequence encoding a first signal peptide, a nucleic acid sequence encoding a heavy chain, a first promoter sequence, a second promoter sequence, a nucleic acid sequence encoding a second signal peptide, and a nucleic acid sequence encoding a light chain in 5′-3′ orientation.

In some aspects, the vector construct or expression construct comprises a nucleic acid sequence encoding a light chain, a first promoter sequence, a second promoter sequence, and a nucleic acid sequence encoding a heavy chain in 5′-3′ orientation.

In some aspects, the vector construct or expression construct comprises a nucleic acid sequence encoding a first signal peptide, a nucleic acid sequence encoding a light chain, a first promoter sequence, a second promoter sequence, a nucleic acid sequence encoding a second signal peptide, and a nucleic acid sequence encoding a heavy chain in 5′-3′ orientation.

In some aspects, the promoter is selected from the group consisting of a CAG promoter, a CBA promoter, a CMV promoter, an EF1α promoter, an EF1α promoter with a CMV enhancer, a CMV promoter with a CMV enhancer (CMVe/p), a CMV promoter with a SV40 intron or tissue specific promoter. In some aspects, the tissue specific promoter is selected from a DES promoter, a HSA promoter, a MCK promoter, a HMCK7 promoter, a dMCK promoter, a tMCK promoter, a CK8e promoter, a SPc5-12 promoter, a SP-301 promoter, a MH promoter, a Sk-CRM promoter, and a Sk-CRM4 promoter.

In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51, 83, or 93. In some aspects, the nucleic acid sequence comprising the promoter can comprises an intron. In some aspects, the intron is selected from the group consisting of an SV40 intron, MVM intron, or a human betaglobin intron. In some aspects, a CMVp is fused to a SV40 intron. In some aspects, a CAG intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 82. In some aspects, a SV40 intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 46.

In some aspects, the first and second promoter are different. In some aspects, the first and second promoter are the same. In some aspects, the first and second promoter initiate transcription in the same direction. In some aspects, the first and second promoter initiate transcription in different directions.

In some aspects, the nucleic acid sequence encoding the first promoter and the nucleic acid sequence encoding the second promoter are operably linked. In some aspects, the nucleic acid sequence encoding the first promoter and the nucleic acid sequence encoding the second promoter are operably linked by a pause element. In some aspects, the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 54.

In some aspects, the signal is selected from the group consisting of an endogenous signal peptide for HGH and variants thereof; an endogenous signal peptide for interferons and variants thereof, including the signal peptide of type I, II and III interferons and variants thereof; an endogenous signal peptides for known cytokines and variants thereof, such as the signal peptide of erythropoietin (EPO), insulin, TGF-β1, TNF, IL1-α, and IL1-β, and variants thereof. In some embodiments, the signal peptide is a modified signal peptide. In some aspects, the signal peptide is an IL-2 signal peptide. In some aspects, the signal peptide is an IL-10 signal peptide. In some aspects the signal peptide comprises and amino acid sequence with at least 85%, 86%, 87%, 88%, 89%, 90%, 91%, 92%, 93%, 94%, 95%, 96%, 97%, 98%, 99%, or 100% identity to SEQ ID NO: 119 or 120. In some aspects, the nucleic acid sequence encoding the signal peptide comprises a nucleic acid sequence at least 85%, 86%, 87%, 88%, 89%, 90%, 91%, 92%, 93%, 94%, 95%, 96%, 97%, 98%, 99%, or 100% identical to any of SEQ ID NOs: 121 or 122.

The vectors for delivery of the DNA of interest can be either viral or non-viral, or can be composed of naked DNA admixed with an adjuvant such as viral particles (e.g., AAV particle) or cationic lipids or liposomes. An “adjuvant” is a substance that does not by itself produce the desired effect, but acts to enhance or otherwise improve the action of the active compound. The precise vector and vector formulation used will depend upon several factors such as the secretory gland targeted for gene transfer.

In some aspects, a composition comprising a delivery vector, e.g., a viral vector, comprising a nucleic acid construct or an expression construct comprising a nucleic acid encoding an anti-IGF-1R antibody (e.g., a monoclonal antibody) or an antigen binding fragment thereof disclosed herein. In some aspects, the delivery vector is suitable for delivery to a periorbital or retroorbital tissue. In some aspects, the tissue is a connective tissue, a muscle tissue, or an adipose tissue.

In some aspects, the vector construct or expression construct comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a construct comprising any one of SEQ ID NOs: 68-76, wherein the construct further comprises one or more of IRES, furin cleavage site, 2a site, or a dual promoter (e.g., promoter-VH-IRES-VL, etc.).

In some aspects, the nucleic acid construct or expression construct comprises a polynucleotide comprising a first promoter, nucleic acid sequence encoding a heavy chain of an anti-IGF-1R antibody or antigen-binding fragment thereof, a F2A site, a light chain of an anti-IGF-1R antibody or antigen-binding fragment thereof, and a poly (A) in 5′-3′ orientation.

In some aspects, the nucleic acid construct or expression construct comprises a polynucleotide comprising a first promoter, nucleic acid sequence encoding a first signal peptide, a heavy chain of an anti-IGF-1R antibody or antigen-binding fragment thereof, a F2A site, a second signal peptide, a light chain of an anti-IGF-1R antibody or antigen-binding fragment thereof, and a poly (A) in 5′-3′ orientation.

In some aspects, the nucleic acid construct or expression construct comprises a polynucleotide comprising a CMV enhancer, a CBA promoter, a CAG intron, a CBA exon, a nucleic acid encoding a heavy chain of an anti-IGF-1R antibody or antigen-binding fragment thereof, a furin cleavage site, a linker, a 2A peptide, a nucleic acid encoding a light chain of an anti-IGF-1R antibody or antigen-binding fragment thereof, and a BGHpA in 5′-3′ orientation.

In some aspects, the nucleic acid construct or expression construct comprises a polynucleotide comprising a CMV enhancer, a CBA promoter, a CAG intron, a CBA exon, a nucleic acid encoding a first signal peptide, a heavy chain of an anti-IGF-1R antibody or antigen-binding fragment thereof, a furin cleavage site, a linker, a 2A peptide, a nucleic acid encoding a second signal peptide, a light chain of an anti-IGF-1R antibody or antigen-binding fragment thereof, and a BGHpA in 5′-3′ orientation.

In some aspects, the nucleic acid construct or expression construct comprises a polynucleotide comprising a first promoter, nucleic acid sequence encoding a heavy chain of an anti-IGF-1R antibody or antigen-binding fragment thereof, an IRES2, a light chain of an anti-IGF-1R antibody or antigen-binding fragment thereof, and a poly (A) in 5′-3′ orientation.

In some aspects, the nucleic acid construct or expression construct comprises a polynucleotide comprising a first promoter, nucleic acid sequence encoding a first signal peptide, a heavy chain of an anti-IGF-1R antibody or antigen-binding fragment thereof, an IRES2, a second signal peptide, a light chain of an anti-IGF-1R antibody or antigen-binding fragment thereof, and a poly (A) in 5′-3′ orientation.

In some aspects, the nucleic acid construct or expression construct comprises a polynucleotide comprising a CMV enhancer, a CBA promoter, a CAG intron, a CBA exon, a nucleic acid encoding a light chain of an anti-IGF-1R antibody or antigen-binding fragment thereof, an IRES2 element, a nucleic acid encoding a heavy chain of an anti-IGF-1R antibody or antigen-binding fragment thereof, and a SynpA in 5′-3′ orientation.

In some aspects, the nucleic acid construct or expression construct comprises a polynucleotide comprising a CMV enhancer, a CBA promoter, a CAG intron, a CBA exon, a nucleic acid encoding a first signal peptide, a light chain of an anti-IGF-1R antibody or antigen-binding fragment thereof, an IRES2 element, a nucleic acid encoding a second signal peptide, a heavy chain of an anti-IGF-1R antibody or antigen-binding fragment thereof, and a SynpA in 5′-3′ orientation.

In some aspects, the nucleic acid construct or expression construct comprises a polynucleotide comprising a first promoter, an intron, a nucleic acid sequence encoding a light chain of an anti-IGF-1R antibody or antigen-binding fragment thereof, a poly (A), a pause element, a second promoter, a 5′ LTR, a chimeric intron, a nucleic acid sequence encoding a heavy chain of an anti-IGF-1R antibody or antigen-binding fragment thereof, and a poly (A) in 5′-3′ orientation.

In some aspects, the nucleic acid construct or expression construct comprises a polynucleotide comprising a first promoter, an intron, a nucleic acid sequence encoding a first signal peptide, a light chain of an anti-IGF-1R antibody or antigen-binding fragment thereof, a poly (A), a pause element, a second promoter, a 5′ LTR, a chimeric intron, a nucleic acid sequence encoding a second signal peptide, a heavy chain of an anti-IGF-1R antibody or antigen-binding fragment thereof, and a poly (A) in 5′-3′ orientation.

In some aspects, the nucleic acid construct or expression construct comprises a polynucleotide comprising a CMV enhancer, a CMV promoter, a SV40 intron, a nucleic acid encoding a light chain of an anti-IGF-1R antibody or antigen-binding fragment thereof, a BGHpA, a pause element, an EF1α promoter, a 5′ LTR, a chimeric human betaglobin and immunoglobulin heavy chain intron, a nucleic acid encoding a heavy chain of an anti-IGF-1R antibody or antigen-binding fragment thereof, and a SynpA in the 5′-3′ orientation.

In some aspects, the nucleic acid construct or expression construct comprises a polynucleotide comprising a CMV enhancer, a CMV promoter, a SV40 intron, a nucleic acid encoding a first signal peptide, a light chain of an anti-IGF-1R antibody or antigen-binding fragment thereof, a BGHpA, a pause element, an EF1α promoter, a 5′ LTR, a chimeric human betaglobin and immunoglobulin heavy chain intron, a nucleic acid encoding a second signal peptide, a heavy chain of an anti-IGF-1R antibody or antigen-binding fragment thereof, and a SynpA in 5′-3′ orientation.

In some aspects, the nucleic acid construct or expression construct comprises a polynucleotide comprising a first promoter, nucleic acid sequence encoding a heavy chain, a poly (A), a pause element, a second promoter, a 5′ LTR, nucleic acid sequence encoding a light chain, and a poly (A) in 5′-3′ orientation.

In some aspects, the nucleic acid construct or expression construct comprises a polynucleotide comprising a first promoter, nucleic acid sequence encoding a first signal peptide, a heavy chain, a poly (A), a pause element, a second promoter, a 5′ LTR, nucleic acid sequence encoding a second signal peptide, a light chain, and a poly (A) in 5′-3′ orientation.

In some aspects, the nucleic acid construct or expression construct comprises a polynucleotide comprising a CMV enhancer, a CMV promoter, a nucleic acid encoding a heavy chain, a BGHpA, a pause element, an EF1α promoter, a 5′ LTR, a nucleic acid encoding a light chain, and a SynpA in 5′-3′ orientation.

In some aspects, the nucleic acid construct or expression construct comprises a polynucleotide comprising a CMV enhancer, a CMV promoter, a nucleic acid encoding a first signal peptide, a heavy chain, a BGHpA, a pause element, an EF1α promoter, a 5′ LTR, a nucleic acid encoding a second signal peptide, a light chain, and a SynpA in the 5′-3′ orientation.

In some aspects, the nucleic acid construct or expression construct comprises a polynucleotide comprising a first promoter, nucleic acid sequence encoding a light chain, a poly (A), a pause element, a second promoter, a 5′ LTR, nucleic acid sequence encoding a heavy chain, and a poly (A) in 5′-3′ orientation.

In some aspects, the nucleic acid construct or expression construct comprises a polynucleotide comprising a first promoter, nucleic acid sequence encoding a first signal peptide, a light chain, a poly (A), a pause element, a second promoter, a 5′ LTR, nucleic acid sequence encoding a second signal peptide, a heavy chain, and a poly (A) in 5′-3′ orientation.

In some aspects, the nucleic acid construct or expression construct comprises a polynucleotide comprising a CMV enhancer, a CMV promoter, a nucleic acid encoding a light chain, a BGHpA, a pause element, an EF1α promoter, a 5′ LTR, a nucleic acid encoding a heavy chain, and a SynpA in 5′-3′ orientation.

In some aspects, the nucleic acid construct or expression construct comprises a polynucleotide comprising a CMV enhancer, a CMV promoter, a nucleic acid encoding a first signal peptide, a light chain, a BGHpA, a pause element, an EF1α promoter, a 5′ LTR, a nucleic acid encoding a second signal peptide, a heavy chain, and a SynpA in the 5′-3′ orientation.

In some aspects, the nucleic acid construct or expression construct comprises a polynucleotide comprising a poly (A), a nucleic acid sequence encoding a light chain, an intron, a 5′ LTR, a first promoter, a second promoter, an intron, a nucleic acid sequence encoding a heavy chain, and a poly (A) in 5′-3′ orientation.

In some aspects, the nucleic acid construct or expression construct comprises a polynucleotide comprising a poly (A), a nucleic acid sequence encoding a first signal peptide, a light chain, an intron, a 5′ LTR, a first promoter, a second promoter, an intron, a nucleic acid sequence encoding a second signal peptide, a heavy chain, and a poly (A) in the 5′-3′ orientation.

In some aspects, the nucleic acid construct or expression construct comprises a polynucleotide comprising a SYNpA, a nucleic acid encoding a light chain, a chimera of a betaglobin intron and a immunoglobulin heavy chain intron, a 5′LTR, an EF1α promoter fused to a CMV enhancer, a CMV promoter fused to a SV40 intron, a nucleic acid sequence encoding a heavy chain, and a BGHpA in 5′-3′ orientation.

In some aspects, the nucleic acid construct or expression construct comprises a polynucleotide comprising a SYNpA, a nucleic acid encoding a first signal peptide, a light chain, a chimera of a betaglobin intron and a immunoglobulin heavy chain intron, a 5′LTR, an EF1α promoter fused to a CMV enhancer, a CMV promoter fused to a SV40 intron, a nucleic acid sequence encoding a second signal peptide, a heavy chain, and a BGHpA in 5′-3′ orientation.

In some aspects, the nucleic acid construct or expression construct comprises a polynucleotide comprising a poly (A), a nucleic acid sequence encoding a heavy chain, an intron, a 5′ LTR, a first promoter, a second promoter, an intron, a nucleic acid sequence encoding a light chain, and a poly (A) in 5′-3′ orientation.

In some aspects, the nucleic acid construct or expression construct comprises a polynucleotide comprising a poly (A), a nucleic acid sequence encoding a first signal peptide, a heavy chain, an intron, a 5′ LTR, a first promoter, a second promoter, an intron, a nucleic acid sequence encoding a second signal peptide, a light chain, and a poly (A) in the 5′-3′ orientation.

In some aspects, the nucleic acid construct or expression construct comprises a polynucleotide comprising a SYNpA, a nucleic acid encoding a heavy chain, a chimera of a betaglobin intron and a immunoglobulin heavy chain intron, a 5′LTR, an EF1α promoter fused to a CMV enhancer, a CMV promoter fused to a SV40 intron, a nucleic acid sequence encoding a light chain, and a BGHpA in 5′-3′ orientation.

In some aspects, the nucleic acid construct or expression construct comprises a polynucleotide comprising a SYNpA, a nucleic acid encoding a first signal peptide, a heavy chain, a chimera of a betaglobin intron and a immunoglobulin heavy chain intron, a 5′LTR, an EF1α promoter fused to a CMV enhancer, a CMV promoter fused to a SV40 intron, a nucleic acid sequence encoding a second signal peptide, a light chain, and a BGHpA in 5′-3′ orientation.

In some aspects, the vector constructs or expression constructs (e.g., antibody expression cassettes) disclosed herein comprise one or more of the elements listed in Table 15.

TABLE 15
Nucleic Acid Sequences of Exemplary Construct Elements.

| SEQ ID | | |
|---|---|---|
| NOs | Name | Nucleic Acid Sequence |
| SEQ ID | IRES | GCCCCTCTCCCTCCCCCCCCCCTAACGTTACTGGC |
| NO: 43 | | CGAAGCCGCTTGGAATAAGGCCGGTGTGCGTTTG |
| | | TCTATATGTTATTTTCCACCATATTGCCGTCTTTTG |
| | | GCAATGTGAGGGCCCGGAAACCTGGCCCTGTCTT |
| | | CTTGACGAGCATTCCTAGGGGTCTTTCCCCTCTCG |
| | | CCAAAGGAATGCAAGGTCTGTTGAATGTCGTGAA |
| | | GGAAGCAGTTCCTCTGGAAGCTTCTTGAAGACAA |
| | | ACAACGTCTGTAGCGACCCTTTGCAGGCAGCGGA |
| | | ACCCCCCACCTGGCGACAGGTGCCTCTGCGGCCA |
| | | AAAGCCACGTGTATAAGATACACCTGCAAAGGCG |
| | | GCACAACCCCAGTGCCACGTTGTGAGTTGGATAG |
| | | TTGTGGAAAGAGTCAAATGGCTCTCCTCAAGCGT |
| | | ATTCAACAAGGGGCTGAAGGATGCCCAGAAGGTA |
| | | CCCCATTGTATGGGATCTGATCTGGGGCCTCGGTA |
| | | CACATGCTTTACATGTGTTTAGTCGAGGTTAAAAA |
| | | AACGTCTAGGCCCCCCGAACCACGGGGACGTGGT |
| | | TTTCCTTTGAAAAACACGATGATAATATGGCCACA |
| SEQ ID | Furin | AGAAAGAGAAGGAGTGGCTCAGGA |
| NO: 44 | cleavage | |
| | site + | |
| | Linker | |
| SEQ ID | F2A site | GCCCCTGTGAAACAGACCCTGAACTTTGACCTCTT |
| NO: 45 | | GAAGCTTGCTGGGGATGTGGAGTCTAATCCTGGTC |
| | | CA |
| SEQ ID | SV40 intron | GTAAGTTTAGTCTTTTTGTCTTTTATTTCAGGTCCC |
| NO: 46 | | GGATCCGGTGGTGGTGCAAATCAAAGAACTGCTC |
| | | CTCAGTGGATGTTGCCTTTACTTCTAG |
| SEQ ID | EF-1α core | GCTCCGGTGCCCGTCAGTGGGCAGAGCGCACATC |
| NO: 47 | promoter | GCCCACAGTCCCCGAGAAGTTGGGGGGAGGGGTC |
| | | GGCAATTGAACCGGTGCCTAGAGAAGGTGGCGCG |
| | | GGGTAAACTGGGAAAGTGATGTCGTGTACTGGCT |
| | | CCGCCTTTTTCCCGAGGGTGGGGGAGAACCGTAT |
| | | ATAAGTGCAGTAGTCGCCGTGAACGTTCTTTTTCG |
| | | CAACGGGTTTGCCGCCAGAACACAG |
| SEQ ID | CMV | GACATTGATTATTGACTAGTTATTAATAGTAATCA |
| NO: 48 | enhancer | ATTACGGGGTCATTAGTTCATAGCCCATATATGGA |
| | | GTTCCGCGTTACATAACTTACGGTAAATGGCCCGC |
| | | CTGGCTGACCGCCCAACGACCCCCGCCCATTGAC |
| | | GTCAATAATGACGTATGTTCCCATAGTAACGCCAA |
| | | TAGGGACTTTCCATTGACGTCAATGGGTGGACTAT |
| | | TTACGGTAAACTGCCCACTTGGCAGTACATCAAGT |
| | | GTATCATATGCCAAGTACGCCCCCTATTGACGTCA |
| | | ATGACGGTAAATGGCCCGCCTGGCATTATGCCCA |
| | | GTACATGACCTTATGGGACTTTCCTACTTGGCAGT |
| | | ACATCTACGTATTAGTCATCGCTATTACCATG |
| SEQ ID | CMV | GTGATGCGGTTTTGGCAGTACATCAATGGGCGTG |
| NO: 49 | promoter | GATAGCGGTTTGACTCACGGGGATTTCCAAGTCTC |
| | | CACCCCATTGACGTCAATGGGAGTTTGTTTTGGCA |
| | | CCAAAATCAACGGGACTTTCCAAAATGTCGTAAC |
| | | AACTCCGCCCCATTGACGCAAATGGGCGGTAGGC |
| | | GTGTACGGTGGGAGGTCTATATAAGCAGAGCTCG |
| | | TTTAGTGAACCG |
| SEQ ID | CMV | GACATTGATTATTGACTAGTTATTAATAGTAATCA |
| NO: 50 | enhancer and | ATTACGGGGTCATTAGTTCATAGCCCATATATGGA |
| | promoter | GTTCCGCGTTACATAACTTACGGTAAATGGCCCGC |
| | (CMVe/p) | CTGGCTGACCGCCCAACGACCCCCGCCCATTGAC |
| | | GTCAATAATGACGTATGTTCCCATAGTAACGCCAA |
| | | TAGGGACTTTCCATTGACGTCAATGGGTGGACTAT |
| | | TTACGGTAAACTGCCCACTTGGCAGTACATCAAGT |
| | | GTATCATATGCCAAGTACGCCCCCTATTGACGTCA |
| | | ATGACGGTAAATGGCCCGCCTGGCATTATGCCCA |
| | | GTACATGACCTTATGGGACTTTCCTACTTGGCAGT |
| | | ACATCTACGTATTAGTCATCGCTATTACCATGGTG |
| | | ATGCGGTTTTGGCAGTACATCAATGGGCGTGGAT |
| | | AGCGGTTTGACTCACGGGGATTTCCAAGTCTCCAC |
| | | CCCATTGACGTCAATGGGAGTTTGTTTTGGCACCA |
| | | AAATCAACGGGACTTTCCAAAATGTCGTAACAAC |
| | | TCCGCCCCATTGACGCAAATGGGCGGTAGGCGTG |
| | | TACGGTGGGAGGTCTATATAAGCAGAGCTCGTTT |
| | | AGTGAACCG |
| SEQ ID | CMV | GTCGACATTGATTATTGACTAGTTATTAATAGTAA |
| NO: 51 | enhancer- | TCAATTACGGGGTCATTAGTTCATAGCCCATATAT |
| | CBA | GGAGTTCCGCGTTACATAACTTACGGTAAATGGCC |
| | promoter- | CGCCTGGCTGACCGCCCAACGACCCCCGCCCATTG |
| | CAG intron | ACGTCAATAATGACGTATGTTCCCATAGTAACGCC |
| | | AATAGGGACTTTCCATTGACGTCAATGGGTGGACT |
| | | ATTTACGGTAAACTGCCCACTTGGCAGTACATCAA |
| | | GTGTATCATATGCCAAGTACGCCCCCTATTGACGT |
| | | CAATGACGGTAAATGGCCCGCCTGGCATTATGCC |
| | | CAGTACATGACCTTATGGGACTTTCCTACTTGGCA |
| | | GTACATCTACGTATTAGTCATCGCTATTACCATGG |
| | | GTCGAGGTGAGCCCCACGTTCTGCTTCACTCTCCC |
| | | CATCTCCCCCCCCTCCCCACCCCCAATTTTGTATTT |
| | | ATTTATTTTTTAATTATTTTGTGCAGCGATGGGGG |
| | | CGGGGGGGGGGGGGGCGCGCGCCAGGCGGGGCG |
| | | GGGCGGGGCGAGGGGCGGGGCGGGGCGAGGCGG |
| | | AGAGGTGCGGCGGCAGCCAATCAGAGCGGCGCGC |
| | | TCCGAAAGTTTCCTTTTATGGCGAGGCGGCGGCGG |
| | | CGGCGGCCCTATAAAAAGCGAAGCGCGCGGCGGG |
| | | CGGGAGTCGCTGCGCGCTGCCTTCGCCCCGTGCCC |
| | | CGCTCCGCCGCCGCCTCGCGCCGCCCGCCCCGGCT |
| | | CTGACTGACCGCGTTACTCCCACAGGTGAGCGGG |
| | | CGGGACGGCCCTTCTCCTCCGGGCTGTAATTAGCG |
| | | CTTGGTTTAATGACGGCTCGTTTCTTTTCTGTGGCT |
| | | GCGTGAAAGCCTTGAGGGGCTCCGGGAGGGCCCT |
| | | TTGTGCGGGGGGAGCGGCTCGGGGGGTGCGTGCG |
| | | TGTGTGTGTGCGTGGGGAGCGCCGCGTGCGGCTC |
| | | CGCGCTGCCCGGCGGCTGTGAGCGCTGCGGGCGC |
| | | GGCGCGGGGCTTTGTGCGCTCCGCAGTGTGCGCG |
| | | AGAGGAGCGCGGCCGGGGGCGGTGCCACGCGGTG |
| | | CGGGGGGGGCTGCGAGGGGAACAAAGGCTGCGT |
| | | GCGGGGTGTGTGCGTGGGGGGGGTGAGCAGGGGG |
| | | TGTGGGCGCGGCGGTCGGGCTGTAACCCCCCCCT |
| | | GCACCCCCCTCCCCGAGTTGCTGAGCACGGCCCG |
| | | GCTTCGGGTGCGGGGCTCCGTGCGGGGCGTGGCG |
| | | CGGGGCTCGCCGTGCCGGGCGGGGGGTGGCGGCG |
| | | GGTGGGGGTGCCGGGCGGGGGGGGGCCGCCTCGG |
| | | GCCGGGGAGGGCTCGGGGGAGGGGCGCGGCGGC |
| | | CCCCGGAGCGCCGGCGGCTGTCGAGGCGCGGCGA |
| | | GCCGCAGCCATTGCCTTTTATGGTAATCGTGCGAG |
| | | AGGGCGCAGGGACTTCCTTTGTCCCAAATCTGTGC |
| | | GGAGCCGAAATCTGGGAGGCGCCGCCGCACCCCC |
| | | TCTAGCGGGCGCGGGGCGAAGCGGTGCGGCGCCG |
| | | GCAGGAAGGAAATGGGCGGGGAGGGCCTTCGTGC |
| | | GTCGCCGCGCCGCCGTCCCCTTCTCCCTCTCCAGC |
| | | CTCGGGGCTGTCCGCGGGGGGACGGCTGCCTTCG |
| | | GGGGGGACGGGGCAGGGCGGGGTTCGGCTTCTGG |
| | | CGTGTGACCGGCGGTCTAGACTCTGCTAACCATGT |
| | | TCATGCCTTCTTCTCTTTCCTACAGCTCCTGGGCAA |
| | | CGTGCTGGTTGTTGTGCTGTCTCATCATTTTGGCA |
| | | AA |
| SEQ ID | Synthetic | AATAAAAGATCTTTATTTTCATTAGATCTGTGTGT |
| NO: 52 | poly(A) | TGGTTTTTTGTGTG |
| SEQ ID | BGH poly(A) | CTGTGCCTTCTAGTTGCCAGCCATCTGTTGTTTGCC |
| NO: 53 | | CCTCCCCCGTGCCTTCCTTGACCCTGGAAGGTGCC |
| | | ACTCCCACTGTCCTTTCCTAATAAAATGAGGAAAT |
| | | TGCATCGCATTGTCTGAGTAGGTGTCATTCTATTC |
| | | TGGGGGGTGGGGTGGGGCAGGACAGCAAGGGGG |
| | | AGGATTGGGAAGACAATAGCAGGCATGCTGGGGA |
| | | TGCGGTGGGCTCTATG |
| SEQ ID | Pause | AACATACGCTCTCCATCAAAACAAAACGAAACAA |
| NO: 54 | element | AACAAACTAGCAAAATAGGCTGTCCCCAGTGCAA |
| | | GTGCAGGTGCCAGAACATTTCTCT |
| SEQ ID | 5′ LTR | GGGGCTCGCATCTCTCCTTCACGCGCCCGCCGCCC |
| NO: 55 | (truncated) | TACCTGAGGCCGCCATCCACGCCGGTTGAGTCGC |
| | | GTTCTGCCGCCTCCCGCCTGTGGTGCCTCCTGAAC |
| | | TGCGTCCGCCGTCTAGGTAAGTTTAAAGCTCAGGT |
| | | CGAGACCGGGCCTTTGTCCGGCGCTCCCTTGGAGC |
| | | CTACCTAGACTCAGCCGGCTCTCCACGCTTTGCCT |
| | | GACCCTGCTTGCTCAACTCTACGTCTTTGTTTCGTT |
| | | TTCTGTTCTGCGCCGTTACAGATC |
| SEQ ID | Intron | GTAAGTATCAAGGTTACAAGACAGGTTTAAGGAG |
| NO: 56 | (chimera of | ACCAATAGAAACTGGGCTTGTCGAGACAGAGAAG |
| | human | ACTCTTGCGTTTCTGATAGGCACCTATTGGTCTTA |
| | betaglobin | CTGACATCCACTTTGCCTTTCTCTCCACAG |
| | intron and | |
| | immunoglobulin | |
| | heavy | |
| | chain intron) | |
| SEQ ID | CAG intron | ggagtcgctgcgcgctgccttcgccccgtgccccgctccgccgccgcctcgcgc |
| NO: 82 | | cgcccgccccggctctgactgaccgcgttactcccacaggtgagcgggcgggac |
| | | ggcccttctcctccgggctgtaattagcgcttggtttaatgacggctcgtttctt |
| | | tggctgcgtgaaagccttgaggggctccgggagggccctttgtgcggggggagc |
| | | ggctcggggggtgcgtgcgtgtgtgtgtgcgtggggagcgccgcgtgcggctcc |
| | | gcgctgcccggcggctgtgagcgctgcgggcgcggcgcggggctttgtgcgct |
| | | ccgcagtgtgcgcgagaggagcgcggccgggggcggtgccacgcggtgcgg |
| | | ggggggctgcgaggggaacaaaggctgcgtgcggggtgtgtgcgtggggggg |
| | | gtgagcagggggtgtgggcgcggcggtcgggctgtaacccccccctgcacccc |
| | | cctccccgagttgctgagcacggcccggcttcgggtgcggggctccgtgcgggg |
| | | cgtggcgcggggctcgccgtgccgggggggggtggcggcgggtgggggtg |
| | | ccgggcggggggggccgcctcgggccggggagggctcgggggaggggcg |
| | | cggcggcccccggagcgccggcggctgtcgaggcgcggcgagccgcagcca |
| | | ttgccttttatggtaatcgtgcgagagggcgcagggacttcctttgtcccaaatc |
| | | tgtgcggagccgaaatctgggaggcgccgccgcaccccctctagcgggcgcgggg |
| | | cgaagcggtgcggcgccggcaggaaggaaatgggcggggagggccttcgtgc |
| | | gtcgccgcgccgccgtccccttctccctctccagcctcggggctgtccgcggggg |
| | | gacggctgccttcgggggggacggggcagggcggggttcggcttctggcgtgt |
| | | gaccggcggtctagactctgctaaccatgttcatgccttcttctctttcctacag |
| | | ctcctgggcaacgtgctggttgttgtgctgtctcatcattttggcaaa |
| SEQ ID | CBA | tegaggtgagccccacgttctgcttcactctccccattcccccccctccccacc |
| NO: 83 | promoter #1 | cccaattttgtatttatttattttttaattattttgtgcagcgatgggggcgggg |
| | | ggggggggggcgcgcgccaggcggcggggggcggggcgaggggggggcggggc |
| | | gaggcggagaggtgcggcggcagccaatcagagcggegcgctccgaaagtttc |
| | | cttttatggcgaggcggcggcggcggcggccct |
| | | ataaaaagcgaagcgcgcggcgggcg |
| SEQ ID | CBA | tcgaggtgagccccacgttctgcttcactctccccatctcccccccctccccac |
| NO: 93 | promoter #2 | ccccaattttgtatttatttattttttaattattttgtgcagcgatgggggcggg |
| | | gggggggggggggcgcgcgccaggcggggcggggcggggcgaggggcggggcggg |
| | | gcgaggcggagaggtgcggggcagccaatcagagcggegcgctccgaaagtttc |
| | | cttttatggcgaggcggcggcggcggcggccctataaaaagcgaagcgcgcgg |
| | | cgggcg |

### III.A. Delivery Vectors

In some aspects, the delivery vector is a viral vector, a non-viral vectors, a plasmid, a lipid, or a lysosome. In some aspects, the therapeutic effect of the antibody or antigen-binding fragment thereof is local, systemic, or both.

In certain aspects, a composition comprising a delivery vector (e.g., a viral vector, a non-viral vectors, a plasmid, a lipid, a protein particle, a bacterial vector, or a lysosome) comprising a nucleic acid encoding or comprising an antibody (e.g., a monoclonal antibody) or an antigen binding fragment thereof disclosed herein.

In some aspects, the delivery vector is suitable for delivery to or near an eye (e.g., one or both eyes), e.g., intraocular, retro- or a peri-orbital, retrobulbar, intramuscular near the eye, to connective tissue near the eye, or any combination thereof. In some aspects, the delivery or administration is to retro or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the composition comprising a delivery vector (e.g., a viral vector, a non-viral vectors, a plasmid, a lipid, a protein particle, a bacterial vector, or a lysosome) comprises a nucleic acid encoding an antibody (e.g., a monoclonal antibody) or an antigen-binding fragment thereof disclosed herein is produced in a cell. In some aspects, the cell is a fibroblast cell, adipocytes cell, myofibroblast cell, myocyte cell, or any combination thereof.

In some aspects, a composition comprising the delivery vector (e.g., a viral vector, a non-viral vectors, a plasmid, a lipid, a protein particle, a bacterial vector, or a lysosome) comprising a nucleic acid encoding or comprising an antibody (e.g., a monoclonal antibody) or an antigen binding fragment thereof disclosed herein is suitable for delivery to connective tissue, muscle tissue, and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node.

In some aspects, the delivery vector comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

### III.A.1 Non-Viral Vectors

The DNA of interest can be administered using a non-viral vector. “Non-viral vector,” as used herein is meant to include naked DNA, chemical formulations containing naked DNA (e.g., a formulation of DNA and cationic compounds (e.g., dextran sulfate)), and naked DNA mixed with an adjuvant such as a viral particle (i.e., the DNA of interest is not contained within the viral particle, but the transforming formulation is composed of both naked DNA and viral particles (e.g., AAV particles) (see e.g., Curiel et al., Am. J. Respir. Cell Mol. Biol. 6:247-52 (1992)). Thus the “non-viral vector” can include vectors composed of DNA plus viral particles where the viral particles do not contain the DNA of interest within the viral genome.

In some aspects, the non-viral vector is a bacterial vector. See e.g., Baban et al., Bioeng Bugs., 1 (6): 385-394 (2010).

In some aspects, the DNA of interest can be complexed with polycationic substances such as poly-L-lysine or DEAC-dextran, targeting ligands, and/or DNA binding proteins (e.g., histones). DNA- or RNA-liposome complex formulations comprise a mixture of lipids which bind to genetic material (DNA or RNA) and facilitate delivery of the nucleic acid into the cell. Liposomes which can be used in accordance with the disclosure include DOPE (dioleyl phosphatidyl ethanol amine), CUDMEDA (N-(5-cholestrum-3-β-ol 3-urethanyl)-N′,N′-dimethylethylene diamine).

Lipids which can be used in accordance with the disclosure include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). The concentration of the liposome solutions range from about 2.5% to 15% volume: volume, preferably about 6% to 12% volume: volume. Further exemplary methods and compositions for formulation of nucleic acid (e.g., DNA, including DNA or RNA not contained within a viral particle) for delivery according to the method of the disclosure are described in U.S. Pat. Nos. 5,892,071; 5,744,625; 5,925,623; 5,527,928; 5,824,812; 5,869,715.

Polymer particles can be used in accordance with the disclosure for polymer-based gene delivery. See e.g., Putnam et al., PNAS 98 (3): 1200-1205 (2001).

The DNA of interest can also be administered as a chemical formulation of DNA or RNA coupled to a carrier molecule (e.g., an antibody or a receptor ligand) which facilitates delivery to host cells for the purpose of altering the biological properties of the host cells. The term “chemical formulations” refers to modifications of nucleic acids to allow coupling of the nucleic acid compounds to a carrier molecule such as a protein or lipid, or derivative thereof. Exemplary protein carrier molecules include antibodies specific to the target cells or receptor ligands, i.e., molecules capable of interacting with receptors associated with a target cell.

In certain aspects, a composition comprising a non-viral delivery vector comprising a nucleic acid encoding or comprising an antibody (e.g., a monoclonal antibody) or an antigen binding fragment thereof disclosed herein suitable for delivery to or near an eye (e.g., one or both eyes), e.g., intraocular, retro or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the composition comprising a non-viral delivery vector comprises a nucleic acid encoding an antibody (e.g., a monoclonal antibody) or an antigen-binding fragment thereof disclosed herein, or a therapeutic peptide that is produced in the target cells. In some aspects, the therapeutic effect of the antibody or antigen-binding fragment thereof is local, systemic, or both.

In some aspects, the non-viral vector comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

### III.A.2 Viral Vectors

In general, viral vectors used in accordance with the disclosure are composed of a viral particle derived from a naturally-occurring virus which has been genetically altered to render the virus replication-defective and to express a recombinant gene of interest in accordance with the disclosure. Once the virus delivers its genetic material to a cell, it does not generate additional infectious virus but does introduce exogenous recombinant genes into the cell, preferably into the genome of the cell.

Numerous viral vectors are well known in the art, including, for example, retrovirus, adenovirus, adeno-associated virus (AAV), herpes simplex virus (HSV), cytomegalovirus (CMV), vaccinia and poliovirus vectors. Retroviral vectors are less preferred since retroviruses require replicating cells and secretory glands are composed of mostly slowly replicating and/or terminally differentiated cells. Adenovirus and AAV are preferred viral vectors since this virus efficiently infects slowly replicating and/or terminally differentiated cells. In some aspects, the delivery vector (e.g., viral vector) is selected from the group consisting of an adeno-associated viral (AAV) vector, an adenoviral vector, a lentiviral vector, or a retroviral vector.

Where a replication-deficient virus is used as the viral vector, the production of infective virus particles containing either DNA or RNA corresponding to the DNA of interest can be produced by introducing the viral construct into a recombinant cell line which provides the missing components essential for viral replication. In some aspects, transformation of the recombinant cell line with the recombinant viral vector will not result in production of replication-competent viruses, e.g., by homologous recombination of the viral sequences of the recombinant cell line into the introduced viral vector. Methods for production of replication-deficient viral particles containing a nucleic acid of interest are well known in the art and are described in, e.g., Rosenfeld et al., Science 252:431-434 (1991) and Rosenfeld et al., Cell 68:143-155 (1992) (adenovirus); U.S. Pat. No. 5,139,941 (adeno-associated virus); U.S. Pat. No. 4,861,719 (retrovirus); and U.S. Pat. No. 5,356,806 (vaccinia virus).

In certain aspects, the viral delivery vector comprising a nucleic acid encoding or comprising an antibody (e.g., a monoclonal antibody) or an antigen binding fragment thereof disclosed herein suitable for delivery to or near an eye (e.g., one or both eyes), e.g., intraocular, retro or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro- or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the viral delivery vector comprises a nucleic acid encoding a therapeutic protein, e.g., an antibody (e.g., a monoclonal antibody) or an antigen-binding fragment thereof disclosed herein is produced in a target cell. In some aspects, the therapeutic effect of the therapeutic antibody or antigen-binding fragment thereof is local, systemic, or both.

In some aspects, the viral vector comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

### IV. Adeno-Associated Virus (AAV)-Mediated Gene Therapy

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing the DNA, a suitable host cell line is transfected with an AAV vector containing the DNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the DNA enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV vectors of the present disclosure can comprise or be derived from any natural or recombinant AAV serotype. According to the present disclosure, the AAV serotype can be, but is not limited to, AAV1, AAV2, AAV3, AAV4, AAV5, AAV6, AAV7, AAV8, AAVrh8, AAV9, AAV10, AAVrh10, AAV11, and AAV12. In some aspects, the AAV serotype is AAV1, AAV2, AAV6, AAV8, or AAV9. In some aspect, the AAV vector is modified relative to the wild-type AAV serotype sequence. In some aspects, the AAV vector is modified relative to wild-type AAV1, AAV2, AAV6, AAV8, or AAV9. In some aspects, the AAV vector serotype is AAV1 or a modified AAV vector derived therefrom. In some aspects, the AAV vector serotype is AAV2 or a modified AAV vector derived therefrom. In some aspects, the AAV vector serotype is AAV6 or a modified AAV vector derived therefrom. In some aspects, the AAV vector serotype is AAV8 or a modified AAV vector derived therefrom. In some aspects, the AAV vector serotype is AAV9 or a modified AAV vector derived therefrom. In some aspects, the AAV vector serotype is AAVMYO (see Weinmann et al. Nat. Comm. 11:5432, 2020).

In certain aspects, a composition comprising an AAV delivery vector comprising a nucleic acid encoding or comprising an antibody (e.g., a monoclonal antibody) or an antigen binding fragment thereof disclosed herein. In some aspects, the AAV delivery vector comprising a nucleic acid encoding or comprising an antibody (e.g., a monoclonal antibody) or an antigen binding fragment thereof disclosed herein suitable for delivery to or near an eye (e.g., one or both eyes), e.g., intraocular, retro or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro- or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the AAV delivery vector comprises a nucleic acid encoding an antibody (e.g., a monoclonal antibody) or an antigen-binding fragment thereof disclosed herein is produced in a fibroblast, myofibroblast, myocyte, and/or adipocyte. In some aspects, the therapeutic effect of the antibody or antigen-binding fragment thereof is local, systemic, or both.

In some aspects, the AAV delivery vector comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

### IV.A. AAV Vector Components

### IV.A.1 Inverted Terminal Repeats (ITRs)

The AAV vectors of the present disclosure comprise a viral genome with at least one ITR region and a payload region, e.g., a polynucleotide (e.g., an antibody expression cassette) comprising a nucleic acid encoding a therapeutic protein, e.g., an antibody (e.g., a monoclonal antibody) or an antigen binding fragment thereof or a fusion protein (e.g., an Fc fusion protein) disclosed herein, or a therapeutic peptide. In some aspects, the AAV vector has two ITRs. These two ITRs flank the payload region at 5′ and 3′ ends. The ITRs function as origins of replication comprising recognition sites for replication. ITRs comprise sequence regions, which can be complementary and symmetrically arranged. ITRs incorporated into AAV vectors of the disclosure can be comprised of naturally occurring polynucleotide sequences or recombinantly derived polynucleotide sequences.

The ITRs can be derived from the same serotype as the capsid, selected from any of the serotypes listed herein, or a derivative thereof. The ITR can be of a different serotype from the capsid. In some aspects, the AAV vector has more than one ITR. In a non-limiting example, the AAV vector has a viral genome comprising two ITRs. In some aspects, the ITRs are of the same serotype as one another. In some aspects, the ITRs are of different serotypes. Non-limiting examples include zero, one or both of the ITRs having the same serotype as the capsid. In some aspects both ITRs of the AAV vector are AAV2 ITRs.

Independently, each ITR can be about 75 to about 175 nucleotides in length. An ITR can be about 100-105 nucleotides in length, about 106-110 nucleotides in length, about 111-115 nucleotides in length, about 116-120 nucleotides in length, about 121-125 nucleotides in length, about 126-130 nucleotides in length, about 131-135 nucleotides in length, about 136-140 nucleotides in length, about 141-145 nucleotides in length or about 146-150 nucleotides in length. In some aspects, the ITRs are about 140-142 nucleotides in length. Non-limiting examples of ITR length are about 102, about 140, about 141, about 142, about 145 nucleotides in length, and those having at least 95% identity thereto.

In some aspects, the AAV vector comprises at least one inverted terminal repeat having a length such as, but not limited to, about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, or about 170-175 nucleotides.

In some aspects, the length of a first and/or a second ITR regions for the AAV vector can be about 75-80, about 75-85, about 75-100, about 80-85, about 80-90, about 80-105, about 85-90, about 85-95, about 85-110, about 90-95, about 90-100, about 90-115, about 95-100, about 95-105, about 95-120, about 100-105, about 100-110, about 100-125, about 105-110, about 105-115, about 105-130, about 110-115, about 110-120, about 110-135, about 115-120, about 115-125, about 115-140, about 120-125, about 120-130, about 120-145, about 125-130, about 125-135, about 125-150, about 130-135, about 130-140, about 130-155, about 135-140, about 135-145, about 135-160, about 140-145, about 140-150, about 140-165, about 145-150, about 145-155, about 145-170, about 150-155, about 150-160, about 150-175, about 155-160, about 155-165, about 160-165, about 160-170, about 165-170, about 165-175, and about 170-175 nucleotides.

In some aspects, the AAV vector comprises a nucleic acid sequence encoding an antibody (e.g., a monoclonal antibody) or an antigen binding fragment thereof disclosed herein which can be located near the 5′ end of the flip ITR in the vector. In some aspects, the AAV vector comprises a nucleic acid sequence encoding an antibody (e.g., a monoclonal antibody) or an antigen binding fragment thereof disclosed herein, which can be located near the 3′ end of the flip ITR in the vector. In some aspects, the AAV vector comprises a nucleic acid sequence encoding an antibody (e.g., a monoclonal antibody) or an antigen binding fragment thereof disclosed herein, which can be located near 5′ end of the flop ITR in the vector. In some aspects, the AAV vector comprises a nucleic acid sequence encoding an antibody (e.g., a monoclonal antibody) or an antigen binding fragment thereof disclosed herein, which can be located near 3′ end of the flop ITR in the vector. In some aspects, the AAV vector comprises a nucleic acid sequence encoding an antibody (e.g., a monoclonal antibody) or an antigen binding fragment thereof disclosed herein, which can be located between 5′ end of the flip ITR and 3′ end of the flop ITR in the vector. In some aspects, the AAV vector comprises a nucleic acid sequence encoding an antibody (e.g., a monoclonal antibody) or an antigen binding fragment thereof disclosed herein, which can be located between (e.g., half-way between the 5′ end of the flip ITR and 3′ end of the flop ITR or 3′ end of the flop ITR and 5′ end of the flip ITR), 3′ end of the flip ITR and 5′ end of the flip ITR in the vector. In some aspects, the AAV vector comprises a nucleic acid sequence encoding an antibody (e.g., a monoclonal antibody) or an antigen binding fragment thereof disclosed herein, which can be located within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or more than about 30 nucleotides downstream or upstream from the 5′ or 3′ end of an ITR (e.g., Flip or Flop ITR) in the vector.

As another non-limiting example, the AAV vector comprises a nucleic acid sequence encoding an antibody (e.g., a monoclonal antibody) or an antigen binding fragment thereof disclosed herein, which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream or upstream from the 5′ or 3′ end of an ITR (e.g., Flip or Flop ITR) in the vector.

In some aspects, the AAV vector comprises a nucleic acid sequence encoding an antibody (e.g., a monoclonal antibody) or an antigen binding fragment thereof disclosed herein, which can be located within the first about 1%, about 2%, about 3%, about 4%, about 5%, about 6%, about 7%, about 8%, about 9%, about 10%, about 15%, about 20%, about 25% or more than about 25% of the nucleotides upstream from the 5′ or 3′ end of an ITR (e.g., Flip or Flop ITR) in the vector.

As another non-limiting example, the AAV vector comprises a nucleic acid sequence encoding an antibody (e.g., a monoclonal antibody) or an antigen binding fragment thereof disclosed herein, which can be located with the first about 1-5%, about 1-10%, about 1-15%, about 1-20%, about 1-25%, about 5-10%, about 5-15%, about 5-20%, about 5-25%, about 10-15%, about 10-20%, about 10-25%, about 15-20%, about 15-25%, or about 20-25% downstream from the 5′ or 3′ end of an ITR (e.g., Flip or Flop ITR) in the vector.

### IV.A.2 Promoters

In some aspects, the payload region of the AAV vector comprises at least one element to enhance the nucleic acid specificity and/or expression. Non-limiting examples of elements to enhance the nucleic acid specificity and expression include, e.g., promoters, endogenous miRNAs, post-transcriptional regulatory elements (PREs), polyadenylation (Poly A) signal sequences and upstream enhancers (USEs), CMV enhancers, and introns. In some aspects, the enhancer is a CMV enhancer. In some aspects, the CMV enhancer comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 35.

Expression of nucleic acid of the present disclosure after delivery to or integration in the genomic DNA of a target cell can require a specific promoter, including but not limited to, a promoter that is species specific, inducible, tissue-specific, or cell cycle-specific (Parr et al., Nat. Med. 3:1145-9 (1997); the contents of which are herein incorporated by reference in their entirety).

In some aspects, the promoter is deemed to be efficient when it drives expression of an antibody (e.g., a monoclonal antibody) or an antigen binding fragment thereof disclosed herein carried in the payload region of the AAV vector. In some aspects, the promoter is a promoter deemed to be efficient when it drives expression of the therapeutic molecule of the present disclosure in the cell being targeted (e.g., fibroblast, myocyte, adipocyte).

Promoters can be naturally occurring or non-naturally occurring. Non-limiting examples of promoters include viral promoters and mammalian promoters. In some aspects, the promoters can be human promoters. In some aspects, the promoter can be truncated. Promoters which drive or promote expression in most tissues include, but are not limited to, human elongation factor 1a-subunit (EF1a), cytomegalovirus (CMV) immediate-early enhancer and/or promoter, chicken β-actin (CBA) and its derivative CAG, B glucuronidase (GUSB), or ubiquitin C (UBC). In some aspects, the promoter is a CMV early enhancer/chicken β actin (CAG) promoter, CAG, CBA, CMV, EF1α, EF1α with a CMV enhancer, a CMV promoter with a CMV enhancer (CMVe/p), a CMV promoter with a SV40 intron, or tissue specific promoter.

In some aspects, tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, HSA promoter, HMCK7 promoter, dMCK promoter, tMCK promoter, CK8e promoter, SPc5-12 promoter, SP-301 promoter, MH promoter, Sk-CRM promoter, or Sk-CRM4 promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety). Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene ηβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter. Non-limiting examples of tissue-specific expression elements for fibroblasts include an insulin-like growth factor binding protein 2 (IGFBP2) promoter, a fibroblast activation protein (FAP) promoter, and fibroblast specific protein 1 (FSP1) promoter.

Non-limiting examples of tissue-specific expression elements for adipocytes include an adiponectin promoter and adipocyte fatty acid-binding protein (AP2) promoter.

In some aspects, the promoter can be less than 1 kb. In some aspects, the promoter can have a length between about 15-20, about 10-50, about 20-30, about 30-40, about 40-50, about 50-60, about 50-100, about 60-70, about 70-80, about 80-90, about 90-100, about 100-110, about 100-150, about 110-120, about 120-130, about 130-140, about 140-150, about 150-160, about 150-200, about 160-170, about 170-180, about 180-190, about 190-200, about 200-210, about 200-250, about 210-220, about 220-230, about 230-240, about 240-250, about 250-260, about 250-300, about 260-270, about 270-280, about 280-290, about 290-300, about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides.

In some aspects, the promoter can be a combination of two or more components of the same or different starting or parental promoters such as, but not limited to, CMV, CAG, EF1α, and CBA. In some aspects, the promoter is a CMV early enhancer/chicken β actin (CAG) promoter, a CAG promoter, a CBA promoter, a human CMV promoter, a mouse CMV promoter, an EF1α promoter, an EF1α promoter with a CMV enhancer, a CMV promoter with a CMV enhancer (CMVe/p), a CMV promoter with a SV40 intron. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51, 83, or 93.

In some aspects, each component in the promoter can have a length between about 200-300, about 200-400, about 200-500, about 200-600, about 200-700, about 200-800, about 300-400, about 300-500, about 300-600, about 300-700, about 300-800, about 400-500, about 400-600, about 400-700, about 400-800, about 500-600, about 500-700, about 500-800, about 600-700, about 600-800 or about 700-800 nucleotides. In some aspects, the promoter is a combination of a 382 nucleotide CMV-enhancer sequence and a 260 nucleotide CBA-promoter sequence.

In some aspects, the AAV vector comprises a ubiquitous promoter. Non-limiting examples of ubiquitous promoters include, e.g., a human CMV promoter, a mouse CMV promoter, a CBA promoter (including derivatives CAG, CBh, etc.), an EF-1a promoter, a PGK promoter, an UBC promoter, a GUSB promoter (hGBp), and an UCOE promoter (promoter of HNRPA2B1-CBX3).

In some aspects, the promoter is not cell specific. In some aspects, the promoter is a ubiquitin c (UBC) promoter. The UBC promoter can have a size of 300-350 nucleotides. In some aspects, the UBC promoter is 332 nucleotides. In some aspects, the promoter is a β-glucuronidase (GUSB) promoter. The GUSB promoter can have a size of 350-400 nucleotides. In some aspects, the GUSB promoter is 378 nucleotides. In some aspects, the promoter is a neurofilament light (NFL) promoter. The NFL promoter can have a size of 600-700 nucleotides. In some aspects, the NFL promoter is 650 nucleotides. In some aspects, the construct can be AAV-promoter-CMV/globin intron-modulatory polynucleotide-RBG, where the AAV can be self-complementary and the AAV can be the DJ serotype.

In some aspects, the AAV vector comprises a Pol III promoter. In some aspects, the AAV vector comprises a PI promoter. In some aspects, the AAV vector comprises a FXN promoter. In some aspects, the promoter is a phosphogly cerate kinase 1 (PGK) promoter. In some aspects, the promoter is a chicken β-actin (CBA) promoter. In some aspects, the promoter is a CAG promoter which is a construct comprising the cytomegalovirus (CMV) enhancer fused to the chicken beta-actin (CBA) promoter with a chimeric intron. In some aspects, the promoter is a cytomegalovirus (CMV) promoter. In some aspects, the promoter is a human cytomegalovirus (CMV) promoter. In some aspects, the promoter is a mouse cytomegalovirus (CMV) promoter. In some aspects, the promoter is a CBA promoter. In some aspects, the promoter is an EF1α promoter. In some aspects, the promoter is an EF1α promoter fused to a CMV enhancer. In some aspects, the promoter is a CMV promoter fused to a CMV enhancer. In some aspects, the promoter is a CMV promoter fused to a SV40 intron. In some aspects, the AAV vector comprises a HI promoter. In some aspects, the AAV vector comprises a U6 promoter. In some aspects, the AAV vector comprises a SP6 promoter. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 34-38.

In some aspects, the promoter is a liver or a skeletal muscle promoter. Non-limiting examples of liver promoters include human a-1-antitrypsin (hAAT) and thyroxine binding globulin (TBG). Non-limiting examples of skeletal muscle promoters include Desmin, MCK or synthetic C5-12. In some aspects, the promoter is an RNA pol III promoter. In some aspects, the RNA pol III promoter is U6. In some aspects, the RNA pol III promoter is HI. In some aspects, the AAV vector comprises two promoters. In some aspects, the promoters are an EF1α promoter and a CMV promoter. Non-limiting examples of fibroblast promoters include insulin-like growth factor binding protein 2 (IGFBP2), a fibroblast activation protein (FAP), and fibroblast specific protein 1 (FSP1).

Non-limiting examples of adipocyte promoters include an adiponectin and adipocyte fatty acid-binding protein (AP2).

In some aspects, the AAV vector comprises an enhancer element, a promoter and/or a 5′UTR intron. The enhancer element, also referred to herein as an “enhancer,” can be, but is not limited to, a CMV enhancer, the promoter can be, but is not limited to, an EF1α, CMV, CBA, UBC, GUSB, NSE, Synapsin, MeCP2, and GFAP promoter and the 5′UTR/intron can be, but is not limited to, SV40, CBA-MVM (Minute virus of mice), human β-globin, immunoglobulin heavy chain, a chimera between the human β-globin and immunoglobin heavy chain gene. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any one of SEQ ID NOs: 46, 56, or 82. In some aspects, the enhancer is a CMV enhancer. In some aspects, the CMV enhancer comprises a comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 48. In some aspects, the enhancer, promoter and/or intron used in combination can be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter, (9) GFAP promoter, (10) HI promoter; (11) U6 promoter; (12) CMV promoter, CMV enhancer; (13) EF1α promoter, CMV enhancer; or (14) CMV promoter, SV40 intron; (15) human β-globin and immunoglobin heavy chain chimera, EF1α promoter, CMV enhancer, CMV promoter, SV40 intron. In some aspects, the promoter is a cytomegalovirus (CMV) promoter. In some aspects, the intron is a SV40 intron, MVM intron or a human betaglobin intron in the vector. In some aspects, the promoter is a CBA promoter. In some aspects, the promoter is an EF1α promoter. In some aspects, the promoter is a CMV promoter fused to a CMV enhancer. In some aspects, the promoter is a CMV enhancer fused to an EF1α promoter. In some aspects, the promoter is a CMV promoter fused to a SV40 intron. In some aspects, the AA vector comprises an engineered promoter. In some aspects, the AAV vector comprises a CMV early enhancer/chicken β actin (CAG) promoter. In some aspects the AAV vector comprises a promoter from a naturally expressed protein.

### IV.A.3 Untranslated Regions (UTRs)

By definition, wild-type untranslated regions (UTRs) of a gene are transcribed but not translated. Generally, 5′ UTR starts at the transcription start site and ends at the start codon and 3′ UTR starts immediately following the stop codon and continues until the termination signal for transcription.

Features typically found in abundantly expressed genes of specific target organs can be engineered into UTRs to enhance transcribed product stability and production. In some aspects, a 5′ UTR from mRNA normally expressed in the liver (e.g., albumin, serum amyloid A, Apolipoprotein A/B/E, transferrin, alpha fetoprotein, erythropoietin, or Factor VIII) can be used in AAV vector of the disclosure to enhance expression, e.g., in brain tissue, and specifically in neuronal cells.

Wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR (A/G) CCAUGG, where R is a purine (adenine or guanine) three bases upstream of the start codon (ATG), which is followed by another ′G. In some aspects, the 5′ UTR in an AAV vector of the present disclosure includes a Kozak sequence. In some aspects, 5′ UTR in an AAV vector of the present disclosure does not include a Kozak sequence.

Wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety). Class I AREs, such as, but not limited to, c-Myc and MyoD, contain several dispersed copies of an AUUUA motif within U-rich regions. Class II AREs, such as, but not limited to, GM-CSF and IGFR-α, possess two or more overlapping UUAUUUA (U/A) (U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some aspects, 3′ UTR of an AAV vector of the present disclosure can include an oligo (dT) sequence for addition of a poly-A tail.

In some aspects, an AAV vector of the present disclosure can be engineered to include, alter or remove at least one miRNA binding site, sequence or seed region.

Any UTR from any gene known in the art can be incorporated into an AAV vector of the present disclosure. These UTRs, or portions thereof, can be placed in the same orientation as in the gene from which they were selected or they can be altered in orientation or location. In some aspects, the UTR used in an AAV vector of the present disclosure can be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR can be altered relative to a wild-type or native UTR by the change in orientation or location as taught above or can be altered by the inclusion of additional nucleotides, deletion of nucleotides, swapping or transposition of nucleotides. In some aspects, an AAV vector of the present disclosure comprises at least one artificial UTRs, which is not a variant of a wild-type UTR. In some aspects, an AAV vector of the present disclosure comprises UTRs, which have been selected from a family of transcripts whose proteins share a common function, structure, feature or property.

### IV.A.4 Polyadenylation Sequence

In some aspects, the AAV vectors of the present disclosure comprise at least one polyadenylation sequence. The AAV vectors of the present disclosure can comprise a polyadenylation sequence between 3′ end of the payload coding sequence and 5′ end of 3′ ITR.

In some aspects, the polyadenylation sequence or “polyA sequence” can range from absent to about 500 nucleotides in length.

In some aspects, the polyadenylation sequence is about 10-100, about 10-90, about 10-80, about 10-70, about 10-60, about 10-55, about 10-50, about 20-100, about 20-90, about 20-80, about 20-70, about 20-60, about 20-55, about 20-50, about 30-100, about 30-90, about 30-80, about 30-70, about 30-60, about 30-55, about 30-50, about 40-100, about 40-90, about 40-80, about 40-70, about 40-60, about 40-55, about 40-50, about 45-100, about 45-90, about 45-80, or about 45-70 about 45-60, about 45-55, about 45-50 nucleotides in length. In some aspects, the polyadenylation sequence is about 49 nucleotides in length.

In some aspects, the AAV vector comprises a nucleic acid sequence encoding an antibody (e.g., a monoclonal antibody) or an antigen binding fragment thereof disclosed herein, which can be located upstream of the polyadenylation sequence in the vector. In some aspects, the AAV vector comprises a nucleic acid sequence encoding an antibody (e.g., a monoclonal antibody) or an antigen binding fragment thereof disclosed herein, which can be located downstream of a promoter such as, but not limited to, EF1α, CMV, U6, CAG, CBA EF1α with a CMV enhancer, CMV promoter with a SV40 intron, CMV promoter with a CMV enhancer, or a CBA promoter with a SV40 intron, MVM intron a human betaglobin intron, immunoglobulin heavy chain intron, or a chimera of a human betaglobin intron and a immunoglobulin heavy chain intron in the vector.

In some aspects, the AAV vector of the present disclosure comprises a nucleic acid sequence encoding an antibody (e.g., a monoclonal antibody) or an antigen binding fragment thereof disclosed herein, which can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

In some aspects, the AAV vector comprises a rabbit globin polyadenylation (poly A) signal sequence. In some aspects, the AAV vector comprises a human growth hormone polyadenylation (poly A) signal sequence. In some aspects, the AAV vector comprises a bovine growth hormone polyadenylation (poly A) signal sequence. In some aspects, the poly A signal sequence has a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 52 or 53.

In some aspects, the AAV vector comprises an SV40 polyadenylation signal sequence (SV40 pA), a bovine growth hormone polyadenylation signal sequence (bGHpA), or a human growth hormone polyadenylation signal sequence (hGHpA).

### IV.A.5 Introns

In some aspects, the payload region of an AAV vector of the present disclosure comprises at least one element to enhance the expression such as one or more introns or portions thereof. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some aspects, the intron or intron portion can be between about 100 and about 500 nucleotides in length. In some aspects, the intron can have a length between about 80-100, about 80-120, about 80-140, about 80-160, about 80-180, about 80-200, about 80-250, about 80-300, about 80-350, about 80-400, about 80-450, about 80-500, about 200-300, about 200-400, about 200-500, about 300-400, about 300-500, or about 400-500 nucleotides.

In some aspects, the AAV vector can comprise a promoter such as, but not limited to, CMV or U6. In some aspects, the promoter for an AAV vector of the present disclosure is a CMV promoter. In some aspects, the promoter for an AAV vector of the present disclosure is a CMV early enhancer/chicken β actin (CAG) promoter. As another non-limiting example, the promoter for an AAV vector of the present disclosure is a U6 promoter. In some aspects, the AAV vector can comprise a CMV and a U6 promoter. In some aspects, the AAV vector can comprise a HI promoter. In some aspects, the AAV vector can comprise a CBA promoter. In some aspects, the AAV vector can comprise a chimeric intron. In some aspects, the AAV vector can comprise a SV40 intron. In some aspects, the AAV vector can comprise an immunoglobulin heavy chain intron. In some aspects, the AAV vector can comprise a human betaglobin intron. In some aspects, the AAV vector can comprise a chimera of a human betaglobin intron and an immunoglobulin heavy chain intron.

In some aspects, the promoter is a CMV early enhancer/chicken β actin (CAG) promoter, EF1α, human CMV, mouse CMV, EF1α promoter fused to CMV enhancer, CMV promoter fused to a SV40 intron, CMV promoter fused to a CMV enhancer, or a tissue specific promoters. In some aspects, the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51.

In some aspects, the encoded antibody (e.g., a monoclonal antibody) or antigen binding fragment thereof disclosed herein can be located downstream of a promoter in an expression vector such as, but not limited to, CMV, U6, HI, CBA, CAG, or a CBA promoter with an intron such as SV40, MVM intron, a human betaglobin intron, human immunoglobulin heavy chain intron, a chimera of a human betaglobin intron and a human immunoglobulin heavy chain intron, or others known in the art. In some aspects, the intron is selected from the group consisting of an SV40 intron, MVM intron, a human betaglobin intron, a human immunoglobulin heavy chain intron, or a chimera of a human immunoglobulin heavy chain intron and a human betaglobin intron. In some aspects, the intron comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 46, 56, or 82.

Further, the encoded antibody or antigen-binding fragment thereof can also be located upstream of the polyadenylation sequence in an expression vector. In some aspects, the encoded a therapeutic protein, e.g., antibody (e.g., a monoclonal antibody) or antigen binding fragment thereof or the fusion protein (e.g., the Fc fusion protein) disclosed herein, or therapeutic peptide can be located within about 1-5, about 1-10, about 1-15, about 1-20, about 1-25, about 1-30, about 5-10, about 5-15, about 5-20, about 5-25, about 5-30, about 10-15, about 10-20, about 10-25, about 10-30, about 15-20, about 15-25, about 15-30, about 20-25, about 20-30 or about 25-30 nucleotides downstream from the promoter and/or upstream of the polyadenylation sequence in the vector.

### IV.A.6 Filler Sequences

In some aspects, the AAV vector comprises one or more filler sequences (also referred to as “stuffer sequences”). In some aspects, the AAV vector comprises one or more filler sequences in order to have the length of the AAV vector be the optimal size for packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb. In some aspects, the vector backbone comprises a filler sequence.

In some aspects, the AAV vector comprises one or more filler sequences in order to reduce the likelihood that a hairpin structure of the vector genome (e.g., a modulatory polynucleotide described herein) can be read as an inverted terminal repeat (ITR) during expression and/or packaging. In some aspects, the AAV vector comprises at least one filler sequence in order to have the length of the AAV vector be about 2.0-2.5 kb, e.g., about 2.3 kb. In some aspects, the AAV vector comprises at least one filler sequence in order to have the length of the AAV vector be about 4.6 kb.

In some aspects, the AAV vector is a single stranded (ss) AAV vector and comprises one or more filler sequences which have a length about between 0.1 kb and about 3.8 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, about 1.5 kb, about 1.6 kb, about 1.7 kb, about 1.8 kb, about 1.9 kb, about 2 kb, about 2.1 kb, about 2.2 kb, about 2.3 kb, about 2.4 kb, about 2.5 kb, about 2.6 kb, about 2.7 kb, about 2.8 kb, about 2.9 kb, about 3 kb, about 3.1 kb, about 3.2 kb, about 3.3 kb, about 3.4 kb, about 3.5 kb, about 3.6 kb, about 3.7 kb, or about 3.8 kb.

In some aspects, the AAV vector is a self-complementary (sc) AAV vector and comprises one or more filler sequences which have a length about between about 0.1 kb and about 1.5 kb, such as, but not limited to, about 0.1 kb, about 0.2 kb, about 0.3 kb, about 0.4 kb, about 0.5 kb, about 0.6 kb, about 0.7 kb, about 0.8 kb, about 0.9 kb, about 1 kb, about 1.1 kb, about 1.2 kb, about 1.3 kb, about 1.4 kb, or about 1.5 kb.

In some aspects, the AAV vector comprises any portion of a filler sequence. The vector can comprise, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of a filler sequence.

In some aspects, the AAV vector is a single stranded (ss) AAV vector and comprises one or more filler sequences in order to have the length of the AAV vector be about 4.6 kb. In some aspects, the AAV vector comprises at least one filler sequence and the filler sequence is located 3′ to 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence. In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to 5′ ITR sequence and the second filler sequence is located 5′ to 5′ ITR sequence.

In some aspects, the AAV vector is a self-complementary (sc) AAV vector and comprises one or more filler sequences in order to have the length of the AAV vector be about 2.3 kb. In some aspects, the AAV vector comprises at least one filler sequence and the filler sequence is located 3′ to 5′ ITR sequence. In some aspects, the AAV vector comprises at least one filler sequence and the filler sequence is located 5′ to a promoter sequence. In some aspects, the AAV vector comprises at least one filler sequence and the filler sequence is located 3′ to the polyadenylation signal sequence. In some aspects, the AAV vector comprises at least one filler sequence and the filler sequence is located 5′ to the 3′ ITR sequence.

In some aspects, the AAV vector comprises at least one filler sequence, and the filler sequence is located between two intron sequences. In some aspects, the AAV vector comprises at least one filler sequence, and the filler sequence is located within an intron sequence. In some aspects, the AAV vector comprises two filler sequences, and the first filler sequence is located 3′ to 5′ ITR sequence and the second filler sequence is located 3′ to the polyadenylation signal sequence. In some aspects, the AAV vector comprises two filler sequences, and the first filler sequence is located 5′ to a promoter sequence and the second filler sequence is located 3′ to the polyadenylation signal sequence. In some aspects, the AAV vector comprises two filler sequences, and the first filler sequence is located 3′ to 5′ ITR sequence and the second filler sequence is located 5′ to 5′ ITR sequence.

In some aspects, the AAV vector can comprise one or more filler sequences between one of more regions of the AAV vector. In some aspects, the filler region can be located before a region such as, but not limited to, a payload region, an ITR, a promoter region, an intron region, an enhancer region, and/or a polyadenylation signal sequence region. In some aspects, the filler region can be located after a region such as, but not limited to, a payload region, an ITR, a promoter region, an intron region, an enhancer region, and/or a polyadenylation signal sequence region. In some aspects, the filler region can be located before and after a region such as, but not limited to, a payload region, an ITR, a promoter region, an intron region, an enhancer region, and/or a polyadenylation signal sequence region.

In some aspects, the AAV vector can comprise one or more filler sequences which bifurcates at least one region of the AAV vector. The bifurcated region of the AVV vector can comprise about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the of the region to 5′ of the filler sequence region.

In some aspects, the filler sequence can bifurcate at least one region so that about 10% of the region is located 5′ to the filler sequence and about 90% of the region is located 3′ to the filler sequence. In some aspects, the filler sequence can bifurcate at least one region so that about 20% of the region is located 5′ to the filler sequence and about 80% of the region is located 3′ to the filler sequence. In some aspects, the filler sequence can bifurcate at least one region so that about 30% of the region is located 5′ to the filler sequence and about 70% of the region is located 3′ to the filler sequence. In some aspects, the filler sequence can bifurcate at least one region so that about 40% of the region is located 5′ to the filler sequence and about 60% of the region is located 3′ to the filler sequence. In some aspects, the filler sequence can bifurcate at least one region so that about 50% of the region is located 5′ to the filler sequence and about 50% of the region is located 3′ to the filler sequence. In some aspects, the filler sequence can bifurcate at least one region so that about 60% of the region is located 5′ to the filler sequence and about 40% of the region is located 3′ to the filler sequence. In some aspects, the filler sequence can bifurcate at least one region so that about 70% of the region is located 5′ to the filler sequence and about 30% of the region is located 3′ to the filler sequence. In some aspects, the filler sequence can bifurcate at least one region so that about 80% of the region is located 5′ to the filler sequence and about 20% of the region is located 3′ to the filler sequence. In some aspects, the filler sequence can bifurcate at least one region so that about 90% of the region is located 5′ to the filler sequence and about 10% of the region is located 3′ to the filler sequence.

In some aspects, the AAV vector comprises a filler sequence after 5′ ITR. In some aspects, the AAV vector comprises a filler sequence after the promoter region. In some aspects, the AAV vector comprises a filler sequence after the payload region. In some aspects, the AAV vector comprises a filler sequence after the intron region. In some aspects, the AAV vector comprises a filler sequence after the enhancer region. In some aspects, the AAV vector comprises a filler sequence after the polyadenylation signal sequence region. In some aspects, the AAV vector comprises a filler sequence before the promoter region. In some aspects, the AAV vector comprises a filler sequence before the payload region. In some aspects, the AAV vector comprises a filler sequence before the intron region.

In some aspects, the AAV vector comprises a filler sequence before the enhancer region. In some aspects, the AAV vector comprises a filler sequence before the polyadenylation signal sequence region. In some aspects, the AAV vector comprises a filler sequence before 3′ ITR. In some aspects, a filler sequence can be located between two regions, such as, but not limited to, 5′ ITR and the promoter region. In some aspects, a filler sequence can be located between two regions, such as, but not limited to, 5′ ITR and the payload region.

In some aspects, a filler sequence can be located between two regions, such as, but not limited to, 5′ ITR and the intron region. In some aspects, a filler sequence can be located between two regions, such as, but not limited to, 5′ ITR and the enhancer region. In some aspects, a filler sequence can be located between two regions, such as, but not limited to, 5′ ITR and the polyadenylation signal sequence region. In some aspects, a filler sequence can be located between two regions, such as, but not limited to, the promoter region and the payload region.

In some aspects, a filler sequence can be located between two regions, such as, but not limited to, the promoter region and the intron region. In some aspects, a filler sequence can be located between two regions, such as, but not limited to, the promoter region and the enhancer region. In some aspects, a filler sequence can be located between two regions, such as, but not limited to, the promoter region and the polyadenylation signal sequence region. In some aspects, a filler sequence can be located between two regions, such as, but not limited to, the promoter region and 3′ ITR.

In some aspects, a filler sequence can be located between two regions, such as, but not limited to, the payload region and the intron region. In some aspects, a filler sequence can be located between two regions, such as, but not limited to, the payload region and the enhancer region. In some aspects, a filler sequence can be located between two regions, such as, but not limited to, the payload region and the polyadenylation signal sequence region. In some aspects, a filler sequence can be located between two regions, such as, but not limited to, the payload region and 3′ ITR.

In some aspects, a filler sequence can be located between two regions, such as, but not limited to, the intron region and the enhancer region. In some aspects, a filler sequence can be located between two regions, such as, but not limited to, the intron region and the polyadenylation signal sequence region. In some aspects, a filler sequence can be located between two regions, such as, but not limited to, the intron region and the 3′ ITR. In some aspects, a filler sequence can be located between two regions, such as, but not limited to, the enhancer region and the polyadenylation signal sequence region. In some aspects, a filler sequence can be located between two regions, such as, but not limited to, the enhancer region and 3′ ITR. In some aspects, a filler sequence can be located between two regions, such as, but not limited to, the polyadenylation signal sequence region and 3′ ITR.

In some aspects, an AAV vector can comprise two filler sequences. The two filler sequences can be located between two regions as described herein.

### IV.A.7 Method for Producing Recombinant AAVs

The present disclosure provides also methods for the generation of AAV particles, by viral genome replication in a viral replication cell comprising contacting the viral replication cell with an AAV polynucleotide or AAV genome (e.g., an AAV vector of the present disclosure). In the context of the present disclosure, the AAV vectors disclosed herein, e.g., AAV vectors comprising at least one polynucleotide encoding an antibody (e.g., a monoclonal antibody) or an antigen binding fragment thereof disclosed herein are considered AAV payload construct vectors.

In some aspects, an AAV particle is produced by a method comprising the steps of: (1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In one aspect, the present disclosure provides a method for producing an AAV particle comprising the steps of (1) simultaneously co-transfecting mammalian cells, such as, but not limited to HEK293 cells, with a payload region (e.g., polynucleotide encoding a therapeutic protein or therapeutic peptide of the disclosure), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide sequence encoding a payload such as an antibody (e.g., a monoclonal antibody) or an antigen binding fragment thereof disclosed herein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65 (2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

### IV.A.8 Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. Patent Application No. 20090275107.

### IV.A.9 AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

### V. Methods of Treatment and Use

Certain aspects of the disclosure are directed to the use of polynucleotides (e.g., antibody expression cassettes), vectors, and rAAV for treating a subject in need thereof. Some aspects of the present disclosure are directed to a method of delivering a gene therapy encoding an anti-IGF-1R antibody or antigen-binding fragment thereof to a subject in need thereof. In some aspects, the method comprises administering to the subject a delivery vector (e.g., a viral vector, a non-viral vectors, a plasmid, a lipid, protein particle, a bacterial vector, or a lysosome). In certain aspects, the methods for disclosed herein comprise delivery or administration of a polynucleotide (e.g., antibody expression cassettes), vector, rAAV, or composition disclosed herein to or near an eye (e.g., one or both eyes), e.g., intraocular, retro or a peri-orbital, retrobulbar, intramuscular near the eye (e.g., to a levator muscle and/or a glabellar muscle), to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the anti-IGF-1R antibody or antigen-binding fragment thereof is expressed in the muscle, connective tissue or adipose tissue. In some aspects, the anti-IGF-1R antibody or antigen-binding fragment thereof is produced in the muscle, connective tissue and adipose tissue.

In some aspects, methods comprising administering a gene therapy encoding an anti-IGF-1R antibody or antigen-binding fragment thereof comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

In some aspects, methods comprise administering a gene therapy construct encoding an anti-IGF-1R antibody (e.g., teprotumumab) is a multicistronic (e.g., bicistronic) construct (e.g., comprising a heavy chain and a light chain). In some aspects, the multicistronic (e.g., bicistronic) construct further comprises an F2A or IRES element.

In some aspects, the anti-IGF-1R antibody is teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or an antigen-binding fragment thereof.

In some aspects, the anti-IGF-1R antibody is VRDN-01100, or VRDN-02700, or an antigen-binding fragment thereof. In some aspects, the anti-IGFR antibody comprises SEQ ID NO: 113 (corresponding to VRDN-01100). In some aspects, the anti-IGFR antibody comprises SEQ ID NO: 116 (corresponding to VRDN-002700).

In some aspects, the anti-IGF-1R antibody is teprotumumab, or an antigen-binding fragment thereof.

In some aspects, the disclosure is directed to a method of delivering a gene therapy to a connective tissue (e.g., peritorbital, retroorbital). In some aspects, the gene therapy is administered periorbitally or retrobulbar (e.g., by injection) and thereafter an antibody or an antigen binding fragment thereof is produced in a fibroblast, myocyte, or adipocyte. In some aspects, the therapeutic effect of the anti-IGF-1R antibody or antigen-binding fragment thereof is local, systemic, or both.

In some aspects, the disclosure is directed to a method of delivering a gene therapy to a subject in need thereof, comprising administering to a muscular, intralymphatic, peritorbital or retrobulbar tissue or other delivery site disclosed herein via injection. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node.

Some aspects of the present disclosure are directed to a method of delivering a nucleic acid to a cell of a subject, comprising administering to a fibroblast, muscle cell, or adipocyte of the subject an adeno-associated virus (AAV) capsid comprising a nucleic acid comprising a promoter operably linked a polynucleotide encoding an antibody (e.g., a monoclonal antibody) or an antigen binding fragment thereof disclosed herein, thereby delivering the nucleic acid to the fibroblast, muscle cell, or adipocyte of the subject.

In some aspects, the methods disclosed herein can be practiced through the administration of the gene therapy composition comprising the polynucleotide (e.g., antibody expression cassette), vector, rAAV particle, or composition of the present disclosure, a cell comprising the polynucleotide (e.g., antibody expression cassette), vector, or rAAV particle of the present disclosure, a cell comprising a nucleic acid encoding an anti-IGF-1R alpha antibody or antigen-binding fragment thereof of the present disclosure integrated into its genomic DNA, or a pharmaceutical compositions comprising any of the above. Thus, methods disclosed herein reciting the administration of the polynucleotide (e.g., antibody expression cassette), vector, or rAAV particle of the present disclosure can be also practiced by administering any of these compositions.

In some aspects, methods disclosed herein can be practiced through the administration of a gene therapy composition comprising a nucleic acid encoding an antibody or antigen binding fragment thereof that binds to an insulin-like growth factor 1 receptor (IGF-1R) or antigen-binding fragments thereof.

In some aspects, methods disclosed herein can be practiced through the administration of a gene therapy composition comprising a nucleic acid encoding an antibody or antigen binding fragment thereof comprising (i) a heavy chain variable region (VH) comprising a complementarity determining region (CDR) 1, a VH CDR2, and a VH CDR3 and (ii) a light chain variable region (VL) comprising a CDR1, a VL CDR2, and a VL CDR3. In some aspects, the VH CDRs 1-3 and VL CDRs 1-3 is from the corresponding CDRs of teprotumumab.

In some aspects, methods disclosed herein can be practiced through the administration of a gene therapy composition comprising a nucleic acid encoding the anti-IGF-1R antibody or antigen-binding fragment thereof having the same amino acid sequence as teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or variant thereof.

In some aspects, methods disclosed herein can be practiced through the administration of a gene therapy composition comprising a nucleic acid encoding the anti-IGF-1R antibody or antigen-binding fragment thereof having the same amino acid sequence as VRDN-01100, or VRDN-02700, or variant thereof. In some aspects, the anti-IGFR antibody or antigen-binding fragment thereof comprises an amino acid sequence of SEQ ID NO: 113 (corresponding to VRDN-01100). In some aspects, the anti-IGFR antibody or antigen-binding fragment thereof comprises an amino acid sequence of SEQ ID NO: 116 (corresponding to VRDN-002700).

In some aspects, methods disclosed herein can be practiced through the administration of a gene therapy composition comprising a nucleic acid encoding the anti-IGF-1R antibody or antigen-binding fragment thereof having the same amino acid sequence as teprotumumab or a variant thereof.

Insulin growth factor (IGF) signaling is mediated by the ligands IGF1 and IGF2 and the receptors IGF-1R, IGF-2R and Insulin Receptor (IR). Downstream signaling from either IGF-1R or 1R is mediated through adaptor protein Insulin receptor substrate-1 (IRS-1) binding to activated growth factor receptor to activate the phosphoinositide-3 kinase signaling pathway. Teprotumumab is believed to lead to IGF activation of IGF-1R and prevents downstream phosphorylation. In some aspects, administration of the gene therapy composition comprising an antibody expression cassette, an AAV vector genome, or an rAAV particle of the present disclosure can lead to IGF activation of IGF-1R and prevents downstream phosphorylation.

Based on the methods disclosed herein, the gene therapy composition comprising an antibody expression cassette, an AAV vector genome, or an rAAV particle of the present disclosure for use in therapy, or for use as a medicament, or for use in treating a disease or disorder a subject in need thereof is contemplated. In some aspect, the disease or disorder to be treated is Graves' Orbitopathy.

Cells isolated from the peripheral blood in Grave's patients are known to express several proinflammatory cytokines, including IL-1B, IL-1 receptor antagonist, IL-6, IL-8 and TNFα and appears to infiltrate and adhere to orbital tissue by the stimulation of TSH (Chen, H., et al., (2014). Teprotumumab, an IGF-1R blocking monoclonal antibody inhibits TSH and IGF-1 action in fibrocytes. The Journal of Clinical Endocrinology & Metabolism, 99 (9), E1635-E1640). Studies have reported that isolated fibrocytes from Grave's patients and treated with teprotumumab in vitro attenuates expression levels and signaling pathways of IGF-1 and TSH receptors (Pritchard, J., et al., (2003). Immunoglobulin activation of T cell chemoattractant expression in fibroblasts from patients with Graves' disease is mediated through the insulin-like growth factor I receptor pathway. The Journal of Immunology, 170 (12), 6348-6354; Smith, T. J., et al., (2008). Unique attributes of orbital fibroblasts and global alterations in IGF-1 receptor signaling could explain thyroid-associated ophthalmopathy. Thyroid, 18 (9), 983-988; Chen, H., et al., (2014). Teprotumumab, an IGF-1R blocking monoclonal antibody inhibits TSH and IGF-1 action in fibrocytes. The Journal of Clinical Endocrinology & Metabolism, 99 (9), E1635-E1640).

In some aspects, administration of the gene therapy composition comprising an antibody expression cassette, an AAV vector genome, or a rAAV particle of the present disclosure can inhibit TSH and IGF-1 action in fibrocytes.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered to or near an eye (e.g., one or both eyes), e.g., intraocular, retro or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions. In some aspects, the subject suffers from a thyroid eye disease (TED) selected from the group consisting of active Graves' Orbitopathy and chronic Graves' Orbitopathy.

Teprotumumab does not cross react with rodent (both mouse and rat) IFG-1R. Recombinant teprotumumab has been tested by using the xenografted mouse model bearing a human lung carcinoma cell line, e.g., showing no significant difference in tumor volume, but significant reduction in IGF-1R protein expression. Thus, mouse xenograph models can be used to assess tumor volume and/or IGF-1R protein expression following treatment with anti-IGF-1R therapies (e.g., the gene therapy composition comprising an antibody expression cassette, an AAV vector genome, or an rAAV particle of the present disclosure).

In some aspects of the present disclosure antibody expression cassettes encoding anti-IGF-1R is delivered via AAV transduction. This allows delivery of the vectorized anti-IGF-1R via intratumoral injection unlike existing anti-IGF-1R monoclonal antibody therapies which are typically administered systemically.

In some aspects, local delivery and expression of anti-IGF-R by administration of an antibody expression cassette, an AAV vector genome, or a rAAV particle of the present disclosure reduces IGFR levels in the tumor. In some aspects, the local delivery of teprotumumab via AAV vector can result in tumor growth delay where the systemic delivery of the antibody does not. In some aspects, this can be assessed by tumor measurement in the different treatment groups.

In some aspects, the antibody expression cassette, an AAV vector genome, or a rAAV particle of the present disclosure can be administered to a subject suffering from a tumor or cancer. In some aspects, the administration is intratumoral. In some aspects, the tumor or cancer is a colon tumor or cancer. In some aspects, the AAV serotype is AAV9. In some aspects, the delivery vector comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, or a dual promoter (e.g., promoter-VH-IRES-VL, etc.).

In some aspects, the therapeutic effect of the antibody or antigen-binding fragment thereof or peptide is local, systemic, or both.

In some aspects, a composition or delivery vector disclosed herein comprising a nucleic acid encoding an anti-IGF-1R antibody or antigen-binding fragment thereof is suitable for delivery to a tissue for treating Graves' Orbitopathy.

In some aspects, the gene therapy composition or AAV capsid disclosed herein is administered by periorbital injection, retrobulbar injection, intramuscular injection, intralymphatic injection, or a combination thereof. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node.

In some aspects, the gene therapy composition or AAV capsid disclosed herein is administered intraocularly, by direct injection to the eye.

### VI. Pharmaceutical Compositions

In some aspects, a pharmaceutical composition disclosed herein comprises a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein and a pharmaceutically-acceptable excipient or carrier. Pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector of the present disclosure, wherein each vector comprises at least one polynucleotide encoding at least one therapeutic molecule disclosed herein.

In some aspects, a pharmaceutical composition comprises (i) one or more delivery vectors disclosed herein (e.g., AAV vectors or AAV capsids), and (ii) one or more therapeutic agents for the treatment of a disorder. In some aspects, the one or more delivery vectors disclosed herein (e.g., AAV vectors or AAV capsids) and the one or more therapeutic agents for a disease or disorder (e.g., Graves' Orbitopathy or Graves' disease) are co-administered in a single pharmaceutical composition.

In some aspects, the pharmaceutical composition comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

In some aspects, the one or more delivery vectors disclosed herein (e.g., antibody expression cassettes or rAAV particles) and the one or more therapeutic agents for the treatment of a disease or disorder (e.g., Graces' Orbitopathy or Graves' disease) are co-administered as separate pharmaceutical compositions.

In some aspects, a pharmaceutical composition comprising one or more delivery vectors disclosed herein (e.g., antibody expression cassettes or rAAV particles) is administered prior to the administration of a pharmaceutical composition comprising one or more therapeutic agents for the treatment of a disease or disorder (e.g., Graves' Orbitopathy or Graves' disease).

In some aspects, a pharmaceutical composition comprising one or more delivery vectors disclosed herein (e.g., antibody expression cassettes or rAAV particles) is administered after the administration of a pharmaceutical composition comprising one or more therapeutic agents for the treatment of a disease or disorder (e.g., Graves' Orbitopathy or Graves' disease).

In some aspects, a pharmaceutical composition comprising one or more delivery vectors disclosed herein (e.g., antibody expression cassettes or rAAV particles) is administered concurrently with a pharmaceutical composition comprising one or more therapeutic agents for the treatment of a disease or disorder (e.g., Graves' Orbitopathy or Graves' disease).

In some aspects, the pharmaceutical composition of the disclosure is formulated for administration to or near an eye (e.g., one or both eyes), e.g., intraocular, retro- or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the delivery or administration is to retro or a peri-orbital fibroblast cells, adipocytes cells, myofibroblast cells, myocyte cells, or any combination thereof. In some aspects, the composition is formulated for delivery or administration is by injection. In some aspects, the composition is formulated for delivery or administration is by infusion. In some aspects, the composition is formulated for delivery or administration is by injection and/or infusion as a single dose. In some aspects, the single dose administration comprising multiple injections or infusions periorbital or retrobullbarl administration. In some aspects, the pharmaceutical composition of the disclosure is formulated for muscular, intralyphatic, periorbital and/or retrobulbar injection. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node.

Also provided herein are pharmaceutical compositions comprising delivery vectors disclosed herein (e.g., antibody expression cassettes or rAAV particles) having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of vectors, e.g., AAV vectors described herein. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

Acceptable carriers, excipients, or stabilizers are nontoxic to recipients (e.g., animals or humans) at the dosages and concentrations employed.

Examples of carriers or diluents include, but are not limited to, water, saline, Ringer's solutions, dextrose solution, and 5% human serum albumin. Except insofar as any conventional media or compound is incompatible with the delivery vectors disclosed herein (e.g., antibody expression cassettes or rAAV particles), use thereof in the compositions is contemplated. In some aspects, a pharmaceutical composition is formulated to be compatible with its intended route of administration. The delivery vectors disclosed herein (e.g., antibody expression cassettes or rAAV particles) can be administered by to or near an eye (e.g., one or both eyes), e.g., intraocular, retro or a peri-orbital, retrobulbar, intramuscular near the eye, to connective tissue near the eye, or any combination thereof. In some aspects, the periorbital or retroorbital tissue is selected from muscle, connective tissue and/or adipose tissue. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is to retro or a peri-orbital fibroblast cells, adipocytes cells, myofibroblast cells, myocyte cells, or any combination thereof. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions.

In some aspects, the pharmaceutical composition comprising the delivery vectors disclosed herein (e.g., antibody expression cassettes or rAAV particles) is administered intravenously, e.g. by injection. In some aspects, the pharmaceutical composition comprising the delivery vectors disclosed herein (e.g., antibody expression cassettes or rAAV particles) is administered intramuscularly. In some aspects, the pharmaceutical composition comprising the delivery vectors disclosed herein (e.g., antibody expression cassettes or rAAV particles) is administered intravitreally (intraocularly). In some aspects, the pharmaceutical composition comprising the delivery vectors disclosed herein (e.g., antibody expression cassettes or rAAV particles) is administered by direct periorbital injection or retrobulbar injection. The delivery vectors disclosed herein (e.g., antibody expression cassettes or rAAV particles) can optionally be administered in combination with other therapeutic agents that are at least partly effective in treating the disease, disorder or condition for which the delivery vectors disclosed herein (e.g., antibody expression cassettes or rAAV particles) are intended.

The delivery vectors disclosed herein (e.g., antibody expression cassettes or rAAV particles) can be formulated using one or more excipients to (1) increase stability; (2) increase cell transfection or transduction; (3) permit the sustained or delayed release; or (4) alter the biodistribution (e.g., target the AAV vector to specific tissues or cell types).

### VII. Administration

The gene therapy compositions and delivery vectors disclosed herein (e.g., antibody expression cassettes or rAAV particles) can be administered by any route which results in a therapeutically effective outcome, e.g., for therapeutic expression of an anti-IGF-1R antibody or antigen-binding fragment thereof disclosed herein. In some aspects, the administration can be to or near an eye (e.g., one or both eyes), e.g., intraocular, retro or a peri-orbital, retrobulbar, intramuscular near the eye, to connective tissue near the eye, intralymphatic, or any combination thereof. In some aspects, the periorbital or retroorbital tissue is selected from muscle, connective tissue and/or adipose tissue. In some aspects, the delivery or administration is to retro- or a peri-orbital fibroblast cells, adipocytes cells, myofibroblast cells, myocyte cells, or any combination thereof. In some aspects, the administration is to an extra-ocular muscle. In some aspects, the extra-ocular muscle is a levantor muscle or a glabellar muscle. In some aspects, the administration is to a connective tissue. In some aspects, the administration is transconjunctival into the periorbital space. In some aspects, the administration is intralymphatic to the pre-auricular or submandibular node. In some aspects, the delivery or administration is by injection. In some aspects, the delivery or administration is by infusion. In some aspects, the delivery or administration is by injection and/or infusion as a single dose. In some aspects, the single dose administration comprising multiple injections or infusions

In some aspects, compositions of delivery vectors disclosed herein (e.g., expression cassettes) or AAV capsids can be administered in a way which facilitates the vectors to enter a periorbital or retrobulbar tissue of the subject. In some aspects, the periorbital or retrobulbar tissue is selected muscle, and/or adipose tissue. In some aspects, compositions of delivery vectors disclosed herein (e.g., expression cassettes) or AAV capsids can be administered into a lymph node. In some aspects, the lymph node is a pre-auricular and/or submandibular lymph node.

In certain aspects, the administration is periorbital or retrobulbar. In some aspects, the delivery vectors disclosed herein (e.g., viral vectors or the non-viral vectors (including naked DNA)) comprising a nucleic acid is introduced into the periorbital or retrobulbar tissue in vivo, e.g., by periorbital or retrobulbar administration, which can be accomplished by perfusion (e.g., continuous injection), or by a single, discontinuous injection. Periorbital or retrobulbar administration can also be accomplished by cannulation, for example, by insertion of a cannula next to the eye or into the periorbital connective tissue. Retrobulbar administration can be accomplished by insertion of a needle, e.g., a 22-27 Gauge needle, at the inferolateral border of the bony orbit, advanced straight back until the tip passes the equator of the eye and then directed medially towards the apex of the orbit. Retrobulbar administration can also be accomplished by inserting a needle through the eyelid and orbital fascia to depose the composition described herein behind the globe. In some aspects, retrobulbar administration provides advantages, e.g., because the vector is presented to the cells in a generally immune privileged microenvironment, the immunological and inflammatory reactions that are commonly observed as a result of the administration of transforming formulations and their adjuvants into blood and interstitial fluid can be avoided.

The amount of nucleic acid to transform a sufficient number of cells and provide for expression of therapeutic levels of the protein can be assessed using an animal model (e.g., a rodent (mouse or rat) or other mammalian animal model) to assess factors such as the efficiency of transformation, the levels of protein expression achieved, the susceptibility of the targeted cells to transformation, and the amounts of vector and/or nucleic acid required to transform target cells.

The precise amount of vector and/or nucleic acid administered will vary greatly according to a number of factors including the susceptibility of the target cells to transformation, the size and weight of the subject, the levels of protein expression desired, and the condition to be treated.

In some aspects, a delivery vector of the present disclosure (e.g., a viral vector, a non-viral vectors, a plasmid, a lipid, a protein particle, a bacterial vector, or a lysosome) comprising a promoter operably linked to a nucleic acid sequence that encodes an antibody (e.g., a monoclonal antibody) or an antigen binding fragment thereof disclosed herein can be administered to a periorbital or retrobulbar tissue or extraorbital muscle or pre-auricular or submandibular lymph node by direct injection.

In some aspects, a delivery vector of the present disclosure (e.g., a viral vector, a non-viral vectors, a plasmid, a lipid, a protein particle, a bacterial vector, or a lysosome) comprising a promoter operably linked to a nucleic acid sequence that encodes an anti-IGF-1R antibody (e.g., a monoclonal antibody) or an antigen binding fragment thereof disclosed herein is administered by direct injection, e.g., to a periorbital or retrobulbar or extraorbital muscle or pre-auricular or submandibular lymph node tissue.

In some aspects, a delivery vector of the present disclosure (e.g., a viral vector, a non-viral vectors, a plasmid, a lipid, or a lysosome) comprising a promoter operably linked to a nucleic acid sequence that encodes an anti-IGF-1R antibody (e.g., a monoclonal antibody) or an antigen binding fragment thereof disclosed herein can be administered by intramuscular injection.

In some aspects, a delivery vector of the present disclosure (e.g., a viral vector, a non-viral vectors, a plasmid, a lipid, or a lysosome) comprising a promoter operably linked to a nucleic acid sequence that encodes an anti-IGF-1R antibody (e.g., a monoclonal antibody) or an antigen binding fragment thereof disclosed herein can be administered intravenously.

In some aspects, a delivery vector of the present disclosure (e.g., a viral vector, a non-viral vectors, a plasmid, a lipid, or a lysosome) comprising a promoter operably linked to a nucleic acid sequence that encodes an anti-IGF-1R antibody (e.g., a monoclonal antibody) or an antigen binding fragment thereof disclosed herein can be administered by periorbital injection.

In some aspects, a delivery vector of the present disclosure (e.g., a viral vector, a non-viral vectors, a plasmid, a lipid, or a lysosome) comprising a promoter operably linked to a nucleic acid sequence that encodes an anti-IGF-1R antibody (e.g., a monoclonal antibody) or an antigen binding fragment thereof disclosed herein can be administered retrobulbar injection.

In some aspects, a delivery vector of the present disclosure (e.g., a viral vector, a non-viral vectors, a plasmid, a lipid, or a lysosome) comprising a promoter operably linked to a nucleic acid sequence that encodes an antibody (e.g., a monoclonal antibody) or an antigen binding fragment thereof disclosed herein can be administered by injection to an adipose tissue, a connective tissue, a muscle, or any combination thereof.

The delivery vectors disclosed herein (e.g., AAV vectors or AAV capsids) can be administered in any suitable form, either as a liquid solution or suspension, as a solid form suitable for liquid solution or suspension in a liquid solution.

In some aspects, the method comprise delivery of a polynucleotide encoding anti-IGF-1R by any method disclosed herein, wherein the polynucleotide comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, or a dual promoter (e.g., promoter-VH-IRES-VL, etc.).

### VIII. Kits

The present disclosure also provides kits, or products of manufacture, comprising (i) the delivery vector of the present disclosure, or a pharmaceutical composition of the present disclosure, and (ii) optionally instructions for use (e.g., a package insert with instructions to perform any of the methods described herein).

In some aspects, the kit or product of manufacture comprises (i) comprising the delivery vectors of the present disclosure (e.g., an AAV vector or expression construct (e.g., antibody expression cassette) comprising a nucleic acid encoding an anti-IGF-1R antibody or antigen-binding fragment thereof disclosed herein), or a pharmaceutical composition of the present disclosure, (ii) optionally, an additional therapeutic agent, and (iii) optionally, instructions for use (e.g., a package insert with instructions to perform any of the methods described herein are also contemplated).

In some aspects, the components of a kit or product of manufacture disclosed herein are in one or more containers. In some aspects, the kit or product of manufacture comprises (i) an AAV vector or expression construct (e.g., antibody expression cassette) comprising a nucleic acid encoding an anti-IGF-1R antibody or antigen-binding fragment thereof disclosed herein, and (ii) a brochure with instructions administer the AAV vector or expression construct (e.g., antibody expression cassette) to a subject.

In some aspects, a kit or product of manufacture of the present disclosure comprises at least one delivery vector (e.g., antibody expression cassettes or rAAV particles). In some aspects, a kit or product of manufacture of the present disclosure comprises at least one polynucleotide encoding at least one anti-IGF-1R antibody or antigen-binding fragment thereof disclosed herein.

In some aspects, the anti-IGF-1R antibody is teprotumumab, VRDN-01100 (SEQ ID NO: 113), VRDN-02700 (SEQ ID NO: 116), ganitumab (AMG 479), figitumumab, CP-751,871, cixutumumab (AMG 655), IMC-A12, dalotuzumab, MK0646, RG1507, robatumumab, SCH 717454, AVE-1642a, MEDI-573, BIIB022, rhuMab IGFR, L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, or L52H52, or variant thereof.

In some aspects, the anti-IGF-1R antibody is VRDN-01100, or VRDN-02700, or variant thereof. In some aspects, the anti-IGFR antibody or antigen-binding fragment thereof comprises an amino acid sequence of SEQ ID NO: 113 (corresponding to VRDN-01100). In some aspects, the anti-IGFR antibody or antigen-binding fragment thereof comprises an amino acid sequence of SEQ ID NO: 116 (corresponding to VRDN-002700).

In some aspects, the anti-IGF-1R antibody is teprotomumab, or variant thereof.

In some aspects, the kit comprises a nucleic acid sequence encoding an anti-IGF-1R antibody or antigen-binding fragment thereof (e.g., teprotomumab) comprising: (i) VH CDRs 1-3 (e.g., SEQ ID NOs: 7-9, 10-12, or 13-15) and VL CDRs 1-3 (e.g., SEQ ID NOs: 16-18, 19-21, or 22-24); (ii) VH (e.g., SEQ ID NOs: 26 or 27) and VL (e.g., SEQ ID NOs: 30 or 31); (iii) HC (e.g., SEQ ID NOs: 36 or 37) and LC (e.g., SEQ ID NOs: 40 or 41); or (iv) a vector construct or expression construct (e.g., antibody expression cassette) comprising any one of SEQ ID NOs: 68-76, wherein the vector construct or expression construct (e.g., antibody expression cassette) further comprises one or more of IRES, furin cleavage site, 2a site, a dual promoter (e.g., promoter-VH-IRES-VL, etc.), or a signal peptide (e.g., an IL-2 or an IL-10 signal peptide).

One skilled in the art will readily recognize that polynucleotides (e.g., antibody expression cassette), vectors, rAAV particles, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

The following examples are offered by way of illustration and not by way of limitation.

### EXAMPLES

### Example 1: Modified Anti-IGF-1R Antibody Nucleic Acids

The following modified anti-IGF-1R antibody ORF nucleic acid sequences (shown in Table 16) corresponding to SEQ ID NOs: 57-67 and 94-97 were designed in silico. The OFRs include nucleic acid molecules encoding an anti-IGF-1R heavy chain and a light chain.

TABLE 16
Modified ORF Nucleic Acid Sequences Encoding anti-IGF-1R Antibody

| SEQ ID | | |
|---|---|---|
| NO/length | Name | Sequence |
| (SEQ ID NO: | ORF | atgcacagctccgccctgctctgctgtttggtgcttctgaccggcgtccgggctcaggttgagttagt |
| 57) | #1 | ggaatctggagggggtgtagtgcaacccggcagatcacagaggctgagttgcgcagcgtcggga |
| Full: 2202 | | ttcacatttagctcctacgggatgcattgggtccgccaggcccctggcaagggtctagagtggggg |
| IL-10 Signal | | ctatcatttggttcgacggatcttcaacttattacgccgatagtgttcgagggcgttttacgataagccg |
| peptide: 1-54 | | ggacaactccaaaaataccctctatctgcaaatgaactctttgagagcagaagatacagccgtgtact |
| HC: 55-1398 | | tctgtgctagggagctgggccgccggtattttgacctttggggcagaggaactctcgtcagcgtatcc |
| Furin: | | tcagcaagtaccaaggggccaagcgtgttcccgctggccccctcttccaaatcgacatcaggtggc |
| 1399-1410 | | accgcggctctgggatgcctcgttaaggattactttcctgaaccagtgactgtcagctggaatagtgg |
| Linker: | | ggccttgacgtctggcgtgcacacattccccgcagtccttcagtccagcggtctgtattcattatccag |
| 1411-1422 | | tgtggttaccgtgccttctagctccctgggaactcagacctacatctgtaacgtaaatcataaaccaag |
| F2A: | | caacacaaaggtggacaagaaagtcgagcccaagtcttgcgataaaacccacacttgtccgccttg |
| 1423-1494 | | cccagcccccgagctactcgggggcccttcagtttttctgttcccacccaagcctaaagacacattga |
| IL-2 Signal | | tgattagtaggacgcccgaagtgacctgtgtcgtggtggatgtttcccatgaggacccagaagtaaa |
| peptide: | | gttcaattggtacgtggatggcgtcgaggtgcacaacgctaagactaaaccgcgcgaagagcagta |
| 1495-1554 | | taatagcacataccgagtcgtgtcggttctgaccgtgcttcaccaagactggctcaacggaaaggag |
| LC: | | tataaatgcaaggtatctaataaggccctgcccgcacctatcgaaaaaactatttcaaaggctaaagg |
| 1555-2198 | | gcagccacgtgagccccaggtctacaccctgcctccatcccgggacgaattaacaaagaaccagg |
| Stop Codon: | | tgagtctcacgtgcctggttaaaggtttttatcccagcgatatcgccgtggagtgggagtctaacggc |
| 2200-2202 | | caaccggaaaataactacaagaccacacctccagtcttggactccgatggaagcttctttctttactca |
| | | aagctgactgtggacaaaagtagatggcagcaggggaatgtgttcagctgttccgttatgcatgagg |
| | | cgctacacaaccattatacccagaagtctctctcgctgtcacccggcaaaagaaagagaaggagtg |
| | | gctcaggagcccctgtgaaacagaccctgaactttgacctcttgaagcttgctggggatgtggagtct |
| | | aatcctggtccaatgtaccggatgcagctgctcagctgcatcgccttgtcccttgctctggtgaccaa |
| | | ctctgagattgtcttaacacaatcacccgcaactctgagtctatcgcctggcgaaagagcgacgctca |
| | | gctgtagggcctcccagtctgtttcaagttatctggcttggtaccagcaaaagccaggacaggcccc |
| | | gcgcttgctgatatatgacgcaagcaaacgagccaccgggatccccgctcgtttctccggttctggc |
| | | agcggaacagattttactcttaccatttcctcactcgagcctgaagacttcgcagtgtactattgccagc |
| | | agcggagtaagtggccaccctggacatttgggcaaggcaccaaagtagagagcaagagaactgt |
| | | ggccgcgccttctgtcttcatctttccaccctccgatgaacagctgaaatcgggtacggcttcagtggt |
| | | ttgtctgctcaataacttctacccgagggaggccaaggtgcagtggaaggtcgacaatgcattgcag |
| | | agcggaaacagtcaagagtctgtaacagaacaggattccaaagacagcacctattcactttccagta |
| | | ctctgaccttatctaaggccgattacgagaaacacaaggtgtacgcttgcgaagttacacatcaggg |
| | | gctgagctcccctgtgaccaaaagctttaatcgcggcgagtgttag |
| (SEQ ID NO: | ORF | ATGCACAGCTCCGCCCTGCTCTGCTGTTTGGTGCTTCTGACA |
| 58) | #2 | GGAGTCAGGGCTCAGGTTGAGTTAGTGGAATCTGGAGGGGG |
| Full: 2202 | | TGTAGTGCAACCTGGCAGATCACAGAGGCTGAGTTGCGCAG |
| IL-10 Signal | | CAAGTGGATTCACATTTAGCTCCTATGGGATGCATTGGGTCC |
| peptide: 1-54 | | GCCAGGCCCCTGGCAAGGGTCTAGAGTGGGTGGCTATCATT |
| HC: 55-1398 | | TGGTTTGATGGATCTTCAACTTATTATGCTGATAGTGTTAGA |
| Furin: | | GGGCGCTTTACTATAAGCAGGGACAACTCCAAAAATACCCT |
| 1399-1410 | | CTATCTGCAAATGAACTCTTTGAGAGCAGAAGATACAGCTG |
| Linker: | | TGTACTTCTGTGCTAGGGAGCTGGGCCGCAGATATTTTGACC |
| 1411-1422 | | TTTGGGGCAGAGGAACTCTTGTCTCTGTATCCTCAGCAAGTA |
| F2A: | | CCAAGGGGCCATCTGTGTTCCCGCTGGCCCCCTCTTCCAAAA |
| 1423-1494 | | GTACATCAGGTGGCACCGCGGCTCTGGGATGCTTGGTTAAG |
| IL-2 Signal | | GATTACTTTCCTGAACCAGTGACTGTCAGCTGGAATAGTGG |
| peptide: | | GGCCTTGACTTCTGGGGTGCACACATTCCCCGCAGTCCTTCA |
| 1495-1554 | | GTCCAGCGGTCTGTATTCATTATCCAGTGTGGTTACAGTGCC |
| LC: | | TTCTAGCTCCCTGGGAACTCAGACCTACATCTGTAATGTAAA |
| 1555-2199 | | TCATAAACCAAGCAACACAAAGGTGGACAAGAAAGTTGAG |
| Stop Codon: | | CCCAAGTCTTGTGATAAAACCCACACTTGTCCGCCTTGCCCA |
| 2200-2202 | | GCCCCTGAGCTACTTGGGGGCCCTTCAGTTTTTCTGTTCCCA |
| | | CCCAAGCCTAAAGACACATTGATGATTAGTAGGACTCCTGA |
| | | AGTGACCTGTGTTGTGGTGGATGTTTCCCATGAGGACCCAG |
| | | AAGTAAAGTTCAATTGGTATGTGGATGGAGTTGAGGTGCAC |
| | | AATGCTAAGACTAAACCGAGGGAAGAGCAGTATAATAGCA |
| | | CATACAGAGTTGTGTCAGTTCTGACAGTGCTTCACCAAGACT |
| | | GGCTCAATGGAAAGGAGTATAAATGCAAGGTATCTAATAAG |
| | | GCCCTGCCCGCACCTATTGAAAAAACTATTTCAAAGGCTAA |
| | | AGGGCAGCCAAGGGAGCCCCAGGTCTACACCCTGCCTCCAT |
| | | CCAGAGATGAATTAACAAAGAACCAGGTGAGTCTCACTTGC |
| | | CTGGTTAAAGGTTTTTATCCCTCTGATATTGCTGTGGAGTGG |
| | | GAGTCTAATGGCCAACCAGAAAATAACTACAAGACCACACC |
| | | TCCAGTCTTGGACTCTGATGGAAGCTTCTTTCTTTACTCAAA |
| | | GCTGACTGTGGACAAAAGTAGATGGCAGCAGGGGAATGTGT |
| | | TCAGCTGTTCTGTTATGCATGAGGCGCTACACAACCATTATA |
| | | CCCAGAAGTCTCTCAGTCTGTCACCTGGCAAAAGAAAGAGA |
| | | AGGAGTGGCTCAGGAGCCCCTGTGAAACAGACCCTGAACTT |
| | | TGACCTCTTGAAGCTTGCTGGGGATGTGGAGTCTAATCCTGG |
| | | TCCAATGTACAGAATGCAGCTGCTCAGCTGCATTGCCTTGTC |
| | | CCTTGCTCTGGTGACCAACTCTGAGATTGTCTTAACACAATC |
| | | ACCCGCAACTCTGAGTCTAAGTCCTGGAGAAAGAGCAACTC |
| | | TCAGCTGTAGGGCCTCCCAGTCTGTTTCAAGTTATCTGGCTT |
| | | GGTACCAGCAAAAGCCAGGACAGGCCCCGCGCTTGCTGATA |
| | | TATGATGCAAGCAAAAGGGCCACAGGGATCCCCGCTAGATT |
| | | CAGCGGTTCTGGCAGCGGAACAGATTTTACTCTTACCATTTC |
| | | CTCACTTGAGCCTGAAGACTTTGCAGTGTACTATTGCCAGCA |
| | | GCGGAGTAAGTGGCCACCCTGGACATTTGGGCAAGGCACCA |
| | | AAGTAGAGAGCAAGAGAACTGTGGCCGCGCCTTCTGTCTTC |
| | | ATCTTTCCACCCTCTGATGAACAGCTGAAATCAGGTACTGCT |
| | | TCAGTGGTTTGTCTGCTCAATAACTTCTACCCAAGGGAGGCC |
| | | AAGGTGCAGTGGAAGGTTGACAATGCATTGCAGAGCGGAA |
| | | ACAGTCAAGAGTCTGTAACAGAACAGGATTCCAAAGACAGC |
| | | ACCTATTCACTTTCCAGTACTCTGACCTTATCTAAGGCTGAT |
| | | TATGAGAAACACAAGGTGTATGCTTGTGAAGTTACACATCA |
| | | GGGGCTGAGCTCCCCTGTGACCAAAAGCTTTAATAGGGGGG |
| | | AGTGTTAG |
| (SEQ ID NO: | ORF | atgcacagctctgccctgctctgctgtttggtgcttctgacaggagtcagggctcaggttgagttagtg |
| 59) | #3 | gaatctggagggggtgtagtgcaacctggcagatcacagaggctgagttgtgcagcaagtggattc |
| Full: 1401 | | acatttagctcctatgggatgcattgggtcagacaggcccctggcaagggtctagagtgggtggcta |
| IL-10 Signal | | tcatttggtttgatggatcttcaacttattatgctgatagtgttagggggagatttactataagcaggga |
| peptide: 1-54 | | caactccaaaaataccctctatctgcaaatgaactctttgagagcagaagatacagctgtgtacttctgt |
| HC: 55-1398 | | gctagggagctgggcagaaggtattttgacctttggggcagaggaactcttgtctctgtatcctcagc |
| Stop Codon: | | aagtaccaaggggccatctgtgttcccactggccccctcttccaaaagtacatcaggtggcacagca |
| 1399-1401 | | gctctgggatgcttggttaaggattactttcctgaaccagtgactgtcagctggaatagtggggccttg |
| | | acttctggggtgcacacattccctgcagtccttcagtcctctggtctgtattcattatccagtgtggtta |
| | | cagtgccttctagctccctgggaactcagacctacatctgtaatgtaaatcataaaccaagcaacacaa |
| | | aggtggacaagaaagttgagcccaagtcttgtgataaaacccacacttgtccaccttgcccagcccc |
| | | tgagctacttgggggcccttcagtttttctgttcccacccaagcctaaagacacattgatgattagtagg |
| | | actcctgaagtgacctgtgttgtggtggatgtttcccatgaggacccagaagtaaagttcaattggtat |
| | | gtggatggagttgaggtgcacaatgctaagactaaaccaagagaagagcagtataatagcacatac |
| | | agggttgtgtcagttctgacagtgcttcaccaagactggctcaatggaaaggagtataaatgcaaggt |
| | | atctaataaggccctgcctgcacctattgaaaaaactatttcaaaggctaaagggcagccaagagag |
| | | ccccaggtctacaccctgcctccatccagggatgaattaacaaagaaccaggtgagtctcacttgcc |
| | | tggttaaaggtttttatccctctgatattgctgtggagtgggagtctaatggccaaccagaaaataacta |
| | | caagaccacacctccagtcttggactctgatggaagcttctttctttactcaaagctgactgtggacaa |
| | | aagtagatggcagcaggggaatgtgttcagctgttctgttatgcatgaggcactacacaaccattata |
| | | cccagaagtctctcagtctgtcacctggcaaatag |
| (SEQ ID NO: | ORF | atgtaccggatgcagctgctcagctgcatcgccttgtcccttgctctggtgaccaactctgagattgtc |
| 60) | #4 | ttaacacaatcacccgcaactctgagtctatcgcctggcgaaagagcgacgctcagctgtagggcct |
| Full: 708 | | cccagtctgtttcaagttatctggcttggtaccagcaaaagccaggacaggccccgcgcttgctgata |
| IL-2 Signal | | tatgacgcaagcaaacgagccaccgggatccccgctcgtttctccggttctggcagcggaacagat |
| peptide: 1-60 | | tttactcttaccatttcctcactcgagcctgaagacttcgcagtgtactattgccagcagcggagtaagt |
| LC: 61-705 | | ggccaccctggacatttgggcaaggcaccaaagtagagagcaagagaactgtggccgcgccttct |
| Stop Codon: | | gtcttcatctttccaccctccgatgaacagctgaaatcgggtacggcttcagtggtttgtctgctcaata |
| 706-708 | | acttctacccgagggaggccaaggtgcagtggaaggtcgacaatgcattgcagagcggaaacagt |
| | | caagagtctgtaacagaacaggattccaaagacagcacctattcactttccagtactctgaccttatct |
| | | aaggccgattacgagaaacacaaggtgtacgcttgcgaagttacacatcaggggctgagctcccct |
| | | gtgaccaaaagctttaatcgcggcgagtgttag |
| (SEQ ID NO: | ORF | atgcacagctccgccctgctctgctgtttggtgcttctgaccggcgtccgggctcaggttgagttagt |
| 61) | #5 | ggaatctggagggggtgtagtgcaacccggcagatcacagaggctgagttgcgcagcgtcggga |
| Full: 1401 | | ttcacatttagctcctacgggatgcattgggtccgccaggcccctggcaagggtctagagtgggtgg |
| IL-10 Signal | | ctatcatttggttcgacggatcttcaacttattacgccgatagtgttcgagggcgttttacgataagccg |
| peptide: 1-54 | | ggacaactccaaaaataccctctatctgcaaatgaactctttgagagcagaagatacagccgtgtact |
| HC: 55-1398 | | tctgtgctagggagctgggccgccggtattttgacctttggggcagaggaactctcgtcagcgtatcc |
| Stop Codon: | | tcagcaagtaccaaggggccaagcgtgttcccgctggccccctcttccaaatcgacatcaggtggc |
| 1399-1401 | | accgcggctctgggatgcctcgttaaggattactttcctgaaccagtgactgtcagctggaatagtgg |
| | | ggccttgacgtctggcgtgcacacattccccgcagtccttcagtccagcggtctgtattcattatccag |
| | | tgtggttaccgtgccttctagctccctgggaactcagacctacatctgtaacgtaaatcataaaccaag |
| | | caacacaaaggtggacaagaaagtcgagcccaagtcttgcgataaaacccacacttgtccgccttg |
| | | cccagcccccgagctactcgggggcccttcagtttttctgttcccacccaagcctaaagacacattga |
| | | tgattagtaggacgcccgaagtgacctgtgtcgtggtggatgtttcccatgaggacccagaagtaaa |
| | | gttcaattggtacgtggatggcgtcgaggtgcacaacgctaagactaaaccgcgcgaagagcagta |
| | | taatagcacataccgagtcgtgtcggttctgaccgtgcttcaccaagactggctcaacggaaaggag |
| | | tataaatgcaaggtatctaataaggccctgcccgcacctatcgaaaaaactatttcaaaggctaaagg |
| | | gcagccacgtgagccccaggtctacaccctgcctccatcccgggacgaattaacaaagaaccagg |
| | | tgagtctcacgtgcctggttaaaggtttttatcccagcgatatcgccgtggagtgggagtctaacggc |
| | | caaccggaaaataactacaagaccacacctccagtcttggactccgatggaagcttctttctttactca |
| | | aagctgactgtggacaaaagtagatggcagcaggggaatgtgttcagctgttccgttatgcatgagg |
| | | cgctacacaaccattatacccagaagtctctctcgctgtcacccggcaaatag |
| (SEQ ID NO: | ORF | atgtacagaatgcagctgctcagctgcattgccttgtcccttgctctggtgaccaactctgagattgtct |
| 62) | #6 | taacacaatcacccgcaactctgagtctaagtcctggagaaagagcaactctcagctgtagggcctc |
| Full: 708 | | ccagtctgtttcaagttatctggcttggtaccagcaaaagccaggacaggccccgcgcttgctgatat |
| IL-2 Signal | | atgatgcaagcaaaagggccacagggatccccgctagattcagcggttctggcagcggaacagat |
| peptide: 1-60 | | tttactcttaccatttcctcacttgagcctgaagactttgcagtgtactattgccagcagcggagtaagt |
| LC: 61-705 | | ggccaccctggacatttgggcaaggcaccaaagtagagagcaagagaactgtggccgcgccttct |
| Stop Codon: | | gtcttcatctttccaccctctgatgaacagctgaaatcaggtactgcttcagtggtttgtctgctcaata |
| 706-708 | | acttctacccaagggaggccaaggtgcagtggaaggttgacaatgcattgcagagcggaaacagtc |
| | | aagagtctgtaacagaacaggattccaaagacagcacctattcactttccagtactctgaccttatcta |
| | | aggctgattatgagaaacacaaggtgtatgcttgtgaagttacacatcaggggctgagctcccctgtg |
| | | accaaaagctttaataggggggagtgttag |
| (SEQ ID NO: | ORF | ATGCACAGCTCCGCCCTGCTCTGCTGTTTGGTGCTTCTGACA |
| 63) | #7 | GGAGTCAGGGCTCAGGTTGAGTTAGTGGAATCTGGAGGGGG |
| Full: 1401 | | TGTAGTGCAACCTGGCAGATCACAGAGGCTGAGTTGCGCAG |
| IL-10 Signal | | CAAGTGGATTCACATTTAGCTCCTATGGGATGCATTGGGTCC |
| peptide: 1-54 | | GCCAGGCCCCTGGCAAGGGTCTAGAGTGGGTGGCTATCATT |
| HC: 55-1398 | | TGGTTTGATGGATCTTCAACTTATTATGCTGATAGTGTTAGA |
| Stop Codon: | | GGGCGCTTTACTATAAGCAGGGACAACTCCAAAAATACCCT |
| 1399-1401 | | CTATCTGCAAATGAACTCTTTGAGAGCAGAAGATACAGCTG |
| | | TGTACTTCTGTGCTAGGGAGCTGGGCCGCAGATATTTTGACC |
| | | TTTGGGGCAGAGGAACTCTTGTCTCTGTATCCTCAGCAAGTA |
| | | CCAAGGGGCCATCTGTGTTCCCGCTGGCCCCCTCTTCCAAAA |
| | | GTACATCAGGTGGCACCGCGGCTCTGGGATGCTTGGTTAAG |
| | | GATTACTTTCCTGAACCAGTGACTGTCAGCTGGAATAGTGG |
| | | GGCCTTGACTTCTGGGGTGCACACATTCCCCGCAGTCCTTCA |
| | | GTCCAGCGGTCTGTATTCATTATCCAGTGTGGTTACAGTGCC |
| | | TTCTAGCTCCCTGGGAACTCAGACCTACATCTGTAATGTAAA |
| | | TCATAAACCAAGCAACACAAAGGTGGACAAGAAAGTTGAG |
| | | CCCAAGTCTTGTGATAAAACCCACACTTGTCCGCCTTGCCCA |
| | | GCCCCTGAGCTACTTGGGGGCCCTTCAGTTTTTCTGTTCCCA |
| | | CCCAAGCCTAAAGACACATTGATGATTAGTAGGACTCCTGA |
| | | AGTGACCTGTGTTGTGGTGGATGTTTCCCATGAGGACCCAG |
| | | AAGTAAAGTTCAATTGGTATGTGGATGGAGTTGAGGTGCAC |
| | | AATGCTAAGACTAAACCGAGGGAAGAGCAGTATAATAGCA |
| | | CATACAGAGTTGTGTCAGTTCTGACAGTGCTTCACCAAGACT |
| | | GGCTCAATGGAAAGGAGTATAAATGCAAGGTATCTAATAAG |
| | | GCCCTGCCCGCACCTATTGAAAAAACTATTTCAAAGGCTAA |
| | | AGGGCAGCCAAGGGAGCCCCAGGTCTACACCCTGCCTCCAT |
| | | CCAGAGATGAATTAACAAAGAACCAGGTGAGTCTCACTTGC |
| | | CTGGTTAAAGGTTTTTATCCCTCTGATATTGCTGTGGAGTGG |
| | | GAGTCTAATGGCCAACCAGAAAATAACTACAAGACCACACC |
| | | TCCAGTCTTGGACTCTGATGGAAGCTTCTTTCTTTACTCAAA |
| | | GCTGACTGTGGACAAAAGTAGATGGCAGCAGGGGAATGTGT |
| | | TCAGCTGTTCTGTTATGCATGAGGCGCTACACAACCATTATA |
| | | CCCAGAAGTCTCTCAGTCTGTCACCTGGCAAATAG |
| (SEQ ID NO: | ORF | ATGCACAGCTCCGCCCTGCTCTGCTGTTTGGTGCTTCTGACA |
| 64) | #8 | GGAGTCAGGGCTCAGGTTGAGTTAGTGGAATCTGGAGGGGG |
| Full: 1401 | | TGTAGTGCAACCTGGCAGATCACAGAGGCTGAGTTGCGCAG |
| IL-10 Signal | | CAAGTGGATTCACATTTAGCTCCTATGGGATGCATTGGGTCC |
| peptide: 1-54 | | GCCAGGCCCCTGGCAAGGGTCTAGAGTGGGTGGCTATCATT |
| HC: 55-1398 | | TGGTTTGATGGATCTTCAACTTATTATGCTGATAGTGTTAGA |
| Stop Codon: | | GGGCGCTTTACTATAAGCAGGGACAACTCCAAAAATACCCT |
| 1399-1401 | | CTATCTGCAAATGAACTCTTTGAGAGCAGAAGATACAGCTG |
| | | TGTACTTCTGTGCTAGGGAGCTGGGCCGCAGATATTTTGACC |
| | | TTTGGGGCAGAGGAACTCTTGTCTCTGTATCCTCAGCAAGTA |
| | | CCAAGGGGCCATCTGTGTTCCCGCTGGCCCCCTCTTCCAAAA |
| | | GTACATCAGGTGGCACCGCGGCTCTGGGATGCTTGGTTAAG |
| | | GATTACTTTCCTGAACCAGTGACTGTCAGCTGGAATAGTGG |
| | | GGCCTTGACTTCTGGGGTGCACACATTCCCCGCAGTCCTTCA |
| | | GTCCAGCGGTCTGTATTCATTATCCAGTGTGGTTACAGTGCC |
| | | TTCTAGCTCCCTGGGAACTCAGACCTACATCTGTAATGTAAA |
| | | TCATAAACCAAGCAACACAAAGGTGGACAAGAAAGTTGAG |
| | | CCCAAGTCTTGTGATAAAACCCACACTTGTCCGCCTTGCCCA |
| | | GCCCCTGAGCTACTTGGGGGCCCTTCAGTTTTTCTGTTCCCA |
| | | CCCAAGCCTAAAGACACATTGATGATTAGTAGGACTCCTGA |
| | | AGTGACCTGTGTTGTGGTGGATGTTTCCCATGAGGACCCAG |
| | | AAGTAAAGTTCAATTGGTATGTGGATGGAGTTGAGGTGCAC |
| | | AATGCTAAGACTAAACCGAGGGAAGAGCAGTATAATAGCA |
| | | CATACAGAGTTGTGTCAGTTCTGACAGTGCTTCACCAAGACT |
| | | GGCTCAATGGAAAGGAGTATAAATGCAAGGTATCTAATAAG |
| | | GCCCTGCCCGCACCTATTGAAAAAACTATTTCAAAGGCTAA |
| | | AGGGCAGCCAAGGGAGCCCCAGGTCTACACCCTGCCTCCAT |
| | | CCAGAGATGAATTAACAAAGAACCAGGTGAGTCTCACTTGC |
| | | CTGGTTAAAGGTTTTTATCCCTCTGATATTGCTGTGGAGTGG |
| | | GAGTCTAATGGCCAACCAGAAAATAACTACAAGACCACACC |
| | | TCCAGTCTTGGACTCTGATGGAAGCTTCTTTCTTTACTCAAA |
| | | GCTGACTGTGGACAAAAGTAGATGGCAGCAGGGGAATGTGT |
| | | TCAGCTGTTCTGTTATGCATGAGGCGCTACACAACCATTATA |
| | | CCCAGAAGTCTCTCAGTCTGTCACCTGGCAAATAG |
| (SEQ ID NO: | ORF | atgcacagctctgccctgctctgctgtttggtgcttctgacaggagtcagggctcaggttgagttagtg |
| 65) | #9 | gaatctggagggggtgtagtgcaacctggcagatcacagaggctgagttgtgcagcaagtggattc |
| Full: 1401 | | acatttagctcctatgggatgcattgggtcagacaggcccctggcaagggtctagagtgggtggcta |
| IL-10 Signal | | tcatttggtttgatggatcttcaacttattatgctgatagtgttagggggagatttactataagcaggga |
| peptide: 1-54 | | caactccaaaaataccctctatctgcaaatgaactctttgagagcagaagatacagctgtgtacttctgt |
| HC: 55-1398 | | gctagggagctgggcagaaggtattttgacctttggggcagaggaactcttgtctctgtatcctcagc |
| Stop Codon: | | aagtaccaaggggccatctgtgttcccactggccccctcttccaaaagtacatcaggtggcacagca |
| 1399-1401 | | gctctgggatgcttggttaaggattactttcctgaaccagtgactgtcagctggaatagtggggccttg |
| | | acttctggggtgcacacattccctgcagtccttcagtcctctggtctgtattcattatccagtgtggtta |
| | | cagtgccttctagctccctgggaactcagacctacatctgtaatgtaaatcataaaccaagcaacacaa |
| | | aggtggacaagaaagttgagcccaagtcttgtgataaaacccacacttgtccaccttgcccagcccc |
| | | tgagctacttgggggcccttcagtttttctgttcccacccaagcctaaagacacattgatgattagtagg |
| | | actcctgaagtgacctgtgttgtggtggatgtttcccatgaggacccagaagtaaagttcaattggtat |
| | | gtggatggagttgaggtgcacaatgctaagactaaaccaagagaagagcagtataatagcacatac |
| | | agggttgtgtcagttctgacagtgcttcaccaagactggctcaatggaaaggagtataaatgcaaggt |
| | | atctaataaggccctgcctgcacctattgaaaaaactatttcaaaggctaaagggcagccaagagag |
| | | ccccaggtctacaccctgcctccatccagggatgaattaacaaagaaccaggtgagtctcacttgcc |
| | | tggttaaaggtttttatccctctgatattgctgtggagtgggagtctaatggccaaccagaaaataacta |
| | | caagaccacacctccagtcttggactctgatggaagcttctttctttactcaaagctgactgtggacaa |
| | | aagtagatggcagcaggggaatgtgttcagctgttctgttatgcatgaggcactacacaaccattata |
| | | cccagaagtctctcagtctgtcacctggcaaatag |
| (SEQ ID NO: | ORF | atgtacagaatgcagctgctcagctgcattgccttgtcccttgctctggtgaccaactctgagattgtct |
| 66) | #10 | taacacaatcacctgcaactctgagtctaagtcctggagaaagagcaactctcagctgtagggcctc |
| Full: 708 | | ccagtctgtttcaagttatctggcttggtaccagcaaaagccaggacaggccccaaggttgctgatat |
| IL-2 Signal | | atgatgcaagcaaaagagccacagggatccctgctaggttctctggttctggctctggaacagatttt |
| peptide: 1-60 | | actcttaccatttcctcacttgagcctgaagactttgcagtgtactattgccagcagagaagtaagtgg |
| LC: 61-705 | | ccaccctggacatttgggcaaggcaccaaagtagagagcaagagaactgtggctgcaccttctgtc |
| Stop Codon: | | ttcatctttccaccctctgatgaacagctgaaatcaggtactgcttcagtggtttgtctgctcaataact |
| 706-708 | | tctacccaagggaggccaaggtgcagtggaaggttgacaatgcattgcagtctggaaacagtcaaga |
| | | gtctgtaacagaacaggattccaaagacagcacctattcactttccagtactctgaccttatctaaggc |
| | | tgattatgagaaacacaaggtgtatgcttgtgaagttacacatcaggggctgagctcccctgtgacca |
| | | aaagctttaataggggggagtgttag |
| (SEQ ID NO: | ORF | atgtacagaatgcagctgctcagctgcattgccttgtcccttgctctggtgaccaactctgagattgtct |
| 67) | #11 | taacacaatcacctgcaactctgagtctaagtcctggagaaagagcaactctcagctgtagggcctc |
| Full: 708 | | ccagtctgtttcaagttatctggcttggtaccagcaaaagccaggacaggccccaaggttgctgatat |
| IL-2 Signal | | atgatgcaagcaaaagagccacagggatccctgctaggttctctggttctggctctggaacagatttt |
| peptide: 1-60 | | actcttaccatttcctcacttgagcctgaagactttgcagtgtactattgccagcagagaagtaagtgg |
| LC: 61-705 | | ccaccctggacatttgggcaaggcaccaaagtagagagcaagagaactgtggctgcaccttctgtc |
| Stop Codon: | | ttcatctttccaccctctgatgaacagctgaaatcaggtactgcttcagtggtttgtctgctcaataact |
| 706-708 | | tctacccaagggaggccaaggtgcagtggaaggttgacaatgcattgcagtctggaaacagtcaaga |
| | | gtctgtaacagaacaggattccaaagacagcacctattcactttccagtactctgaccttatctaaggc |
| | | tgattatgagaaacacaaggtgtatgcttgtgaagttacacatcaggggctgagctcccctgtgacca |
| | | aaagctttaataggggggagtgttag |
| (SEQ ID NO: | ORF | atgtaccggatgcagctgctcagctgcatcgccttgtcccttgctctggtgaccaactctgagattgtc |
| 94) | #12 | ttaacacaatcacccgcaactctgagtctatcgcctggcgaaagagcgacgctcagctgtagggcct |
| Full: 2694 | | cccagtctgtttcaagttatctggcttggtaccagcaaaagccaggacaggccccgcgcttgctgata |
| IL-2 Signal | | tatgacgcaagcaaacgagccaccgggatccccgctcgtttctccggttctggcagcggaacagat |
| peptide: 1-60 | | tttactcttaccatttcctcactcgagcctgaagacttcgcagtgtactattgccagcagcggagtaagt |
| LC: 61-705 | | ggccaccctggacatttgggcaaggcaccaaagtagagagcaagagaactgtggccgcgccttct |
| Stop Codon: | | gtcttcatctttccaccctccgatgaacagctgaaatcgggtacggcttcagtggtttgtctgctcaata |
| 706-708 | | acttctacccgagggaggccaaggtgcagtggaaggtcgacaatgcattgcagagcggaaacagt |
| IRES: | | caagagtctgtaacagaacaggattccaaagacagcacctattcactttccagtactctgaccttatct |
| 709-1293 | | aaggccgattacgagaaacacaaggtgtacgcttgcgaagttacacatcaggggctgagctcccct |
| IL-10 Signal | | gtgaccaaaagctttaatcgcggcgagtgttaggcccctctccctcccccccccctaacgttactggc |
| peptide: | | cgaagccgcttggaataaggccggtgtgcgtttgtctatatgttattttccaccatattgccgtcttttg |
| 1294-1347 | | gcaatgtgagggcccggaaacctggccctgtcttcttgacgagcattcctaggggtctttcccctctcg |
| HC: | | ccaaaggaatgcaaggtctgttgaatgtcgtgaaggaagcagttcctctggaagcttcttgaagaca |
| 1348-2691 | | aacaacgtctgtagcgaccctttgcaggcagcggaaccccccacctggcgacaggtgcctctgcg |
| Stop Codon: | | gccaaaagccacgtgtataagatacacctgcaaaggcggcacaaccccagtgccacgttgtgagtt |
| 2692-2694 | | ggatagttgtggaaagagtcaaatggctctcctcaagcgtattcaacaaggggctgaaggatgccc |
| | | agaaggtaccccattgtatgggatctgatctggggcctcggtacacatgctttacatgtgtttagtcga |
| | | ggttaaaaaaacgtctaggccccccgaaccacggggacgtggttttcctttgaaaaacacgatgata |
| | | atatggccacaatgcacagctccgccctgctctgctgtttggtgcttctgaccggcgtccgggctcag |
| | | gttgagttagtggaatctggagggggtgtagtgcaacccggcagatcacagaggctgagttgcgca |
| | | gcgtcgggattcacatttagctcctacgggatgcattgggtccgccaggcccctggcaagggtctag |
| | | agtgggtggctatcatttggttcgacggatcttcaacttattacgccgatagtgttcgagggcgttttac |
| | | gataagccgggacaactccaaaaataccctctatctgcaaatgaactctttgagagcagaagataca |
| | | gccgtgtacttctgtgctagggagctgggccgccggtattttgacctttggggcagaggaactctcgt |
| | | cagcgtatcctcagcaagtaccaaggggccaagcgtgttcccgctggccccctcttccaaatcgac |
| | | atcaggtggcaccgcggctctgggatgcctcgttaaggattactttcctgaaccagtgactgtcagct |
| | | ggaatagtggggccttgacgtctggcgtgcacacattccccgcagtccttcagtccagcggtctgtat |
| | | tcattatccagtgtggttaccgtgccttctagctccctgggaactcagacctacatctgtaacgtaaatc |
| | | ataaaccaagcaacacaaaggtggacaagaaagtcgagcccaagtcttgcgataaaacccacactt |
| | | gtccgccttgcccagcccccgagctactcgggggcccttcagtttttctgttcccacccaagcctaaa |
| | | gacacattgatgattagtaggacgcccgaagtgacctgtgtcgtggtggatgtttcccatgaggacc |
| | | cagaagtaaagttcaattggtacgtggatggcgtcgaggtgcacaacgctaagactaaaccgcgcg |
| | | aagagcagtataatagcacataccgagtcgtgtcggttctgaccgtgcttcaccaagactggctcaa |
| | | cggaaaggagtataaatgcaaggtatctaataaggccctgcccgcacctatcgaaaaaactatttca |
| | | aaggctaaagggcagccacgtgagccccaggtctacaccctgcctccatcccgggacgaattaac |
| | | aaagaaccaggtgagtctcacgtgcctggttaaaggtttttatcccagcgatatcgccgtggagtgg |
| | | gagtctaacggccaaccggaaaataactacaagaccacacctccagtcttggactccgatggaagc |
| | | ttctttctttactcaaagctgactgtggacaaaagtagatggcagcaggggaatgtgttcagctgttcc |
| | | gttatgcatgaggcgctacacaaccattatacccagaagtctctctcgctgtcacccggcaaatag |
| (SEQ ID NO: | ORF | atgtacagaatgcagctgctcagctgcattgccttgtcccttgctctggtgaccaactctgagattgtct |
| 95) | #13 | taacacaatcacccgcaactctgagtctaagtcctggagaaagagcaactctcagctgtagggcctc |
| Full: 2700 | | ccagtctgtttcaagttatctggcttggtaccagcaaaagccaggacaggccccgcgcttgctgatat |
| IL-2 Signal | | atgatgcaagcaaaagggccacagggatccccgctagattcagcggttctggcagcggaacagat |
| peptide: 1-60 | | tttactcttaccatttcctcacttgagcctgaagactttgcagtgtactattgccagcagcggagtaagt |
| LC: 91-705 | | ggccaccctggacatttgggcaaggcaccaaagtagagagcaagagaactgtggccgcgccttct |
| IRES: | | gtcttcatctttccaccctctgatgaacagctgaaatcaggtactgcttcagtggtttgtctgctcaata |
| 709-1293 | | acttctacccaagggaggccaaggtgcagtggaaggttgacaatgcattgcagagcggaaacagtc |
| IL-10 Signal | | aagagtctgtaacagaacaggattccaaagacagcacctattcactttccagtactctgaccttatcta |
| peptide: | | aggctgattatgagaaacacaaggtgtatgcttgtgaagttacacatcaggggctgagctcccctgtg |
| 1294-1347 | | accaaaagctttaataggggggagtgttaggcccctctccctcccccccccctaacgttactggccg |
| HC: | | aagccgcttggaataaggccggtgtgcgtttgtctatatgttattttccaccatattgccgtcttttggc |
| 1294-2697 | | aatgtgagggcccggaaacctggccctgtcttcttgacgagcattcctaggggtctttcccctctcgcc |
| Stop codon: | | aaaggaatgcaaggtctgttgaatgtcgtgaaggaagcagttcctctggaagcttcttgaagacaaa |
| 2698-2700- | | caacgtctgtagcgaccctttgcaggcagcggaaccccccacctggcgacaggtgcctctgcggc |
| | | caaaagccacgtgtataagatacacctgcaaaggcggcacaaccccagtgccacgttgtgagttgg |
| | | atagttgtggaaagagtcaaatggctctcctcaagcgtattcaacaaggggctgaaggatgcccaga |
| | | aggtaccccattgtatgggatctgatctggggcctcggtacacatgctttacatgtgtttagtcgaggtt |
| | | aaaaaaacgtctaggccccccgaaccacggggacgtggttttcctttgaaaaacacgatgataatat |
| | | ggccacaatgcacagctccgccctgctctgctgtttggtgcttctgacaggagtcagggctcaggtt |
| | | gagttagtggaatctggagggggtgtagtgcaacctggcagatcacagaggctgagttgcgcagc |
| | | aagtggattcacatttagctcctatgggatgcattgggtccgccaggcccctggcaagggtctagag |
| | | tgggtggctatcatttggtttgatggatcttcaacttattatgctgatagtgttagagggcgctttacta |
| | | taagcagggacaactccaaaaataccctctatctgcaaatgaactctttgagagcagaagatacagctg |
| | | tgtacttctgtgctagggagctgggccgcagatattttgacctttggggcagaggaactcttgtctctgt |
| | | atcctcagcaagtaccaaggggccatctgtgttcccgctggccccctcttccaaaagtacatcaggt |
| | | ggcaccgcggctctgggatgcttggttaaggattactttcctgaaccagtgactgtcagctggaatag |
| | | tggggccttgacttctggggtgcacacattccccgcagtccttcagtccagcggtctgtattcattatc |
| | | cagtgtggttacagtgccttctagctccctgggaactcagacctacatctgtaatgtaaatcataaacc |
| | | aagcaacacaaaggtggacaagaaagttgagcccaagtcttgtgataaaacccacacttgtccgcc |
| | | ttgcccagcccctgagctacttgggggcccttcagtttttctgttcccacccaagcctaaagacacatt |
| | | gatgattagtaggactcctgaagtgacctgtgttgtggtggatgtttcccatgaggacccagaagtaa |
| | | agttcaattggtatgtggatggagttgaggtgcacaatgctaagactaaaccgagggaagagcagta |
| | | taatagcacatacagagttgtgtcagttctgacagtgcttcaccaagactggctcaatggaaaggagt |
| | | ataaatgcaaggtatctaataaggccctgcccgcacctattgaaaaaactatttcaaaggctaaaggg |
| | | cagccaagggagccccaggtctacaccctgcctccatccagagatgaattaacaaagaaccaggt |
| | | gagtctcacttgcctggttaaaggtttttatccctctgatattgctgtggagtgggagtctaatggccaa |
| | | ccagaaaataactacaagaccacacctccagtcttggactctgatggaagcttctttctttactcaaag |
| | | ctgactgtggacaaaagtagatggcagcaggggaatgtgttcagctgttctgttatgcatgaggcgct |
| | | acacaaccattatacccagaagtctctcagtctgtcaccggcaaatagcaattga |
| (SEQ ID NO: | ORF | atgcacagctctgccctgctctgctgtttggtgcttctgacaggagtcagggctcaggttgagttagtg |
| 96 | #14 | gaatctggagggggtgtagtgcaacctggcagatcacagaggctgagttgtgcagcaagtggattc |
| Full: 2211 | | acatttagctcctatgggatgcattgggtcagacaggcccctggcaagggtctagagtgggtggcta |
| IL-10 Signal | | tcatttggtttgatggatcttcaacttattatgctgatagtgttagggggagatttactataagcaggga |
| peptide: 1-54 | | caactccaaaaataccctctatctgcaaatgaactctttgagagcagaagatacagctgtgtacttctgt |
| HC: 55-1398 | | gctagggagctgggcagaaggtattttgacctttggggcagaggaactcttgtctctgtatcctcagc |
| Stop Codon: | | aagtaccaaggggccatctgtgttcccactggccccctcttccaaaagtacatcaggtggcacagca |
| 1399-1402 | | gctctgggatgcttggttaaggattactttcctgaaccagtgactgtcagctggaatagtggggccttg |
| Linker: | | acttctggggtgcacacattccctgcagtccttcagtcctctggtctgtattcattatccagtgtggtta |
| 1403-1407 | | cagtgccttctagctccctgggaactcagacctacatctgtaatgtaaatcataaaccaagcaacacaa |
| Furin: | | aggtggacaagaaagttgagcccaagtcttgtgataaaacccacacttgtccaccttgcccagcccc |
| 1408-1419 | | tgagctacttgggggcccttcagtttttctgttcccacccaagcctaaagacacattgatgattagtagg |
| Linker: | | actcctgaagtgacctgtgttgtggtggatgtttcccatgaggacccagaagtaaagttcaattggtat |
| 1420-1431 | | gtggatggagttgaggtgcacaatgctaagactaaaccaagagaagagcagtataatagcacatac |
| F2A: | | agggttgtgtcagttctgacagtgcttcaccaagactggctcaatggaaaggagtataaatgcaaggt |
| 1432-1503 | | atctaataaggccctgcctgcacctattgaaaaaactatttcaaaggctaaagggcagccaagagag |
| IL-2 Signal | | ccccaggtctacaccctgcctccatccagggatgaattaacaaagaaccaggtgagtctcacttgcc |
| peptide: | | tggttaaaggtttttatccctctgatattgctgtggagtgggagtctaatggccaaccagaaaataacta |
| 1505-1563 | | caagaccacacctccagtcttggactctgatggaagcttctttctttactcaaagctgactgtggacaa |
| LC: | | aagtagatggcagcaggggaatgtgttcagctgttctgttatgcatgaggcactacacaaccattata |
| 1564-2208 | | cccagaagtctctcagtctgtcacctggcaaatagggcaaaagaaagagaaggagtggctcagga |
| Stop Codon: | | gcccctgtgaaacagaccctgaactttgacctcttgaagcttgctggggatgtggagtctaatcctgg |
| 2209-2211 | | tccaatgtacagaatgcagctgctcagctgcattgccttgtcccttgctctggtgaccaactctgagatt |
| | | gtcttaacacaatcacctgcaactctgagtctaagtcctggagaaagagcaactctcagctgtaggg |
| | | cctcccagtctgtttcaagttatctggcttggtaccagcaaaagccaggacaggccccaaggttgctg |
| | | atatatgatgcaagcaaaagagccacagggatccctgctaggttctctggttctggctctggaacag |
| | | attttactcttaccatttcctcacttgagcctgaagactttgcagtgtactattgccagcagagaagtaa |
| | | gtggccaccctggacatttgggcaaggcaccaaagtagagagcaagagaactgtggctgcaccttct |
| | | gtcttcatctttccaccctctgatgaacagctgaaatcaggtactgcttcagtggtttgtctgctcaata |
| | | acttctacccaagggaggccaaggtgcagtggaaggttgacaatgcattgcagtctggaaacagtca |
| | | agagtctgtaacagaacaggattccaaagacagcacctattcactttccagtactctgaccttatctaa |
| | | ggctgattatgagaaacacaaggtgtatgcttgtgaagttacacatcaggggctgagctcccctgtga |
| | | ccaaaagctttaataggggggagtgttag |
| (SEQ ID NO: | ORF | atgtacagaatgcagctgctcagctgcattgccttgtcccttgctctggtgaccaactctgagattgtct |
| 97) | #15 | taacacaatcacctgcaactctgagtctaagtcctggagaaagagcaactctcagctgtagggcctc |
| Full: 2694 | | ccagtctgtttcaagttatctggcttggtaccagcaaaagccaggacaggccccaaggttgctgatat |
| IL-2 Signal | | atgatgcaagcaaaagagccacagggatccctgctaggttctctggttctggctctggaacagatttt |
| peptide: 1-60 | | actcttaccatttcctcacttgagcctgaagactttgcagtgtactattgccagcagagaagtaagtgg |
| LC: 61-705 | | ccaccctggacatttgggcaaggcaccaaagtagagagcaagagaactgtggctgcaccttctgtc |
| Stop Codon: | | ttcatctttccaccctctgatgaacagctgaaatcaggtactgcttcagtggtttgtctgctcaataact |
| 706-708 | | tctacccaagggaggccaaggtgcagtggaaggttgacaatgcattgcagtctggaaacagtcaaga |
| IRES: | | gtctgtaacagaacaggattccaaagacagcacctattcactttccagtactctgaccttatctaaggc |
| 709-1293 | | tgattatgagaaacacaaggtgtatgcttgtgaagttacacatcaggggctgagctcccctgtgacca |
| IL-10 Signal | | aaagctttaataggggggagtgttaggcccctctccctcccccccccctaacgttactggccgaagc |
| peptide: | | cgcttggaataaggccggtgtgcgtttgtctatatgttattttccaccatattgccgtcttttggcaatg |
| 1294-1347 | | tgagggcccggaaacctggccctgtcttcttgacgagcattcctaggggtctttcccctctcgccaaag |
| HC: | | gaatgcaaggtctgttgaatgtcgtgaaggaagcagttcctctggaagcttcttgaagacaaacaac |
| 1348-2691 | | gtctgtagcgaccctttgcaggcagcggaaccccccacctggcgacaggtgcctctgcggccaaa |
| Stop Codon: | | agccacgtgtataagatacacctgcaaaggcggcacaaccccagtgccacgttgtgagttggatag |
| 2692-2694 | | ttgtggaaagagtcaaatggctctcctcaagcgtattcaacaaggggctgaaggatgcccagaagg |
| | | taccccattgtatgggatctgatctggggcctcggtacacatgctttacatgtgtttagtcgaggttaaa |
| | | aaaacgtctaggccccccgaaccacggggacgtggttttcctttgaaaaacacgatgataatatggc |
| | | cacaatgcacagctctgccctgctctgctgtttggtgcttctgacaggagtcagggctcaggttgagtt |
| | | agtggaatctggagggggtgtagtgcaacctggcagatcacagaggctgagttgtgcagcaagtg |
| | | gattcacatttagctcctatgggatgcattgggtcagacaggcccctggcaagggtctagagtgggt |
| | | ggctatcatttggtttgatggatcttcaacttattatgctgatagtgttagggggagatttactataagc |
| | | agggacaactccaaaaataccctctatctgcaaatgaactctttgagagcagaagatacagctgtgtac |
| | | ttctgtgctagggagctgggcagaaggtattttgacctttggggcagaggaactcttgtctctgtatcct |
| | | cagcaagtaccaaggggccatctgtgttcccactggccccctcttccaaaagtacatcaggtggcac |
| | | agcagctctgggatgcttggttaaggattactttcctgaaccagtgactgtcagctggaatagtgggg |
| | | ccttgacttctggggtgcacacattccctgcagtccttcagtcctctggtctgtattcattatccagtgt |
| | | ggttacagtgccttctagctccctgggaactcagacctacatctgtaatgtaaatcataaaccaagcaac |
| | | acaaaggtggacaagaaagttgagcccaagtcttgtgataaaacccacacttgtccaccttgcccag |
| | | cccctgagctacttgggggcccttcagtttttctgttcccacccaagcctaaagacacattgatgatta |
| | | gtaggactcctgaagtgacctgtgttgtggtggatgtttcccatgaggacccagaagtaaagttcaatt |
| | | ggtatgtggatggagttgaggtgcacaatgctaagactaaaccaagagaagagcagtataatagca |
| | | catacagggttgtgtcagttctgacagtgcttcaccaagactggctcaatggaaaggagtataaatgc |
| | | aaggtatctaataaggccctgcctgcacctattgaaaaaactatttcaaaggctaaagggcagccaa |
| | | gagagccccaggtctacaccctgcctccatccagggatgaattaacaaagaaccaggtgagtctca |
| | | cttgcctggttaaaggtttttatccctctgatattgctgtggagtgggagtctaatggccaaccagaaaa |
| | | taactacaagaccacacctccagtcttggactctgatggaagcttctttctttactcaaagctgactgtg |
| | | gacaaaagtagatggcagcaggggaatgtgttcagctgttctgttatgcatgaggcactacacaacc |
| | | attatacccagaagtctctcagtctgtcacctggcaaatag |

The following modified anti-IGF-1R antibody expression cassette nucleic acid sequences (shown in Table 17) corresponding to SEQ ID NOs: 68-76 were designed in silico. The expression constructs include nucleic acid molecules encoding a promoter, a heavy chain and a light chain. Some sequences also include a furin cleavage site, a F2A peptide, a linker, an IRES, and a second promoter.

TABLE 17
Expression Cassettes encoding an IGFR-Alpha Antibody

| SEQ ID NO/ | | |
|---|---|---|
| length | Name | Sequence |
| (SEQ ID | EC #1 | CTGCGCGCTCGCTCGCTCACTGAGGCCGCCCGGGCAAAGCCC |
| NO: 68) | H- | GGGCGTCGGGCGACCTTTGGTCGCCCGGCCTCAGTGAGCGAG |
| Full: 4479 | F2A-L | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-10 signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1951 | | TTTGTATTTATTTATTTTTTAATTATTTTGTGCAGCGATGGGGG |
| HC: | | CGGGGGGGGGGGGGGCGCGCGCCAGGCGGGGCGGGGGGGG |
| 1952-3295 | | GCGAGGGGCGGGGCGGGGCGAGGCGGAGAGGTGCGGCGGC |
| Furin | | AGCCAATCAGAGCGGCGCGCTCCGAAAGTTTCCTTTTATGGC |
| cleavage site: | | GAGGCGGCGGCGGCGGCGGCCCTATAAAAAGCGAAGCGCGC |
| 3296-3307 | | GGCGGGCGGGAGTCGCTGCGCGCTGCCTTCGCCCCGTGCCCC |
| Linker: | | GCTCCGCCGCCGCCTCGCGCCGCCCGCCCCGGCTCTGACTGA |
| 3308-3319 | | CCGCGTTACTCCCACAGGTGAGCGGGCGGGACGGCCCTTCTC |
| F2A: | | CTCCGGGCTGTAATTAGCGCTTGGTTTAATGACGGCTCGTTTC |
| 3320-3391 | | TTTTCTGTGGCTGCGTGAAAGCCTTGAGGGGCTCCGGGAGGG |
| IL-2 signal | | CCCTTTGTGCGGGGGGAGCGGCTCGGGGGGTGCGTGCGTGTG |
| peptide: | | TGTGTGCGTGGGGAGCGCCGCGTGCGGCTCCGCGCTGCCCGG |
| 3392-3451 | | CGGCTGTGAGCGCTGCGGGCGCGGCGCGGGGCTTTGTGCGCT |
| LC: | | CCGCAGTGTGCGCGAGAGGAGCGCGGCCGGGGGCGGTGCCA |
| 3452-4096 | | CGCGGTGCGGGGGGGGCTGCGAGGGGAACAAAGGCTGCGTG |
| Stop Codon: | | CGGGGTGTGTGCGTGGGGGGGGTGAGCAGGGGGTGTGGGCG |
| 4097-4099 | | CGGCGGTCGGGCTGTAACCCCCCCCTGCACCCCCCTCCCCGA |
| bGHpA: | | GTTGCTGAGCACGGCCCGGCTTCGGGTGCGGGGCTCCGTGCG |
| 4106-4329 | | GGGCGTGGCGCGGGGCTCGCCGTGCCGGGCGGGGGGTGGCG |
| ITR: | | GCGGGTGGGGGTGCCGGGCGGGGCGGGGCCGCCTCGGGCCG |
| 4339-4479 | | GGGAGGGCTCGGGGGAGGGGCGCGGCGGCCCCCGGAGCGCC |
| | | GGCGGCTGTCGAGGCGCGGCGAGCCGCAGCCATTGCCTTTTA |
| | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGCACAGCTCCGCCCTGCTCTGCTGTTTGG |
| | | TGCTTCTGACCGGCGTCCGGGCTCAGGTTGAGTTAGTGGAAT |
| | | CTGGAGGGGGTGTAGTGCAACCCGGCAGATCACAGAGGCTG |
| | | AGTTGCGCAGCGTCGGGATTCACATTTAGCTCCTACGGGATG |
| | | CATTGGGTCCGCCAGGCCCCTGGCAAGGGTCTAGAGTGGGTG |
| | | GCTATCATTTGGTTCGACGGATCTTCAACTTATTACGCCGATA |
| | | GTGTTCGAGGGCGTTTTACGATAAGCCGGGACAACTCCAAAA |
| | | ATACCCTCTATCTGCAAATGAACTCTTTGAGAGCAGAAGATA |
| | | CAGCCGTGTACTTCTGTGCTAGGGAGCTGGGCCGCCGGTATT |
| | | TTGACCTTTGGGGCAGAGGAACTCTCGTCAGCGTATCCTCAG |
| | | CAAGTACCAAGGGGCCAAGCGTGTTCCCGCTGGCCCCCTCTT |
| | | CCAAATCGACATCAGGTGGCACCGCGGCTCTGGGATGCCTCG |
| | | TTAAGGATTACTTTCCTGAACCAGTGACTGTCAGCTGGAATA |
| | | GTGGGGCCTTGACGTCTGGCGTGCACACATTCCCCGCAGTCC |
| | | TTCAGTCCAGCGGTCTGTATTCATTATCCAGTGTGGTTACCGT |
| | | GCCTTCTAGCTCCCTGGGAACTCAGACCTACATCTGTAACGT |
| | | AAATCATAAACCAAGCAACACAAAGGTGGACAAGAAAGTCG |
| | | AGCCCAAGTCTTGCGATAAAACCCACACTTGTCCGCCTTGCC |
| | | CAGCCCCCGAGCTACTCGGGGGCCCTTCAGTTTTTCTGTTCCC |
| | | ACCCAAGCCTAAAGACACATTGATGATTAGTAGGACGCCCGA |
| | | AGTGACCTGTGTCGTGGTGGATGTTTCCCATGAGGACCCAGA |
| | | AGTAAAGTTCAATTGGTACGTGGATGGCGTCGAGGTGCACAA |
| | | CGCTAAGACTAAACCGCGCGAAGAGCAGTATAATAGCACAT |
| | | ACCGAGTCGTGTCGGTTCTGACCGTGCTTCACCAAGACTGGC |
| | | TCAACGGAAAGGAGTATAAATGCAAGGTATCTAATAAGGCC |
| | | CTGCCCGCACCTATCGAAAAAACTATTTCAAAGGCTAAAGGG |
| | | CAGCCACGTGAGCCCCAGGTCTACACCCTGCCTCCATCCCGG |
| | | GACGAATTAACAAAGAACCAGGTGAGTCTCACGTGCCTGGTT |
| | | AAAGGTTTTTATCCCAGCGATATCGCCGTGGAGTGGGAGTCT |
| | | AACGGCCAACCGGAAAATAACTACAAGACCACACCTCCAGT |
| | | CTTGGACTCCGATGGAAGCTTCTTTCTTTACTCAAAGCTGACT |
| | | GTGGACAAAAGTAGATGGCAGCAGGGGAATGTGTTCAGCTG |
| | | TTCCGTTATGCATGAGGCGCTACACAACCATTATACCCAGAA |
| | | GTCTCTCTCGCTGTCACCCGGCAAAAGAAAGAGAAGGAGTGG |
| | | CTCAGGAGCCCCTGTGAAACAGACCCTGAACTTTGACCTCTT |
| | | GAAGCTTGCTGGGGATGTGGAGTCTAATCCTGGTCCAATGTA |
| | | CCGGATGCAGCTGCTCAGCTGCATCGCCTTGTCCCTTGCTCTG |
| | | GTGACCAACTCTGAGATTGTCTTAACACAATCACCCGCAACT |
| | | CTGAGTCTATCGCCTGGCGAAAGAGCGACGCTCAGCTGTAGG |
| | | GCCTCCCAGTCTGTTTCAAGTTATCTGGCTTGGTACCAGCAAA |
| | | AGCCAGGACAGGCCCCGCGCTTGCTGATATATGACGCAAGCA |
| | | AACGAGCCACCGGGATCCCCGCTCGTTTCTCCGGTTCTGGCA |
| | | GCGGAACAGATTTTACTCTTACCATTTCCTCACTCGAGCCTGA |
| | | AGACTTCGCAGTGTACTATTGCCAGCAGCGGAGTAAGTGGCC |
| | | ACCCTGGACATTTGGGCAAGGCACCAAAGTAGAGAGCAAGA |
| | | GAACTGTGGCCGCGCCTTCTGTCTTCATCTTTCCACCCTCCGA |
| | | TGAACAGCTGAAATCGGGTACGGCTTCAGTGGTTTGTCTGCT |
| | | CAATAACTTCTACCCGAGGGAGGCCAAGGTGCAGTGGAAGG |
| | | TCGACAATGCATTGCAGAGCGGAAACAGTCAAGAGTCTGTAA |
| | | CAGAACAGGATTCCAAAGACAGCACCTATTCACTTTCCAGTA |
| | | CTCTGACCTTATCTAAGGCCGATTACGAGAAACACAAGGTGT |
| | | ACGCTTGCGAAGTTACACATCAGGGGCTGAGCTCCCCTGTGA |
| | | CCAAAAGCTTTAATCGCGGCGAGTGTTAGCAATTGCTGTGCC |
| | | TTCTAGTTGCCAGCCATCTGTTGTTTGCCCCTCCCCCGTGCCT |
| | | TCCTTGACCCTGGAAGGTGCCACTCCCACTGTCCTTTCCTAAT |
| | | AAAATGAGGAAATTGCATCGCATTGTCTGAGTAGGTGTCATT |
| | | CTATTCTGGGGGGTGGGGTGGGGCAGGACAGCAAGGGGGAG |
| | | GATTGGGAAGACAATAGCAGGCATGCTGGGGATGCGGTGGG |
| | | CTCTATGGAGCTCGAGAGGAACCCCTAGTGATGGAGTTGGCC |
| | | ACTCCCTCTCTGCGCGCTCGCTCGCTCACTGAGGCCGGGCGA |
| | | CCAAAGGTCGCCCGACGCCCGGGCTTTGCCCGGGCGGCCTCA |
| | | GTGAGCGAGCGAGCGCGCAGCTGCAGATCTG |
| (SEQ ID | EC #2 | CTGCGCGCTCGCTCGCTCACTGAGGCCGCCCGGGCAAAGCCC |
| NO: 69) | L- | GGGCGTCGGGCGACCTTTGGTCGCCCGGCCTCAGTGAGCGAG |
| Full: 4799 | IRES-H | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-2 signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1957 | | TTTGTATTTATTTATTTTTTAATTATTTTGTGCAGCGATGGGGG |
| LC: | | CGGGGGGGGGGGGGGCGCGCGCCAGGCGGGGCGGGGGGGG |
| 1958-2602 | | GCGAGGGGCGGGGCGGGGCGAGGCGGAGAGGTGCGGCGGC |
| Stop Codon: | | AGCCAATCAGAGCGGCGCGCTCCGAAAGTTTCCTTTTATGGC |
| 2603-2605 | | GAGGCGGCGGCGGCGGCGGCCCTATAAAAAGCGAAGCGCGC |
| IRES2: | | GGCGGGCGGGAGTCGCTGCGCGCTGCCTTCGCCCCGTGCCCC |
| 2606-3190 | | GCTCCGCCGCCGCCTCGCGCCGCCCGCCCCGGCTCTGACTGA |
| IL-10 signal | | CCGCGTTACTCCCACAGGTGAGCGGGCGGGACGGCCCTTCTC |
| peptide: | | CTCCGGGCTGTAATTAGCGCTTGGTTTAATGACGGCTCGTTTC |
| 3191-3244 | | TTTTCTGTGGCTGCGTGAAAGCCTTGAGGGGCTCCGGGAGGG |
| HC: | | CCCTTTGTGCGGGGGGAGCGGCTCGGGGGGTGCGTGCGTGTG |
| 3245-4588 | | TGTGTGCGTGGGGAGCGCCGCGTGCGGCTCCGCGCTGCCCGG |
| Stop Codon: | | CGGCTGTGAGCGCTGCGGGCGCGGCGCGGGGCTTTGTGCGCT |
| 4589-4591 | | CCGCAGTGTGCGCGAGAGGAGCGCGGCCGGGGGCGGTGCCA |
| Synthetic | | CGCGGTGCGGGGGGGGCTGCGAGGGGAACAAAGGCTGCGTG |
| poly(A): | | CGGGGTGTGTGCGTGGGGGGGGTGAGCAGGGGGTGTGGGCG |
| 4601-4649 | | CGGCGGTCGGGCTGTAACCCCCCCCTGCACCCCCCTCCCCGA |
| ITR: | | GTTGCTGAGCACGGCCCGGCTTCGGGTGCGGGGCTCCGTGCG |
| 4659-4799 | | GGGCGTGGCGCGGGGCTCGCCGTGCCGGGCGGGGGGTGGCG |
| | | GCGGGTGGGGGTGCCGGGCGGGGGGGGGCCGCCTCGGGCCG |
| | | GGGAGGGCTCGGGGGAGGGGCGCGGCGGCCCCCGGAGCGCC |
| | | GGCGGCTGTCGAGGCGCGGCGAGCCGCAGCCATTGCCTTTTA |
| | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGTACCGGATGCAGCTGCTCAGCTGCATC |
| | | GCCTTGTCCCTTGCTCTGGTGACCAACTCTGAGATTGTCTTAA |
| | | CACAATCACCCGCAACTCTGAGTCTATCGCCTGGCGAAAGAG |
| | | CGACGCTCAGCTGTAGGGCCTCCCAGTCTGTTTCAAGTTATCT |
| | | GGCTTGGTACCAGCAAAAGCCAGGACAGGCCCCGCGCTTGCT |
| | | GATATATGACGCAAGCAAACGAGCCACCGGGATCCCCGCTCG |
| | | TTTCTCCGGTTCTGGCAGCGGAACAGATTTTACTCTTACCATT |
| | | TCCTCACTCGAGCCTGAAGACTTCGCAGTGTACTATTGCCAG |
| | | CAGCGGAGTAAGTGGCCACCCTGGACATTTGGGCAAGGCACC |
| | | AAAGTAGAGAGCAAGAGAACTGTGGCCGCGCCTTCTGTCTTC |
| | | ATCTTTCCACCCTCCGATGAACAGCTGAAATCGGGTACGGCT |
| | | TCAGTGGTTTGTCTGCTCAATAACTTCTACCCGAGGGAGGCC |
| | | AAGGTGCAGTGGAAGGTCGACAATGCATTGCAGAGCGGAAA |
| | | CAGTCAAGAGTCTGTAACAGAACAGGATTCCAAAGACAGCA |
| | | CCTATTCACTTTCCAGTACTCTGACCTTATCTAAGGCCGATTA |
| | | CGAGAAACACAAGGTGTACGCTTGCGAAGTTACACATCAGG |
| | | GGCTGAGCTCCCCTGTGACCAAAAGCTTTAATCGCGGCGAGT |
| | | GTTAGGCCCCTCTCCCTCCCCCCCCCCTAACGTTACTGGCCGA |
| | | AGCCGCTTGGAATAAGGCCGGTGTGCGTTTGTCTATATGTTAT |
| | | TTTCCACCATATTGCCGTCTTTTGGCAATGTGAGGGCCCGGAA |
| | | ACCTGGCCCTGTCTTCTTGACGAGCATTCCTAGGGGTCTTTCC |
| | | CCTCTCGCCAAAGGAATGCAAGGTCTGTTGAATGTCGTGAAG |
| | | GAAGCAGTTCCTCTGGAAGCTTCTTGAAGACAAACAACGTCT |
| | | GTAGCGACCCTTTGCAGGCAGCGGAACCCCCCACCTGGCGAC |
| | | AGGTGCCTCTGCGGCCAAAAGCCACGTGTATAAGATACACCT |
| | | GCAAAGGCGGCACAACCCCAGTGCCACGTTGTGAGTTGGATA |
| | | GTTGTGGAAAGAGTCAAATGGCTCTCCTCAAGCGTATTCAAC |
| | | AAGGGGCTGAAGGATGCCCAGAAGGTACCCCATTGTATGGG |
| | | ATCTGATCTGGGGCCTCGGTACACATGCTTTACATGTGTTTAG |
| | | TCGAGGTTAAAAAAACGTCTAGGCCCCCCGAACCACGGGGA |
| | | CGTGGTTTTCCTTTGAAAAACACGATGATAATATGGCCACAA |
| | | TGCACAGCTCCGCCCTGCTCTGCTGTTTGGTGCTTCTGACCGG |
| | | CGTCCGGGCTCAGGTTGAGTTAGTGGAATCTGGAGGGGGTGT |
| | | AGTGCAACCCGGCAGATCACAGAGGCTGAGTTGCGCAGCGTC |
| | | GGGATTCACATTTAGCTCCTACGGGATGCATTGGGTCCGCCA |
| | | GGCCCCTGGCAAGGGTCTAGAGTGGGTGGCTATCATTTGGTT |
| | | CGACGGATCTTCAACTTATTACGCCGATAGTGTTCGAGGGCG |
| | | TTTTACGATAAGCCGGGACAACTCCAAAAATACCCTCTATCT |
| | | GCAAATGAACTCTTTGAGAGCAGAAGATACAGCCGTGTACTT |
| | | CTGTGCTAGGGAGCTGGGCCGCCGGTATTTTGACCTTTGGGG |
| | | CAGAGGAACTCTCGTCAGCGTATCCTCAGCAAGTACCAAGGG |
| | | GCCAAGCGTGTTCCCGCTGGCCCCCTCTTCCAAATCGACATC |
| | | AGGTGGCACCGCGGCTCTGGGATGCCTCGTTAAGGATTACTT |
| | | TCCTGAACCAGTGACTGTCAGCTGGAATAGTGGGGCCTTGAC |
| | | GTCTGGCGTGCACACATTCCCCGCAGTCCTTCAGTCCAGCGG |
| | | TCTGTATTCATTATCCAGTGTGGTTACCGTGCCTTCTAGCTCC |
| | | CTGGGAACTCAGACCTACATCTGTAACGTAAATCATAAACCA |
| | | AGCAACACAAAGGTGGACAAGAAAGTCGAGCCCAAGTCTTG |
| | | CGATAAAACCCACACTTGTCCGCCTTGCCCAGCCCCCGAGCT |
| | | ACTCGGGGGCCCTTCAGTTTTTCTGTTCCCACCCAAGCCTAAA |
| | | GACACATTGATGATTAGTAGGACGCCCGAAGTGACCTGTGTC |
| | | GTGGTGGATGTTTCCCATGAGGACCCAGAAGTAAAGTTCAAT |
| | | TGGTACGTGGATGGCGTCGAGGTGCACAACGCTAAGACTAAA |
| | | CCGCGCGAAGAGCAGTATAATAGCACATACCGAGTCGTGTCG |
| | | GTTCTGACCGTGCTTCACCAAGACTGGCTCAACGGAAAGGAG |
| | | TATAAATGCAAGGTATCTAATAAGGCCCTGCCCGCACCTATC |
| | | GAAAAAACTATTTCAAAGGCTAAAGGGCAGCCACGTGAGCC |
| | | CCAGGTCTACACCCTGCCTCCATCCCGGGACGAATTAACAAA |
| | | GAACCAGGTGAGTCTCACGTGCCTGGTTAAAGGTTTTTATCC |
| | | CAGCGATATCGCCGTGGAGTGGGAGTCTAACGGCCAACCGG |
| | | AAAATAACTACAAGACCACACCTCCAGTCTTGGACTCCGATG |
| | | GAAGCTTCTTTCTTTACTCAAAGCTGACTGTGGACAAAAGTA |
| | | GATGGCAGCAGGGGAATGTGTTCAGCTGTTCCGTTATGCATG |
| | | AGGCGCTACACAACCATTATACCCAGAAGTCTCTCTCGCTGT |
| | | CACCCGGCAAATAGTGACAATTGAATAAAAGATCTTTATTTT |
| | | CATTAGATCTGTGTGTTGGTTTTTTGTGTGGAGCTCGAGAGGA |
| | | ACCCCTAGTGATGGAGTTGGCCACTCCCTCTCTGCGCGCTCGC |
| | | TCGCTCACTGAGGCCGGGCGACCAAAGGTCGCCCGACGCCCG |
| | | GGCTTTGCCCGGGCGGCCTCAGTGAGCGAGCGAGCGCGCAGC |
| | | TGCAGATCTG |
| (SEQ ID | EC #3 | CTGCGCGCTCGCTCGCTCACTGAGGCCGCCCGGGCAAAGCCC |
| NO: 70) | Dual | GGGCGTCGGGCGACCTTTGGTCGCCCGGCCTCAGTGAGCGAG |
| Full: 4288 | | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CMV | | TGACGTATGTTCCCATAGTAACGCCAATAGGGACTTTCCATT |
| promoter: | | GACGTCAATGGGTGGACTATTTACGGTAAACTGCCCACTTGG |
| 529-732 | | CAGTACATCAAGTGTATCATATGCCAAGTACGCCCCCTATTG |
| SV40 intron: | | ACGTCAATGACGGTAAATGGCCCGCCTGGCATTATGCCCAGT |
| 791-887 | | ACATGACCTTATGGGACTTTCCTACTTGGCAGTACATCTACGT |
| IL-12 signal | | ATTAGTCATCGCTATTACCATGGTGATGCGGTTTTGGCAGTAC |
| peptide: | | ATCAATGGGCGTGGATAGCGGTTTGACTCACGGGGATTTCCA |
| 954-1013 | | AGTCTCCACCCCATTGACGTCAATGGGAGTTTGTTTTGGCACC |
| LC: | | AAAATCAACGGGACTTTCCAAAATGTCGTAACAACTCCGCCC |
| 1014-1658 | | CATTGACGCAAATGGGCGGTAGGCGTGTACGGTGGGAGGTCT |
| Stop Codon: | | ATATAAGCAGAGCTCGTTTAGTGAACCGTCAGATCGCCTGGA |
| 1659-1661 | | GAAGGAACTGAAAAACCAGAAAGTTAACTGGTAAGTTTAGT |
| bGH | | CTTTTTGTCTTTTATTTCAGGTCCCGGATCCGGTGGTGGTGCA |
| poly(A): | | AATCAAAGAACTGCTCCTCAGTGGATGTTGCCTTTACTTCTAG |
| 1668-1842 | | GCCTGTACGGAAGTGTTACTTCTGCTCTAAAAGCTGCGGAAT |
| Pause site: | | TGTAGCCCGGTTAATTAAGCCACCATGTACCGGATGCAGCTG |
| 1857-1948 | | CTCAGCTGCATCGCCTTGTCCCTTGCTCTGGTGACCAACTCTG |
| EF-1α | | AGATTGTCTTAACACAATCACCCGCAACTCTGAGTCTATCGC |
| Promoter: | | CTGGCGAAAGAGCGACGCTCAGCTGTAGGGCCTCCCAGTCTG |
| 1967-2196 | | TTTCAAGTTATCTGGCTTGGTACCAGCAAAAGCCAGGACAGG |
| 5′ LTR | | CCCCGCGCTTGCTGATATATGACGCAAGCAAACGAGCCACCG |
| (truncated): | | GGATCCCCGCTCGTTTCTCCGGTTCTGGCAGCGGAACAGATTT |
| 2209-2477 | | TACTCTTACCATTTCCTCACTCGAGCCTGAAGACTTCGCAGTG |
| HBG/IgHC | | TACTATTGCCAGCAGCGGAGTAAGTGGCCACCCTGGACATTT |
| chimeric | | GGGCAAGGCACCAAAGTAGAGAGCAAGAGAACTGTGGCCGC |
| intron: | | GCCTTCTGTCTTCATCTTTCCACCCTCCGATGAACAGCTGAAA |
| 2504-2636 | | TCGGGTACGGCTTCAGTGGTTTGTCTGCTCAATAACTTCTACC |
| IL-10 signal | | CGAGGGAGGCCAAGGTGCAGTGGAAGGTCGACAATGCATTG |
| peptide: | | CAGAGCGGAAACAGTCAAGAGTCTGTAACAGAACAGGATTC |
| 2686-2739 | | CAAAGACAGCACCTATTCACTTTCCAGTACTCTGACCTTATCT |
| HC: | | AAGGCCGATTACGAGAAACACAAGGTGTACGCTTGCGAAGTT |
| 2739-4083 | | ACACATCAGGGGCTGAGCTCCCCTGTGACCAAAAGCTTTAAT |
| Stop Codon: | | CGCGGCGAGTGTTAGGCATGCCTGTGCCTTCTAGTTGCCAGC |
| 4084-4086 | | CATCTGTTGTTTGCCCCTCCCCCGTGCCTTCCTTGACCCTGGA |
| Synthetic | | AGGTGCCACTCCCACTGTCCTTTCCTAATAAAATGAGGAAAT |
| poly(A): | | TGCATCGCATTGTCTGAGTAGGTGTCATTCTATTCTGGGGGGT |
| 4093-4141 | | GGGGTGGGGCAGGACAGCAAGGGGGAGGATTCTCAGAACTA |
| ITR: | | ACATACGCTCTCCATCAAAACAAAACGAAACAAAACAAACT |
| 4148-4288 | | AGCAAAATAGGCTGTCCCCAGTGCAAGTGCAGGTGCCAGAA |
| | | CATTTCTCTATCGAAGGATCTGCGATCGCTCCGGTGCCCGTCA |
| | | GTGGGCAGAGCGCACATCGCCCACAGTCCCCGAGAAGTTGG |
| | | GGGGAGGGGTCGGCAATTGAACCGGTGCCTAGAGAAGGTGG |
| | | CGCGGGGTAAACTGGGAAAGTGATGTCGTGTACTGGCTCCGC |
| | | CTTTTTCCCGAGGGTGGGGGAGAACCGTATATAAGTGCAGTA |
| | | GTCGCCGTGAACGTTCTTTTTCGCAACGGGTTTGCCGCCAGA |
| | | ACACAGCTGAAGCTTCGAGGGGCTCGCATCTCTCCTTCACGC |
| | | GCCCGCCGCCCTACCTGAGGCCGCCATCCACGCCGGTTGAGT |
| | | CGCGTTCTGCCGCCTCCCGCCTGTGGTGCCTCCTGAACTGCGT |
| | | CCGCCGTCTAGGTAAGTTTAAAGCTCAGGTCGAGACCGGGCC |
| | | TTTGTCCGGCGCTCCCTTGGAGCCTACCTAGACTCAGCCGGCT |
| | | CTCCACGCTTTGCCTGACCCTGCTTGCTCAACTCTACGTCTTT |
| | | GTTTCGTTTTCTGTTCTGCGCCGTTACAGATCGAAGTTGGTCG |
| | | TGAGGCACTGGGCAGGTAAGTATCAAGGTTACAAGACAGGTT |
| | | TAAGGAGACCAATAGAAACTGGGCTTGTCGAGACAGAGAAG |
| | | ACTCTTGCGTTTCTGATAGGCACCTATTGGTCTTACTGACATC |
| | | CACTTTGCCTTTCTCTCCACAGGTGTCCACTCCCAGTTCAATT |
| | | ACAGCTCTTAAGGCTAGTTTAAACCACCATGCACAGCTCCGC |
| | | CCTGCTCTGCTGTTTGGTGCTTCTGACCGGCGTCCGGGCTCAG |
| | | GTTGAGTTAGTGGAATCTGGAGGGGGTGTAGTGCAACCCGGC |
| | | AGATCACAGAGGCTGAGTTGCGCAGCGTCGGGATTCACATTT |
| | | AGCTCCTACGGGATGCATTGGGTCCGCCAGGCCCCTGGCAAG |
| | | GGTCTAGAGTGGGTGGCTATCATTTGGTTCGACGGATCTTCA |
| | | ACTTATTACGCCGATAGTGTTCGAGGGCGTTTTACGATAAGC |
| | | CGGGACAACTCCAAAAATACCCTCTATCTGCAAATGAACTCT |
| | | TTGAGAGCAGAAGATACAGCCGTGTACTTCTGTGCTAGGGAG |
| | | CTGGGCCGCCGGTATTTTGACCTTTGGGGCAGAGGAACTCTC |
| | | GTCAGCGTATCCTCAGCAAGTACCAAGGGGCCAAGCGTGTTC |
| | | CCGCTGGCCCCCTCTTCCAAATCGACATCAGGTGGCACCGCG |
| | | GCTCTGGGATGCCTCGTTAAGGATTACTTTCCTGAACCAGTG |
| | | ACTGTCAGCTGGAATAGTGGGGCCTTGACGTCTGGCGTGCAC |
| | | ACATTCCCCGCAGTCCTTCAGTCCAGCGGTCTGTATTCATTAT |
| | | CCAGTGTGGTTACCGTGCCTTCTAGCTCCCTGGGAACTCAGA |
| | | CCTACATCTGTAACGTAAATCATAAACCAAGCAACACAAAGG |
| | | TGGACAAGAAAGTCGAGCCCAAGTCTTGCGATAAAACCCAC |
| | | ACTTGTCCGCCTTGCCCAGCCCCCGAGCTACTCGGGGGCCCTT |
| | | CAGTTTTTCTGTTCCCACCCAAGCCTAAAGACACATTGATGAT |
| | | TAGTAGGACGCCCGAAGTGACCTGTGTCGTGGTGGATGTTTC |
| | | CCATGAGGACCCAGAAGTAAAGTTCAATTGGTACGTGGATGG |
| | | CGTCGAGGTGCACAACGCTAAGACTAAACCGCGCGAAGAGC |
| | | AGTATAATAGCACATACCGAGTCGTGTCGGTTCTGACCGTGC |
| | | TTCACCAAGACTGGCTCAACGGAAAGGAGTATAAATGCAAG |
| | | GTATCTAATAAGGCCCTGCCCGCACCTATCGAAAAAACTATT |
| | | TCAAAGGCTAAAGGGCAGCCACGTGAGCCCCAGGTCTACACC |
| | | CTGCCTCCATCCCGGGACGAATTAACAAAGAACCAGGTGAGT |
| | | CTCACGTGCCTGGTTAAAGGTTTTTATCCCAGCGATATCGCCG |
| | | TGGAGTGGGAGTCTAACGGCCAACCGGAAAATAACTACAAG |
| | | ACCACACCTCCAGTCTTGGACTCCGATGGAAGCTTCTTTCTTT |
| | | ACTCAAAGCTGACTGTGGACAAAAGTAGATGGCAGCAGGGG |
| | | AATGTGTTCAGCTGTTCCGTTATGCATGAGGCGCTACACAAC |
| | | CATTATACCCAGAAGTCTCTCTCGCTGTCACCCGGCAAATAG |
| | | CCTAGGAATAAAAGATCTTTATTTTCATTAGATCTGTGTGTTG |
| | | GTTTTTTGTGTGCTCGAGAGGAACCCCTAGTGATGGAGTTGG |
| | | CCACTCCCTCTCTGCGCGCTCGCTCGCTCACTGAGGCCGGGC |
| | | GACCAAAGGTCGCCCGACGCCCGGGCTTTGCCCGGGCGGCCT |
| | | CAGTGAGCGAGCGAGCGCGCAGCTGCAGATCTG |
| (SEQ ID | EC #4 | CTGCGCGCTCGCTCGCTCACTGAGGCCGCCCGGGCAAAGCCC |
| NO: 71) | H- | GGGCGTCGGGCGACCTTTGGTCGCCCGGCCTCAGTGAGCGAG |
| Full: 4479 | F2A-L | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-10 signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1951 | | TTTGTATTTATTTATTTTTTAATTATTTTGTGCAGCGATGGGGG |
| HC: | | CGGGGGGGGGGGGGGCGCGCGCCAGGCGGGGCGGGGGGGG |
| 1952-3295 | | GCGAGGGGCGGGGCGGGGCGAGGCGGAGAGGTGCGGCGGC |
| Furin: | | AGCCAATCAGAGCGGCGCGCTCCGAAAGTTTCCTTTTATGGC |
| 3296-3307 | | GAGGCGGCGGCGGCGGCGGCCCTATAAAAAGCGAAGCGCGC |
| Linker: | | GGCGGGCGGGAGTCGCTGCGCGCTGCCTTCGCCCCGTGCCCC |
| 3308-3319 | | GCTCCGCCGCCGCCTCGCGCCGCCCGCCCCGGCTCTGACTGA |
| F2A: | | CCGCGTTACTCCCACAGGTGAGCGGGCGGGACGGCCCTTCTC |
| 3320-3391 | | CTCCGGGCTGTAATTAGCGCTTGGTTTAATGACGGCTCGTTTC |
| IL-2 signal | | TTTTCTGTGGCTGCGTGAAAGCCTTGAGGGGCTCCGGGAGGG |
| peptide: | | CCCTTTGTGCGGGGGGAGCGGCTCGGGGGGTGCGTGCGTGTG |
| 3392-3451 | | TGTGTGCGTGGGGAGCGCCGCGTGCGGCTCCGCGCTGCCCGG |
| LC: | | CGGCTGTGAGCGCTGCGGGCGCGGCGCGGGGCTTTGTGCGCT |
| 3452-4096 | | CCGCAGTGTGCGCGAGAGGAGCGCGGCCGGGGGCGGTGCCA |
| Stop Codon: | | CGCGGTGCGGGGGGGGCTGCGAGGGGAACAAAGGCTGCGTG |
| 4097-4099 | | CGGGGTGTGTGCGTGGGGGGGGTGAGCAGGGGGTGTGGGCG |
| bGH | | CGGCGGTCGGGCTGTAACCCCCCCCTGCACCCCCCTCCCCGA |
| poly(A): | | GTTGCTGAGCACGGCCCGGCTTCGGGTGCGGGGCTCCGTGCG |
| 4106-4329 | | GGGCGTGGCGCGGGGCTCGCCGTGCCGGGCGGGGGGTGGCG |
| ITR: | | GCGGGTGGGGGTGCCGGGCGGGGCGGGGCCGCCTCGGGCCG |
| 4339-4479 | | GGGAGGGCTCGGGGGAGGGGCGCGGCGGCCCCCGGAGCGCC |
| | | GGCGGCTGTCGAGGCGCGGCGAGCCGCAGCCATTGCCTTTTA |
| | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGCACAGCTCCGCCCTGCTCTGCTGTTTGG |
| | | TGCTTCTGACAGGAGTCAGGGCTCAGGTTGAGTTAGTGGAAT |
| | | CTGGAGGGGGTGTAGTGCAACCTGGCAGATCACAGAGGCTG |
| | | AGTTGCGCAGCAAGTGGATTCACATTTAGCTCCTATGGGATG |
| | | CATTGGGTCCGCCAGGCCCCTGGCAAGGGTCTAGAGTGGGTG |
| | | GCTATCATTTGGTTTGATGGATCTTCAACTTATTATGCTGATA |
| | | GTGTTAGAGGGCGCTTTACTATAAGCAGGGACAACTCCAAAA |
| | | ATACCCTCTATCTGCAAATGAACTCTTTGAGAGCAGAAGATA |
| | | CAGCTGTGTACTTCTGTGCTAGGGAGCTGGGCCGCAGATATT |
| | | TTGACCTTTGGGGCAGAGGAACTCTTGTCTCTGTATCCTCAGC |
| | | AAGTACCAAGGGGCCATCTGTGTTCCCGCTGGCCCCCTCTTCC |
| | | AAAAGTACATCAGGTGGCACCGCGGCTCTGGGATGCTTGGTT |
| | | AAGGATTACTTTCCTGAACCAGTGACTGTCAGCTGGAATAGT |
| | | GGGGCCTTGACTTCTGGGGTGCACACATTCCCCGCAGTCCTTC |
| | | AGTCCAGCGGTCTGTATTCATTATCCAGTGTGGTTACAGTGCC |
| | | TTCTAGCTCCCTGGGAACTCAGACCTACATCTGTAATGTAAAT |
| | | CATAAACCAAGCAACACAAAGGTGGACAAGAAAGTTGAGCC |
| | | CAAGTCTTGTGATAAAACCCACACTTGTCCGCCTTGCCCAGC |
| | | CCCTGAGCTACTTGGGGGCCCTTCAGTTTTTCTGTTCCCACCC |
| | | AAGCCTAAAGACACATTGATGATTAGTAGGACTCCTGAAGTG |
| | | ACCTGTGTTGTGGTGGATGTTTCCCATGAGGACCCAGAAGTA |
| | | AAGTTCAATTGGTATGTGGATGGAGTTGAGGTGCACAATGCT |
| | | AAGACTAAACCGAGGGAAGAGCAGTATAATAGCACATACAG |
| | | AGTTGTGTCAGTTCTGACAGTGCTTCACCAAGACTGGCTCAA |
| | | TGGAAAGGAGTATAAATGCAAGGTATCTAATAAGGCCCTGCC |
| | | CGCACCTATTGAAAAAACTATTTCAAAGGCTAAAGGGCAGCC |
| | | AAGGGAGCCCCAGGTCTACACCCTGCCTCCATCCAGAGATGA |
| | | ATTAACAAAGAACCAGGTGAGTCTCACTTGCCTGGTTAAAGG |
| | | TTTTTATCCCTCTGATATTGCTGTGGAGTGGGAGTCTAATGGC |
| | | CAACCAGAAAATAACTACAAGACCACACCTCCAGTCTTGGAC |
| | | TCTGATGGAAGCTTCTTTCTTTACTCAAAGCTGACTGTGGACA |
| | | AAAGTAGATGGCAGCAGGGGAATGTGTTCAGCTGTTCTGTTA |
| | | TGCATGAGGCGCTACACAACCATTATACCCAGAAGTCTCTCA |
| | | GTCTGTCACCTGGCAAAAGAAAGAGAAGGAGTGGCTCAGGA |
| | | GCCCCTGTGAAACAGACCCTGAACTTTGACCTCTTGAAGCTT |
| | | GCTGGGGATGTGGAGTCTAATCCTGGTCCAATGTACAGAATG |
| | | CAGCTGCTCAGCTGCATTGCCTTGTCCCTTGCTCTGGTGACCA |
| | | ACTCTGAGATTGTCTTAACACAATCACCCGCAACTCTGAGTCT |
| | | AAGTCCTGGAGAAAGAGCAACTCTCAGCTGTAGGGCCTCCCA |
| | | GTCTGTTTCAAGTTATCTGGCTTGGTACCAGCAAAAGCCAGG |
| | | ACAGGCCCCGCGCTTGCTGATATATGATGCAAGCAAAAGGGC |
| | | CACAGGGATCCCCGCTAGATTCAGCGGTTCTGGCAGCGGAAC |
| | | AGATTTTACTCTTACCATTTCCTCACTTGAGCCTGAAGACTTT |
| | | GCAGTGTACTATTGCCAGCAGCGGAGTAAGTGGCCACCCTGG |
| | | ACATTTGGGCAAGGCACCAAAGTAGAGAGCAAGAGAACTGT |
| | | GGCCGCGCCTTCTGTCTTCATCTTTCCACCCTCTGATGAACAG |
| | | CTGAAATCAGGTACTGCTTCAGTGGTTTGTCTGCTCAATAACT |
| | | TCTACCCAAGGGAGGCCAAGGTGCAGTGGAAGGTTGACAAT |
| | | GCATTGCAGAGCGGAAACAGTCAAGAGTCTGTAACAGAACA |
| | | GGATTCCAAAGACAGCACCTATTCACTTTCCAGTACTCTGAC |
| | | CTTATCTAAGGCTGATTATGAGAAACACAAGGTGTATGCTTG |
| | | TGAAGTTACACATCAGGGGCTGAGCTCCCCTGTGACCAAAAG |
| | | CTTTAATAGGGGGGAGTGTTAGCAATTGCTGTGCCTTCTAGTT |
| | | GCCAGCCATCTGTTGTTTGCCCCTCCCCCGTGCCTTCCTTGAC |
| | | CCTGGAAGGTGCCACTCCCACTGTCCTTTCCTAATAAAATGA |
| | | GGAAATTGCATCGCATTGTCTGAGTAGGTGTCATTCTATTCTG |
| | | GGGGGTGGGGTGGGGCAGGACAGCAAGGGGGAGGATTGGGA |
| | | AGACAATAGCAGGCATGCTGGGGATGCGGTGGGCTCTATGG |
| | | AGCTCGAGAGGAACCCCTAGTGATGGAGTTGGCCACTCCCTC |
| | | TCTGCGCGCTCGCTCGCTCACTGAGGCCGGGCGACCAAAGGT |
| | | CGCCCGACGCCCGGGCTTTGCCCGGGCGGCCTCAGTGAGCGA |
| | | GCGAGCGCGCAGCTGCAGATCTG |
| (SEQ ID | EC #5 | CTGCGCGCTCGCTCGCTCACTGAGGCCGCCCGGGCAAAGCCC |
| NO: 72) | L- | GGGCGTCGGGCGACCTTTGGTCGCCCGGCCTCAGTGAGCGAG |
| Full: 4795 | IRES-H | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-2 signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1957 | | TTTGTATTTATTTATTTTTTAATTATTTTGTGCAGCGATGGGGG |
| LC: | | CGGGGGGGGGGGGGGCGCGCGCCAGGCGGGGCGGGGGGGG |
| 1958-2602 | | GCGAGGGGGGGGCGGGGCGAGGCGGAGAGGTGCGGCGGC |
| Stop Codon: | | AGCCAATCAGAGCGGCGCGCTCCGAAAGTTTCCTTTTATGGC |
| 2602-2605 | | GAGGCGGCGGCGGCGGCGGCCCTATAAAAAGCGAAGCGCGC |
| IRES2: | | GGCGGGCGGGAGTCGCTGCGCGCTGCCTTCGCCCCGTGCCCC |
| 2606-3190 | | GCTCCGCCGCCGCCTCGCGCCGCCCGCCCCGGCTCTGACTGA |
| IL-10 signal | | CCGCGTTACTCCCACAGGTGAGCGGGCGGGACGGCCCTTCTC |
| peptide: | | CTCCGGGCTGTAATTAGCGCTTGGTTTAATGACGGCTCGTTTC |
| 3191-3245 | | TTTTCTGTGGCTGCGTGAAAGCCTTGAGGGGCTCCGGGAGGG |
| HC: | | CCCTTTGTGCGGGGGGAGCGGCTCGGGGGGTGCGTGCGTGTG |
| 3191-4594 | | TGTGTGCGTGGGGAGCGCCGCGTGCGGCTCCGCGCTGCCCGG |
| Stop Codon: | | CGGCTGTGAGCGCTGCGGGCGCGGCGCGGGGCTTTGTGCGCT |
| 4595-4597 | | CCGCAGTGTGCGCGAGAGGAGCGCGGCCGGGGGCGGTGCCA |
| Synthetic | | CGCGGTGCGGGGGGGGCTGCGAGGGGAACAAAGGCTGCGTG |
| poly(A): | | CGGGGTGTGTGCGTGGGGGGGGTGAGCAGGGGGTGTGGGCG |
| 4597-4645 | | CGGCGGTCGGGCTGTAACCCCCCCCTGCACCCCCCTCCCCGA |
| ITR: | | GTTGCTGAGCACGGCCCGGCTTCGGGTGCGGGGCTCCGTGCG |
| 4655-4795 | | GGGCGTGGCGCGGGGCTCGCCGTGCCGGGCGGGGGGGGCG |
| | | GCGGGTGGGGGTGCCGGGCGGGGCGGGGCCGCCTCGGGCCG |
| | | GGGAGGGCTCGGGGGAGGGGCGCGGCGGCCCCCGGAGCGCC |
| | | GGCGGCTGTCGAGGCGCGGCGAGCCGCAGCCATTGCCTTTTA |
| | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGTACAGAATGCAGCTGCTCAGCTGCATT |
| | | GCCTTGTCCCTTGCTCTGGTGACCAACTCTGAGATTGTCTTAA |
| | | CACAATCACCCGCAACTCTGAGTCTAAGTCCTGGAGAAAGAG |
| | | CAACTCTCAGCTGTAGGGCCTCCCAGTCTGTTTCAAGTTATCT |
| | | GGCTTGGTACCAGCAAAAGCCAGGACAGGCCCCGCGCTTGCT |
| | | GATATATGATGCAAGCAAAAGGGCCACAGGGATCCCCGCTA |
| | | GATTCAGCGGTTCTGGCAGCGGAACAGATTTTACTCTTACCA |
| | | TTTCCTCACTTGAGCCTGAAGACTTTGCAGTGTACTATTGCCA |
| | | GCAGCGGAGTAAGTGGCCACCCTGGACATTTGGGCAAGGCA |
| | | CCAAAGTAGAGAGCAAGAGAACTGTGGCCGCGCCTTCTGTCT |
| | | TCATCTTTCCACCCTCTGATGAACAGCTGAAATCAGGTACTGC |
| | | TTCAGTGGTTTGTCTGCTCAATAACTTCTACCCAAGGGAGGCC |
| | | AAGGTGCAGTGGAAGGTTGACAATGCATTGCAGAGCGGAAA |
| | | CAGTCAAGAGTCTGTAACAGAACAGGATTCCAAAGACAGCA |
| | | CCTATTCACTTTCCAGTACTCTGACCTTATCTAAGGCTGATTA |
| | | TGAGAAACACAAGGTGTATGCTTGTGAAGTTACACATCAGGG |
| | | GCTGAGCTCCCCTGTGACCAAAAGCTTTAATAGGGGGGAGTG |
| | | TTAGGCCCCTCTCCCTCCCCCCCCCCTAACGTTACTGGCCGAA |
| | | GCCGCTTGGAATAAGGCCGGTGTGCGTTTGTCTATATGTTATT |
| | | TTCCACCATATTGCCGTCTTTTGGCAATGTGAGGGCCCGGAA |
| | | ACCTGGCCCTGTCTTCTTGACGAGCATTCCTAGGGGTCTTTCC |
| | | CCTCTCGCCAAAGGAATGCAAGGTCTGTTGAATGTCGTGAAG |
| | | GAAGCAGTTCCTCTGGAAGCTTCTTGAAGACAAACAACGTCT |
| | | GTAGCGACCCTTTGCAGGCAGCGGAACCCCCCACCTGGCGAC |
| | | AGGTGCCTCTGCGGCCAAAAGCCACGTGTATAAGATACACCT |
| | | GCAAAGGCGGCACAACCCCAGTGCCACGTTGTGAGTTGGATA |
| | | GTTGTGGAAAGAGTCAAATGGCTCTCCTCAAGCGTATTCAAC |
| | | AAGGGGCTGAAGGATGCCCAGAAGGTACCCCATTGTATGGG |
| | | ATCTGATCTGGGGCCTCGGTACACATGCTTTACATGTGTTTAG |
| | | TCGAGGTTAAAAAAACGTCTAGGCCCCCCGAACCACGGGGA |
| | | CGTGGTTTTCCTTTGAAAAACACGATGATAATATGGCCACAA |
| | | TGCACAGCTCCGCCCTGCTCTGCTGTTTGGTGCTTCTGACAGG |
| | | AGTCAGGGCTCAGGTTGAGTTAGTGGAATCTGGAGGGGGTGT |
| | | AGTGCAACCTGGCAGATCACAGAGGCTGAGTTGCGCAGCAA |
| | | GTGGATTCACATTTAGCTCCTATGGGATGCATTGGGTCCGCC |
| | | AGGCCCCTGGCAAGGGTCTAGAGTGGGTGGCTATCATTTGGT |
| | | TTGATGGATCTTCAACTTATTATGCTGATAGTGTTAGAGGGCG |
| | | CTTTACTATAAGCAGGGACAACTCCAAAAATACCCTCTATCT |
| | | GCAAATGAACTCTTTGAGAGCAGAAGATACAGCTGTGTACTT |
| | | CTGTGCTAGGGAGCTGGGCCGCAGATATTTTGACCTTTGGGG |
| | | CAGAGGAACTCTTGTCTCTGTATCCTCAGCAAGTACCAAGGG |
| | | GCCATCTGTGTTCCCGCTGGCCCCCTCTTCCAAAAGTACATCA |
| | | GGTGGCACCGCGGCTCTGGGATGCTTGGTTAAGGATTACTTT |
| | | CCTGAACCAGTGACTGTCAGCTGGAATAGTGGGGCCTTGACT |
| | | TCTGGGGTGCACACATTCCCCGCAGTCCTTCAGTCCAGCGGT |
| | | CTGTATTCATTATCCAGTGTGGTTACAGTGCCTTCTAGCTCCC |
| | | TGGGAACTCAGACCTACATCTGTAATGTAAATCATAAACCAA |
| | | GCAACACAAAGGTGGACAAGAAAGTTGAGCCCAAGTCTTGT |
| | | GATAAAACCCACACTTGTCCGCCTTGCCCAGCCCCTGAGCTA |
| | | CTTGGGGGCCCTTCAGTTTTTCTGTTCCCACCCAAGCCTAAAG |
| | | ACACATTGATGATTAGTAGGACTCCTGAAGTGACCTGTGTTG |
| | | TGGTGGATGTTTCCCATGAGGACCCAGAAGTAAAGTTCAATT |
| | | GGTATGTGGATGGAGTTGAGGTGCACAATGCTAAGACTAAAC |
| | | CGAGGGAAGAGCAGTATAATAGCACATACAGAGTTGTGTCA |
| | | GTTCTGACAGTGCTTCACCAAGACTGGCTCAATGGAAAGGAG |
| | | TATAAATGCAAGGTATCTAATAAGGCCCTGCCCGCACCTATT |
| | | GAAAAAACTATTTCAAAGGCTAAAGGGCAGCCAAGGGAGCC |
| | | CCAGGTCTACACCCTGCCTCCATCCAGAGATGAATTAACAAA |
| | | GAACCAGGTGAGTCTCACTTGCCTGGTTAAAGGTTTTTATCCC |
| | | TCTGATATTGCTGTGGAGTGGGAGTCTAATGGCCAACCAGAA |
| | | AATAACTACAAGACCACACCTCCAGTCTTGGACTCTGATGGA |
| | | AGCTTCTTTCTTTACTCAAAGCTGACTGTGGACAAAAGTAGA |
| | | TGGCAGCAGGGGAATGTGTTCAGCTGTTCTGTTATGCATGAG |
| | | GCGCTACACAACCATTATACCCAGAAGTCTCTCAGTCTGTCA |
| | | CCGGCAAATAGCAATTGAATAAAAGATCTTTATTTTCATTAG |
| | | ATCTGTGTGTTGGTTTTTTGTGTGGAGCTCGAGAGGAACCCCT |
| | | AGTGATGGAGTTGGCCACTCCCTCTCTGCGCGCTCGCTCGCTC |
| | | ACTGAGGCCGGGCGACCAAAGGTCGCCCGACGCCCGGGCTTT |
| | | GCCCGGGCGGCCTCAGTGAGCGAGCGAGCGCGCAGCTGCAG |
| | | ATCTG |
| (SEQ ID | EC #6 | CTGCGCGCTCGCTCGCTCACTGAGGCCGCCCGGGCAAAGCCC |
| NO: 73) | Dual | GGGCGTCGGGCGACCTTTGGTCGCCCGGCCTCAGTGAGCGAG |
| Full: 4288 | | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CMV | | TGACGTATGTTCCCATAGTAACGCCAATAGGGACTTTCCATT |
| promoter: | | GACGTCAATGGGTGGACTATTTACGGTAAACTGCCCACTTGG |
| 529-732 | | CAGTACATCAAGTGTATCATATGCCAAGTACGCCCCCTATTG |
| SV40 intron: | | ACGTCAATGACGGTAAATGGCCCGCCTGGCATTATGCCCAGT |
| 791-887 | | ACATGACCTTATGGGACTTTCCTACTTGGCAGTACATCTACGT |
| IL-2 signal | | ATTAGTCATCGCTATTACCATGGTGATGCGGTTTTGGCAGTAC |
| peptide: | | ATCAATGGGCGTGGATAGCGGTTTGACTCACGGGGATTTCCA |
| 954-1013 | | AGTCTCCACCCCATTGACGTCAATGGGAGTTTGTTTTGGCACC |
| LC: | | AAAATCAACGGGACTTTCCAAAATGTCGTAACAACTCCGCCC |
| 1014-1658 | | CATTGACGCAAATGGGCGGTAGGCGTGTACGGTGGGAGGTCT |
| Stop Codon: | | ATATAAGCAGAGCTCGTTTAGTGAACCGTCAGATCGCCTGGA |
| 1659-1661 | | GAAGGAACTGAAAAACCAGAAAGTTAACTGGTAAGTTTAGT |
| bGH | | CTTTTTGTCTTTTATTTCAGGTCCCGGATCCGGTGGTGGTGCA |
| poly(A): | | AATCAAAGAACTGCTCCTCAGTGGATGTTGCCTTTACTTCTAG |
| 1668-1842 | | GCCTGTACGGAAGTGTTACTTCTGCTCTAAAAGCTGCGGAAT |
| pause site: | | TGTAGCCCGGTTAATTAAGCCACCATGTACAGAATGCAGCTG |
| 1857-1948 | | CTCAGCTGCATTGCCTTGTCCCTTGCTCTGGTGACCAACTCTG |
| EF-1α | | AGATTGTCTTAACACAATCACCCGCAACTCTGAGTCTAAGTC |
| Promoter: | | CTGGAGAAAGAGCAACTCTCAGCTGTAGGGCCTCCCAGTCTG |
| 1967-2196 | | TTTCAAGTTATCTGGCTTGGTACCAGCAAAAGCCAGGACAGG |
| 5′ LTR | | CCCCGCGCTTGCTGATATATGATGCAAGCAAAAGGGCCACAG |
| (truncated): | | GGATCCCCGCTAGATTCAGCGGTTCTGGCAGCGGAACAGATT |
| 2209-2477 | | TTACTCTTACCATTTCCTCACTTGAGCCTGAAGACTTTGCAGT |
| HGB/IgHC | | GTACTATTGCCAGCAGCGGAGTAAGTGGCCACCCTGGACATT |
| chimeric | | TGGGCAAGGCACCAAAGTAGAGAGCAAGAGAACTGTGGCCG |
| intron: | | CGCCTTCTGTCTTCATCTTTCCACCCTCTGATGAACAGCTGAA |
| 2504-2636 | | ATCAGGTACTGCTTCAGTGGTTTGTCTGCTCAATAACTICTAC |
| IL-10 signal | | CCAAGGGAGGCCAAGGTGCAGTGGAAGGTTGACAATGCATT |
| peptide: | | GCAGAGCGGAAACAGTCAAGAGTCTGTAACAGAACAGGATT |
| 2686-2739 | | CCAAAGACAGCACCTATTCACTTTCCAGTACTCTGACCTTATC |
| HC: | | TAAGGCTGATTATGAGAAACACAAGGTGTATGCTTGTGAAGT |
| 2740-4083 | | TACACATCAGGGGCTGAGCTCCCCTGTGACCAAAAGCTTTAA |
| Stop Codon: | | TAGGGGGGAGTGTTAGGCATGCCTGTGCCTTCTAGTTGCCAG |
| 4084-4086 | | CCATCTGTTGTTTGCCCCTCCCCCGTGCCTTCCTTGACCCTGG |
| Synthetic | | AAGGTGCCACTCCCACTGTCCTTTCCTAATAAAATGAGGAAA |
| poly(A): | | TTGCATCGCATTGTCTGAGTAGGTGTCATTCTATTCTGGGGGG |
| 4093-4141 | | TGGGGGGGGCAGGACAGCAAGGGGGAGGATTCTCAGAACT |
| ITR: | | AACATACGCTCTCCATCAAAACAAAACGAAACAAAACAAAC |
| 4148-4288 | | TAGCAAAATAGGCTGTCCCCAGTGCAAGTGCAGGTGCCAGAA |
| | | CATTTCTCTATCGAAGGATCTGCGATCGCTCCGGTGCCCGTCA |
| | | GTGGGCAGAGCGCACATCGCCCACAGTCCCCGAGAAGTTGG |
| | | GGGGAGGGGTCGGCAATTGAACCGGTGCCTAGAGAAGGTGG |
| | | CGCGGGGTAAACTGGGAAAGTGATGTCGTGTACTGGCTCCGC |
| | | CTTTTTCCCGAGGGTGGGGGAGAACCGTATATAAGTGCAGTA |
| | | GTCGCCGTGAACGTTCTTTTTCGCAACGGGTTTGCCGCCAGA |
| | | ACACAGCTGAAGCTTCGAGGGGCTCGCATCTCTCCTTCACGC |
| | | GCCCGCCGCCCTACCTGAGGCCGCCATCCACGCCGGTTGAGT |
| | | CGCGTTCTGCCGCCTCCCGCCTGTGGTGCCTCCTGAACTGCGT |
| | | CCGCCGTCTAGGTAAGTTTAAAGCTCAGGTCGAGACCGGGCC |
| | | TTTGTCCGGCGCTCCCTTGGAGCCTACCTAGACTCAGCCGGCT |
| | | CTCCACGCTTTGCCTGACCCTGCTTGCTCAACTCTACGTCTTT |
| | | GTTTCGTTTTCTGTTCTGCGCCGTTACAGATCGAAGTTGGTCG |
| | | TGAGGCACTGGGCAGGTAAGTATCAAGGTTACAAGACAGGTT |
| | | TAAGGAGACCAATAGAAACTGGGCTTGTCGAGACAGAGAAG |
| | | ACTCTTGCGTTTCTGATAGGCACCTATTGGTCTTACTGACATC |
| | | CACTTTGCCTTTCTCTCCACAGGTGTCCACTCCCAGTTCAATT |
| | | ACAGCTCTTAAGGCTAGTTTAAACCACCATGCACAGCTCCGC |
| | | CCTGCTCTGCTGTTTGGTGCTTCTGACAGGAGTCAGGGCTCAG |
| | | GTTGAGTTAGTGGAATCTGGAGGGGGTGTAGTGCAACCTGGC |
| | | AGATCACAGAGGCTGAGTTGCGCAGCAAGTGGATTCACATTT |
| | | AGCTCCTATGGGATGCATTGGGTCCGCCAGGCCCCTGGCAAG |
| | | GGTCTAGAGTGGGTGGCTATCATTTGGTTTGATGGATCTTCAA |
| | | CTTATTATGCTGATAGTGTTAGAGGGCGCTTTACTATAAGCA |
| | | GGGACAACTCCAAAAATACCCTCTATCTGCAAATGAACTCTT |
| | | TGAGAGCAGAAGATACAGCTGTGTACTTCTGTGCTAGGGAGC |
| | | TGGGCCGCAGATATTTTGACCTTTGGGGCAGAGGAACTCTTG |
| | | TCTCTGTATCCTCAGCAAGTACCAAGGGGCCATCTGTGTTCCC |
| | | GCTGGCCCCCTCTTCCAAAAGTACATCAGGTGGCACCGCGGC |
| | | TCTGGGATGCTTGGTTAAGGATTACTTTCCTGAACCAGTGACT |
| | | GTCAGCTGGAATAGTGGGGCCTTGACTTCTGGGGTGCACACA |
| | | TTCCCCGCAGTCCTTCAGTCCAGCGGTCTGTATTCATTATCCA |
| | | GTGTGGTTACAGTGCCTTCTAGCTCCCTGGGAACTCAGACCT |
| | | ACATCTGTAATGTAAATCATAAACCAAGCAACACAAAGGTGG |
| | | ACAAGAAAGTTGAGCCCAAGTCTTGTGATAAAACCCACACTT |
| | | GTCCGCCTTGCCCAGCCCCTGAGCTACTTGGGGGCCCTTCAGT |
| | | TTTTCTGTTCCCACCCAAGCCTAAAGACACATTGATGATTAGT |
| | | AGGACTCCTGAAGTGACCTGTGTTGTGGTGGATGTTTCCCAT |
| | | GAGGACCCAGAAGTAAAGTTCAATTGGTATGTGGATGGAGTT |
| | | GAGGTGCACAATGCTAAGACTAAACCGAGGGAAGAGCAGTA |
| | | TAATAGCACATACAGAGTTGTGTCAGTTCTGACAGTGCTTCA |
| | | CCAAGACTGGCTCAATGGAAAGGAGTATAAATGCAAGGTAT |
| | | CTAATAAGGCCCTGCCCGCACCTATTGAAAAAACTATTTCAA |
| | | AGGCTAAAGGGCAGCCAAGGGAGCCCCAGGTCTACACCCTG |
| | | CCTCCATCCAGAGATGAATTAACAAAGAACCAGGTGAGTCTC |
| | | ACTTGCCTGGTTAAAGGTTTTTATCCCTCTGATATTGCTGTGG |
| | | AGTGGGAGTCTAATGGCCAACCAGAAAATAACTACAAGACC |
| | | ACACCTCCAGTCTTGGACTCTGATGGAAGCTTCTTTCTTTACT |
| | | CAAAGCTGACTGTGGACAAAAGTAGATGGCAGCAGGGGAAT |
| | | GTGTTCAGCTGTTCTGTTATGCATGAGGCGCTACACAACCATT |
| | | ATACCCAGAAGTCTCTCAGTCTGTCACCTGGCAAATAGCCTA |
| | | GGAATAAAAGATCTTTATTTTCATTAGATCTGTGTGTTGGTTT |
| | | TTTGTGTGCTCGAGAGGAACCCCTAGTGATGGAGTTGGCCAC |
| | | TCCCTCTCTGCGCGCTCGCTCGCTCACTGAGGCCGGGCGACC |
| | | AAAGGTCGCCCGACGCCCGGGCTTTGCCCGGGCGGCCTCAGT |
| | | GAGCGAGCGAGCGCGCAGCTGCAGATCTG |
| (SEQ ID | EC #7 | CTGCGCGCTCGCTCGCTCACTGAGGCCGCCCGGGCAAAGCCC |
| NO: 74) | H- | GGGCGTCGGGCGACCTTTGGTCGCCCGGCCTCAGTGAGCGAG |
| Full: 4488 | F2A-L | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-10 signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1951 | | TTTGTATTTATTTATTTTTTAATTATTTTGTGCAGCGATGGGGG |
| HC: | | CGGGGGGGGGGGGGGCGCGCGCCAGGCGGGGCGGGGGGGG |
| 1952-3295 | | GCGAGGGGCGGGGCGGGGCGAGGCGGAGAGGTGCGGCGGC |
| Stop Codon: | | AGCCAATCAGAGCGGCGCGCTCCGAAAGTTTCCTTTTATGGC |
| 3296-3298 | | GAGGCGGCGGCGGCGGCGGCCCTATAAAAAGCGAAGCGCGC |
| Furin: | | GGCGGGCGGGAGTCGCTGCGCGCTGCCTTCGCCCCGTGCCCC |
| 3305-3316 | | GCTCCGCCGCCGCCTCGCGCCGCCCGCCCCGGCTCTGACTGA |
| Linker: | | CCGCGTTACTCCCACAGGTGAGCGGGCGGGACGGCCCTTCTC |
| 3317-3328 | | CTCCGGGCTGTAATTAGCGCTTGGTTTAATGACGGCTCGTTTC |
| F2A: | | TTTTCTGTGGCTGCGTGAAAGCCTTGAGGGGCTCCGGGAGGG |
| 3329-3400 | | CCCTTTGTGCGGGGGGAGCGGCTCGGGGGGTGCGTGCGTGTG |
| IL-2 signal | | TGTGTGCGTGGGGAGCGCCGCGTGCGGCTCCGCGCTGCCCGG |
| peptide: | | CGGCTGTGAGCGCTGCGGGCGCGGCGCGGGGCTTTGTGCGCT |
| 3401-3460 | | CCGCAGTGTGCGCGAGAGGAGCGCGGCCGGGGGCGGTGCCA |
| LC: | | CGCGGTGCGGGGGGGGCTGCGAGGGGAACAAAGGCTGCGTG |
| 3461-4105 | | CGGGGTGTGTGCGTGGGGGGGGTGAGCAGGGGGTGTGGGCG |
| Stop Codon: | | CGGCGGTCGGGCTGTAACCCCCCCCTGCACCCCCCTCCCCGA |
| 4106-4108 | | GTTGCTGAGCACGGCCCGGCTTCGGGTGCGGGGCTCCGTGCG |
| bGH | | GGGCGTGGCGCGGGGCTCGCCGTGCCGGGCGGGGGGTGGCG |
| poly(A): | | GCGGGTGGGGGTGCCGGGCGGGGCGGGGCCGCCTCGGGCCG |
| 4115-4338 | | GGGAGGGCTCGGGGGAGGGGCGCGGCGGCCCCCGGAGCGCC |
| ITR: | | GGCGGCTGTCGAGGCGCGGCGAGCCGCAGCCATTGCCTTTTA |
| 4348-4488 | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGCACAGCTCTGCCCTGCTCTGCTGTTTGG |
| | | TGCTTCTGACAGGAGTCAGGGCTCAGGTTGAGTTAGTGGAAT |
| | | CTGGAGGGGGTGTAGTGCAACCTGGCAGATCACAGAGGCTG |
| | | AGTTGTGCAGCAAGTGGATTCACATTTAGCTCCTATGGGATG |
| | | CATTGGGTCAGACAGGCCCCTGGCAAGGGTCTAGAGTGGGTG |
| | | GCTATCATTTGGTTTGATGGATCTTCAACTTATTATGCTGATA |
| | | GTGTTAGGGGGAGATTTACTATAAGCAGGGACAACTCCAAAA |
| | | ATACCCTCTATCTGCAAATGAACTCTTTGAGAGCAGAAGATA |
| | | CAGCTGTGTACTTCTGTGCTAGGGAGCTGGGCAGAAGGTATT |
| | | TTGACCTTTGGGGCAGAGGAACTCTTGTCTCTGTATCCTCAGC |
| | | AAGTACCAAGGGGCCATCTGTGTTCCCACTGGCCCCCTCTTCC |
| | | AAAAGTACATCAGGTGGCACAGCAGCTCTGGGATGCTTGGTT |
| | | AAGGATTACTTTCCTGAACCAGTGACTGTCAGCTGGAATAGT |
| | | GGGGCCTTGACTTCTGGGGTGCACACATTCCCTGCAGTCCTTC |
| | | AGTCCTCTGGTCTGTATTCATTATCCAGTGTGGTTACAGTGCC |
| | | TTCTAGCTCCCTGGGAACTCAGACCTACATCTGTAATGTAAAT |
| | | CATAAACCAAGCAACACAAAGGTGGACAAGAAAGTTGAGCC |
| | | CAAGTCTTGTGATAAAACCCACACTTGTCCACCTTGCCCAGC |
| | | CCCTGAGCTACTTGGGGGCCCTTCAGTTTTTCTGTTCCCACCC |
| | | AAGCCTAAAGACACATTGATGATTAGTAGGACTCCTGAAGTG |
| | | ACCTGTGTTGTGGTGGATGTTTCCCATGAGGACCCAGAAGTA |
| | | AAGTTCAATTGGTATGTGGATGGAGTTGAGGTGCACAATGCT |
| | | AAGACTAAACCAAGAGAAGAGCAGTATAATAGCACATACAG |
| | | GGTTGTGTCAGTTCTGACAGTGCTTCACCAAGACTGGCTCAA |
| | | TGGAAAGGAGTATAAATGCAAGGTATCTAATAAGGCCCTGCC |
| | | TGCACCTATTGAAAAAACTATTTCAAAGGCTAAAGGGCAGCC |
| | | AAGAGAGCCCCAGGTCTACACCCTGCCTCCATCCAGGGATGA |
| | | ATTAACAAAGAACCAGGTGAGTCTCACTTGCCTGGTTAAAGG |
| | | TTTTTATCCCTCTGATATTGCTGTGGAGTGGGAGTCTAATGGC |
| | | CAACCAGAAAATAACTACAAGACCACACCTCCAGTCTTGGAC |
| | | TCTGATGGAAGCTTCTTTCTTTACTCAAAGCTGACTGTGGACA |
| | | AAAGTAGATGGCAGCAGGGGAATGTGTTCAGCTGTTCTGTTA |
| | | TGCATGAGGCACTACACAACCATTATACCCAGAAGTCTCTCA |
| | | GTCTGTCACCTGGCAAATAGGGCAAAAGAAAGAGAAGGAGT |
| | | GGCTCAGGAGCCCCTGTGAAACAGACCCTGAACTTTGACCTC |
| | | TTGAAGCTTGCTGGGGATGTGGAGTCTAATCCTGGTCCAATG |
| | | TACAGAATGCAGCTGCTCAGCTGCATTGCCTTGTCCCTTGCTC |
| | | TGGTGACCAACTCTGAGATTGTCTTAACACAATCACCTGCAA |
| | | CTCTGAGTCTAAGTCCTGGAGAAAGAGCAACTCTCAGCTGTA |
| | | GGGCCTCCCAGTCTGTTTCAAGTTATCTGGCTTGGTACCAGCA |
| | | AAAGCCAGGACAGGCCCCAAGGTTGCTGATATATGATGCAA |
| | | GCAAAAGAGCCACAGGGATCCCTGCTAGGTTCTCTGGTTCTG |
| | | GCTCTGGAACAGATTTTACTCTTACCATTTCCTCACTTGAGCC |
| | | TGAAGACTTTGCAGTGTACTATTGCCAGCAGAGAAGTAAGTG |
| | | GCCACCCTGGACATTTGGGCAAGGCACCAAAGTAGAGAGCA |
| | | AGAGAACTGTGGCTGCACCTTCTGTCTTCATCTTTCCACCCTC |
| | | TGATGAACAGCTGAAATCAGGTACTGCTTCAGTGGTTTGTCT |
| | | GCTCAATAACTTCTACCCAAGGGAGGCCAAGGTGCAGTGGAA |
| | | GGTTGACAATGCATTGCAGTCTGGAAACAGTCAAGAGTCTGT |
| | | AACAGAACAGGATTCCAAAGACAGCACCTATTCACTTTCCAG |
| | | TACTCTGACCTTATCTAAGGCTGATTATGAGAAACACAAGGT |
| | | GTATGCTTGTGAAGTTACACATCAGGGGCTGAGCTCCCCTGT |
| | | GACCAAAAGCTTTAATAGGGGGGAGTGTTAGCAATTGCTGTG |
| | | CCTTCTAGTTGCCAGCCATCTGTTGTTTGCCCCTCCCCCGTGC |
| | | CTTCCTTGACCCTGGAAGGTGCCACTCCCACTGTCCTTTCCTA |
| | | ATAAAATGAGGAAATTGCATCGCATTGTCTGAGTAGGTGTCA |
| | | TTCTATTCTGGGGGGTGGGGTGGGGCAGGACAGCAAGGGGG |
| | | AGGATTGGGAAGACAATAGCAGGCATGCTGGGGATGCGGTG |
| | | GGCTCTATGGAGCTCGAGAGGAACCCCTAGTGATGGAGTTGG |
| | | CCACTCCCTCTCTGCGCGCTCGCTCGCTCACTGAGGCCGGGC |
| | | GACCAAAGGTCGCCCGACGCCCGGGCTTTGCCCGGGCGGCCT |
| | | CAGTGAGCGAGCGAGCGCGCAGCTGCAGATCTG |
| (SEQ ID | EC #8 | CTGCGCGCTCGCTCGCTCACTGAGGCCGCCCGGGCAAAGCCC |
| NO: 75) | LC- | GGGCGTCGGGCGACCTTTGGTCGCCCGGCCTCAGTGAGCGAG |
| Full: 4796 | IRES-HC | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CBA | | TGACGTATGTTCCCATAGTAACGCCAATAGGGACTTTCCATT |
| promoter: | | GACGTCAATGGGTGGACTATTTACGGTAAACTGCCCACTTGG |
| 531-806 | | CAGTACATCAAGTGTATCATATGCCAAGTACGCCCCCTATTG |
| CAG intron: | | ACGTCAATGACGGTAAATGGCCCGCCTGGCATTATGCCCAGT |
| 807-1870 | | ACATGACCTTATGGGACTTTCCTACTTGGCAGTACATCTACGT |
| IL-2signal | | ATTAGTCATCGCTATTACCATGGGTCGAGGTGAGCCCCACGT |
| peptide: | | TCTGCTTCACTCTCCCCATCTCCCCCCCCTCCCCACCCCCAAT |
| 1898-1957 | | TTTGTATTTATTTATTTTTTAATTATTTTGTGCAGCGATGGGGG |
| LC: | | CGGGGGGGGGGGGGGCGCGCGCCAGGCGGGGGGGGGGGG |
| 1958-2602 | | GCGAGGGGCGGGGCGGGGCGAGGCGGAGAGGTGCGGCGGC |
| Stop Codon: | | AGCCAATCAGAGCGGCGCGCTCCGAAAGTTTCCTTTTATGGC |
| 2603-2605 | | GAGGCGGCGGCGGCGGCGGCCCTATAAAAAGCGAAGCGCGC |
| IRES2: | | GGCGGGCGGGAGTCGCTGCGCGCTGCCTTCGCCCCGTGCCCC |
| 2606-3190 | | GCTCCGCCGCCGCCTCGCGCCGCCCGCCCCGGCTCTGACTGA |
| IL-10 signal | | CCGCGTTACTCCCACAGGTGAGCGGGCGGGACGGCCCTTCTC |
| peptide: | | CTCCGGGCTGTAATTAGCGCTTGGTTTAATGACGGCTCGTTTC |
| 3191-3244 | | TTTTCTGTGGCTGCGTGAAAGCCTTGAGGGGCTCCGGGAGGG |
| HC: | | CCCTTTGTGCGGGGGGAGCGGCTCGGGGGGTGCGTGCGTGTG |
| 3245-4588 | | TGTGTGCGTGGGGAGCGCCGCGTGCGGCTCCGCGCTGCCCGG |
| Stop Codon: | | CGGCTGTGAGCGCTGCGGGCGCGGCGCGGGGCTTTGTGCGCT |
| 4589-4591 | | CCGCAGTGTGCGCGAGAGGAGCGCGGCCGGGGGCGGTGCCA |
| Synthetic | | CGCGGTGCGGGGGGGGCTGCGAGGGGAACAAAGGCTGCGTG |
| poly(A): | | CGGGGTGTGTGCGTGGGGGGGGTGAGCAGGGGGTGTGGGCG |
| 4598-4646 | | CGGCGGTCGGGCTGTAACCCCCCCCTGCACCCCCCTCCCCGA |
| ITR: | | GTTGCTGAGCACGGCCCGGCTTCGGGTGCGGGGCTCCGTGCG |
| 4656-4796 | | GGGCGTGGCGCGGGGCTCGCCGTGCCGGGCGGGGGGTGGCG |
| | | GCGGGTGGGGGTGCCGGGCGGGGCGGGGCCGCCTCGGGCCG |
| | | GGGAGGGCTCGGGGGAGGGGCGCGGCGGCCCCCGGAGCGCC |
| | | GGCGGCTGTCGAGGCGCGGCGAGCCGCAGCCATTGCCTTTTA |
| | | TGGTAATCGTGCGAGAGGGCGCAGGGACTTCCTTTGTCCCAA |
| | | ATCTGTGCGGAGCCGAAATCTGGGAGGCGCCGCCGCACCCCC |
| | | TCTAGCGGGCGCGGGGCGAAGCGGTGCGGCGCCGGCAGGAA |
| | | GGAAATGGGCGGGGAGGGCCTTCGTGCGTCGCCGCGCCGCC |
| | | GTCCCCTTCTCCCTCTCCAGCCTCGGGGCTGTCCGCGGGGGG |
| | | ACGGCTGCCTTCGGGGGGGACGGGGCAGGGCGGGGTTCGGC |
| | | TTCTGGCGTGTGACCGGCGGTCTAGACTCTGCTAACCATGTTC |
| | | ATGCCTTCTTCTCTTTCCTACAGCTCCTGGGCAACGTGCTGGT |
| | | TGTTGTGCTGTCTCATCATTTTGGCAAAGAATTCAAGCTTCCA |
| | | GCTAGCGCCACCATGTACAGAATGCAGCTGCTCAGCTGCATT |
| | | GCCTTGTCCCTTGCTCTGGTGACCAACTCTGAGATTGTCTTAA |
| | | CACAATCACCTGCAACTCTGAGTCTAAGTCCTGGAGAAAGAG |
| | | CAACTCTCAGCTGTAGGGCCTCCCAGTCTGTTTCAAGTTATCT |
| | | GGCTTGGTACCAGCAAAAGCCAGGACAGGCCCCAAGGTTGCT |
| | | GATATATGATGCAAGCAAAAGAGCCACAGGGATCCCTGCTA |
| | | GGTTCTCTGGTTCTGGCTCTGGAACAGATTTTACTCTTACCAT |
| | | TTCCTCACTTGAGCCTGAAGACTTTGCAGTGTACTATTGCCAG |
| | | CAGAGAAGTAAGTGGCCACCCTGGACATTTGGGCAAGGCAC |
| | | CAAAGTAGAGAGCAAGAGAACTGTGGCTGCACCTTCTGTCTT |
| | | CATCTTTCCACCCTCTGATGAACAGCTGAAATCAGGTACTGCT |
| | | TCAGTGGTTTGTCTGCTCAATAACTTCTACCCAAGGGAGGCC |
| | | AAGGTGCAGTGGAAGGTTGACAATGCATTGCAGTCTGGAAAC |
| | | AGTCAAGAGTCTGTAACAGAACAGGATTCCAAAGACAGCAC |
| | | CTATTCACTTTCCAGTACTCTGACCTTATCTAAGGCTGATTAT |
| | | GAGAAACACAAGGTGTATGCTTGTGAAGTTACACATCAGGGG |
| | | CTGAGCTCCCCTGTGACCAAAAGCTTTAATAGGGGGGAGTGT |
| | | TAGGCCCCTCTCCCTCCCCCCCCCCTAACGTTACTGGCCGAAG |
| | | CCGCTTGGAATAAGGCCGGTGTGCGTTTGTCTATATGTTATTT |
| | | TCCACCATATTGCCGTCTTTTGGCAATGTGAGGGCCCGGAAA |
| | | CCTGGCCCTGTCTTCTTGACGAGCATTCCTAGGGGTCTTTCCC |
| | | CTCTCGCCAAAGGAATGCAAGGTCTGTTGAATGTCGTGAAGG |
| | | AAGCAGTTCCTCTGGAAGCTTCTTGAAGACAAACAACGTCTG |
| | | TAGCGACCCTTTGCAGGCAGCGGAACCCCCCACCTGGCGACA |
| | | GGTGCCTCTGCGGCCAAAAGCCACGTGTATAAGATACACCTG |
| | | CAAAGGCGGCACAACCCCAGTGCCACGTTGTGAGTTGGATAG |
| | | TTGTGGAAAGAGTCAAATGGCTCTCCTCAAGCGTATTCAACA |
| | | AGGGGCTGAAGGATGCCCAGAAGGTACCCCATTGTATGGGAT |
| | | CTGATCTGGGGCCTCGGTACACATGCTTTACATGTGTTTAGTC |
| | | GAGGTTAAAAAAACGTCTAGGCCCCCCGAACCACGGGGACG |
| | | TGGTTTTCCTTTGAAAAACACGATGATAATATGGCCACAATG |
| | | CACAGCTCTGCCCTGCTCTGCTGTTTGGTGCTTCTGACAGGAG |
| | | TCAGGGCTCAGGTTGAGTTAGTGGAATCTGGAGGGGGTGTAG |
| | | TGCAACCTGGCAGATCACAGAGGCTGAGTTGTGCAGCAAGTG |
| | | GATTCACATTTAGCTCCTATGGGATGCATTGGGTCAGACAGG |
| | | CCCCTGGCAAGGGTCTAGAGTGGGTGGCTATCATTTGGTTTG |
| | | ATGGATCTTCAACTTATTATGCTGATAGTGTTAGGGGGAGAT |
| | | TTACTATAAGCAGGGACAACTCCAAAAATACCCTCTATCTGC |
| | | AAATGAACTCTTTGAGAGCAGAAGATACAGCTGTGTACTTCT |
| | | GTGCTAGGGAGCTGGGCAGAAGGTATTTTGACCTTTGGGGCA |
| | | GAGGAACTCTTGTCTCTGTATCCTCAGCAAGTACCAAGGGGC |
| | | CATCTGTGTTCCCACTGGCCCCCTCTTCCAAAAGTACATCAGG |
| | | TGGCACAGCAGCTCTGGGATGCTTGGTTAAGGATTACTTTCCT |
| | | GAACCAGTGACTGTCAGCTGGAATAGTGGGGCCTTGACTTCT |
| | | GGGGTGCACACATTCCCTGCAGTCCTTCAGTCCTCTGGTCTGT |
| | | ATTCATTATCCAGTGTGGTTACAGTGCCTTCTAGCTCCCTGGG |
| | | AACTCAGACCTACATCTGTAATGTAAATCATAAACCAAGCAA |
| | | CACAAAGGTGGACAAGAAAGTTGAGCCCAAGTCTTGTGATA |
| | | AAACCCACACTTGTCCACCTTGCCCAGCCCCTGAGCTACTTG |
| | | GGGGCCCTTCAGTTTTTCTGTTCCCACCCAAGCCTAAAGACAC |
| | | ATTGATGATTAGTAGGACTCCTGAAGTGACCTGTGTTGTGGT |
| | | GGATGTTTCCCATGAGGACCCAGAAGTAAAGTTCAATTGGTA |
| | | TGTGGATGGAGTTGAGGTGCACAATGCTAAGACTAAACCAAG |
| | | AGAAGAGCAGTATAATAGCACATACAGGGTTGTGTCAGTTCT |
| | | GACAGTGCTTCACCAAGACTGGCTCAATGGAAAGGAGTATAA |
| | | ATGCAAGGTATCTAATAAGGCCCTGCCTGCACCTATTGAAAA |
| | | AACTATTTCAAAGGCTAAAGGGCAGCCAAGAGAGCCCCAGG |
| | | TCTACACCCTGCCTCCATCCAGGGATGAATTAACAAAGAACC |
| | | AGGTGAGTCTCACTTGCCTGGTTAAAGGTTTTTATCCCTCTGA |
| | | TATTGCTGTGGAGTGGGAGTCTAATGGCCAACCAGAAAATAA |
| | | CTACAAGACCACACCTCCAGTCTTGGACTCTGATGGAAGCTT |
| | | CTTTCTTTACTCAAAGCTGACTGTGGACAAAAGTAGATGGCA |
| | | GCAGGGGAATGTGTTCAGCTGTTCTGTTATGCATGAGGCACT |
| | | ACACAACCATTATACCCAGAAGTCTCTCAGTCTGTCACCTGG |
| | | CAAATAGCAATTGAATAAAAGATCTTTATTTTCATTAGATCTG |
| | | TGTGTTGGTTTTTTGTGTGGAGCTCGAGAGGAACCCCTAGTG |
| | | ATGGAGTTGGCCACTCCCTCTCTGCGCGCTCGCTCGCTCACTG |
| | | AGGCCGGGCGACCAAAGGTCGCCCGACGCCCGGGCTTTGCCC |
| | | GGGCGGCCTCAGTGAGCGAGCGAGCGCGCAGCTGCAGATCT |
| | | G |
| (SEQ ID | EC #9 | CTGCGCGCTCGCTCGCTCACTGAGGCCGCCCGGGCAAAGCCC |
| NO: 76) | Dual | GGGCGTCGGGCGACCTTTGGTCGCCCGGCCTCAGTGAGCGAG |
| Full: 4288 | | CGAGCGCGCAGAGAGGGAGTGGCCAACTCCATCACTAGGGG |
| ITR: 1-130 | | TTCCTTAGTATTAAACGCGTGTCGACATTGATTATTGACTAGT |
| CMV | | TATTAATAGTAATCAATTACGGGGTCATTAGTTCATAGCCCAT |
| enhancer: | | ATATGGAGTTCCGCGTTACATAACTTACGGTAAATGGCCCGC |
| 149-528 | | CTGGCTGACCGCCCAACGACCCCCGCCCATTGACGTCAATAA |
| CMV | | TGACGTATGTTCCCATAGTAACGCCAATAGGGACTTTCCATT |
| promoter: | | GACGTCAATGGGTGGACTATTTACGGTAAACTGCCCACTTGG |
| 529-732 | | CAGTACATCAAGTGTATCATATGCCAAGTACGCCCCCTATTG |
| SV40 intron: | | ACGTCAATGACGGTAAATGGCCCGCCTGGCATTATGCCCAGT |
| 791-887 | | ACATGACCTTATGGGACTTTCCTACTTGGCAGTACATCTACGT |
| IL-2 signal | | ATTAGTCATCGCTATTACCATGGTGATGCGGTTTTGGCAGTAC |
| peptide: | | ATCAATGGGCGTGGATAGCGGTTTGACTCACGGGGATTTCCA |
| 960-1013 | | AGTCTCCACCCCATTGACGTCAATGGGAGTTTGTTTTGGCACC |
| LC: | | AAAATCAACGGGACTTTCCAAAATGTCGTAACAACTCCGCCC |
| 1014-1658 | | CATTGACGCAAATGGGCGGTAGGCGTGTACGGTGGGAGGTCT |
| Stop Codon: | | ATATAAGCAGAGCTCGTTTAGTGAACCGTCAGATCGCCTGGA |
| 1659-1661 | | GAAGGAACTGAAAAACCAGAAAGTTAACTGGTAAGTTTAGT |
| bGH | | CTTTTTGTCTTTTATTTCAGGTCCCGGATCCGGTGGTGGTGCA |
| poly(A): | | AATCAAAGAACTGCTCCTCAGTGGATGTTGCCTTTACTTCTAG |
| 1668-1842 | | GCCTGTACGGAAGTGTTACTTCTGCTCTAAAAGCTGCGGAAT |
| pause site: | | TGTAGCCCGGTTAATTAAGCCACCATGTACAGAATGCAGCTG |
| 1857-1948 | | CTCAGCTGCATTGCCTTGTCCCTTGCTCTGGTGACCAACTCTG |
| EF-1α | | AGATTGTCTTAACACAATCACCTGCAACTCTGAGTCTAAGTC |
| Promoter: | | CTGGAGAAAGAGCAACTCTCAGCTGTAGGGCCTCCCAGTCTG |
| 1967-2196 | | TTTCAAGTTATCTGGCTTGGTACCAGCAAAAGCCAGGACAGG |
| 5′ LTR | | CCCCAAGGTTGCTGATATATGATGCAAGCAAAAGAGCCACAG |
| (truncated): | | GGATCCCTGCTAGGTTCTCTGGTTCTGGCTCTGGAACAGATTT |
| 2209-2477 | | TACTCTTACCATTTCCTCACTTGAGCCTGAAGACTTTGCAGTG |
| HBG/IgHC | | TACTATTGCCAGCAGAGAAGTAAGTGGCCACCCTGGACATTT |
| chimeric | | GGGCAAGGCACCAAAGTAGAGAGCAAGAGAACTGTGGCTGC |
| intron: | | ACCTTCTGTCTTCATCTTTCCACCCTCTGATGAACAGCTGAAA |
| 2504-2636 | | TCAGGTACTGCTTCAGTGGTTTGTCTGCTCAATAACTTCTACC |
| IL-10 signal | | CAAGGGAGGCCAAGGTGCAGTGGAAGGTTGACAATGCATTG |
| peptide: | | CAGTCTGGAAACAGTCAAGAGTCTGTAACAGAACAGGATTCC |
| 2686-2739 | | AAAGACAGCACCTATTCACTTTCCAGTACTCTGACCTTATCTA |
| HC: | | AGGCTGATTATGAGAAACACAAGGTGTATGCTTGTGAAGTTA |
| 2740-4083 | | CACATCAGGGGCTGAGCTCCCCTGTGACCAAAAGCTTTAATA |
| Stop Codon: | | GGGGGGAGTGTTAGGCATGCCTGTGCCTTCTAGTTGCCAGCC |
| 4084-4086 | | ATCTGTTGTTTGCCCCTCCCCCGTGCCTTCCTTGACCCTGGAA |
| Synthetic | | GGTGCCACTCCCACTGTCCTTTCCTAATAAAATGAGGAAATT |
| poly(A): | | GCATCGCATTGTCTGAGTAGGTGTCATTCTATTCTGGGGGGTG |
| 4093-4141 | | GGGTGGGGCAGGACAGCAAGGGGGAGGATTCTCAGAACTAA |
| ITR: | | CATACGCTCTCCATCAAAACAAAACGAAACAAAACAAACTA |
| 4148-4288 | | GCAAAATAGGCTGTCCCCAGTGCAAGTGCAGGTGCCAGAAC |
| | | ATTTCTCTATCGAAGGATCTGCGATCGCTCCGGTGCCCGTCAG |
| | | TGGGCAGAGCGCACATCGCCCACAGTCCCCGAGAAGTTGGG |
| | | GGGAGGGGTCGGCAATTGAACCGGTGCCTAGAGAAGGTGGC |
| | | GCGGGGTAAACTGGGAAAGTGATGTCGTGTACTGGCTCCGCC |
| | | TTTTTCCCGAGGGTGGGGGAGAACCGTATATAAGTGCAGTAG |
| | | TCGCCGTGAACGTTCTTTTTCGCAACGGGTTTGCCGCCAGAAC |
| | | ACAGCTGAAGCTTCGAGGGGCTCGCATCTCTCCTTCACGCGC |
| | | CCGCCGCCCTACCTGAGGCCGCCATCCACGCCGGTTGAGTCG |
| | | CGTTCTGCCGCCTCCCGCCTGTGGTGCCTCCTGAACTGCGTCC |
| | | GCCGTCTAGGTAAGTTTAAAGCTCAGGTCGAGACCGGGCCTT |
| | | TGTCCGGCGCTCCCTTGGAGCCTACCTAGACTCAGCCGGCTCT |
| | | CCACGCTTTGCCTGACCCTGCTTGCTCAACTCTACGTCTTTGT |
| | | TTCGTTTTCTGTTCTGCGCCGTTACAGATCGAAGTTGGTCGTG |
| | | AGGCACTGGGCAGGTAAGTATCAAGGTTACAAGACAGGTTTA |
| | | AGGAGACCAATAGAAACTGGGCTTGTCGAGACAGAGAAGAC |
| | | TCTTGCGTTTCTGATAGGCACCTATTGGTCTTACTGACATCCA |
| | | CTTTGCCTTTCTCTCCACAGGTGTCCACTCCCAGTTCAATTAC |
| | | AGCTCTTAAGGCTAGTTTAAACCACCATGCACAGCTCTGCCC |
| | | TGCTCTGCTGTTTGGTGCTTCTGACAGGAGTCAGGGCTCAGGT |
| | | TGAGTTAGTGGAATCTGGAGGGGGTGTAGTGCAACCTGGCAG |
| | | ATCACAGAGGCTGAGTTGTGCAGCAAGTGGATTCACATTTAG |
| | | CTCCTATGGGATGCATTGGGTCAGACAGGCCCCTGGCAAGGG |
| | | TCTAGAGTGGGTGGCTATCATTTGGTTTGATGGATCTTCAACT |
| | | TATTATGCTGATAGTGTTAGGGGGAGATTTACTATAAGCAGG |
| | | GACAACTCCAAAAATACCCTCTATCTGCAAATGAACTCTTTG |
| | | AGAGCAGAAGATACAGCTGTGTACTTCTGTGCTAGGGAGCTG |
| | | GGCAGAAGGTATTTTGACCTTTGGGGCAGAGGAACTCTTGTC |
| | | TCTGTATCCTCAGCAAGTACCAAGGGGCCATCTGTGTTCCCA |
| | | CTGGCCCCCTCTTCCAAAAGTACATCAGGTGGCACAGCAGCT |
| | | CTGGGATGCTTGGTTAAGGATTACTTTCCTGAACCAGTGACT |
| | | GTCAGCTGGAATAGTGGGGCCTTGACTTCTGGGGTGCACACA |
| | | TTCCCTGCAGTCCTTCAGTCCTCTGGTCTGTATTCATTATCCA |
| | | GTGTGGTTACAGTGCCTTCTAGCTCCCTGGGAACTCAGACCT |
| | | ACATCTGTAATGTAAATCATAAACCAAGCAACACAAAGGTGG |
| | | ACAAGAAAGTTGAGCCCAAGTCTTGTGATAAAACCCACACTT |
| | | GTCCACCTTGCCCAGCCCCTGAGCTACTTGGGGGCCCTTCAGT |
| | | TTTTCTGTTCCCACCCAAGCCTAAAGACACATTGATGATTAGT |
| | | AGGACTCCTGAAGTGACCTGTGTTGTGGTGGATGTTTCCCAT |
| | | GAGGACCCAGAAGTAAAGTTCAATTGGTATGTGGATGGAGTT |
| | | GAGGTGCACAATGCTAAGACTAAACCAAGAGAAGAGCAGTA |
| | | TAATAGCACATACAGGGTTGTGTCAGTTCTGACAGTGCTTCA |
| | | CCAAGACTGGCTCAATGGAAAGGAGTATAAATGCAAGGTAT |
| | | CTAATAAGGCCCTGCCTGCACCTATTGAAAAAACTATTTCAA |
| | | AGGCTAAAGGGCAGCCAAGAGAGCCCCAGGTCTACACCCTG |
| | | CCTCCATCCAGGGATGAATTAACAAAGAACCAGGTGAGTCTC |
| | | ACTTGCCTGGTTAAAGGTTTTTATCCCTCTGATATTGCTGTGG |
| | | AGTGGGAGTCTAATGGCCAACCAGAAAATAACTACAAGACC |
| | | ACACCTCCAGTCTTGGACTCTGATGGAAGCTTCTTTCTTTACT |
| | | CAAAGCTGACTGTGGACAAAAGTAGATGGCAGCAGGGGAAT |
| | | GTGTTCAGCTGTTCTGTTATGCATGAGGCACTACACAACCATT |
| | | ATACCCAGAAGTCTCTCAGTCTGTCACCTGGCAAATAGCCTA |
| | | GGAATAAAAGATCTTTATTTTCATTAGATCTGTGTGTTGGTTT |
| | | TTTGTGTGCTCGAGAGGAACCCCTAGTGATGGAGTTGGCCAC |
| | | TCCCTCTCTGCGCGCTCGCTCGCTCACTGAGGCCGGGCGACC |
| | | AAAGGTCGCCCGACGCCCGGGCTTTGCCCGGGCGGCCTCAGT |
| | | GAGCGAGCGAGCGCGCAGCTGCAGATCTG |

### Example 2: In Vitro Assessment and Characterization Anti-IGF-1R Protein from Plasmid Constructs

### IGg Protein Expression

Cells were seeded in cell culture-treated 48-well plates in regular media with 10% Fetal Bovine Serum and 1% Penicillin Streptomycin at 40-75% confluency depending on cell type and cell doubling. 24 hours after seeding, when cells were 80-90% confluent, cell media was exchanged for 300 μL fresh media per well, and plasmids to be transfected were diluted in OptiMEM and incubated with Lipofectamine 3000 (Invitrogen) according to the manufacturer's instructions. Diluted plasmids and Lipofectamine mix were added to cell culture in triplicate at three concentrations (0.83 μg/mL, 0.42 μg/mL, and 0.21 μg/mL per well; reported as 250 ng, 125 ng, and 62.5 ng/well in a 48 well plate). After 72 hours, supernatant was collected and cells were washed for RNA extraction with PBS three times. Both media supernatant and the cells were stored at −80° C. until further analyzed or processed.

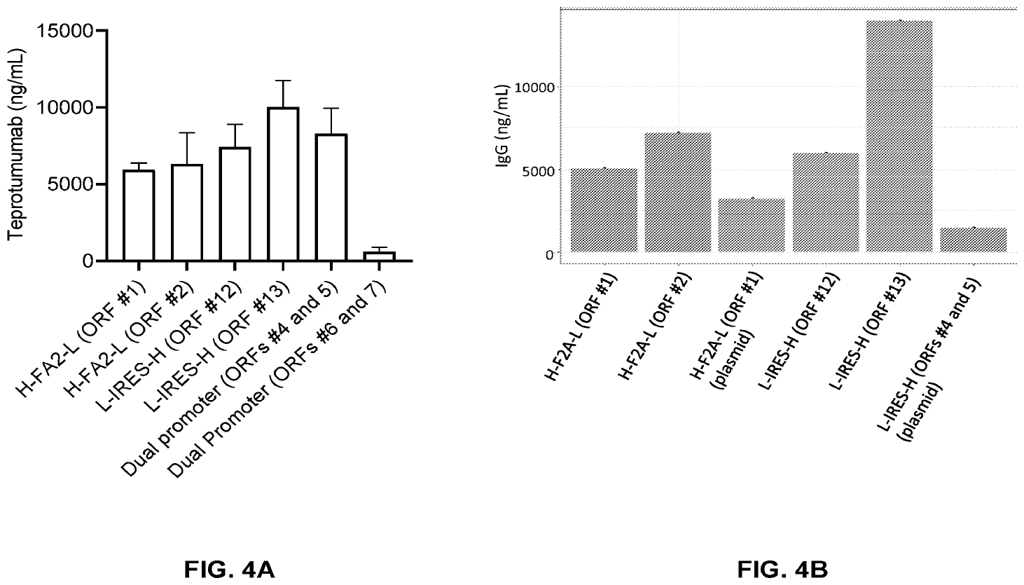

In the first experiment, HEK293 cells were transfected as described above with plasmids containing the following teprotumumab constructs in an oversized backbone: H-F2A-L (ORF #1), H-F2A-L (ORF #2), L-IRES-H (ORF #12), L-IRES-H (ORF #13), Dual promoter (ORFs #4 and 5), or Dual promoter (ORFs #6 and 7), and the total Teprotumumab IgG secretion was quantified (FIG. 4A). In the second experiment HEK293 cells were transfected as described above with plasmids containing the following teprotumumab constructs in an oversized backbone: H-F2A-L (ORF #1), H-F2A-L (ORF #2), L-IRES-H (ORF #12) or L-IRES-H (ORF #13), or with the following teprotumumab plasmid constructs in a pUC57 backbone: H-F2A-L (ORF #1) (plasmid) or L-IRES-H (ORF #12) (plasmid) (FIG. 4B).

HuIgG expression was determined by performing immunoassays carried out using a Gyrolab xPlore and Gyrolab huIgG low titer kit (Gyrolab). Kit included Bioaffy 1000HC CD, biotinylated capture and Alexafluor647 detection antibodies, wash buffers, and standard diluent Reagent E. All supernatants containing huIgG were centrifuged at 1,000 g for 30 minutes at 4° C. and tested neat. To quantify huIgG within each supernatant, a standard curve was prepared using Gyrolab huIgG standard starting at 25,000 μg/mL and diluted as recommended by kit literature in Reagent E. Concentration determined using the kit specific three-step (capture-analyte-detection), two wash solution, 0.05% PMT method.

In the first experiment, IgG was secreted between 5 and 10 μg/mL with all teprotumumab AAV transfected plasmids, exception made for the Dual promoter (ORFs #6 and 7) plasmid where expression was significantly lower (FIG. 4A). In the second experiment, high levels of teprotumumab secretion were also observed in cell transfected with the teprotumumab plasmids using oversized backbones, while lower levels were observed in cell transfected with the teprotumumab plasmids in pUC57 backbones, with >5 μg/ml of teprotumumab secreted from HEK293 cells at 72 hours post-transfection. A dose response was observed for all constructs. Additionally, the order of heavy and light chains, the linker sequence (F2A or IRES), appeared to affect teprotumumab expression levels (FIG. 4B).

The secreted antibodies were analyzed by Western blot to estimate heavy chain (HC) and light chain (LC) expression patterns. Unique antibodies were used to detect LC and HC, so relative quantification was not possible. However, no excess of LC bands in non-reducing blots, nor any additional bands in either blot for these transfections were observed, indicating the absence of partial species (FIG. 4C).

Supernatant collected from HEK293T cell culture were analyzed for molecular weight and purity by polyacrylamide gel electrophoresis (PAGE) followed by a Western Blot. The samples centrifuged and supernatant was collected and treated with HALT protease inhibitor cocktail. Recombinant Teprotumumab (Invitrogen and Proteogenix) and IgG1/Kappa Isotype control antibody (Abcam) were used as positive control samples, and the same volume of sample supernatant was loaded in each well. Samples were then loaded into precast Tris-Glycine gels: 4-20% Tris-Glycine gels were used for reducing conditions and 8% Tris-Glycine gels for non-reducing conditions. Pre-stained protein ladders were also loaded into one well of each gel. After running, gels were transferred to nitrocellulose membranes and blocked with Protein-Free TBS Blocking buffer (Invitrogen) at room temperature for 1 hour with gentle shaking. After blocking, all blots were incubated with a combination of two antibodies, diluted 1:400 (reducing conditions) or 1:1000 (non-reducing conditions) in fresh blocking buffer: AF647-conjugated rabbit monoclonal anti-human Kappa Light Chain antibody (Abcam), and HRP-conjugated mouse monoclonal anti-IgG1 Fc Antibody (Clone 43D17, Novus Biologicals). These antibodies were incubated with the blots at 4° C. overnight with very gentle rocking. After antibody incubation, blots were rinsed with TBS buffer 3 times for 10 minutes each. Blots were then incubated with Pierce ECL Western Blotting substrate and imaged in iBright using Universal Mode and Autodetect with two channels: Pierce ECL chemiluminescence and AF647 Fluorescence. Images of individual channels, merged, and of the prestained protein ladder were taken for each blot.

mRNA Expression

mRNA levels were measured to compare transcription and translation. Light and heavy chain expression levels were also compared, as well as mRNA levels from the different plasmids (FIG. 4D).

Total RNA was isolated from transfected cells using the Zymo Quick-RNA 96 Kit (Zymo Research, catalog #R1052) following the manufacturer's instructions. Isolated RNA was then subjected to DNase treatment/purification using the RNA Clean & Concentrator-96 Kit (Zymo Research, catalog #R1080) following the manufacturer's instructions. The concentration of isolated RNA was measured via UV/VIS spectroscopy using the NanoDropOne instrument (ThermoFisher, catalog #13-400-518). Transgenic RNA was quantified via 1-step reverse transcription quantitative PCR (RT-qPCR) using a 5′ endonuclease activity assay. Two separate RT-qPCR assays were used to quantify RNA containing the protein coding sequence for the heavy chain and light chain of teprotumumab. Restriction enzyme linearized plasmid DNA containing both the teprotumumab heavy chain and light chain DNA sequences was used as a quantification standard for both heavy chain and light chain RT-qPCR assays.

All primer RT-qPCR primers and probes were purchased from Integrated DNA Technologies.

Heavy-Chain-Forward:
(SEQ ID NO: 98)
5′ TCTCACGTGCCTGGTTAAAG 3′
Heavy-Chain-Reverse:
(SEQ ID NO: 99)
5′ GGAGGTGTGGTCTTGTAGTTATT 3′
Heavy-Chain-Probe:
(SEQ ID NO: 100)
5′ TATCGCCGTGGAGTGGGAGTCTAA 3′..
Probe modifications:
5′ 6-FAM (Fluorescein), 3′ ZEN/Iowa Black
fluorescence quenchers.
Light-Chain-Forward:
(SEQ ID NO: 101)
5′ CGCCTTCTGTCTTCATCTTTCC 3′
Light-Chain-Reverse:
(SEQ ID NO: 102)
5′ CCTCCCTCGGGTAGAAGTTATT 3′
Light-Chain-Probe:
(SEQ ID NO: 103)
5′ TACGGCTTCAGTGGTTTGTCTGCT3′.
Probe modifications:
5′ 6-FAM (Fluorescein), 3′ ZEN/Iowa Black
fluorescence quenchers.

TaqMan™ Fast Virus 1-Step Master Mix (ThermoFisher, catalog #4444434) was used to create 1-step RT-qPCR master mix. QuantStudio 5 (ThermoFisher, catalog #A28568) PCR platform was used for RT-qPCR, using “Fast” instrument settings. Thermocycling conditions were as follows, 1) 50° C. for 5 minutes; 2) 95° C. for 20 seconds; 3) 95° C. for 3 seconds; 4) 60° C. for 30 seconds; and Repeat steps 3-4 39 times.

### IGF-1R Binding Assay

The binding characteristics of the protein produced from transfected cells were also measured. The ability of expressed and secreted Teprotumumab to bind IGF-1R was assessed using the Gyros platform and a biotinylated recombinant IGF-1R protein with an anti-hIgG detection antibody. IGF-1R binding immunoassay for screening of Teprotumumab (anti-IGF-1R) was carried out using a Gyrolab xPlore (Gyrolab). Bioaffy 1000HC CD, wash buffers and standard diluent Reagent E from the Gyrolab huIgG low titer kit were utilized. Biotinylated human IGF-1R (Acro Biosystems) prepared at a concentration of 700 nM in sterile water served as the capture protein. Alexafluor647 anti-human IgG Fc prepared at a concentration of 5 μg/mL in Rexxip F buffer (Gyrolab) served as the detection antibody. All supernatants containing anti-IGF-1R were centrifuged at 1,000 g for 30 minutes at 4° C. and tested neat. To quantify anti-IGF-1R within each supernatant, a standard curve was prepared using biosimilar Teprotumumab (Proteogenix) starting at 8,000 μg/mL and diluted step 1:5 in Reagent E. Concentration determined using the huIgG low titer kit specific three-step (capture-analyte-detection), two wash solution, 0.05% PMT method.

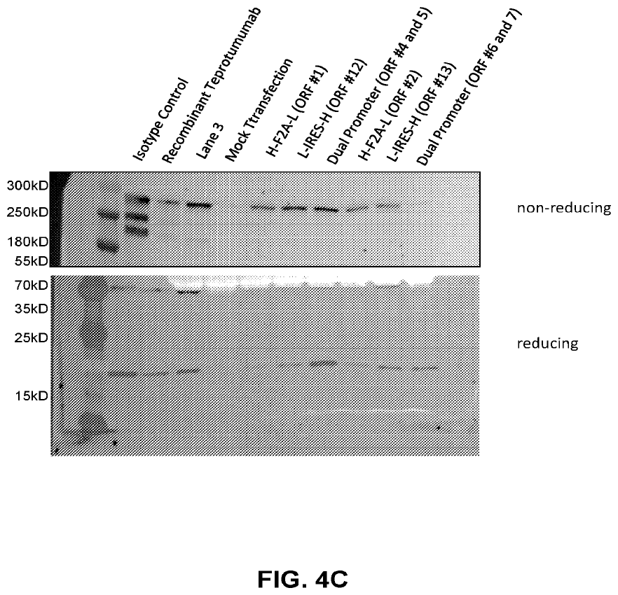

The results showed that vectorized Teprotumumab bound its target (IGF-1R) in a concentration dependent manner (FIG. 5A).

### IGF-1R Activity Blocking Assay

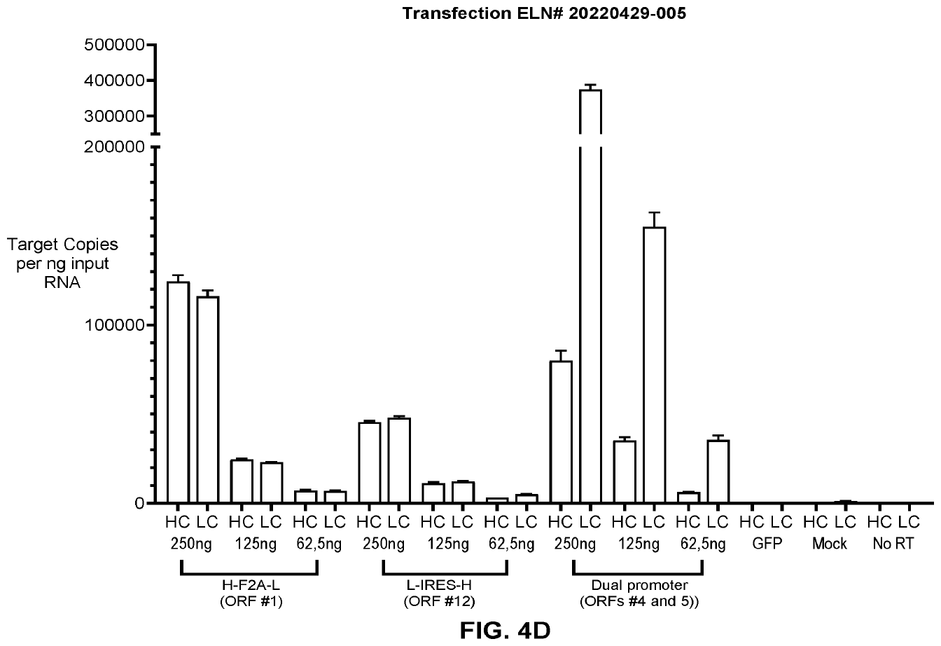

Multiple proteins are phosphorylated downstream of IGF-1R after activation with Insulin-like growth factor (IGF) I or II. Teprotumumab blocks IGF activation of IGF-1R and prevents downstream phosphorylation. Vectorized Teprotumumab activity was a measured by its ability to block phosphorylation of IGF-1R. A dose dependent reduction in IGF-1R activation by insulin growth factor (IGF) was observed when HT-1080 cells were exposed to vectorized Teprotumumab and then treated with 500 ng/ml IGF. A 4-fold reduction in activation was observed at a dose of 500 ng/ml of vectorized teprotumumab. These results showed that recombinant teprotumumab and vectorized (plasmid-secreted) teprotumumab had similar dose-response curves for reduction in IGF-1R phosphorylation (FIG. 6).

Cells were seeded 3E5 cells per well in a 6 well plate. At 90% confluency, cells were pre-treated for 30 minutes with recombinant Teprotumumab (Invitrogen) or vectorized Teprotumumab from cell culture supernatant 72 hours after transfection with Teprotumumab plasmids at a concentration of 100 ng-2 μg/mL (100 ng-2 μg/well). After 30 minutes, some wells were also treated with recombinant Insulin-like Growth Factor (rIGF-I) (Invitrogen) for 15 minutes at a concentration of 500 ng-1 μg/mL (500 ng-1 μg/well). After 15 minutes, all cell supernatant was removed and the cell layers were rinsed with ice-cold PBS. 600 μL of complete MSD Lysis Buffer made with MSD Tris Lysis Buffer and Phosphatase inhibitors from the Insulin Signaling Panel (Phospho Protein) Whole Cell Lysate Kit (Meso Scale Discovery, cat #K15151C-1) was added to the cell layer and the plate was incubated on ice for 30 minutes. After 30 minutes, a pipette was used to mix each well of cells and MSD Complete Lysis Buffer, then the mix from each well was collected in 1.5 mL microcentrifuge tubes and spun at 14,000×g at 4° C. for 10 minutes. After spinning, the supernatant was collected.

The collected protein samples were used in the BCA Assay (Pierce) to determine the concentration of protein in each sample in μg/mL according to the manufacturer's instructions.

The MSD assay was run in accordance with the manufacturer's instructions using the Insulin Signaling Panel (Phospho Protein) Kit. Briefly, a 96-well 4-spot MSD plate was blocked at room temperature on a plate shaker at 700 rpm for 1 hour. After blocking, the plate was washed 3 times with Tris Wash Buffer. Next, 7.5 μg total protein (300 ng/μL) from each sample, diluted in PBS, was loaded per well. The MSD plate loaded with samples and controls in triplicated, sealed, and incubated at room temperature on a plate shaker at 700 rpm for 1 hour. After sample incubation, the plate was washed 3 times with Tris Wash Buffer. Next, each well used was loaded with detection antibody Solution (Sulfo TAG Anti-Py20 antibody, MSD Kit reagent, in Tris Wash Buffer+Blocking Buffer) and the plate was incubated at room temperature on a plate shaker at 700 rpm for 1 hour. After detection antibody incubation, the plate was washed 3 times with Tris Wash Buffer. 150 μL of Read Buffer T (MSD Kit reagent) was added to each well, and the plate was read on an MESO QuickPlex SQ 120 instrument, where each well was assigned 3 raw values, one for each target detected: phosphorylated Insulin-like Growth Factor Receptor-I; phosphorylated IRS-1, and phosphorylated Insulin Receptor.

Data from each of the 3 protein targets was analyzed separately from one another. Per target, a fold change value was determined for each treated sample by using the equation:

$$\frac{\begin{matrix}( Individual sample raw signal value - \\ PBS only mean raw signal value )\end{matrix}}{\begin{matrix}( No Treatment sample raw signal value - \\ PBS only mean raw signal value )\end{matrix}}$$

Statistics were run on fold change values using one-way ANOVA.

Both recombinant Teprotumumab and vectorized Teprotumumab inhibited IGF-1R phosphorylation in a concentration dependent manner and to a similar extent. Inhibition of phosphorylation was statistically significant relative to untreated controls for all concentrations tested for both recombinant and vectorized Teprotumumab, with vectorized Teprotumumab tending to be more potent than the recombinant antibody. Results are presented in FIG. 6.

### Example 3: In Vitro Assessment and Characterization of Anti-IGF-1R Protein Expressed from an AAV Vector

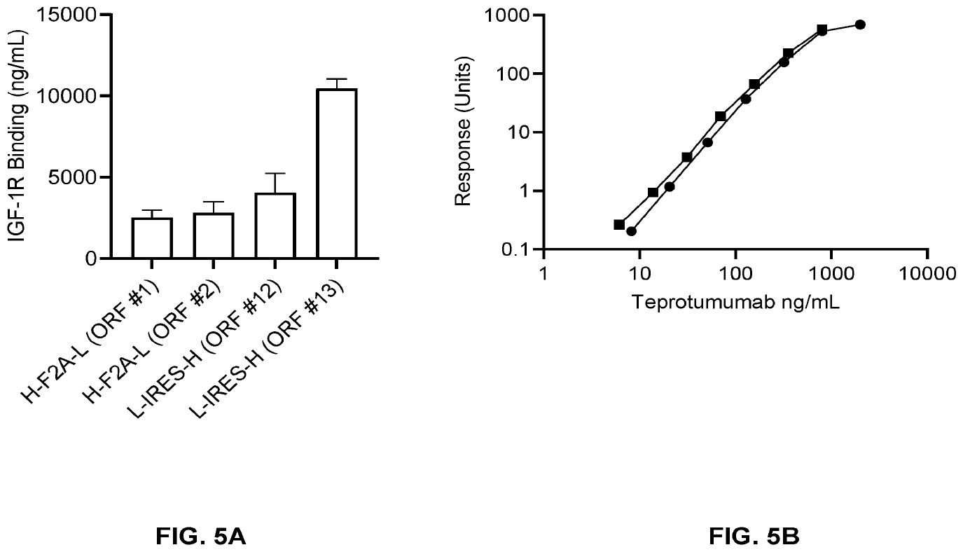

Treatment of HT-1080 and Colo205 cells with recombinant Teprotumumab or Teprotumumab from HEK293T cell culture supernatant collected 72 hours after transduction with AAV2 H-F2A-L (ORF #2) Teprotumumab showed decreased total and phospho-IGF-1R expression levels (FIGS. 7A-B).

HEK293 cells were grown to optimal confluency in 6-well plates. Cells were then transduced with various MOIs (Multiplicity Of Infection. MOI=number of viral particles per cell) of AAV-anti-IGF-1R (MOI 5E±3 to 5E±5) and supernatants from each well were collected at 72 hours post transfection.

Cells were seeded at 1E5 (HT-1080) or 2E5 (Colo205) cells per well in a 24-well plate. At 50% confluence, all cells were serum starved overnight. Cells were then treated with either recombinant Teprotumumab (Invitrogen) or Teprotumumab from HEK293T cell culture supernatant 72 hours after transduction with AAV-delivered H-F2A-L (ORF #2) Teprotumumab (UMass, Lot #VCAV-08721) at 50 ng/ml, 100 ng/ml, or 500 ng/mL. The control well for each cell line was treated with cell culture supernatant 72 hours after a mock transduction and referred to as “mock”. Cells were incubated for 48 hours. At 48 hours, supernatant was removed and the cell layers were rinsed with ice-cold cell-culture grade PBS. 200 μL of complete MSD Lysis Buffer from the Insulin Signaling Panel (Phospho Protein) and (Total Protein) Whole Cell Lysate Kits received from Meso Scale Discovery (Cat #K15151C and Cat #K15152C, respectively) were added to the cell layer and the plate was incubated on ice for 30 minutes. After 30 minutes, a pipette was used to mix each well of cells and MSD Complete Lysis Buffer, then the mix from each well was collected in 1.5 mL microcentrifuge tubes and spun at 14,000×g at 4° C. for 10 minutes. After spinning, the supernatant (HT-1080 or Colo205 whole cell lysate) was collected and total protein quantification was performed using the BCA Assay Kit (Pierce) using protein standard albumin as the standard curve to determine protein concentration for each sample. The Phospho and Total protein Insulin Signaling Panels were then run with the whole cell lysates from both cell lines as described in Example 2.

Treatment of both HT-1080 and COLO205 cancer cells with both recombinant Teprotumumab or an AAV-delivered antibody expression cassette encoding Teprotumumab resulted in significant decrease in both total IFG-1R and phosphorylated IGF-1R. Results are presented in FIG. 7A and FIG. 7B.

Methods will be also employed to assess the binding characteristics of the protein produced from transduced cells. These methods will include protein quantification, protein binding to IGFR ligand, and assessment of affinity and binding kinetics of anti-IGF-1R protein generated as described herein as compared to the commercially available anti-IGF-1R protein (Teprotumumab, commercially known as Tepezza).

Anti-IGF-1R protein binding will be tested using Enzyme-linked Immunosorbent Assay (ELISA) and Surface plasmon resonance for binding to IGFR and compared to binding of commercially available Teprotumumab.

### Example 4: In Vitro Assessment of Cytokine Production and IGF-1R Phosphorylation Levels in Normal and Graves' Patient Fibrocytes Treated with Recombinant or Vectorized Teprotumumab

Isolation and Passaging of Human Fibrocytes from Peripheral Blood

Peripheral whole blood samples collected in EDTA collection tubes from healthy donors or Graves' Disease (GD) patients were overlayed over Ficoll (Ficoll-Paque, Cytiva, 1.078 g/mL density) to isolate peripheral blood mononuclear cells (PBMC) and then centrifuged at 400×g for 40 minutes (no brake). The PBMC layer was isolated (along with a sample of plasma), and the Donor/GD Patient cells were then washed with three times their volume in phosphate buffered saline (without Ca⁺⁺ or Mg⁺⁺), for a total of three washes, by centrifuging initially at 400×g for the first wash, and at 180×g for the subsequent washes to remove any residual platelets. After the final wash, the PBMCs were resuspended in Fibrocyte Culture Media (Dulbecco's modified Eagle's medium (DMEM) with 10% fetal bovine serum (FBS), 1% Penicillin-Streptomycin (1000 U/mL), 1% L-Glutamine (200 mM)), and counted using a Neubauer Improved Hemacytometer. The PBMCs were resuspended at 1×10⁷ cells/mL and cells were plated with 1×10⁷ cells per well in 6-well tissue culture plates. Seven days later, the non-adherent cells were removed, and the adherent cells (fibrocytes) were washed 3 times in PBS before addition of fresh Fibrocyte culture media to each well. Cultures were fed with fresh media every 3-4 days, and the confluency of the cells was monitored to determine when the cells should be passaged. To passage cells, adherent cells (fibrocytes) were washed with PBS and treated with 0.25% Trypsin/EDTA solution or StemPro Accutase cell dissociation reagent (Gibco) to release cells from tissue culture plate, harvested, well washed with PBS or fibrocyte media, and centrifuged at 140×g for 7-10 minutes. Cells were then resuspended in fresh Fibrocyte culture media and either seeded at 1.0-1.4×10⁴ cells/cm² in either plates or flasks or utilized for subsequent experiments (i.e., flow cytometry, immunocytochemistry, etc.).

### Insulin/IGF1R Signaling Blockade Assay

Vectorized Teprotumumab activity was measured by its ability to decrease total IGF-1R, 1R or IRS-1 protein concentration in fibrocytes derived from the peripheral blood of Graves' disease patients or normal donors. Fibrocytes were seeded at 2×10+ cells/cm² in a 24-well or 48-well plate with Fibrocyte Culture Media (Dulbecco's modified Eagle's medium (DMEM) with 10% fetal bovine serum (FBS), 1% Penicillin-Streptomycin (1000 U/mL) 1% L-Glutamine (200 mM)). Once the cells reached ˜70-80% confluency, 1% FBS serum starving Fibrocyte Media (Dulbecco's modified Eagle's medium (DMEM) with 1% fetal bovine serum (FBS), 1% Penicillin-Streptomycin (1000 U/mL)) was added to each well overnight (<20 hours). Cells were then treated with either recombinant Teprotumumab (Invitrogen), or vectorized Teprotumumab from HEK293T cell culture supernatant 72 hours after transduction with H-F2A-L (ORF #2) Teprotumumab at 500 ng/mL. The control well for each cell line was treated with cell culture supernatant 72 hours after a mock transduction and referred to as “mock” or media for the recombinant Teprotumumab treated conditions. After 24 or 48 hour(s), the cells were utilized for generation of cell lysates for evaluation of total or phosphorylated IGFR, 1R, or IRS-1 in Insulin Signaling Panel (Phospho Protein) and (Total Protein) Whole Cell Lysate Kits received from Meso Scale Discovery (Cat #K15151C and Cat #K15152C, respectively).

Stimulation of Fibrocytes with Insulin Growth Factor or Thyroid Stimulating Hormone with Teprotumumab Blockade (Prophetic)

Fibrocytes are seeded at 2×10⁴ cells/cm² in a 24-well or 48-i plate with Fibrocyte Culture Media (Dulbecco's modified Eagle's medium (DMEM) with 10% fetal bovine serum (FBS), 1% Penicillin-Streptomycin (1000 U/mL), 1% L-Glutamine (200 mM)). Once the cells reach ˜70-80% confluency, 1% FBS serum starving Fibrocyte Media (Dulbecco's modified Eagle's medium (DMEM) with 1% fetal bovine serum (FBS), 1% Penicillin-Streptomycin (1000 U/mL) is added to each well overnight (<20 hours). Cells are then treated with either recombinant Teprotumumab (Invitrogen), or Teprotumumab from HEK293T cell culture supernatant 72 hours after transduction with an AAV-delivered antibody expression cassette encoding Teprotumumab at 50 ng/ml, 100 ng/ml, 500 ng/mL, 5,000 ng/mL. The control well for each cell line is treated with cell culture supernatant 72 hours after a mock transduction and referred to as “mock”. Designated treatments groups are stimulated with Thyroid Stimulating Hormone (TSH; 5 mU/mL) (R&D Systems) or Insulin Growth Factor-1 (IGF-1; 100 ng/mL) (R&D Systems). After 1, 24, and/or 48 hour(s), the supernatant is collected for inflammatory cytokine evaluation (IL-1B, IL-6, IL-8 and TNF-α) via MSD proinflammatory kit and cells are utilized either for immunocytochemistry or for generation of cell lysates for evaluation of total or phosphorylated IGFR in Insulin Signaling Panel (Phospho Protein) and (Total Protein) Whole Cell Lysate Kits (Meso Scale Discovery, Cat #K15151C and Cat #K15152C, respectively).

Evaluation of Pro-Inflammatory Cytokine Production by Fibrocytes Following with Insulin Growth Factor or Thyroid Stimulating Hormone with Teprotumumab Blockade (Prophetic)

Tissue culture supernatants from fibrocyte stimulation cultures are diluted a minimum of 2-fold using Diluent 2 from the V-PLEX Plus Human Proinflammatory Panel II kit (4-Plex) (Mesoscale Discovery (MSD), catalog #K15053G) per manufacturer's instructions. Briefly, lyophilized calibrator (supplied by MSD) is reconstituted in 1 mL of Diluent 2 and inverted several times. The solution is left to equilibrate to room temperature (RT) for 15-30 minutes and then vortexed briefly. A 4-fold, 8-point series dilution is then made using the calibrator as the highest concentration per manufacturer's instructions. Diluent 2 is used as the zero calibrator. Control 1, 2, and 3 (supplied by MSD) are reconstituted by added 250 μL of Diluent 2 to each vial. Each control stock solution is left for 15-30 minutes at RT before vortexing and diluting controls to a 2-fold solution using Diluent 2. 1× Wash Buffer and 2× Read Buffer are also prepared per manufacturer's instructions. The MSD plate is washed 3 times with 150 μL/well of Wash Buffer before adding 50 μL of prepared samples, calibrators, and controls per well. The plate was sealed and incubated at RT for 2 hours on a plate shaker at 700 rpm. During the incubation period, the antibody solution is made by adding 60 μL each of IL-1B, IL-6, IL-8 and TNF-α detection antibody to 2,760 μL of Diluent 3. The solution is vortexed thoroughly. Once the 2-hour incubation is complete, the plate is washed 3 times using 150 μL/well of Wash Buffer. 25 μL of detection antibody solution are added to each of the wells before sealing the plate and placing on the plate shaker at 700 rpm for 2 hours at RT. After incubation, the plate is washed with 150 μL/well of Wash Buffer 3 times. Read Buffer (150 μL) is added to each of the wells, and the plate is read on an MESO QuickPlex SQ 120 instrument.

### Whole Cell Lysate Collection

After 1, 24, and/or 48 hour(s) treatment of fibrocytes as described above, cell lysates were harvested according to MSD protocol with the following modifications. Briefly, cell supernatants were removed, and cell monolayers were rinsed with ice-cold cell-culture grade PBS. 150-200 μL of complete MSD Lysis Buffer from the Insulin Signaling Panel (Phospho Protein) and (Total Protein) Whole Cell Lysate Kits received from Meso Scale Discovery (Cat #K15151C and Cat #K15152C, respectively) was added to cell monolayers and the plate was incubated on ice for 5 minutes. Cell monolayers were then scraped to bring treated fibrocytes into suspension in the complete lysis butter. Cells were then incubated for an additional 25 minutes on ice. After incubation, a pipette was used to mix each well of cells and MSD Complete Lysis Buffer, then the mix from each well was collected in 1.5 mL microcentrifuge tubes and spun at 14,000×g at 4° C. for 10 minutes. Lysate was then subaliquotted and stored at −80° C.

### Total Protein Quantification and MSD Multi-Spot Insulin Signaling Assay

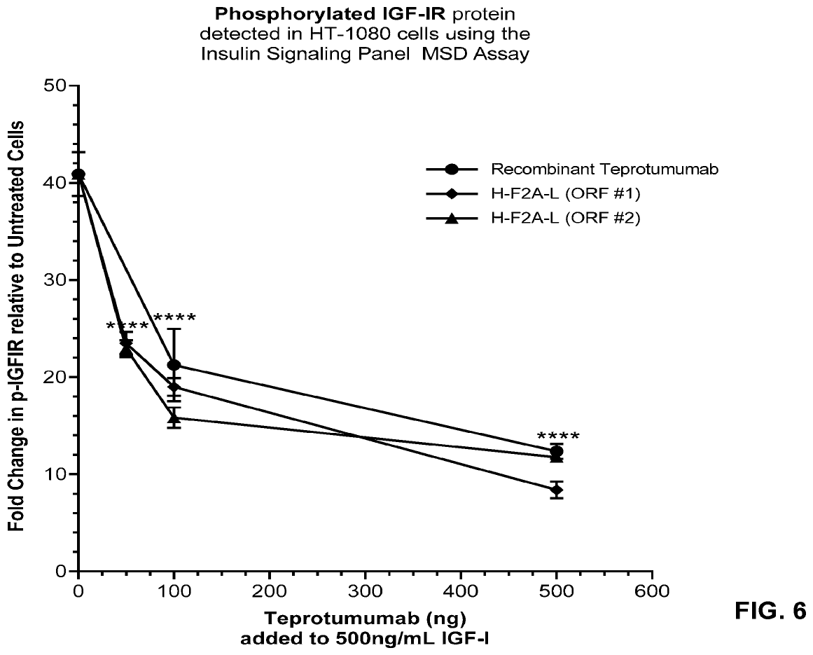

Total protein quantification was performed using the DC Protein Assay Kit II (Bio-Rad) with protein standard albumin as the standard curve to determine protein concentration for each sample. The Phospho and Total protein Insulin Signaling Panels were then run with the whole cell lysates to quantify levels of total and/or phosphorylated IGF-1R, IRS, and IR. Briefly, in the 96-well 4-spot MSD plate provided by the Insulin Signaling Panel (Phospho Protein) Kit, 150 μL blocking solution was added to each well to be used, the plate was sealed using an adhesive plate seal, and the plate was incubated at room temperature on a plate shaker at 750 rpm for 1 hour. After blocking, the plate was washed 3 times with Tris Wash Buffer. Next, 5-7.5 μg of total protein (200-300 ng/μL) diluted in PBS was loaded per well, with each sample loaded in triplicate. 3-4 wells were also loaded with PBS alone as blank/background. The MSD plate loaded with samples in duplicate was then sealed using an adhesive seal and incubated at room temperature on a plate shaker at 750 rpm for 1 hour. After sample incubation, the plate was washed 3 times with Tris Wash Buffer. Next, each well used was loaded with detection antibody Solution according to the respective kit instructions, and the plate was incubated at room temperature on a plate shaker at 750 rpm for 1 hour. After detection antibody incubation, the plate was washed 3 times with Tris Wash Buffer. 150 μL of diluted 1×Read Buffer T (MSD Kit reagent) was added to each well, and the plate was read on an MESO QuickPlex SQ 120 instrument, where each well was assigned raw values for the target detected. Phospho and Total protein results were analyzed separately using MSD Discovery Workbench software. For each protein, fold change value was determined for each treated sample by using the equation: (Individual sample raw signal value-PBS only mean raw signal value)/(Mock Treatment sample raw signal value-PBS only mean raw signal value). The percent reduction in protein expression following treatment with vectorized or recombinant Teprotumumab in comparison to control (mock or media, respectively) was calculated using either mock transduction as 100% expression for vectorized Teprotumumab or media alone as 100% expression level. Treatment of either Graves' disease (GD) donor fibrocytes or normal donor fibrocytes with vectorized Teprotumumab resulted in a decrease in total IGF-1R protein (FIG. 8A). Treatment with vectorized Teprotumumab also reduced Insulin receptor expression in both normal and GD patient fibrocytes (FIG. 8B). Vectorized Teprotumumab also decreased IRS-1 protein expression in Graves' disease fibrocytes (FIG. 8C). In contrast, recombinant Teprotumumab had limited impact on normal donor fibrocyte IGF-1R protein expression and did not impact GD patient fibrocyte's IGF-1R expression.

### Example 5: In Vitro Assessment of Cell Migration Normal and Graves' Patient Fibroblasts Treated with Recombinant or Vectorized Teprotumumab

The ability of IGF1 to induce T cell migration will be tested. To this end, T cells will be incubated in the presence of IGF-1, IL-1B, Graves' disease IgG, Graves' disease IgG and anti-IGF-1R or Graves' disease IgG and isotype control antibodies. Migration of T cells will be assessed and compared to untreated control T cells.

Using cells isolated from orbital fat of TED patients and control patients with no history of TED, the effect of anti-IGF-1R protein generated and purified from supernatants of plasmid transfected cells, AAV transduced cells or both as described in Example 2 or 3 will be assessed.

To this end, orbital adipose tissue explants will be obtained from TED patients during orbital fat decompression and control individuals with no history of TED during blepharoplasty. Tissue explants will be chopped and treated with collagenase. After digestion, the tissues will be placed directly in culture dishes with DMEM/F12 containing 20% fetal bovine serum. The cells will be serially passaged, and cells of the fifth to eighth cell passage will be used for the experiments.

Primary fibroblast cells will be used to measure IGF-1R levels by MSD, and to determine efficacious dose using supernatants from cells transduced at different MOIs of vectorized Teprotumumab for 24-48 hours. After treatment, downstream signaling pathways including detecting IGF-1R, 1R, IRS-1 (phospho insulin signaling panel), AKT-mediated phosphorylation, and measuring cytokine levels using human proinflammatory panel II (MSD) including IL-16B, IL-6, IL-8 and TNF-α will be assessed.

Normal and TED cells will be transduced with AAV-anti-IGF-1R constructs generated as described in Example 1 and the supernatant of AAV-transduced cells and untransduced control cells will be tested in a T cell migration assay as described above.

### Example 6: In Vitro Assessment of Normal and Graves' Patient Fibrocytes Isolated from Peripheral Blood and Treated with AAV-Anti IGF-1R Vectors

Fibrocytes from bone-marrow-derived progenitor cells of the monocyte lineage play an important role in the pathogenesis of thyroid associated-ophthalmology. Fibrocytes from healthy and Grave's patients will be isolated, evaluated for surface marker expression by flow cytometry with CD34+, CD45+, CXCR4+, collagen 1, IGF-1R and TSHR and assessed for anti-IGF-1R protein effect generated from supernatants of AAV transduced cells. First, fibrocytes from healthy donors or Graves' disease patients with and without orbitopathy will be treated with IGF or TSH to stimulate activity in the fibrocyte cells. Then, fibrocytes will be treated with supernatant from cells transduced at different MOIs of vectorized Teprotumumab for 24-48 hours. Experiments will be conducted to determine efficacy of vectorized Teprotumumab and establish effective concentrations of anti-IGF-1R antibody including IGF-1R expression levels by measuring IGF-1R signaling (MSD), and IGF-1R changes by flow cytometry or immunocytochemistry, detecting IGF-1R, 1R, IRS-1 phosphorylation by the phospho insulin signaling panel (MSD), and measuring cytokine levels such as IL-6B, IL-6, IL-8 and TNF-α using human proinflammatory panel II (MSD).

### Example 7: A Graves' Disease Mouse Model

A GO mouse model will be used according to the methods reported in Zhang et al. Thyroid, 31:638-648 and Moshkelgosha et al. Endocrinology, 154:3008-3015, 2013. To this end, BALB/c female mice will be immunized with a plasmid overexpressing the hTSHR A-subunit protein. About 22 weeks after the first immunization, the animals will be bled to evaluate the induced anti-TSHR antibodies.

The animals with anti-hTHSR antibodies will be divided into the following groups: a treatment group injected with AAV-anti-IGF-1R, a treatment group with Teprotumumab, and a sham group.

AAV-anti-IGF-1R vectors will be injected by intra-orbital injection into the left orbit. Animals will be sacrificed at 2, 4, 8, and 12 weeks post initial dosing. Blood will be collected for serum analysis, and orbital tissue will be excised for histopathological analyses. Orbital tissues will also be assessed for T cell infiltration as well as levels of glycosaminoglycans.

Animals with anti-hTHSR antibodies and age-matched normal mice will be evaluated via MRI pre and post administration of AAV-anti-IGF-1R vector therapy to monitor effect of treatment on proptosis.

### Example 8: A Xenograft Tumor Mouse Model Bearing the Human Colorectal-Derived Cells, Colo205

Without being bound to a particular theory, it is believed that Teprotumumab binds to IGF-R1 and blocks its activation and downstream signaling pathway. This leads to the downregulation of IGF-R1. Reduced levels of IGF-R1 can be used as indicative of biological activity of an anti-IGF-1R drug.

A xenograft tumor mouse model using a Colo205 cell line was chosen as a model to assess efficacy of AAV1, AAV2, and AAV9 vectors carrying expression cassettes encoding anti-IGF-1R antibody.

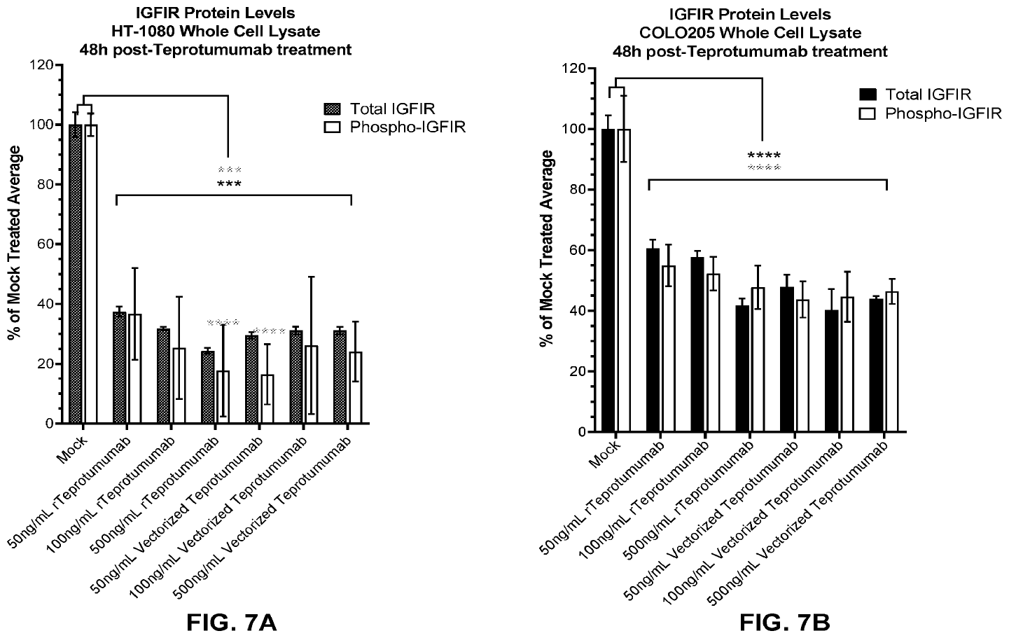

The study to evaluate efficacy of rAAV-mediated exogeneous expression of anti-IGF-1R antibody after intratumoral injection was performed in xenografted nude mice bearing the Colo205 cell line. The Colo205 xenograft has high IGF1-R expression and demonstrates growth kinetic that are suitable for a 1-month study. Expression of IGF-1R and reduction of IGF-1R expression in Colo205 cells in vitro upon treatment with recombinant Trepotumumab was confirmed by MSD (FIG. 7B) and by immunohistochemistry (FIG. 9).

Colo205 cells were seeded at 40,000 cells per well in a black 96-well clear-bottom plate and grown to 90% confluency. For Teprotumumab treatment, Colo205 cells were treated with 500 ng/mL recombinant Teprotumumab (Invitrogen) in culture medium and incubated for 24-48 hours. Culture media was removed from each well and wells were washed with PBS. Cells were fixed using 4% Formaldehyde solution for 10 minutes at room temperature followed by washes with PBS. If cell membranes were permeabilized, cells were treated with 0.1% Triton X-100 in PBS for 10 min followed by washing with PBS. Cells were blocked with 1% BSA in PBS for 1 hour, and then incubated for 1 hour at room temperature with anti-IGF-1R antibody (R&D Systems) diluted in 1% BSA in PBS. Cells were washed with PBS, and then incubated for 1 hour at room temperature in the dark with anti-mouse antibody APC-conjugated (R&D Systems) diluted in 1% BSA in PBS. Cells were washed with PBS. Cell nuclei were stained using 3 μM DAPI (Invitrogen) in PBS for 5 minutes. Cells were washed and stored in PBS for imaging using Leica DMI8 fluorescence microscope with a 20× HC PL APO C52 0.75 dry objective. Image files were processed using ImageJ software.

### Colo205 Xenografted Mouse Model

Subcutaneous human Colo205 tumors were established in female athymic nude mice. When tumors reached a median volume of approximately 100 mm³, mice were randomized based on tumor volume into groups of 8 animals. Tumors were treated with a single intratumoral injection containing an AAV-delivered antibody expression cassettes encoding Teprotumumab (designated as AAVX.Teprotumumab (where X was either AAV 1, 2, or 9)) at doses indicated in Table 18 formulated in 25 μL of a vehicle consisting of 350 mM NaCl, 5% D-sorbitol in PBS pH 7.4. Control animals received either no treatment or systemic Teprotumumab. Mice were randomly divided into efficacy cohorts (Table 18) and sampling cohorts (Table 19). The efficacy cohorts continued in the study until Day 31 to monitor tumor growth. At day 31 these mice were sacrificed. Three mice for each group in the sampling cohorts were sacrificed at Day 7, 14, and 28. Relevant tissues were collected at necropsy to measure PK, PD and biodistribution (Table 20).

Tumors were measured twice weekly using calipers and the tumor volume calculated using the following equation:

$$tumor volume ( {mm}^{3} ) = ( length * {width}^{2} ) * 0.5$$

In addition to tumor volumes, individual body weights were measured at least twice weekly along with clinical observations.

TABLE 18
Treatment assignments for efficacy groups

| | | | | | Route of | |
|---|---|---|---|---|---|---|
| Group | N | Agent | Active Dose | | Administration | Schedule |
| 1 | 8 | Untreated | N/A | | — | — |
| 2 | 8 | Teprotumumab | 6 | mg/kg | IP | every 7 days |
| | | | | | | to end |
| 3 | 8 | AAV1. Teprotumumab | 1.5E12 | vg/mouse | IT | 1x on study |
| | | | | | | day 1 |
| 4 | 8 | AAV9. Teprotumumab | 1.5E12 | vg/mouse | IT | 1x on study |
| | | | | | | day 1 |
| 5 | 8 | AAV2. Teprotumumab | 9.5E11 | vg/mouse | IT | 1x on study |

day 1

TABLE 19
Treatment assignments for sampling groups

| | | | | Route of | |
|---|---|---|---|---|---|
| Group | N | Agent | Active Dose | Administration | Schedule |
| 1 | 9 | No treatment | N/A | — | — |
| 2 | 9 | AAV1. Teprotumumab | 1.5E12 vg/mouse | IT | 1x on study |
| | | | | | day 1 |
| 3 | 9 | AAV9. Teprotumumab | 1.5E12 vg/mouse | IT | 1x on study |
| | | | | | day 1 |
| 4 | 9 | AAV2. Teprotumumab | 9.5E11 vg/mouse | IT | 1x on study |
| | | | | | day 1 |

IT = intratumoral injection;
IP = intraperitoneal

TABLE 20
Tissue Collection and Evaluation

| Tissue | Analysis |
|---|---|
| Serum | Protein analysis for Teprotumumab |
| Tumor | Weight; |
| | Protein analysis for Teprotumumab and IGF-R1; |
| | RNA analysis for AAV VCN, |
| | Teprotumumab and IGF-R1 mRNA |
| Liver and Spleen | RNA analysis for AAV VCN, |
| | Teprotumumab and IGF-R1 mRNA |

### Tumor Growth

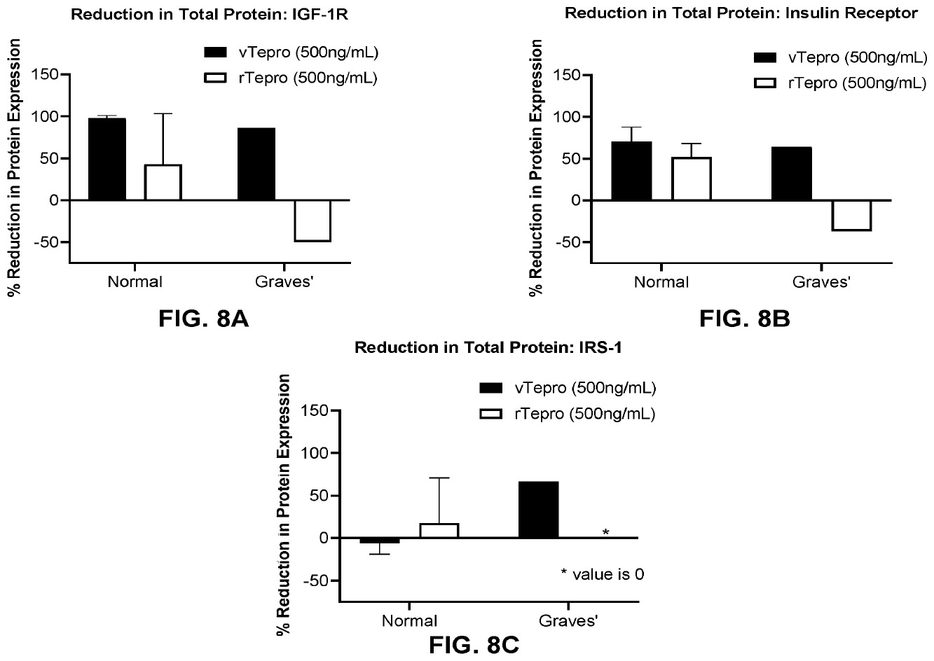

Intratumoral delivery of antibody expression cassettes encoding Teprotumumab via AAV1, AAV9 or AAV2 was well tolerated. No meaningful effect on body weight and no gross clinical adverse signs outside of those commonly associated with tumor burden were observed. The systemically delivered recombinant Teprotumumab had no effect on tumor growth in this study. This was expected based on the drug's performance and limited effect in xenograft models of other cancers with high IGF1-R expression (see, e.g., U.S. Pat. No. 7,572,897). In the current study, the Colo205 xenograft model was used because Colo205 cells have high IGF1-R expression and demonstrate faster growth kinetic compared to H322M used in other models. Without being bound by theory, this may be a factor contributing to the lack of an observed effect with recombinant Teprotumumab even at the same dose regimen of 6 mg/kg every 7 days. It is also of note that the AAV-delivered antibody expression cassettes encoding Teprotumumab were administered once at the beginning of the study, while systemic recombinant Teprotumumab was delivered weekly. Intratumoral AAV1 and AAV2-delivered antibody expression cassettes encoding Teprotumumab also appeared to have no effect on tumor growth over the course of the study. However, intratumoral AAV9-delivered antibody expression cassettes encoding Teprotumumab treatment of established Colo205 tumors led to some tumor growth inhibition. Unexpectedly, mean tumor measurements at days 11, 14 17 and 31 post AAV9 treatment. AAV-delivered antibody expression cassettes encoding Teprotumumab treatment were significantly smaller compared to untreated controls as well as systemically delivered recombinant Teprotumumab (FIGS. 10A-C). Furthermore, evaluation of the overall response using area-under-the-curves calculated from the tumor volume-time curves also demonstrated a significant effect relative to the control arm for AAV9-delivered antibody expression cassettes encoding Teprotumumab. No impact on AUC was observed for recombinant Teprotumumab, or the AAV1- or AAV2-delivered antibody expression cassettes encoding Teprotumumab (Table 21).

TABLE 21
AUC demonstrates Tumor Growth Delay
in AAV9-Teprotumumab Treated Mice
Efficacy arm only

| Treatment | AUC d1-31 |
|---|---|
| Control | 171 ± 12 |
| Tepro | 164 ± 18 |
| AAV1 | 153 ± 21 |
| AAV9 | 141 ± 6* |
| AAV2 | 185 ± 35 |

*p < 0.05 (1-sided t test)

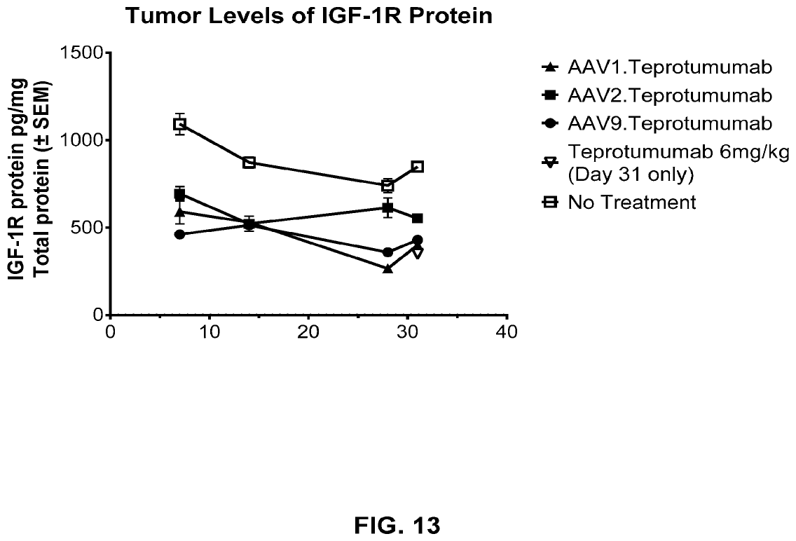

These results demonstrated the effect of single dose intratumoral (IT) delivered gene therapies compared to repeat dosing of monoclonal therapies. Together, local administration of AAV9-delivered antibody expression cassettes encoding Teprotumumab showed superiority to systemic administration of the antibody in this model of colon cancer. Further, early signs of efficacy may have been reduced at later timepoints due to the dilution of AAV within the rapidly dividing cells of the tumor. As shown in FIGS. 14-16, both vector genomes and vector mRNA decreased over the course of the experiment, though the reduction in vector mRNA expression from AAV9 was much less than AAV1 or AAV2 vectors.

### Tumor Homogenization

Harvested tissues from the animal study site were flash frozen and stored at −80° C. Frozen tumors were transferred to Covaris tissueTUBEs (a closed vessel for cryopulverization), chilled rapidly in liquid nitrogen and pulverized on a Covaris cryoPREP Dry Pulverizer. Frozen tumor powder was aliquoted and processed separately for molecular and protein assays.

### DNA/RNA Isolation

Frozen tumor powder was weighed and an appropriate volume of RLT Plus Lysis Buffer (Qiagen) containing 2-mercaptoethanol was added at a ratio of 20 μL of lysis buffer/mg of tumor powder. The mixture was added to a reinforced 2 mL tube containing zirconium oxide beads on wet ice and rapidly oscillated at 6500 RPM for 3 cycles of 30 seconds each using a Precellys 24 Tissue Homogenizer (Bertin Technologies). Remaining tumor debris were removed via centrifugation at 21,100×g for 3 minutes at 4° C. DNA/RNA was then isolated from the clarified tumor homogenates using the AllPrep DNA/RNA Mini Kit (Qiagen, cat. #80204) according to the manufacturer's instructions. Isolated DNA/RNA stock concentrations were quantified using a NanoDrop One (ThermoFisher). Working stocks of study DNA and RNA samples were diluted to uniform concentrations in low EDTA TE buffer prior to analysis.

### Vector Genome DNA (@PCR) Copy Quantifications

Vector genome copies in tumor tissue were determined using a qPCR assay to target the F2A link region located between the Teprotumumab heavy and light chain encoding transgenes found within the H-FA2-L (ORF #1) vector. Primers and probe (Table 22) unique to this region were designed and the specificity of the assay was confirmed in qualification. Probe modifications: 5′ 6-FAM (Fluorescein), 3′ Minor Groove Binder Nonfluorescent Quencher (MGBNFQ). Up to 500 ng of total sample DNA were analyzed.

TABLE 22
qPCR Primers and Probe

| | | Assay |
|---|---|---|
| | | Conc. |
| Reagent ID | Sequence | (nM) |
| Forward Primer- | 5′-CCTGTGAAACAGACCCTGAA-3′ (SEQ ID NO: 104) | 600 |
| F2A link | | |
| Probe-F2A link | 5′--TGACCTCTTGAAGCTTGC-3′ (SEQ ID NO: 105) | 250 |
| Reverse Primer- | 5′-TGGACCAGGATTAGACTCCA-3′ (SEQ ID NO: 106) | 900 |
| F2 link | | |

Linearized plasmid containing the F2A link sequence was serially diluted to use as the assay standard for quantification purposes. The appropriate standards, quality controls, and diluted DNA samples were added to a PCR plate containing a reaction mixture of the F2A link primers and probe. Targets were amplified using set qPCR thermocycling parameters (Table 23) in a QuantStudio 5 Real-Time PCR System instrument (Applied Biosystems).

TABLE 23
qPCR Thermocycling Parameters

| Cycle Step | Temperature (° C.) | Time | | |
|---|---|---|---|---|
| 1 | 95 | 1 | minute | |
| 2 | 95 | 1 | second | 40 cycles |
| 3 | 60 | 20 | seconds | |

Vector genome copies were reported as copies/μg host genomic DNA.

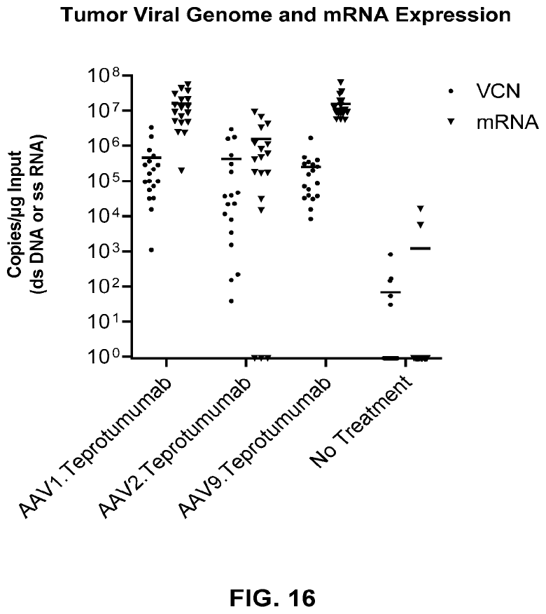

Vector genome copies (VCN) showed a decrease over time in tumors obtained from animals treated with AAV1-delivered antibody expression cassettes encoding Teprotumumab, AAV2-delivered antibody expression cassettes encoding Teprotumumab and AAV9-delivered antibody expression cassettes encoding Teprotumumab, with the lowest levels at day 28 in tumors obtained from animals treated with AAV2-delivered antibody expression cassettes encoding Teprotumumab. In tumors obtained from animals treated with AAV1-delivered antibody expression cassettes encoding Teprotumumab, AAV2-delivered antibody expression cassettes encoding Teprotumumab and AAV9-delivered antibody expression cassettes encoding Teprotumumab the VCN consistently exceeded 10⁴ from day 7 to day 28, with only the VCN being lower than 10⁴ only in tumors obtained from animals treated with AAV2-delivered antibody expression cassettes encoding Teprotumumab at day 28 (FIG. 14 and FIG. 16).

Vector mRNA Expression (RT-qPCR) Copy Quantifications

Vector mRNA expression in tumor tissue was determined using a one-step RT-qPCR assay to target the F2A link region located between the Teprotumumab heavy and light chain encoding transgenes found within the H-F2A-L (ORF #1) vector. Primers and probe (Table 24) unique to this region were designed and the specificity of the assay was confirmed in qualification. Up to 100 ng of total sample RNA was analyzed.

The RT-qPCR assay was performed as a duplex assay, also targeting an endogenous mouse HPRT1 gene using a primers and probe (Table 24) set to monitor mRNA integrity and potential sample matrix inhibition.

Probe modifications: 5′ 6-FAM (Fluorescein), 3′ Minor Groove Binder Nonfluorescent Quencher (MGBNFQ) (Probe-F2A link), and 5′ VIC dye-labeled, 3′ QSY (Probe-mHPRT1).

TABLE 24
RT-qPCR Primers and Probes

| | | Assay |
|---|---|---|
| | | Conc. |
| Reagent ID | Sequence | (nM) |
| Forward Primer- | 5′-CCTGTGAAACAGACCCTGAA-3′ (SEQ ID NO: 107) | 300 |
| F2A link | | |
| Probe-F2A link | 5′-TGACCTCTTGAAGCTTGC-3′ (SEQ ID NO: 108) | 200 |
| Reverse Primer- | 5′-TGGACCAGGATTAGACTCCA-3′ (SEQ ID NO: 109) | 600 |
| F2A link | | |
| Forward Primer- | 5′-GTTAAGCAGTACAGCCCCAAAATG-3′ (SEQ ID | 150 |
| mHPRT1 | NO: 110) | |
| Probe-mHPRT1 | 5′-AGCTTGCTGGTGAAAAGGACCTCTCGAAGT-3′ | 150 |
| | (SEQ ID NO: 111) | |
| Reverse Primer- | 5′-AAATCCAACAAAGTCTGGCCTGTA-3′ (SEQ ID NO: | 150 |
| mHPRT1 | 112) | |

Linearized plasmid containing the F2A link sequence was serially diluted to use as the assay standard for quantification purposes. The appropriate standards, quality controls, and diluted RNA samples were added to a PCR plate containing a reaction mixture of the F2A link primers and probe. Targets were amplified using set RT-qPCR thermocycling parameters (Table 25) in a QuantStudio 5 Real-Time PCR System instrument (Applied Biosystems). Data was captured in the QuantStudio software (Applied Biosystems, v1.5.2), target cycle thresholds were set, and the data was exported for further analysis in Microsoft Excel (v2209).

TABLE 25
RT-qPCR Thermocycling Parameters

| Cycle Step | Temperature (° C.) | Time | | |
|---|---|---|---|---|
| 1 | 50 | 5 | minutes | |
| 2 | 95 | 20 | seconds | |
| 3 | 95 | 3 | seconds | 40 cycles |
| 4 | 60 | 30 | seconds | |

mRNA expression was reported single stranded copies/μg host RNA.

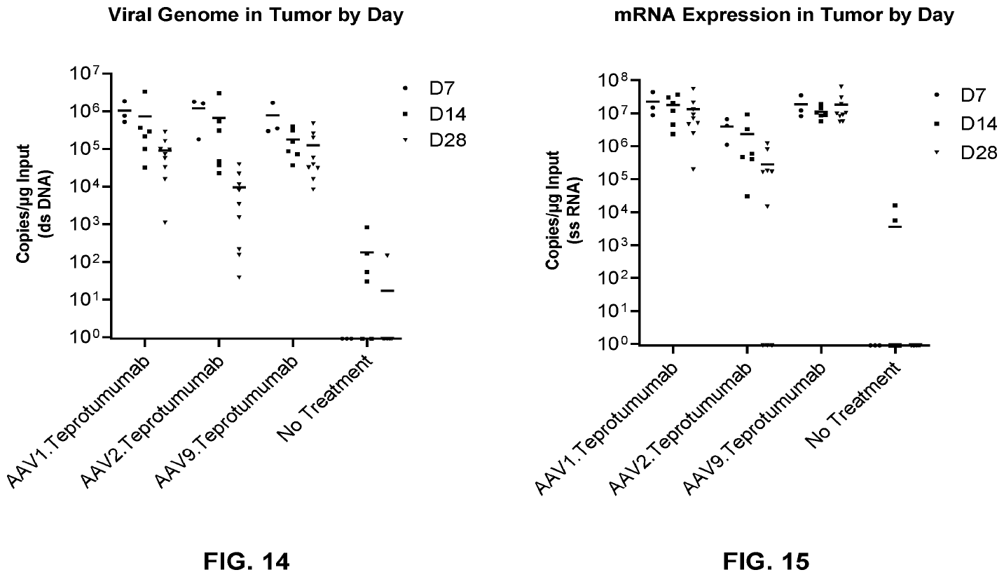

mRNA expression in tumors obtained from animals treated with AAV1-delivered antibody expression cassette encoding Teprotumumab and AAV9-delivered antibody expression cassette encoding Teprotumumab did not show significant variation over time from day 7 to day 28, while mRNA expression in tumors obtained from animals treated with AAV2-delivered antibody expression cassette encoding Teprotumumab showed a progressive decrease over time from day 7 to day 28. mRNA copies were constantly greater that 10⁷ in tumors obtained from animals treated with AAV9 (FIG. 15).

### Protein Assays

Frozen tumor powder was weighed and added to an appropriate volume of Lysis buffer containing Protease and Phosphatase inhibitor cocktail (10 μL of lysis buffer/mg of powder). The mixture was incubated on ice for 30 minutes with intermittent but thorough vortexing. Supernatant was separated by centrifugation at 14,000 RPM for 15 minutes at 4° C. The resulting lysate was assayed for total protein concentration using DC Protein Assay (Bio Rad), aliquoted and stored at −80° C.

### PK Levels of Tepro in the Tumor/Serum

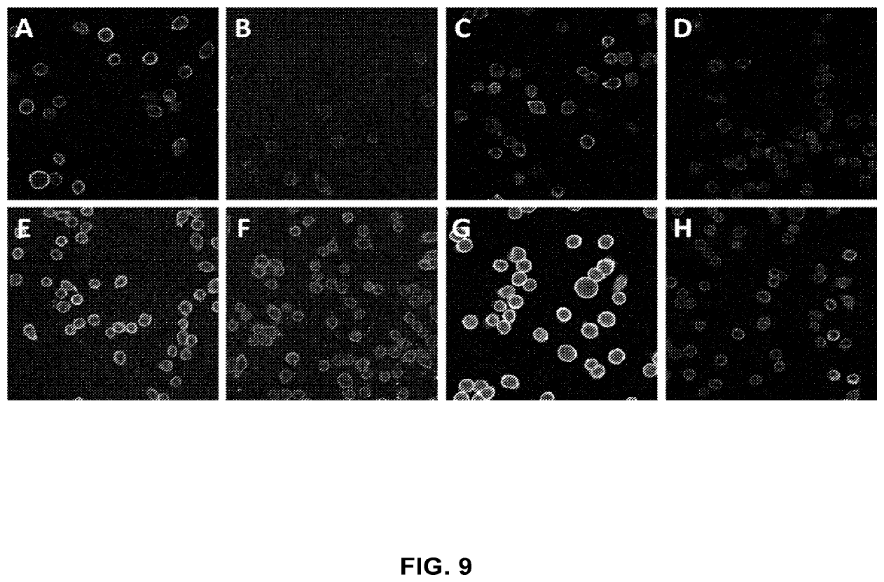

Teprotumumab concentration in tumor and serum samples was measured using MSD's Human/NHP IgG sandwich Immuno assay kit (K150JLD-2) with modifications. Absence of cross reactivity with mouse IgG was confirmed. Briefly, recombinant teprotumumab controls, quality controls, and appropriately diluted serum (MRD 1:100) and tumor lysates (0.125 mg/mL total protein) were applied to a plate precoated with mouse anti-human IgG antibody. The plate was incubated with shaking for approximately 2 hours at room temperature. Following incubation, the plate was washed and incubated with shaking with a sulfo-tag labeled mouse anti-human IgG antibody. Following incubation, the plate was washed and 2× MSD Read Buffer was added to all wells of the plate and immediately read on the MESO QuickPlex SQ 120 MM Reader (Meso Scale Diagnostics, Rockville MD). Data were captured in Methodical Mind software and analyzed in MSD Discovery Workbench software and Excel (v2201). Tumor levels of Teprotumumab in tumors obtained from animals treated with AAV1-delivered antibody expression cassettes encoding Teprotumumab and AAV9-delivered antibody expression cassettes encoding Teprotumumab were consistently higher than tumors levels of Teprotumumab in tumors obtained from animals treated AAV2-delivered antibody expression cassettes encoding Teprotumumab at day 7, 14 and 28. Tumor levels of Teprotumumab in tumors obtained from animals treated with AAV1-delivered antibody expression cassettes encoding Teprotumumab and AAV9-delivered antibody expression cassettes encoding Teprotumumab were also higher than tumors levels of Teprotumumab in tumors obtained from animals treated with recombinant Teprotumumab (6 mg/kg every 7 days) at day 31, with Teprotumumab levels being the highest in tumors obtained from animals treated with AAV9-delivered antibody expression cassettes encoding Teprotumumab (FIG. 11).

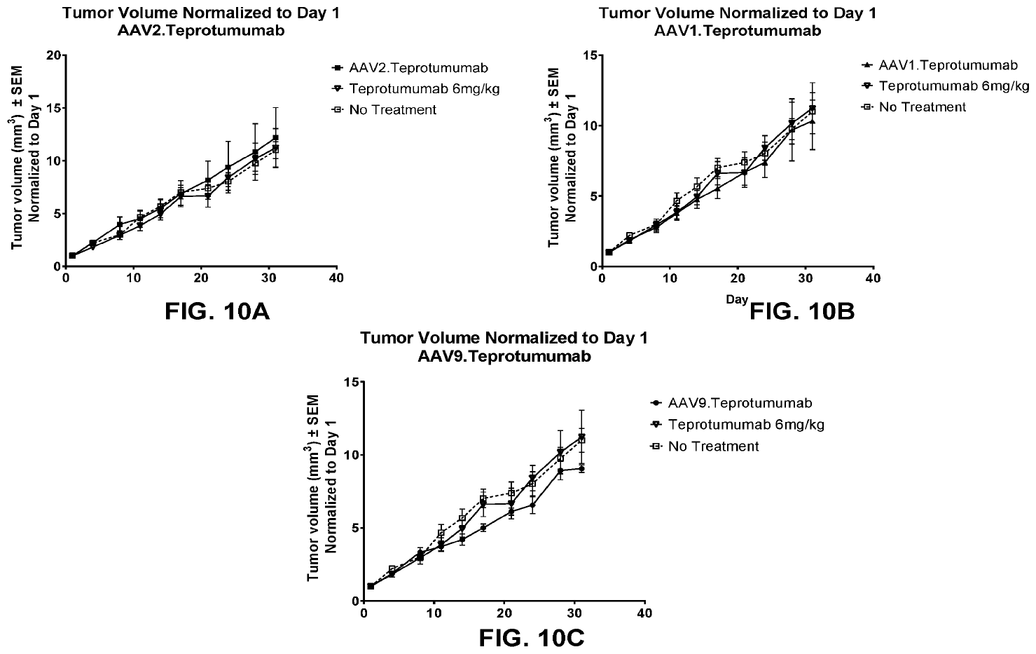

Serum levels of Teprotumumab in animals treated with AAV1-delivered antibody expression cassettes encoding Teprotumumab and AAV9-delivered antibody expression cassettes encoding Teprotumumab were consistently higher than serum levels of Teprotumumab in animals treated AAV2-delivered antibody expression cassettes encoding Teprotumumab at day 7, 14 and 28. Serum levels of Teprotumumab in animals treated with AAV1-delivered antibody expression cassettes encoding Teprotumumab and AAV9-delivered antibody expression cassettes encoding Teprotumumab were also higher than serum levels of Teprotumumab in animals treated with recombinant Teprotumumab (6 mg/kg, every 7 days) at day 31, with Teprotumumab levels being the highest in the serum of animals treated with AAV9-delivered antibody expression cassettes encoding Teprotumumab (FIG. 12 and Tables 26A-B).

TABLE 26A
Serum Levels of Teprotumumab Protein (ng/mL) by day

| | | | | | | | | | | Teprotumumab 6 | | |
|---|---|---|---|---|---|---|---|---|---|---|---|---|
| | AAV1. | | | AAV2. | | | AAV9. | | | mg/kg every 7 days | | |
| | Teprotumumab | | | Teprotumumab | | | Teprotumumab | | | (Day 31 only) | | |
| | Mean | SEM | n | Mean | SEM | n | Mean | SEM | n | Mean | SEM | n |
| Day 7 | 2,266 | 1,572 | 3 | 56.5 | 15.5 | 3 | 173,379 | 28,011 | 3 | — | — | — |
| Day 14 | 23,073 | 12,436 | 3 | 262.3 | 55 | 3 | 364,037 | 34,840 | 3 | — | — | — |
| Day 31 | 84,566 | 29,138 | 8 | 8,921 | 2,700 | 8 | 462,008 | 40,985 | 7 | 60,768 | 6,512 | 8 |

TABLE 26B
Serum Levels of Teprotumumab Protein (ng/mL) at day 31

| Treatment | Mean | SEM | N |
|---|---|---|---|
| AAV1.Teprotumumab | 84,566 | 29,138 | 8 |
| AAV2.Teprotumumab | 8,921 | 2,700 | 8 |
| AAV9.Teprotumumab | 462,008 | 40,985 | 8 |
| Teprotumumab 6 mg/kg | 60,768 | 6512 | 8 |
| every 7 days | | | |
| No Treatment | 0 | 0 | 8 |

### IGF-1R Levels

IGF-1R concentration in tumor was measured using Invitrogen Human IGF-1R ELISA kit (EHIGF1R) with modifications. IGF-1R standards, quality controls, and appropriately diluted tumor lysates (0.5 mg/mL Total Protein) were applied to a plate pre coated with Anti-IGF1R antibody. The plate was incubated overnight (approximately 18 hours) at 4° C. with gentle shaking. Following incubation, the plate was washed and incubated with shaking for approximately 1 Hour at room temperature with a biotinylated Anti IGF-1R antibody. Following a wash, the biotinylated anti-IGF-1R antibody was bound by Streptavidin-HRP conjugate by shaking the plate for approximately 45 minutes at room temperature. Following another wash, IGF-1R was detected by addition of TMB substrate (1-step Ultra TMB, ThermoFisher). The reaction was terminated, and the chromogenic product was measured using SpectraMax 5 (Molecular Devices).

The levels of IGF-1R in tumors obtained from animals treated with AAV1-delivered antibody expression cassettes encoding Teprotumumab, AAV2-delivered antibody expression cassettes encoding Teprotumumab and AAV9-delivered antibody expression cassettes encoding Teprotumumab were consistently lower than levels of IGF-1R in tumors obtained from untreated control animals at day 7, 14 and 28, and this difference was statistically significant at all timepoints. Levels of IGF-1R in tumors obtained from animals treated with AAV1-delivered antibody expression cassettes encoding Teprotumumab, AAV2-delivered antibody expression cassettes encoding Teprotumumab and AAV9-delivered antibody expression cassettes encoding Teprotumumab were slightly higher than in tumors obtained from animals treated with recombinant Teprotumumab (6 mg/kg every 7 days) at day 31, with IGF-1R levels being the lowest in tumors obtained from animals treated with AAV1-delivered antibody expression cassettes encoding Teprotumumab and AAV9-delivered antibody expression cassettes encoding Teprotumumab (see FIGS. 12A-B and Table 27). The difference at day 31 in IGF-1R levels between animals treated with AAV1 and AAV9 delivered Teprotumab and animals treated with recombinant Teprotumumab was not statistically different.

TABLE 27
Tumor Levels of IGF-1R Protein

| | AAV1. | | | AAV2. | | | AAV9. | | | Teprotumumab | | | No | | |
|---|---|---|---|---|---|---|---|---|---|---|---|---|---|---|---|
| | Teprotumumab | | | Teprotumumab | | | Teprotumumab | | | (6 mg/kg) | | | Treatment | | |
| | Mean | | | Mean | | | Mean | | | Mean | | | Mean | | |
| Day | (pg/mg) | SEM | N | (pg/mg) | SEM | N | (pg/mg) | SEM | N | (pg/mg) | SEM | N | (pg/mg) | SEM | N |
| 7 | 591 | 71 | 3 | 693 | 41 | 3 | 461 | 27 | 3 | | | | 1090 | 60 | 3 |
| 14 | 530 | 22 | 6 | 522 | 43 | 6 | 513 | 33 | 6 | | | | 873 | 32 | 6 |
| 28 | 266 | 24 | 9 | 613 | 56 | 9 | 359 | 10 | 9 | | | | 740 | 40 | 9 |
| 31 | 400 | 19 | 16 | 553 | 22 | 16 | 429 | 11 | 16 | 350 | 17 | 16 | 848 | 19 | 14 |

What is claimed is:

1. A recombinant adeno-associated virus (rAAV) particle comprising a capsid and a vector genome, the vector genome comprising an inverted terminal repeat (ITR) and an antibody expression cassette, wherein the antibody expression cassette comprises (a) a promoter, (b) a nucleic acid sequence encoding a heavy chain variable region (VH) of an anti-insulin-like growth factor 1 receptor (anti-IGF-1R) antibody or an antigen-binding fragment thereof, and (c) a nucleic acid sequence encoding a light chain variable region (VL) of an anti-IGF-1R antibody or an antigen-binding fragment thereof.

2. The rAAV particle of claim 1, wherein the nucleic acid sequence encoding the VH of the anti-IGF-1R antibody or an antigen-binding fragment thereof comprises (i) a nucleic acid encoding a VH complementary determining region (CDR) 1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10, (ii) a nucleic acid encoding a VH CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14, and (iii) a nucleic acid encoding a VH CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15; and the nucleic acid sequence encoding the VL of the anti-IGF-1R antibody or an antigen-binding fragment thereof comprises (i) a nucleic acid sequence comprising VL CDR1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16, (ii) a nucleic acid sequence comprising a VL CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23, and (iii) a nucleic acid sequence comprising a VL CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24.

3. A vector comprising an antibody expression cassette comprising:
(a) a promoter;
(b) a nucleic acid sequence encoding a heavy chain variable region (VH) of an anti-insulin-like growth factor 1 receptor (anti-IGF-1R) antibody or an antigen-binding fragment thereof comprising (i) a nucleic acid encoding a VH complementary determining region (CDR) 1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 7 or 10, (ii) a nucleic acid encoding a VH CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 8, 11 or 14, and (iii) a nucleic acid encoding a VH CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 9, 12 or 15; (c) a nucleic acid sequence encoding a light chain variable region (VL) of an anti-IGF-1R antibody or an antigen-binding fragment thereof comprising (i) a nucleic acid sequence comprising VL CDR1 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 16, (ii) a nucleic acid sequence comprising a VL CDR2 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 17, 20 or 23, and (iii) a nucleic acid sequence comprising a VL CDR3 comprising a nucleotide sequence with at least 85%, 86%, 87%, 88%, 89%, 90%, 91%, 92%, 93%, 94%, 95%, 96%, 97%, 98%, 99%, or 100% identity to SEQ ID NO: 18, 21 or 24.

4. The rAAV particle or the vector of any one of the previous claims, wherein the antibody expression cassette comprises a signal peptide.

5. The rAAV particle or the vector of any one of the previous claims, wherein the antibody expression cassette comprises a linker sequence selected from an internal ribosome entry site (IRES) sequence, a proteolytic cleavage site, or a combination thereof.

6. The rAAV particle or the vector of claim 5, wherein the proteolytic cleavage site comprises a furin cleavage site, a 2A cleavage site, or a combination thereof.

7. The rAAV particle or the vector of claim 5, wherein the antibody expression cassette comprises the promoter, the nucleic acid sequence encoding the VH, the IRES, and the nucleic acid sequence encoding the VL in 5′-3′ orientation.

8. The rAAV particle or the vector of claim 5, wherein the antibody expression cassette comprises the promoter, the nucleic acid sequence encoding the first signal peptide, the nucleic acid sequence encoding the VH, the IRES, the nucleic acid sequence encoding the second signal peptide, and the nucleic acid sequence encoding the VL in 5′-3′ orientation.

9. The rAAV particle or the vector of claim 5, wherein the antibody expression cassette comprises the promoter, the nucleic acid sequence encoding the VL, the IRES, and the nucleic acid sequence encoding the VH in 5′-3′ orientation.

10. The rAAV particle or the vector of claim 5 or 6, wherein the antibody expression cassette comprises the promoter, the nucleic acid sequence encoding the VH, the proteolytic cleavage site, and the nucleic acid sequence encoding the VL in 5′-3′ orientation.

11. The rAAV particle or the vector of claim 5 or 6, wherein the antibody expression cassette comprises the promoter, the nucleic acid sequence encoding the first signal peptide, the nucleic acid sequence encoding the VH, the proteolytic cleavage site, the nucleic acid sequence encoding the second signal peptide, and the nucleic acid sequence encoding the VL in 5′-3′ orientation.

12. The rAAV particle or the vector of claim 5 or 6, wherein the antibody expression cassette comprises the promoter, the nucleic acid sequence encoding the VL, the proteolytic cleavage site, and the nucleic acid sequence encoding the VH in 5′-3′ orientation.

13. The rAAV particle or the vector of claim 5 or 6, wherein the antibody expression cassette comprises the promoter, the nucleic acid sequence encoding the first signal peptide, the nucleic acid sequence encoding the VL, the proteolytic cleavage site, the nucleic acid sequence encoding the second signal peptide, and the nucleic acid sequence encoding the VH in 5′-3′ orientation.

14. The rAAV particle or the vector of any one of claim 4 or 6-10, wherein the IRES comprises a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 43.

15. The rAAV particle or the vector of any one of claims 4-5 and 11-14, wherein the proteolytic cleavage comprises a furin cleavage site comprising a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 44.

16. The rAAV particle or the vector of any one of claims 4-5 and 11-149, wherein the proteolytic cleavage comprises a 2A cleavage site comprising a nucleic acid having a sequence at least 85%, 86%, 87%, 88%, 89%, 90%, 91%, 92%, 93%, 94%, 95%, 96%, 97%, 98%, 99%, or 100% identical to SEQ ID NO: 45.

17. The rAAV particle or the vector of any one of the previous claims, wherein the signal peptide is an IL-2 or an IL-10 signal peptide.

18. The rAAV particle or the vector of claim 17, wherein the signal peptide comprises an amino acid sequence having at least 85%, 86%, 87%, 88%, 89%, 90%, 91%, 92%, 93%, 94%, 95%, 96%, 97%, 98%, 99%, or 100% identical to SEQ ID NO: 119 or 120.

19. The rAAV particle or the vector of claim 17 or 18, wherein the signal peptide is encoded by a nucleotide sequences having at least 85%, 86%, 87%, 88%, 89%, 90%, 91%, 92%, 93%, 94%, 95%, 96%, 97%, 98%, 99%, or 100% identical to SEQ ID NO: 121 or 122.

20. The rAAV particle or the vector of any one of the previous claims, wherein the antibody expression cassette comprises a second promoter.

21. The rAAV particle or the vector of claim 20, wherein the antibody expression cassette comprises the promoter (a first promoter), the nucleic acid sequence encoding the VL, the second promoter, and the nucleic acid sequence encoding the VH in 5′-3′ orientation.

22. The rAAV particle or the vector of claim 20, wherein the antibody expression cassette comprises the promoter (a first promoter), the nucleic acid sequence encoding the first signal peptide, the nucleic acid sequence encoding the VL, the second promoter, the nucleic acid sequence encoding the second signal peptide, and the nucleic acid sequence encoding the VH in 5′-3′ orientation.

23. The rAAV particle or the vector of claim 20, wherein the antibody expression cassette comprises the promoter (a first promoter), the nucleic acid sequence encoding the VH, the second promoter, and the nucleic acid sequence encoding the VL in 5′-3′ orientation.

24. The rAAV particle or the vector of claim 20, wherein the antibody expression cassette comprises the promoter (a first promoter), the nucleic acid sequence encoding the first signal peptide, the nucleic acid sequence encoding the VH, the second promoter, the nucleic acid sequence encoding the second signal peptide, and the nucleic acid sequence encoding the VL in 5′-3′ orientation.

25. The rAAV particle or the vector of claim 20, wherein the antibody expression cassette comprises the nucleic acid sequence encoding a VH, the promoter (a first promoter), the second promoter, and the nucleic acid sequence encoding the VL in 5′-3′ orientation.

26. The rAAV particle or the vector of claim 20, wherein the antibody expression cassette comprises the nucleic acid sequence encoding the first signal peptide, the nucleic acid sequence encoding a VH, the promoter (a first promoter), the second promoter, the nucleic acid sequence encoding the second signal peptide, and the nucleic acid sequence encoding the VL in 5′-3′ orientation.

27. The rAAV particle or the vector of claim 20, wherein the antibody expression cassette comprises the nucleic acid sequence encoding a VL, the promoter (a first promoter), the second promoter, and the nucleic acid sequence encoding the VH in 5′-3′ orientation.

28. The rAAV particle or the vector of claim 20, wherein the antibody expression cassette comprises the nucleic acid sequence encoding the first signal peptide, the nucleic acid sequence encoding a VL, the promoter (a first promoter), the second promoter, the nucleic acid sequence encoding the second signal peptide, and the nucleic acid sequence encoding the VH in 5′-3′ orientation.

29. The rAAV particle or the vector of any one of the previous claims, wherein the promoter and/or the second promoter is a constitutively active promoter, a cell-type specific promoter, a synthetic promoter, or an inducible promoter.

30. The rAAV particle or the vector of any one of the previous claims, wherein the promoter and/or the second promoter is a CBA promoter, a CMV promoter (optionally, a human CMV promoter or a mouse CMV promoter), an EF1α promoter, a CAG promoter, or a tissue specific promoter.

31. The rAAV particle or the vector of any one of the previous claims, wherein the promoter comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 47-51, 83, or 93.

32. The rAAV particle or the vector of any one of claims 1-30, wherein the promoter is a muscle specific promoter.

33. The rAAV particle or the vector of claim 32, wherein the muscle specific promoter is selected from a desmin (DES) promoter, a human skeletal muscle α-actin (HSA) promoter, α myosin creatine kinase (MCK) promoter, a α myosin heavy chain myosine creatine kinase 7 (HMCK7) promoter, a dual MCK enhancer MCK (dMCK) promoter, a triple MCK enhancer MCK (tMCK) promoter, a dual MCK enhancer muscle-type creatine kinase 8 e (CK8e) promoter, a SPc5-12 promoter, a SP-301 promoter, a α myosin heavy chain (MHC) promoter, a Sk-CRM promoter, or a Sk-CRM4 promoter.

34. The rAAV particle or the vector of any one of the previous claims, wherein the antibody expression cassette comprises an intron.

35. The rAAV particle or the vector of claim 34, wherein the intron is a CAG intron, an SV40 intron, MVM intron, or a human beta-globin intron, or any combination thereof.

36. The rAAV particle or the vector of claim 34 or 35, wherein the intron comprises a nucleotide sequence at least 85%, 86%, 87%, 88%, 89%, 90%, 91%, 92%, 93%, 94%, 95%, 96%, 97%, 98%, 99%, or 100% identical to SEQ ID NOs: 46 or 82.

37. The rAAV particle or the vector of any one of the previous claims, wherein the promoter comprises a first and a promoter which are different.

38. The rAAV particle or the vector of claim 37, wherein first and second promoter initiate transcription in the same direction.

39. The rAAV particle or the vector of claim 37, wherein first and second promoter initiate transcription in different directions.

40. The rAAV particle or the vector of claim 37, wherein the antibody expression cassette comprises a pause element between the first and second promoter.

41. The rAAV particle or the vector of claim 40, wherein the pause element comprises a nucleotide sequence at least 85%, 86%, 87%, 88%, 89%, 90%, 91%, 92%, 93%, 94%, 95%, 96%, 97%, 98%, 99%, or 100% identical to SEQ ID NO: 54.

42. The rAAV particle or the vector of any one of the previous claims, wherein the anti-IGF-1R antibody is a monoclonal antibody.

43. The rAAV particle or the vector of any one of the previous claims, wherein the VH CDRs 1-3 correspond to the CDRs of teprotumumab.

44. The rAAV particle or the vector of any one of the previous claims, wherein the VL CDRs 1-3 correspond to the CDRs of teprotumumab.

45. The rAAV particle or the vector of any one of the previous claims, wherein the anti-IGF-1R antibody is teprotumumab.

46. The rAAV particle or the vector of any one of the previous claims, wherein the nucleic acid sequence encoding the VH comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 25-27.

47. The rAAV particle or the vector of any one of the previous claims, wherein the nucleic acid sequence encoding the VL comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 29-31.

48. The rAAV particle or the vector of any one of the previous claims, wherein the nucleic acid sequence encoding the having chain (HC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 35-37.

49. The rAAV particle or the vector of any one of the previous claims, wherein the nucleic acid sequence encoding the light chain (LC) comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 39-41.

50. The rAAV particle or the vector of any one of the previous claims, wherein the anti-IGF-1R antibody is teprotumumab.

51. The rAAV particle or the vector of any one of the previous claims, wherein the antibody expression cassette comprises a poly (A) sequence.

52. The rAAV particle or the vector of claim 51, wherein the poly(A) sequence is selected from a bGHpA, a hGHpA, a SV40 pA, or a synthetic pA.

53. The rAAV particle or the vector of claim 51 or 52, wherein the poly (A) comprises a nucleotide sequence at least 85%, 86%, 87%, 88%, 89%, 90%, 91%, 92%, 93%, 94%, 95%, 96%, 97%, 98%, 99%, or 100% identical to SEQ ID NOs: 52 or 53.

54. The rAAV particle or the vector of any one of the previous claims, wherein antibody expression cassette comprises an open reading frame (ORF) comprising a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 57-67 and 94-97.

55. The AAV particle or the vector of any one of the previous claims, wherein the antibody expression cassette comprises a nucleotide sequence at least 85%, 86%, 87%, 88%, 89%, 90%, 91%, 92%, 93%, 94%, 95%, 96%, 97%, 98%, 99%, or 100% identical to any of SEQ ID NOs: 68-76.

56. The vector of any one of claims 3-46 which comprises an inverted terminal repeats (ITR).

57. The rAAV or the vector of any one of the previous claims, wherein the AAV ITR comprises a pair of ITRs flanking the antibody expression cassette.

58. The rAAV particle or the vector of claim 48, wherein the ITRs are of the same serotype as one another.

59. The rAAV particle or the vector of claim 49, wherein the ITRs are of the AAV2 serotype.

60. The vector of any one of claims 3-59, wherein the vector is packaged in an AAV capsid.

61. The rAAV particle or the vector of any one of the previous claims, wherein AAV capsid serotype is selected from the group consisting of AAV1, AAV2, AAV3, AAV4, AAV5, AAV6, AAV7, AAV8, AAVRh8, AAVrh9, AAV9, AAVrh10, AAV10, AAV11, AAV12, and a modified version thereof.

62. The rAAV particle or the vector of any one of the previous claims, wherein the AAV capsid serotype is selected from the group consisting of AAV1, AAV2, AAV6, AAV8, AAV9, and a modified version thereof.

63. A host cell comprising the rAAV particle or the vector of any one of the previous claims.

64. A composition comprising the rAAV particle or the vector of any one of the previous claims and a carrier.

65. The composition of claim 64, wherein the carrier is water or saline.

66. A method of expressing an anti-IGF-1R antibody or antigen-binding fragment thereof in a cell, comprising administering to the cell the rAAV particle, the vector, or the composition of any one of the previous claims, thereby expressing the anti-IGF-1R antibody or antigen-binding fragment thereof in the cell.

67. The method of claim 66, wherein the cell is a fibroblast cell, an adipocyte cell, a myofibroblast cell, a myocyte cell, a muscle cell, or any combination thereof.

68. The method of claim 66 or 67, wherein the administration is in vitro.

69. The method of claim 66 or 67, wherein the administration is in vivo.

70. A method of expressing an anti-IGF-1R antibody or antigen-binding fragment thereof in a subject in need thereof, comprising administering to the subject the rAAV particle, the vector, or the composition of any one of the previous claims, thereby expressing the anti-IGF-1R antibody or antigen-binding fragment thereof in the subject.

71. The method of claim 70, wherein the subject suffers from a thyroid eye disease selected from active Graves' Orbitopathy and chronic Graves' Orbitopathy.

72. A method of treating a thyroid eye disease in a subject in need thereof comprising administering to the subject the rAAV particle, the vector, or the composition of any one of the previous claims, thereby expressing the anti-IGF-1R antibody or antigen-binding fragment thereof in the subject and treating the thyroid eye disease.

73. The method of claim 72, wherein the thyroid eye disease selected from active Graves' Orbitopathy and chronic Graves' Orbitopathy.

74. The method of any one of claims 72-73, wherein the administration is suitable for delivery of the rAAV particle or the vector to an ocular delivery site, a retro or a peri-orbital delivery site, a retrobulbar delivery site, an extra-ocular muscle delivery site, a connective tissue delivery site, or any combination of delivery sites thereof.

75. The method of any one of claims 72-74, wherein the administration is by injection or infusion.

76. The method of any one of claims 72-75, wherein the administration is intramuscular (IM), intravenous (IV), intralymphatic, intraocular, retroorbital, periorbital, retrobulbar, or any combination thereof.

77. The method of any one of claims 72-76, wherein the administration is suitable for delivery to retro or a peri-orbital fibroblast cells, adipocytes cells, myofibroblast cells, myocyte cells, or any combination thereof.

78. The method of any one of claims 72-77, wherein the administration is to an extra-ocular muscle.

79. The method of claim 78, wherein the extra-ocular muscle is a levantor muscle or a glabellar muscle.

80. The method of any one of claims 72-77, wherein the administration is to a connective tissue.

81. The method of any one of claims 72-77, wherein administration is transconjunctival into the periorbital space.

82. The method of any one of claims 72-77, wherein the administration is intralymphatic comprising injection or infusion to the pre-auricular or submandibular node.

83. The method of any one of claims 72-82, wherein the administration is a single dose.

84. The method of claim 83, wherein the single dose multiple injections and/or infusions.
